SEQUENCE LISTING (303 sequences)

SEQ ID NO: 1 - protein, 1344 aa, Streptococcus mutans
KKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1367 aa, Streptococcus pyogenes
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1387 aa, Streptococcus thermophilus
TKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 4 - protein, 1333 aa, Listeria innocua
KKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 5 - RNA, 96 nt, synthetic construct
  variation 1..20: note = a, c, u, g, unknown or other
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 6 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 7 - protein, 1368 aa, Streptococcus pyogenes [sequence omitted]

SEQ ID NO: 8 - skipped

SEQ ID NO: 9 - protein, 12 aa, synthetic construct
  REGION 1..12: note = Description of Artificial Sequence: Synthetic peptide
  MOD_RES 2: note = Ile, Val, Met, Leu or Thr
  MOD_RES 4: note = Thr, Ile, Val, Ser, Asn, Tyr, Glu or Leu
  MOD_RES 5: note = Asn, Ser, Gly, Ala, Asp, Thr, Arg, Met or Phe
  MOD_RES 7: note = Val, Ile, Leu, Cys, Thr or Phe
  MOD_RES 9: note = Trp, Phe, Val, Tyr, Ser or Leu
  MOD_RES 10: note = Ala, Ser, Cys, Val or Gly
  MOD_RES 11: note = Val, Ile, Leu, Ala, Met or His
  MOD_RES 12: note = Any amino acid or is absent
DXGXXSXGXXXX

SEQ ID NO: 10 - protein, 12 aa, synthetic construct
  REGION 1..12: note = Description of Artificial Sequence: Synthetic peptide
  MOD_RES 4: note = Thr, Ile, Val, Ser, Asn, Tyr, Glu or Leu
  MOD_RES 5: note = Asn, Ser, Gly, Ala, Asp, Thr, Arg, Met or Phe
  MOD_RES 11: note = Val, Ile, Leu, Ala, Met or His
  MOD_RES 12: note = Any amino acid or is absent
DIGXXSVGWAXX

SEQ ID NO: 11 - protein, 12 aa, synthetic construct
  REGION 1..12: note = Description of Artificial Sequence: Synthetic peptide
  MOD_RES 12: note = Any non-polar alkyl amino acid or a hydroxyl amino acid
DIGTNSVGWAVX

SEQ ID NO: 12 - protein, 8 aa, synthetic construct
  REGION 1..8: note = Description of Artificial Sequence: Synthetic peptide
  MOD_RES 2: note = Val or His
  MOD_RES 3: note = Ile, Leu or Val
  MOD_RES 5: note = Met or Thr
IXXEXARE

SEQ ID NO: 13 - protein, 8 aa, synthetic construct
  REGION 1..8: note = Description of Artificial Sequence: Synthetic peptide
  MOD_RES 3: note = Ile, Leu or Val
IVXEMARE

SEQ ID NO: 14 - protein, 8 aa, synthetic construct
  REGION 1..8: note = Description of Artificial Sequence: Synthetic peptide
  MOD_RES 4: note = His or Leu
  MOD_RES 7: note = Arg or Val
  MOD_RES 8: note = Glu or Val
HHAXDAXX

SEQ ID NO: 15 - protein, 8 aa, synthetic construct
  REGION 1..8: note = Description of Artificial Sequence: Synthetic peptide
HHAHDAYL

SEQ ID NO: 16 - protein, 30 aa, synthetic construct
  REGION 1..30: note = Description of Artificial Sequence: Synthetic polypeptide
  MOD_RES 2: note = Lys or Pro
  MOD_RES 4: note = Val, Leu, Ile or Phe
  MOD_RES 5: note = Gly, Ala or Ser
  MOD_RES 6: note = Leu, Ile, Val or Phe
  MOD_RES 7..26: note = Any amino acid and this region may encompass 5-20 residues wherein some positions may be absent,
  MOD_RES 29: note = Asp, Glu, Asn or Gln
  REGION 1..30: note = See specification as filed for detailed description of substitutions and preferred embodiments
KXYXXXXXXXXXXXXXXXXXXXXXXXTDXY

SEQ ID NO: 17 - protein, 27 aa, synthetic construct
  REGION 1..27: note = Description of Artificial Sequence: Synthetic peptide
  MOD_RES 1: note = Asp, Glu, Gln or Asn
  MOD_RES 2: note = Leu, Ile, Arg, Gln, Val, Met or Lys
  MOD_RES 3: note = Asp or Glu
  MOD_RES 5: note = Ile, Val, Thr, Ala or Leu
  MOD_RES 6: note = Val, Tyr, Ile, Leu, Phe or Trp
  MOD_RES 8: note = Gln, His, Arg, Lys, Tyr, Ile, Leu, Phe or Trp
  MOD_RES 9: note = Ser, Ala, Asp, Thr or Lys
  MOD_RES 10: note = Phe, Leu, Val, Lys, Tyr, Met, Ile, Arg, Ala, Glu, Asp or Gln
  MOD_RES 11: note = Leu, Arg, Thr, Ile, Val, Ser, Cys, Tyr, Lys, Phe or Gly
  MOD_RES 12: note = Lys, Gln, Tyr, Thr, Phe, Leu, Trp, Met, Ala, Glu, Gly or Ser
  MOD_RES 13: note = Asp, Ser, Asn, Arg, Leu or Thr
  MOD_RES 14: note = Asp, Asn or Ser
  MOD_RES 15: note = Ser, Ala, Thr, Gly or Arg
  MOD_RES 16: note = Ile, Leu, Phe, Ser, Arg, Tyr, Gln, Trp, Asp, Lys or His
  MOD_RES 17: note = Asp, Ser, Ile, Asn, Glu, Ala, His, Phe, Leu, Gln, Met, Gly, Tyr or Val
  MOD_RES 19: note = Lys, Leu, Arg, Met, Thr or Phe
  MOD_RES 20: note = Val, Leu, Ile, Ala or Thr
  MOD_RES 21: note = Leu, Ile, Val or Ala
  MOD_RES 22: note = Thr, Val, Cys, Glu, Ser or Ala
  MOD_RES 23: note = Arg, Phe, Thr, Trp, Glu, Leu, Asn, Cys, Lys, Val, Ser, Gln, Ile, Tyr, His or Ala
  MOD_RES 24: note = Ser, Pro, Arg, Lys, Asn, Ala, His, Gln, Gly or Leu
  MOD_RES 25: note = Asp, Gly, Thr, Asn, Ser, Lys, Ala, Ile, Glu, Leu, Gln, Arg or Tyr
  MOD_RES 26: note = Lys, Val, Ala, Glu, Tyr, Ile, Cys, Leu, Ser, Thr, Gly, Lys, Met, Asp or Phe
XXXHXXPXXXXXXXXXXNXXXXXXXXN

SEQ ID NO: 18 - protein, 27 aa, synthetic construct
  REGION 1..27: note = Description of Artificial Sequence: Synthetic peptide
  MOD_RES 1: note = Asp or Glu
  MOD_RES 2: note = Leu, Ile, Arg, Gln, Val, Met or Lys
  MOD_RES 3: note = Asp or Glu
  MOD_RES 5: note = Ile, Val, Thr, Ala or Leu
  MOD_RES 6: note = Val, Tyr, Ile, Leu, Phe or Trp
  MOD_RES 8: note = Gln, His, Arg, Lys, Tyr, Ile, Leu, Phe or Trp
  MOD_RES 10: note = Phe, Leu, Val, Lys, Tyr, Met, Ile, Arg, Ala, Glu, Asp or Gln
  MOD_RES 11: note = Leu, Arg, Thr, Ile, Val, Ser, Cys, Tyr, Lys, Phe or Gly
  MOD_RES 12: note = Lys, Gln, Tyr, Thr, Phe, Leu, Trp, Met, Ala, Glu, Gly or Ser
  MOD_RES 16: note = Ile, Leu, Phe, Ser, Arg, Tyr, Gln, Trp, Asp, Lys or His
  MOD_RES 17: note = Asp, Ser, Ile, Asn, Glu, Ala, His, Phe, Leu, Gln, Met, Gly, Tyr or Val
  MOD_RES 22: note = Thr, Val, Cys, Glu, Ser or Ala
  MOD_RES 23: note = Arg, Phe, Thr, Trp, Glu, Leu, Asn, Cys, Lys, Val, Ser, Gln, Ile, Tyr, His or Ala
  MOD_RES 24: note = Ser, Pro, Arg, Lys, Asn, Ala, His, Gln, Gly or Leu
  MOD_RES 25: note = Asp, Gly, Thr, Asn, Ser, Lys, Ala, Ile, Glu, Leu, Gln, Arg or Tyr
  MOD_RES 26: note = Lys, Val, Ala, Glu, Tyr, Ile, Cys, Leu, Ser, Thr, Gly, Lys, Met, Asp or Phe
XXXHXXPXSXXXDDSXXNKVLXXXXXN

SEQ ID NO: 19 - protein, 27 aa, synthetic construct
  REGION 1..27: note = Description of Artificial Sequence: Synthetic peptide
  MOD_RES 1: note = Asp or Glu
  MOD_RES 3: note = Asp or Glu
  MOD_RES 8: note = Gln, His, Arg, Lys, Tyr, Ile, Leu or Trp
  MOD_RES 10: note = Phe, Leu, Val, Lys, Tyr, Met, Ile, Arg, Ala, Glu, Asp or Gln
  MOD_RES 11: note = Leu, Arg, Thr, Ile, Val, Ser, Cys, Tyr, Lys, Phe or Gly
  MOD_RES 12: note = Lys, Gln, Tyr, Thr, Phe, Leu, Trp, Met, Ala, Glu, Gly or Ser
  MOD_RES 16: note = Ile, Leu, Phe, Ser, Arg, Tyr, Gln, Trp, Asp, Lys or His
  MOD_RES 17: note = Asp, Ser, Ile, Asn, Glu, Ala, His, Phe, Leu, Gln, Met, Gly, Tyr or Val
  MOD_RES 23: note = Arg, Phe, Thr, Trp, Glu, Leu, Asn, Cys, Lys, Val, Ser, Gln, Ile, Tyr, His or Ala
  MOD_RES 24: note = Ser, Pro, Arg, Lys, Asn, Ala, His, Gln, Gly or Leu
  MOD_RES 25: note = Asp, Gly, Thr, Asn, Ser, Lys, Ala, Ile, Glu, Leu, Gln, Arg or Tyr
  MOD_RES 26: note = Lys, Val, Ala, Glu, Tyr, Ile, Cys, Leu, Ser, Thr, Gly, Lys, Met, Asp or Phe
XVXHIVPXSXXXDDSXXNKVLTXXXXN

SEQ ID NO: 20 - protein, 27 aa, synthetic construct
  REGION 1..27: note = Description of Artificial Sequence: Synthetic peptide
  MOD_RES 2: note = Ile or Val
  MOD_RES 6: note = Ile or Val
  MOD_RES 9: note = Ala or Ser
  MOD_RES 11: note = Ile or Leu
  MOD_RES 12: note = Lys or Thr
  MOD_RES 14: note = Asp or Asn
  MOD_RES 19: note = Arg, Lys or Leu
  MOD_RES 22: note = Thr or Val
  MOD_RES 23: note = Ser or Arg
  MOD_RES 25: note = Lys, Asp or Ala
  MOD_RES 26: note = Glu, Lys, Gly or Asn
DXDHIXPQXFXXDXSIDNXVLXXSXXN

SEQ ID NO: 21 - protein, 73 aa, synthetic construct
  REGION 1..73: note = Description of Artificial Sequence: Synthetic polypeptide
  MOD_RES 8: note = Lys or Arg
  MOD_RES 12: note = Val or Thr
  MOD_RES 13: note = Gly or Asp
  MOD_RES 14: note = Glu, Gln or Asp
  MOD_RES 15: note = Glu or Asp
  MOD_RES 19: note = Asp, Asn or His
  MOD_RES 20: note = Tyr, Arg or Asn
  MOD_RES 23: note = Gln, Asp or Asn
  MOD_RES 25..64: note = Any amino acid and this region may encompass 15-40 residues wherein some positions may be absent
  MOD_RES 67: note = Gly or Glu
  MOD_RES 69: note = Ser or Gly
  MOD_RES 71: note = Asp or Asn
  REGION 1..73: note = See specification as filed for detailed description of substitutions and preferred embodiments
LYYLQNGXDMYXXXXLDIXXLSXYXXXXXXXXXXXXXXXXXXXXXXXXXXXXXXXXXXXXXXXXNRXKXDXVP

SEQ ID NO: 22 - DNA, 4107 bp, synthetic construct
  misc_feature 1..4107: note = Description of Artificial Sequence: Synthetic polynucleotide
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 23 - protein, 1368 aa, Streptococcus pyogenes [sequence omitted]

SEQ ID NO: 24 - DNA, 3249 bp, synthetic construct
  misc_feature 1..3249: note = Description of Artificial Sequence: Synthetic polynucleotide
  source 1..3249: mol_type = other DNA
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 25 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 26 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 27 - RNA, 104 nt, synthetic construct
  variation 1..20: note = a, c, u, g, unknown or other
  source 1..104: mol_type = other RNA
nnnnnnnnnnnnnnnnnnnngttttagagctatgctgaaaagcatagcaagttaaaataaggctagtccgttatcaacttgaaaaagtggcaccgagtcggtgc

SEQ ID NO: 28 - RNA, 106 nt, synthetic construct
  variation 1..20: note = a, c, u, g, unknown or other
  source 1..106: mol_type = other RNA
nnnnnnnnnnnnnnnnnnnngttttagagctatgctggaaacagcatagcaagttaaaataaggctagtccgttatcaacttgaaaaagtggcaccgagtcggtgc

SEQ ID NO: 29 - RNA, 116 nt, synthetic construct
  variation 1..20: note = a, c, u, g, unknown or other
  source 1..116: mol_type = other RNA
nnnnnnnnnnnnnnnnnnnngttttagagctatgctgttttggaaacaaaacagcatagcaagttaaaataaggctagtccgttatcaacttgaaaaagtggcaccgagtcggtgc

SEQ ID NO: 30 - RNA, 96 nt, synthetic construct
  variation 1..20: note = a, c, u, g, unknown or other
  source 1..96: mol_type = other RNA
nnnnnnnnnnnnnnnnnnnngtattagagctagaaatagcaagttaatataaggctagtccgttatcaacttgaaaaagtggcaccgagtcggtgc

SEQ ID NO: 31 - RNA, 96 nt, synthetic construct
  variation 1..20: note = a, c, u, g, unknown or other
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 32 - RNA, 116 nt, synthetic construct
  variation 1..20: note = a, c, u, g, unknown or other
  source 1..116: mol_type = other RNA
nnnnnnnnnnnnnnnnnnnngtattagagctatgctgtattggaaacaatacagcatagcaagttaatataaggctagtccgttatcaacttgaaaaagtggcaccgagtcggtgc

SEQ ID NO: 33 - RNA, 47 nt, synthetic construct
  misc_feature 1..47: note = Description of Artificial Sequence: Synthetic oligonucleotide
  source 1..47: mol_type = other RNA
aaggctagtccgttatcaacttgaaaaagtggcaccgagtcggtgct

SEQ ID NO: 34 - RNA, 49 nt, synthetic construct
  misc_feature 1..49: note = Description of Artificial Sequence: Synthetic oligonucleotide
  source 1..49: mol_type = other RNA
aaggctagtccgttatcaacttgaaaaagtggcaccgagtcggtggtgc

SEQ ID NO: 35 - RNA, 51 nt, synthetic construct
  misc_feature 1..51: note = Description of Artificial Sequence: Synthetic oligonucleotide
  source 1..51: mol_type = other RNA
aaggctagtccgttatcaacttgaaaaagtggcaccgagtcggtgcggatc

SEQ ID NO: 36 - RNA, 31 nt, synthetic construct
  misc_feature 1..31: note = Description of Artificial Sequence: Synthetic oligonucleotide
  source 1..31: mol_type = other RNA
aaggctagtccgttatcaacttgaaaaagtg

SEQ ID NO: 37 - RNA, 18 nt, synthetic construct
  misc_feature 1..18: note = Description of Artificial Sequence: Synthetic oligonucleotide
  source 1..18: mol_type = other RNA
aaggctagtccgttatca

SEQ ID NO: 38 - RNA, 12 nt, synthetic construct
  misc_feature 1..12: note = Description of Artificial Sequence: Synthetic oligonucleotide
  source 1..12: mol_type = other RNA
aaggctagtccg

SEQ ID NO: 39 - protein, 4 aa, unidentified
  REGION 1..4: note = Description of Unknown: "DEAH" motif peptide
DEAH

SEQ ID NO: 40 - protein, 4 aa, unidentified
  REGION 1..4: note = Description of Unknown: "DEAD" motif peptide
DEAD

SEQ ID NO: 41 - protein, 14 aa, synthetic construct
  REGION 1..14: note = Description of Artificial Sequence: Synthetic peptide
  MOD_RES 2..3: note = Any amino acid
  MOD_RES 5..8: note = Any amino acid
  MOD_RES 10..13: note = Any amino acid
CXXCXXXXHXXXXC

SEQ ID NO: 42 - RNA, 42 nt, synthetic construct
  variation 1..20: note = a, c, u, g, unknown or other
  source 1..42: mol_type = other RNA
nnnnnnnnnnnnnnnnnnnngttttagagctatgctgttttg

SEQ ID NO: 43 - RNA, 85 nt, Streptococcus pyogenes
  source 1..85: mol_type = other RNA
ggaaccattcaaaacagcatagcaagttaaaataaggctagtccgttatcaacttgaaaaagtggcaccgagtcggtgctttttt

SEQ ID NO: 44 - RNA, 62 nt, synthetic construct
  variation 1..20: note = a, c, u, g, unknown or other
  source 1..62: mol_type = other RNA
nnnnnnnnnnnnnnnnnnnngttttagagctagaaatagcaagttaaaataaggctagtccg

SEQ ID NO: 45 - RNA, 102 nt, synthetic construct
  variation 1..20: note = a, c, u, g, unknown or other
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 46 - RNA, 75 nt, synthetic construct
  variation 1..20: note = a, c, u, g, unknown or other
  source 1..75: mol_type = other RNA
nnnnnnnnnnnnnnnnnnnngttttagagctatgctgaaaagcatagcaagttaaaataaggctagtccgttatc

SEQ ID NO: 47 - RNA, 87 nt, synthetic construct
  variation 1..20: note = a, c, u, g, unknown or other
  source 1..87: mol_type = other RNA
nnnnnnnnnnnnnnnnnnnngttttagagctatgctgttttggaaacaaaacagcatagcaagttaaaataaggctagtccgttatc

SEQ ID NO: 48 - RNA, 42 nt, synthetic construct
  variation 1..20: note = a, c, u, g, unknown or other
  source 1..42: mol_type = other RNA
nnnnnnnnnnnnnnnnnnnngttttagagctgtgttgtttcg

SEQ ID NO: 49 - RNA, 78 nt, Streptococcus thermophilus
  source 1..78: mol_type = other RNA
gggcgaaacaacacagcgagttaaaataaggcttagtccgtactcaacttgaaaaggtggcaccgattcggtgttttt

SEQ ID NO: 50 - RNA, 42 nt, synthetic construct; identical to SEQ ID NO: 42
  variation 1..20: note = a, c, u, g, unknown or other
  source 1..42: mol_type = other RNA

SEQ ID NO: 51 - RNA, 42 nt, synthetic construct; identical to SEQ ID NO: 48
  variation 1..20: note = a, c, u, g, unknown or other
  source 1..42: mol_type = other RNA

SEQ ID NO: 52 - RNA, 85 nt, Streptococcus pyogenes
  source 1..85: mol_type = other RNA
gaaccattcaaaacagcatagcaagttaaaataaggctagtccgttatcaacttgaaaaagtggcaccgagtcggtgcttttttt

SEQ ID NO: 53 - RNA, 78 nt, Streptococcus thermophilus; identical to SEQ ID NO: 49
  source 1..78: mol_type = other RNA

SEQ ID NO: 54 - protein, 12 aa, synthetic construct
  REGION 1..12: note = Description of Artificial Sequence: Synthetic peptide
DIGTNSVGWAVT

SEQ ID NO: 55 - protein, 12 aa, synthetic construct
  REGION 1..12: note = Description of Artificial Sequence: Synthetic peptide
DIGTNSVGWAVT

SEQ ID NO: 56 - protein, 12 aa, synthetic construct
  REGION 1..12: note = Description of Artificial Sequence: Synthetic peptide
DVGTNSVGWAVT

SEQ ID NO: 57 - protein, 12 aa, synthetic construct
  REGION 1..12: note = Description of Artificial Sequence: Synthetic peptide
DVGTNSVGWAVT

SEQ ID NO: 58 - protein, 12 aa, synthetic construct
  REGION 1..12: note = Description of Artificial Sequence: Synthetic peptide
DMGTNSVGWAVT

SEQ ID NO: 59 - protein, 12 aa, synthetic construct
  REGION 1..12: note = Description of Artificial Sequence: Synthetic peptide
DVGTSSVGWAVT

SEQ ID NO: 60 - protein, 12 aa, synthetic construct
  REGION 1..12: note = Description of Artificial Sequence: Synthetic peptide
DIGTASVGWAVT

SEQ ID NO: 61 - protein, 12 aa, synthetic construct
  REGION 1..12: note = Description of Artificial Sequence: Synthetic peptide
DVGTGSVGWAVT

SEQ ID NO: 62 - protein, 12 aa, synthetic construct
  REGION 1..12: note = Description of Artificial Sequence: Synthetic peptide
DIGTNSVGWAVV

SEQ ID NO: 63 - protein, 12 aa, synthetic construct
  REGION 1..12: note = Description of Artificial Sequence: Synthetic peptide
DIGTNSVGWAVI

SEQ ID NO: 64 - protein, 12 aa, synthetic construct
  REGION 1..12: note = Description of Artificial Sequence: Synthetic peptide
DIGTNSVGWAVL

SEQ ID NO: 65 - protein, 12 aa, synthetic construct
  REGION 1..12: note = Description of Artificial Sequence: Synthetic peptide
DLGTNSIGWAVV

SEQ ID NO: 66 - protein, 11 aa, synthetic construct
  REGION 1..11: note = Description of Artificial Sequence: Synthetic peptide
DLGTNSIGWAI

SEQ ID NO: 67 - protein, 12 aa, synthetic construct
  REGION 1..12: note = Description of Artificial Sequence: Synthetic peptide
DLGTNSIGWALV

SEQ ID NO: 68 - protein, 12 aa, synthetic construct
  REGION 1..12: note = Description of Artificial Sequence: Synthetic peptide
DIGTNSVGWCVT

SEQ ID NO: 69 - protein, 12 aa, synthetic construct
  REGION 1..12: note = Description of Artificial Sequence: Synthetic peptide
DIGTNSVGYAVT

SEQ ID NO: 70 - protein, 12 aa, synthetic construct
  REGION 1..12: note = Description of Artificial Sequence: Synthetic peptide
DMGTGSLGWAVT

SEQ ID NO: 71 - protein, 12 aa, synthetic construct
  REGION 1..12: note = Description of Artificial Sequence: Synthetic peptide
DIGTSSVGWAAI

SEQ ID NO: 72 - protein, 12 aa, synthetic construct
  REGION 1..12: note = Description of Artificial Sequence: Synthetic peptide
DLGTGSVGWAVV

SEQ ID NO: 73 - protein, 12 aa, synthetic construct
  REGION 1..12: note = Description of Artificial Sequence: Synthetic peptide
DLGVGSVGWAIV

SEQ ID NO: 74 - protein, 12 aa, synthetic construct
  REGION 1..12: note = Description of Artificial Sequence: Synthetic peptide
DLGIASIGWAII

SEQ ID NO: 75 - protein, 12 aa, synthetic construct
  REGION 1..12: note = Description of Artificial Sequence: Synthetic peptide
DLGIASVGWAIV

SEQ ID NO: 76 - protein, 12 aa, synthetic construct
  REGION 1..12: note = Description of Artificial Sequence: Synthetic peptide
DLGVASVGWSIV

SEQ ID NO: 77 - protein, 12 aa, synthetic construct
  REGION 1..12: note = Description of Artificial Sequence: Synthetic peptide
DIGIASVGWAIL

SEQ ID NO: 78 - protein, 12 aa, synthetic construct
  REGION 1..12: note = Description of Artificial Sequence: Synthetic peptide
DLGISSVGWSVI

SEQ ID NO: 79 - protein, 12 aa, synthetic construct
  REGION 1..12: note = Description of Artificial Sequence: Synthetic peptide
DIGIASVGWSVI

SEQ ID NO: 80 - protein, 12 aa, synthetic construct
  REGION 1..12: note = Description of Artificial Sequence: Synthetic peptide
DVGIGSIGWAVI

SEQ ID NO: 81 - protein, 12 aa, synthetic construct
  REGION 1..12: note = Description of Artificial Sequence: Synthetic peptide
DLGVGSIGFAIV

SEQ ID NO: 82 - protein, 12 aa, synthetic construct
  REGION 1..12: note = Description of Artificial Sequence: Synthetic peptide
DIGYASIGWAVI

SEQ ID NO: 83 - protein, 12 aa, synthetic construct
  REGION 1..12: note = Description of Artificial Sequence: Synthetic peptide
DTGTNSLGWAIV

SEQ ID NO: 84 - protein, 12 aa, synthetic construct
  REGION 1..12: note = Description of Artificial Sequence: Synthetic peptide
DLGTNSIGWCLL

SEQ ID NO: 85 - protein, 12 aa, synthetic construct
  REGION 1..12: note = Description of Artificial Sequence: Synthetic peptide
DIGTDSLGWAVF

SEQ ID NO: 86 - protein, 12 aa, synthetic construct
  REGION 1..12: note = Description of Artificial Sequence: Synthetic peptide
DIGSNSIGFAVV

SEQ ID NO: 87 - protein, 12 aa, synthetic construct
  REGION 1..12: note = Description of Artificial Sequence: Synthetic peptide
DLGVGSIGVAVA

SEQ ID NO: 88 - protein, 12 aa, synthetic construct
  REGION 1..12: note = Description of Artificial Sequence: Synthetic peptide
DLGIASCGWGVV

SEQ ID NO: 89 - protein, 12 aa, synthetic construct
  REGION 1..12: note = Description of Artificial Sequence: Synthetic peptide
DLGIASVGWCLT

SEQ ID NO: 90 - protein, 12 aa, synthetic construct
  REGION 1..12: note = Description of Artificial Sequence: Synthetic peptide
DIGIGSVGVGIL

SEQ ID NO: 91 - protein, 12 aa, synthetic construct
  REGION 1..12: note = Description of Artificial Sequence: Synthetic peptide
DIGITSVGYGLI

SEQ ID NO: 92 - protein, 12 aa, synthetic construct
  REGION 1..12: note = Description of Artificial Sequence: Synthetic peptide
DIGITSVGFGII

SEQ ID NO: 93 - protein, 12 aa, synthetic construct
  REGION 1..12: note = Description of Artificial Sequence: Synthetic peptide
DVGITSTGYAVL

SEQ ID NO: 94 - protein, 12 aa, synthetic construct
  REGION 1..12: note = Description of Artificial Sequence: Synthetic peptide
DLGITSFGYAIL

SEQ ID NO: 95 - protein, 12 aa, synthetic construct
  REGION 1..12: note = Description of Artificial Sequence: Synthetic peptide
DIGNASVGWSAF

SEQ ID NO: 96 - protein, 12 aa, synthetic construct
  REGION 1..12: note = Description of Artificial Sequence: Synthetic peptide
DVGTNSCGWVAM

SEQ ID NO: 97 - protein, 12 aa, synthetic construct
  REGION 1..12: note = Description of Artificial Sequence: Synthetic peptide
DVGERSIGLAAV

SEQ ID NO: 98 - protein, 12 aa, synthetic construct
  REGION 1..12: note = Description of Artificial Sequence: Synthetic peptide
DVGLNSVGLAAV

SEQ ID NO: 99 - protein, 12 aa, synthetic construct
  REGION 1..12: note = Description of Artificial Sequence: Synthetic peptide
DVGLMSVGLAAI

SEQ ID NO: 100 - protein, 12 aa, synthetic construct
  REGION 1..12: note = Description of Artificial Sequence: Synthetic peptide
DVGTFSVGLAAI

SEQ ID NO: 101 - protein, 12 aa, synthetic construct
  REGION 1..12: note = Description of Artificial Sequence: Synthetic peptide
DIGTGSVGYACM

SEQ ID NO: 102 - protein, 12 aa, synthetic construct
  REGION 1..12: note = Description of Artificial Sequence: Synthetic peptide
DLGTTSIGFAHI

SEQ ID NO: 103 - protein, 12 aa, synthetic construct
  REGION 1..12: note = Description of Artificial Sequence: Synthetic peptide
DLGTNSIGSSVR

SEQ ID NO: 104 - protein, 12 aa, synthetic construct
  REGION 1..12: note = Description of Artificial Sequence: Synthetic peptide
DIGTNSVGWAVT

SEQ ID NO: 105 - protein, 12 aa, synthetic construct
  REGION 1..12: note = Description of Artificial Sequence: Synthetic peptide
DIGTNSVGWAVT

SEQ ID NO: 106 - protein, 12 aa, synthetic construct
  REGION 1..12: note = Description of Artificial Sequence: Synthetic peptide
DVGTNSVGWAVT

SEQ ID NO: 107 - protein, 12 aa, synthetic construct
  REGION 1..12: note = Description of Artificial Sequence: Synthetic peptide
DVGTNSVGWAVT

SEQ ID NO: 108 - protein, 12 aa, synthetic construct
  REGION 1..12: note = Description of Artificial Sequence: Synthetic peptide
DMGTNSVGWAVT

SEQ ID NO: 109 - protein, 12 aa, synthetic construct
  REGION 1..12: note = Description of Artificial Sequence: Synthetic peptide
DVGTSSVGWAVT

SEQ ID NO: 110 - protein, 12 aa, synthetic construct
  REGION 1..12: note = Description of Artificial Sequence: Synthetic peptide
DIGTASVGWAVT

SEQ ID NO: 111 - protein, 12 aa, synthetic construct
  REGION 1..12: note = Description of Artificial Sequence: Synthetic peptide
DVGTGSVGWAVT

SEQ ID NO: 112 - protein, 12 aa, synthetic construct
  REGION 1..12: note = Description of Artificial Sequence: Synthetic peptide
DIGTNSVGWAVV

SEQ ID NO: 113 - protein, 12 aa, synthetic construct
  REGION 1..12: note = Description of Artificial Sequence: Synthetic peptide
DIGTNSVGWAVI

SEQ ID NO: 114 - protein, 12 aa, synthetic construct
  REGION 1..12: note = Description of Artificial Sequence: Synthetic peptide
DIGTNSIGWAVI

SEQ ID NO: 115 - protein, 12 aa, synthetic construct
  REGION 1..12: note = Description of Artificial Sequence: Synthetic peptide
DIGTNSVGWAVL

SEQ ID NO: 116 - protein, 12 aa, synthetic construct
  REGION 1..12: note = Description of Artificial Sequence: Synthetic peptide
DLGTNSIGWAVV

SEQ ID NO: 117 - protein, 11 aa, synthetic construct
  REGION 1..11: note = Description of Artificial Sequence: Synthetic peptide
DLGTNSIGWAI

SEQ ID NO: 118 - protein, 12 aa, synthetic construct
  REGION 1..12: note = Description of Artificial Sequence: Synthetic peptide
DLGTNSIGWALV

SEQ ID NO: 119 - protein, 12 aa, synthetic construct
  REGION 1..12: note = Description of Artificial Sequence: Synthetic peptide
DIGTNSVGWCVT

SEQ ID NO: 120 - protein, 12 aa, synthetic construct
  REGION 1..12: note = Description of Artificial Sequence: Synthetic peptide
DIGTNSVGYAVT

SEQ ID NO: 121 - protein, 12 aa, synthetic construct
  REGION 1..12: note = Description of Artificial Sequence: Synthetic peptide
DMGTGSLGWAVT

SEQ ID NO: 122 - protein, 12 aa, synthetic construct
  REGION 1..12: note = Description of Artificial Sequence: Synthetic peptide
DIGTSSVGWAAI

SEQ ID NO: 123 - protein, 12 aa, synthetic construct
  REGION 1..12: note = Description of Artificial Sequence: Synthetic peptide
DLGTGSVGWAVV

SEQ ID NO: 124 - protein, 12 aa, synthetic construct
  REGION 1..12: note = Description of Artificial Sequence: Synthetic peptide
DLGVGSVGWAIV

SEQ ID NO: 125 - protein, 12 aa, synthetic construct
  REGION 1..12: note = Description of Artificial Sequence: Synthetic peptide
DLGIASIGWAII

SEQ ID NO: 126 - protein, 12 aa, synthetic construct
  REGION 1..12: note = Description of Artificial Sequence: Synthetic peptide
DLGIASVGWAIV

SEQ ID NO: 127 - protein, 12 aa, synthetic construct
  REGION 1..12: note = Description of Artificial Sequence: Synthetic peptide
DLGIASVGWAVV

SEQ ID NO: 128 - protein, 12 aa, synthetic construct
  REGION 1..12: note = Description of Artificial Sequence: Synthetic peptide
DLGVASVGWSIV

SEQ ID NO: 129 - protein, 12 aa, synthetic construct
  REGION 1..12: note = Description of Artificial Sequence: Synthetic peptide
DIGIASVGWAIL

SEQ ID NO: 130 - protein, 12 aa, synthetic construct
  REGION 1..12: note = Description of Artificial Sequence: Synthetic peptide
DIGIASVGWAVL

SEQ ID NO: 131 - protein, 12 aa, synthetic construct
  REGION 1..12: note = Description of Artificial Sequence: Synthetic peptide
DIGIASIGWAVI

SEQ ID NO: 132 - protein, 12 aa, synthetic construct
  REGION 1..12: note = Description of Artificial Sequence: Synthetic peptide
DIGIASVGWAII

SEQ ID NO: 133 - protein, 12 aa, synthetic construct
  REGION 1..12: note = Description of Artificial Sequence: Synthetic peptide
DVGIASVGWAVI

SEQ ID NO: 134 - protein, 11 aa, synthetic construct
  REGION 1..11: note = Description of Artificial Sequence: Synthetic peptide
DIGIASVGWAL

SEQ ID NO: 135 - protein, 12 aa, synthetic construct
  REGION 1..12: note = Description of Artificial Sequence: Synthetic peptide
DIGIASVGWAMV

SEQ ID NO: 136 - protein, 12 aa, synthetic construct
  REGION 1..12: note = Description of Artificial Sequence: Synthetic peptide
DIGIASVGWSVI

SEQ ID NO: 137 - protein, 12 aa, synthetic construct
  REGION 1..12: note = Description of Artificial Sequence: Synthetic peptide
DLGISSVGWSVI

SEQ ID NO: 138 - protein, 12 aa, synthetic construct
  REGION 1..12: note = Description of Artificial Sequence: Synthetic peptide
DIGITSVGWAVI

SEQ ID NO: 139 - protein, 12 aa, synthetic construct
  REGION 1..12: note = Description of Artificial Sequence: Synthetic peptide
DVGIGSIGWAVI

SEQ ID NO: 140 - protein, 12 aa, synthetic construct
  REGION 1..12: note = Description of Artificial Sequence: Synthetic peptide
DLGISSLGWAIV

SEQ ID NO: 141 - protein, 12 aa, synthetic construct
  REGION 1..12: note = Description of Artificial Sequence: Synthetic peptide
DLGVGSIGFAIV

SEQ ID NO: 142 - protein, 12 aa, synthetic construct
  REGION 1..12: note = Description of Artificial Sequence: Synthetic peptide
DIGYASIGWAVI

SEQ ID NO: 143 - protein, 12 aa, synthetic construct
  REGION 1..12: note = Description of Artificial Sequence: Synthetic peptide
DLGTNSIGWCLL

SEQ ID NO: 144 - protein, 12 aa, synthetic construct
  REGION 1..12: note = Description of Artificial Sequence: Synthetic peptide
DLGTNSIGWGLL

SEQ ID NO: 145 - protein, 12 aa, synthetic construct
  REGION 1..12: note = Description of Artificial Sequence: Synthetic peptide
DTGTNSLGWAIV

SEQ ID NO: 146 - protein, 12 aa, synthetic construct
  REGION 1..12: note = Description of Artificial Sequence: Synthetic peptide
DIGTDSLGWAVF

SEQ ID NO: 147 - protein, 12 aa, synthetic construct
  REGION 1..12: note = Description of Artificial Sequence: Synthetic peptide
DLGSTSLGWAIF

SEQ ID NO: 148 - protein, 12 aa, synthetic construct
  REGION 1..12: note = Description of Artificial Sequence: Synthetic peptide
DIGISSIGWAFS

SEQ ID NO: 149 - protein, 12 aa, synthetic construct
  REGION 1..12: note = Description of Artificial Sequence: Synthetic peptide
DLGIASVGWCLT

SEQ ID NO: 150 - protein, 12 aa, synthetic construct
  REGION 1..12: note = Description of Artificial Sequence: Synthetic peptide
DLGIASCGWGVV

SEQ ID NO: 151 - protein, 12 aa, synthetic construct
  REGION 1..12: note = Description of Artificial Sequence: Synthetic peptide
DIGSNSIGFAVV

SEQ ID NO: 152 - protein, 12 aa, synthetic construct
  REGION 1..12: note = Description of Artificial Sequence: Synthetic peptide
DIGTTSIGFSVI

SEQ ID NO: 153 - protein, 12 aa, synthetic construct
  REGION 1..12: note = Description of Artificial Sequence: Synthetic peptide
DIGITSVGYGLI

SEQ ID NO: 154 - protein, 12 aa, synthetic construct
  REGION 1..12: note = Description of Artificial Sequence: Synthetic peptide
DIGITSVGFGII

SEQ ID NO: 155 - protein, 12 aa, synthetic construct
  REGION 1..12: note = Description of Artificial Sequence: Synthetic peptide
DLGTTSIGFAHI

SEQ ID NO: 156 - protein, 12 aa, synthetic construct
  REGION 1..12: note = Description of Artificial Sequence: Synthetic peptide
DIGIGSVGVGIL

SEQ ID NO: 157 - protein, 12 aa, synthetic construct
  REGION 1..12: note = Description of Artificial Sequence: Synthetic peptide
DLGVGSIGVAVA

SEQ ID NO: 158 - protein, 12 aa, synthetic construct
  REGION 1..12: note = Description of Artificial Sequence: Synthetic peptide
DVGITSTGYAVL

SEQ ID NO: 159 - protein, 12 aa, synthetic construct
  REGION 1..12: note = Description of Artificial Sequence: Synthetic peptide
DLGITSFGYAIL

SEQ ID NO: 160 - protein, 12 aa, synthetic construct
  REGION 1..12: note = Description of Artificial Sequence: Synthetic peptide
DIGTSSIGWWLY

SEQ ID NO: 161 - protein, 12 aa, synthetic construct
  REGION 1..12: note = Description of Artificial Sequence: Synthetic peptide
DLGSNSLGWFVT

SEQ ID NO: 162 - protein, 12 aa, synthetic construct
  REGION 1..12: note = Description of Artificial Sequence: Synthetic peptide
DLGANSLGWFVV

SEQ ID NO: 163 - protein, 12 aa, synthetic construct
  REGION 1..12: note = Description of Artificial Sequence: Synthetic peptide
DIGNASVGWSAF

SEQ ID NO: 164 - protein, 12 aa, synthetic construct
  REGION 1..12: note = Description of Artificial Sequence: Synthetic peptide
DVGTNSCGWVAM

SEQ ID NO: 165 - protein, 12 aa, synthetic construct
  REGION 1..12: note = Description of Artificial Sequence: Synthetic peptide
DVGERSIGLAAV

SEQ ID NO: 166 - protein, 12 aa, synthetic construct
  REGION 1..12: note = Description of Artificial Sequence: Synthetic peptide
DVGLNSVGLAAV

SEQ ID NO: 167 - protein, 12 aa, synthetic construct
  REGION 1..12: note = Description of Artificial Sequence: Synthetic peptide
DVGLMSVGLAAI

SEQ ID NO: 168 - protein, 12 aa, synthetic construct
  REGION 1..12: note = Description of Artificial Sequence: Synthetic peptide
DVGTFSVGLAAI

SEQ ID NO: 169 - protein, 12 aa, synthetic construct
  REGION 1..12: note = Description of Artificial Sequence: Synthetic peptide
DIGTGSVGYACM

SEQ ID NO: 170 - protein, 12 aa, synthetic construct
  REGION 1..12: note = Description of Artificial Sequence: Synthetic peptide
DLGTNSIGSSVR

SEQ ID NO: 171 - protein, 15 aa, synthetic construct
  REGION 1..15: note = Description of Artificial Sequence: Synthetic peptide
DIGLRIGITSCGWSI

SEQ ID NO: 172 - protein, 12 aa, synthetic construct
  REGION 1..12: note = Description of Artificial Sequence: Synthetic peptide
DMGAKYTGVFYA

SEQ ID NO: 173 - protein, 12 aa, synthetic construct
  REGION 1..12: note = Description of Artificial Sequence: Synthetic peptide
DLGGKNTGFFSF

SEQ ID NO: 174 - protein, 12 aa, synthetic construct
  REGION 1..12: note = Description of Artificial Sequence: Synthetic peptide
DLGVKNTGVFSA

SEQ ID NO: 175 - protein, 12 aa, synthetic construct
  REGION 1..12: note = Description of Artificial Sequence: Synthetic peptide
DLGAKFTGVALY

SEQ ID NO: 176 - protein, 12 aa, synthetic construct
  REGION 1..12: note = Description of Artificial Sequence: Synthetic peptide
DLGGKFTGVCLS

SEQ ID NO: 177 - protein, 12 aa, synthetic construct
  REGION 1..12: note = Description of Artificial Sequence: Synthetic peptide
DLGGTYTGTFIT

SEQ ID NO: 178 - protein, 28 aa, synthetic construct
  REGION 1..28: note = Description of Artificial Sequence: Synthetic peptide
YDIDHIYPRSLTKDDSFDNLVLCERTAN

SEQ ID NO: 179 - protein, 28 aa, synthetic construct
  REGION 1..28: note = Description of Artificial Sequence: Synthetic peptide
DIDHIYPRSKVIKDDSFDNLVLVLKNEN

SEQ ID NO: 180 - protein, 27 aa, synthetic construct
  REGION 1..27: note = Description of Artificial Sequence: Synthetic peptide
DRDHIYPQSKIKDDSIDNLVLVNKTYN

SEQ ID NO: 181 - protein, 27 aa, synthetic construct
  REGION 1..27: note = Description of Artificial Sequence: Synthetic peptide
DIDHIYPRSKIKDDSITNRVLVEKDIN

SEQ ID NO: 182 - protein, 27 aa, synthetic construct
  REGION 1..27: note = Description of Artificial Sequence: Synthetic peptide
DIDHIYPQSKIKDDSISNRVLVCSSCN

SEQ ID NO: 183 - protein, 27 aa, synthetic construct
  REGION 1..27: note = Description of Artificial Sequence: Synthetic peptide
DIDHIYPQSKTMDDSLNNRVLVKKNYN

SEQ ID NO: 184 - protein, 27 aa, synthetic construct
  REGION 1..27: note = Description of Artificial Sequence: Synthetic peptide
DQDHIYPKSKIYDDSLENRVLVKKNLN

SEQ ID NO: 185 - protein, 27 aa, synthetic construct
  REGION 1..27: note = Description of Artificial Sequence: Synthetic peptide
QIDHIVPQSLVKDDSFDNRVLVVPSEN

SEQ ID NO: 186 - protein, 27 aa, synthetic construct
  REGION 1..27: note = Description of Artificial Sequence: Synthetic peptide
DIDHIIPQAFIKDNSIDNRVLTSSKEN

SEQ ID NO: 187 - protein, 27 aa, synthetic construct
  REGION 1..27: note = Description of Artificial Sequence: Synthetic peptide
DIDHIIPQAFLKDNSIDNKVLVSSASN

SEQ ID NO: 188 - protein, 27 aa, synthetic construct
  REGION 1..27: note = Description of Artificial Sequence: Synthetic peptide
DIDHIIPQAYTKDNSLDNRVLVSNITN

SEQ ID NO: 189 - protein, 27 aa, synthetic construct
  REGION 1..27: note = Description of Artificial Sequence: Synthetic peptide
DIDHIVPQSFITDNSIDNLVLTSSAGN

SEQ ID NO: 190 - protein, 27 aa, synthetic construct
  REGION 1..27: note = Description of Artificial Sequence: Synthetic peptide
DVDHIVPQSFLKDDSIDNKVLTRSDKN

SEQ ID NO: 191 - protein, 27 aa, synthetic construct
  REGION 1..27: note = Description of Artificial Sequence: Synthetic peptide
NIDHIYPQSMVKDDSLDNKVLVQSEIN

SEQ ID NO: 192 - protein, 27 aa, synthetic construct
  REGION 1..27: note = Description of Artificial Sequence: Synthetic peptide
DIDHILPQSLIKDDSLDNRVLVNATIN

SEQ ID NO: 193 - protein, 27 aa, synthetic construct
  REGION 1..27: note = Description of Artificial Sequence: Synthetic peptide
DIDHILPQSFIKDDSLENRVLVKKAVN

SEQ ID NO: 194 - protein, 27 aa, synthetic construct
  REGION 1..27: note = Description of Artificial Sequence: Synthetic peptide
EVDHIFPRSFIKDDSIDNKVLVIKKMN

SEQ ID NO: 195 - protein, 27 aa, synthetic construct
  REGION 1..27: note = Description of Artificial Sequence: Synthetic peptide
EVDHIIPRSYIKDDSFENKVLVYREEN

SEQ ID NO: 196 - protein, 27 aa, synthetic construct
  REGION 1..27: note = Description of Artificial Sequence: Synthetic peptide
DIDHIIPQAVTQNDSIDNRVLVARAEN

SEQ ID NO: 197 - protein, 27 aa, synthetic construct
  REGION 1..27: note = Description of Artificial Sequence: Synthetic peptide
EIDHIIPYSISFDDSSSNKLLVLAESN

SEQ ID NO: 198 - protein, 27 aa, synthetic construct
  REGION 1..27: note = Description of Artificial Sequence: Synthetic peptide
EIDHIIPYSLCFDDSSANKVLVHKQSN

SEQ ID NO: 199 - protein, 27 aa, synthetic construct
  REGION 1..27: note = Description of Artificial Sequence: Synthetic peptide
DIDHIIPYSRSMDDSYSNKVLVLSGEN

SEQ ID NO: 200 - protein, 27 aa, synthetic construct
  REGION 1..27: note = Description of Artificial Sequence: Synthetic peptide
DIDHIIPYSKSMDDSFNNKVLCLAEEN

SEQ ID NO: 201 - protein, 27 aa, synthetic construct
  REGION 1..27: note = Description of Artificial Sequence: Synthetic peptide
EIDHIYPYSRSFDDSYMNKVLVFTKQN

SEQ ID NO: 202 - protein, 27 aa, synthetic construct
  REGION 1..27: note = Description of Artificial Sequence: Synthetic peptide
QIDHIYPYSRSMDDSYMNKVLVLTDEN

SEQ ID NO: 203 - protein, 27 aa, synthetic construct
  REGION 1..27: note = Description of Artificial Sequence: Synthetic peptide
EIDHIIPFSRSFDDSLSNKILVLGSEN

SEQ ID NO: 204 - protein, 27 aa, synthetic construct
  REGION 1..27: note = Description of Artificial Sequence: Synthetic peptide
EIDHALPFSRTWDDSFNNKVLVLGSEN

SEQ ID NO: 205 - protein, 27 aa, synthetic construct
  REGION 1..27: note = Description of Artificial Sequence: Synthetic peptide
EIDHALPFSRTWDDSFNNKVLVLASEN

SEQ ID NO: 206 - protein, 27 aa, synthetic construct
  REGION 1..27: note = Description of Artificial Sequence: Synthetic peptide
EIDHIIPISISLDDSINNKVLVLSKAN

SEQ ID NO: 207 - protein, 27 aa, synthetic construct
  REGION 1..27: note = Description of Artificial Sequence: Synthetic peptide
EVDHIIPISISLDDSITNKVLVTHREN

SEQ ID NO: 208 - protein, 27 aa, synthetic construct
  REGION 1..27: note = Description of Artificial Sequence: Synthetic peptide
QVDHALPYSRSYDDSKNNKVLVLTHEN

SEQ ID NO: 209 - protein, 27 aa, synthetic construct
  REGION 1..27: note = Description of Artificial Sequence: Synthetic peptide
EVDHILPLSITFDDSLANKVLVYATAN

SEQ ID NO: 210 - protein, 27 aa, synthetic construct
  REGION 1..27: note = Description of Artificial Sequence: Synthetic peptide
EIDHIIPRSISFDDARSNKVLVYRSEN

SEQ ID NO: 211 - protein, 27 aa, synthetic construct
  REGION 1..27: note = Description of Artificial Sequence: Synthetic peptide
EVDHIIPRSVSFDNSYHNKVLVKQSEN

SEQ ID NO: 212 - protein, 27 aa, synthetic construct
  REGION 1..27: note = Description of Artificial Sequence: Synthetic peptide
DIDHILPYSITFDDSFRNKVLVTSQEN

SEQ ID NO: 213 - protein, 27 aa, synthetic construct
  REGION 1..27: note = Description of Artificial Sequence: Synthetic peptide
EIDHILPRSRSADDSFANKVLCLARAN

SEQ ID NO: 214 - protein, 27 aa, synthetic construct
  REGION 1..27: note = Description of Artificial Sequence: Synthetic peptide
EIEHLLPFSLTLDDSMANKTVCFRQAN

SEQ ID NO: 215 - protein, 27 aa, synthetic construct
  REGION 1..27: note = Description of Artificial Sequence: Synthetic peptide
DIDHILPFSVSLDDSAANKVVCLREAN

SEQ ID NO: 216 - protein, 27 aa, synthetic construct
  REGION 1..27: note = Description of Artificial Sequence: Synthetic peptide
DIDHLIPFSISWDDSAANKVVCMRYAN

SEQ ID NO: 217 - protein, 27 aa, synthetic construct
  REGION 1..27: note = Description of Artificial Sequence: Synthetic peptide
DIDHILPVAMTLDDSPANKIICMRYAN

SEQ ID NO: 218 - protein, 27 aa, synthetic construct
  REGION 1..27: note = Description of Artificial Sequence: Synthetic peptide
DVDHILPYSRTLDDSFPNRTLCLREAN

SEQ ID NO: 219 - protein, 27 aa, synthetic construct
  REGION 1..27: note = Description of Artificial Sequence: Synthetic peptide
EIEHILPFSRTLDDSLNNRTVAMRRAN

SEQ ID NO: 220 - protein, 27 aa, synthetic construct
  REGION 1..27: note = Description of Artificial Sequence: Synthetic peptide
EVDHIIPYSISWDDSYTNKVLTSAKCN

SEQ ID NO: 221 - protein, 27 aa, synthetic construct
  REGION 1..27: note = Description of Artificial Sequence: Synthetic peptide
QVDHILPWSRFGDDSYLNKTLCTARSN

SEQ ID NO: 222 - protein, 27 aa, synthetic construct
  REGION 1..27: note = Description of Artificial Sequence: Synthetic peptide
QVDHILPFSKTLDDSFANKVLAQHDAN

SEQ ID NO: 223 - protein, 27 aa, synthetic construct
  REGION 1..27: note = Description of Artificial Sequence: Synthetic peptide
QIDHAFPLSRSLDDSQSNKVLCLTSSN

SEQ ID NO: 224 - protein, 27 aa, synthetic construct
  REGION 1..27: note = Description of Artificial Sequence: Synthetic peptide
DIDHIVPRSISFDDSFSNLVIVNKLDN

SEQ ID NO: 225 - protein, 27 aa, synthetic construct
  REGION 1..27: note = Description of Artificial Sequence: Synthetic peptide
EIEHIIPYSMSYDNSQANKILTEKAEN

SEQ ID NO: 226 - protein, 27 aa, synthetic construct
  REGION 1..27: note = Description of Artificial Sequence: Synthetic peptide
EIDHVIPYSKSADDSWFNKLLVKKSTN

SEQ ID NO: 227 - protein, 27 aa, synthetic construct
  REGION 1..27: note = Description of Artificial Sequence: Synthetic peptide
EMDHILPYSRSLDNGWHNRVLVHGKDN

SEQ ID NO: 228 - protein, 27 aa, synthetic construct
  REGION 1..27: note = Description of Artificial Sequence: Synthetic peptide
EVDHIVPYSLILDNTINNKALVYAEEN

SEQ ID NO: 229 - protein, 27 aa, synthetic construct
  REGION 1..27: note = Description of Artificial Sequence: Synthetic peptide
EIEHVIPQSLYFDDSFSNKVICEAEVN

SEQ ID NO: 230 - protein, 27 aa, synthetic construct
  REGION 1..27: note = Description of Artificial Sequence: Synthetic peptide
DIEHIIPQARLFDDSFSNKTLEARSVN

SEQ ID NO: 231 - protein, 27 aa, synthetic construct
  REGION 1..27: note = Description of Artificial Sequence: Synthetic peptide
EIEHIVPKARVFDDSFSNKTLTFHRIN

SEQ ID NO: 232 - protein, 28 aa, synthetic construct
  REGION 1..28: note = Description of Artificial Sequence: Synthetic peptide
DKDHIIPQSMKKDDSIINNLVLVNKNAN

SEQ ID NO: 233 - protein, 27 aa, synthetic construct
  REGION 1..27: note = Description of Artificial Sequence: Synthetic peptide
EVEHIWPRSRSFDNSPRNKTLCRKDVN

SEQ ID NO: 234 - protein, 27 aa, synthetic construct
  REGION 1..27: note = Description of Artificial Sequence: Synthetic peptide
IVNHIIPYNRSFDDTYHNRVLTLTETK

SEQ ID NO: 235 - protein, 27 aa, synthetic construct
  REGION 1..27: note = Description of Artificial Sequence: Synthetic peptide
DMEHTIPKSISFDNSDQNLTLCESYYN

SEQ ID NO: 236 - protein, 27 aa, synthetic construct
  REGION 1..27: note = Description of Artificial Sequence: Synthetic peptide
DIEHTIPRSAGGDSTKMNLTLCSSRFN

SEQ ID NO: 237 - protein, 27 aa, synthetic construct
  REGION 1..27: note = Description of Artificial Sequence: Synthetic peptide
DIEHTIPRSISQDNSQMNKTLCSLKFN

SEQ ID NO: 238 - protein, 27 aa, synthetic construct
  REGION 1..27: note = Description of Artificial Sequence: Synthetic peptide
DIDHVIPLARGGRDSLDNMVLCQSDAN

SEQ ID NO: 239 - protein, 27 aa, synthetic construct
  REGION 1..27: note = Description of Artificial Sequence: Synthetic peptide
DIEHLFPIAESEDNGRNNLVISHSACN

SEQ ID NO: 240 - protein, 27 aa, synthetic construct
  REGION 1..27: note = Description of Artificial Sequence: Synthetic peptide
DVDHIFPRDDTADNSYGNKVVAHRQCN

SEQ ID NO: 241 - protein, 27 aa, synthetic construct
  REGION 1..27: note = Description of Artificial Sequence: Synthetic peptide
DIEHIVPQSLGGLSTDYNTIVTLKSVN

SEQ ID NO: 242 - protein, 27 aa, synthetic construct
  REGION 1..27: note = Description of Artificial Sequence: Synthetic peptide
ELDHIVPRTDGGSNRHENLAITCGACN

SEQ ID NO: 243 - protein, 28 aa, synthetic construct
  REGION 1..28: note = Description of Artificial Sequence: Synthetic peptide
EMDHIVPRKGVGSTNTRTNFAAVCAECN

SEQ ID NO: 244 - protein, 28 aa, synthetic construct
  REGION 1..28: note = Description of Artificial Sequence: Synthetic peptide
EMDHIVPRKGVGSTNTRVNLAAACAACN

SEQ ID NO: 245 - protein, 28 aa, synthetic construct
  REGION 1..28: note = Description of Artificial Sequence: Synthetic peptide
EMDHIVPRAGQGSTNTRENLVAVCHRCN

SEQ ID NO: 246 - protein, 33 aa, synthetic construct
  REGION 1..33: note = Description of Artificial Sequence: Synthetic polypeptide
EIDHILPRSLIKDARGIVFNAEPNLIYASSRGN

SEQ ID NO: 247 - protein, 33 aa, synthetic construct
  REGION 1..33: note = Description of Artificial Sequence: Synthetic polypeptide
EIDHIIPRSLTGRTKKTVFNSEANLIYCSSKGN

SEQ ID NO: 248 - protein, 33 aa, synthetic construct
  REGION 1..33: note = Description of Artificial Sequence: Synthetic polypeptide
EIDHIIPRSLTLKKSESIYNSEVNLIFVSAQGN

SEQ ID NO: 249 - protein, 33 aa, synthetic construct
  REGION 1..33: note = Description of Artificial Sequence: Synthetic polypeptide
EIDHIYPRSLSKKHFGVIFNSEVNLIYCSSQGN

SEQ ID NO: 250 - protein, 33 aa, synthetic construct
  REGION 1..33: note = Description of Artificial Sequence: Synthetic polypeptide
EIDHILPRSHTLKIYGTVFNPEGNLIYVHQKCN

SEQ ID NO: 251 - protein, 30 aa, synthetic construct
  REGION 1..30: note = Description of Artificial Sequence: Synthetic polypeptide
ELDHIIPRSHKKYGTLNDEANLICVTRGDN

SEQ ID NO: 252 - protein, 27 aa, synthetic construct
  REGION 1..27: note = Description of Artificial Sequence: Synthetic peptide
ELEHIVPHSFRQSNALSSLVLTWPGVN

SEQ ID NO: 253 - protein, 28 aa, synthetic construct
  REGION 1..28: note = Description of Artificial Sequence: Synthetic peptide
YDIDHIYPRSLTKDDSFDNLVLCERTAN

SEQ ID NO: 254 - protein, 28 aa, synthetic construct
  REGION 1..28: note = Description of Artificial Sequence: Synthetic peptide
DIDHIYPRSKVIKDDSFDNLVLVLKNEN

SEQ ID NO: 255 - protein, 27 aa, synthetic construct
  REGION 1..27: note = Description of Artificial Sequence: Synthetic peptide
DRDHIYPQSKIKDDSIDNLVLVNKTYN

SEQ ID NO: 256 - protein, 27 aa, synthetic construct
  REGION 1..27: note = Description of Artificial Sequence: Synthetic peptide
DIDHIYPRSKIKDDSITNRVLVEKDIN

SEQ ID NO: 257 - protein, 27 aa, synthetic construct
  REGION 1..27: note = Description of Artificial Sequence: Synthetic peptide
DIDHIYPQSKIKDDSISNRVLVCSSCN

SEQ ID NO: 258 - protein, 27 aa, synthetic construct
  REGION 1..27: note = Description of Artificial Sequence: Synthetic peptide
DIDHIYPQSKTMDDSLNNRVLVKKNYN

SEQ ID NO: 259 - protein, 27 aa, synthetic construct
  REGION 1..27: note = Description of Artificial Sequence: Synthetic peptide
DQDHIYPKSKIYDDSLENRVLVKKNLN

SEQ ID NO: 260 - protein, 27 aa, synthetic construct
  REGION 1..27: note = Description of Artificial Sequence: Synthetic peptide
QIDHIVPQSLVKDDSFDNRVLVVPSEN

SEQ ID NO: 261 - protein, 27 aa, synthetic construct
  REGION 1..27: note = Description of Artificial Sequence: Synthetic peptide
DIDHIIPQAFIKDNSIDNRVLTSSKEN

SEQ ID NO: 262 - protein, 27 aa, synthetic construct
  REGION 1..27: note = Description of Artificial Sequence: Synthetic peptide
DIDHIIPQAFLKDNSIDNKVLVSSASN

SEQ ID NO: 263 - protein, 27 aa, synthetic construct
  REGION 1..27: note = Description of Artificial Sequence: Synthetic peptide
DIDHIIPQAYTKDNSLDNRVLVSNITN

SEQ ID NO: 264 - protein, 27 aa, synthetic construct
  REGION 1..27: note = Description of Artificial Sequence: Synthetic peptide
DIDHIVPQSFITDNSIDNLVLTSSAGN

SEQ ID NO: 265 - protein, 27 aa, synthetic construct
  REGION 1..27: note = Description of Artificial Sequence: Synthetic peptide
DVDHIVPQSFLKDDSIDNKVLTRSDKN

SEQ ID NO: 266 - protein, 27 aa, synthetic construct
  REGION 1..27: note = Description of Artificial Sequence: Synthetic peptide
NIDHIYPQSMVKDDSLDNKVLVQSEIN

SEQ ID NO: 267 - protein, 27 aa, synthetic construct
  REGION 1..27: note = Description of Artificial Sequence: Synthetic peptide
DIDHILPQSLIKDDSLDNRVLVNATIN

SEQ ID NO: 268 - protein, 27 aa, synthetic construct
  REGION 1..27: note = Description of Artificial Sequence: Synthetic peptide
DIDHILPQSFIKDDSLENRVLVKKAVN

SEQ ID NO: 269 - protein, 27 aa, synthetic construct
  REGION 1..27: note = Description of Artificial Sequence: Synthetic peptide
EVDHIFPRSFIKDDSIDNKVLVIKKMN

SEQ ID NO: 270 - protein, 27 aa, synthetic construct
  REGION 1..27: note = Description of Artificial Sequence: Synthetic peptide
EVDHIIPRSYIKDDSFENKVLVYREEN

SEQ ID NO: 271 - protein, 27 aa, synthetic construct
  REGION 1..27: note = Description of Artificial Sequence: Synthetic peptide
DIDHIIPQAVTQNDSIDNRVLVARAEN

SEQ ID NO: 272 - protein, 27 aa, synthetic construct
  REGION 1..27: note = Description of Artificial Sequence: Synthetic peptide
DIDHIVPRSISFDDSFSNLVIVNKLDN

SEQ ID NO: 273 - protein, 27 aa, synthetic construct
  REGION 1..27: note = Description of Artificial Sequence: Synthetic peptide
EIDHIIPYSISFDDSSSNKLLVLAESN

SEQ ID NO: 274 - protein, 27 aa, synthetic construct
  REGION 1..27: note = Description of Artificial Sequence: Synthetic peptide
EIDHIIPYSLCFDDSSANKVLVHKQSN

SEQ ID NO: 275 - protein, 27 aa, synthetic construct
  REGION 1..27: note = Description of Artificial Sequence: Synthetic peptide
EIDHIIPISISLDDSINNKVLVLSKAN

SEQ ID NO: 276 - protein, 27 aa, synthetic construct
  REGION 1..27: note = Description of Artificial Sequence: Synthetic peptide
EVDHIIPISISLDDSITNKVLVTHREN

SEQ ID NO: 277 - protein, 27 aa, synthetic construct
  REGION 1..27: note = Description of Artificial Sequence: Synthetic peptide
EVDHILPLSITFDDSLANKVLVYATAN

SEQ ID NO: 278 - protein, 27 aa, synthetic construct
  REGION 1..27: note = Description of Artificial Sequence: Synthetic peptide
EIDHIIPRSISFDDARSNKVLVYRSEN

SEQ ID NO: 279 - protein, 27 aa, synthetic construct
  REGION 1..27: note = Description of Artificial Sequence: Synthetic peptide
EVDHIIPRSVSFDNSYHNKVLVKQSEN

SEQ ID NO: 280 - protein, 27 aa, synthetic construct
  REGION 1..27: note = Description of Artificial Sequence: Synthetic peptide
EVDHIIPYSISWDDSYTNKVLTSAKCN

SEQ ID NO: 281 - protein, 27 aa, synthetic construct
  REGION 1..27: note = Description of Artificial Sequence: Synthetic peptide
DIDHIIPYSRSMDDSYSNKVLVLSGEN

SEQ ID NO: 282 - protein, 27 aa, synthetic construct
  REGION 1..27: note = Description of Artificial Sequence: Synthetic peptide
EIEHIIPYSMSYDNSQANKILTEKAEN

SEQ ID NO: 283 - protein, 27 aa, synthetic construct
  REGION 1..27: note = Description of Artificial Sequence: Synthetic peptide
EVDHIVPYSLILDNTINNKALVYAEEN

SEQ ID NO: 284 - protein, 27 aa, synthetic construct
  REGION 1..27: note = Description of Artificial Sequence: Synthetic peptide
EIDHVIPYSKSADDSWFNKLLVKKSTN

SEQ ID NO: 285 - protein, 27 aa, synthetic construct
  REGION 1..27: note = Description of Artificial Sequence: Synthetic peptide
EMDHILPYSRSLDNGWHNRVLVHGKDN

SEQ ID NO: 286 - protein, 27 aa, synthetic construct
  REGION 1..27: note = Description of Artificial Sequence: Synthetic peptide
EIEHVIPQSLYFDDSFSNKVICEAEVN

SEQ ID NO: 287 - protein, 27 aa, synthetic construct
  REGION 1..27: note = Description of Artificial Sequence: Synthetic peptide
DIEHIIPQARLFDDSFSNKTLEARSVN

SEQ ID NO: 288 - protein, 27 aa, synthetic construct
  REGION 1..27: note = Description of Artificial Sequence: Synthetic peptide
EIEHIVPKARVFDDSFSNKTLTFHRIN

SEQ ID NO: 289 - protein, 28 aa, synthetic construct
  REGION 1..28: note = Description of Artificial Sequence: Synthetic peptide
DKDHIIPQSMKKDDSIINNLVLVNKNAN

SEQ ID NO: 290 - protein, 27 aa, synthetic construct
  REGION 1..27: note = Description of Artificial Sequence: Synthetic peptide
QVDHILPWSRFGDDSYLNKTLCTARSN

SEQ ID NO: 291 - protein, 27 aa, synthetic construct
  REGION 1..27: note = Description of Artificial Sequence: Synthetic peptide
DIDHVIPLARGGRDSLDNMVLCQSDAN

SEQ ID NO: 292 - protein, 27 aa, synthetic construct
  REGION 1..27: note = Description of Artificial Sequence: Synthetic peptide
DMEHTIPKSISFDNSDQNLTLCESYYN

SEQ ID NO: 293 - protein, 27 aa, synthetic construct
  REGION 1..27: note = Description of Artificial Sequence: Synthetic peptide
DIEHTIPRSAGGDSTKMNLTLCSSRFN

SEQ ID NO: 294 - protein, 27 aa, synthetic construct
  REGION 1..27: note = Description of Artificial Sequence: Synthetic peptide
DIEHTIPRSISQDNSQMNKTLCSLKFN

SEQ ID NO: 295 - protein, 27 aa, synthetic construct
  REGION 1..27: note = Description of Artificial Sequence: Synthetic peptide
DIEHLFPIAESEDNGRNNLVISHSACN

SEQ ID NO: 296 - protein, 27 aa, synthetic construct
  REGION 1..27: note = Description of Artificial Sequence: Synthetic peptide
DVDHIFPRDDTADNSYGNKVVAHRQCN

SEQ ID NO: 297 - protein, 27 aa, synthetic construct
  REGION 1..27: note = Description of Artificial Sequence: Synthetic peptide
DIEHIVPQSLGGLSTDYNTIVTLKSVN

SEQ ID NO: 298 - protein, 27 aa, synthetic construct
  REGION 1..27: note = Description of Artificial Sequence: Synthetic peptide
ELDHIVPRTDGGSNRHENLAITCGACN

SEQ ID NO: 299 - protein, 28 aa, synthetic construct
  REGION 1..28: note = Description of Artificial Sequence: Synthetic peptide
EMDHIVPRKGVGSTNTRTNFAAVCAECN

SEQ ID NO: 300 - protein, 28 aa, synthetic construct
  REGION 1..28: note = Description of Artificial Sequence: Synthetic peptide
EMDHIVPRKGVGSTNTRVNLAAACAACN

SEQ ID NO: 301 - protein, 28 aa, synthetic construct
  REGION 1..28: note = Description of Artificial Sequence: Synthetic peptide
EMDHIVPRAGQGSTNTRENLVAVCHRCN

SEQ ID NO: 302 - protein, 27 aa, synthetic construct
  REGION 1..27: note = Description of Artificial Sequence: Synthetic peptide
ELEHIVPHSFRQSNALSSLVLTWPGVN

SEQ ID NO: 303 - DNA, 3333 bp, synthetic construct
  misc_feature 1..3333: note = Description of Artificial Sequence: Synthetic polynucleotide
  source 1..3333: mol_type = other DNA
atggtgcctaagaagaagagaaaggtggctgccttcaaacctaattcaatcaactacatcctcggcctcgatatcggcatcgcatccgtcggctgggcgatggtagaaattgacgaagaagaaaaccccatccgcctgattgatttgggcgtgcgcgtatttgagcgtgccgaagtaccgaaaacaggcgactcccttgccatggcaaggcgtttggcgcgcagtgttcgccgcctgacccgccgtcgcgcccaccgcctgcttcggacccgccgcctattgaaacgcgaaggcgtattacaagccgccaattttgacgaaaacggcttgattaaatccttaccgaatacaccatggcaacttcgcgcagccgcattagaccgcaaactgacgcctttagagtggtcggcagtcttgttgcatttaatcaaacatcgcggctatttatcgcaacggaaaaacgagggcgaaactgccgataaggagcttggcgctttgcttaaaggcgtagccggcaatgcccatgccttacagacaggcgatttccgcacaccggccgaattggctttaaataaatttgagaaagaaagcggccatatccgcaatcagcgcagcgattattcgcatacgttcagccgcaaagatttacaggcggagctgattttgctgtttgaaaaacaaaaagaatttggcaatccgcatgtttcaggcggccttaaagaaggtattgaaaccctactgatgacgcaacgccctgccctgtccggcgatgccgttcaaaaaatgttggggcattgcaccttcgaaccggcagagccgaaagccgctaaaaacacctacacagccgaacgtttcatctggctgaccaagctgaacaacctgcgtattttagagcaaggcagcgagcggccattgaccgataccgaacgcgccacgcttatggacgagccatacagaaaatccaaactgacttacgcacaagcccgtaagctgctgggtttagaagataccgcctttttcaaaggcttgcgctatggtaaagacaatgccgaagcctcaacattgatggaaatgaaggcctaccatgccatcagccgtgcactggaaaaagaaggattgaaagacaaaaaatccccattaaacctttctcccgaattacaagacgaaatcggcacggcattctccctgttcaaaaccgatgaagacattacaggccgtctgaaagaccgtatacagcccgaaatcttagaagcgctgttgaaacacatcagcttcgataagttcgtccaaatttccttgaaagcattgcgccgaattgtgcctctaatggaacaaggcaaacgttacgatgaagcctgcgccgaaatctacggagaccattacggcaagaagaatacggaagaaaagatttatctgccgccgattcccgccgacgaaatccgcaaccccgtcgtcttgcgcgccttatctcaagcacgtaaggtcattaacggcgtggtacgccgttacggctccccagctcgtatccatattgaaactgcaagggaagtaggtaaatcgtttaaagaccgcaaagaaattgagaaacgccaagaagaaaaccgcaaagaccgggaaaaagccgccgccaaattccgagagtatttccccaattttgtcggagaacccaaatccaaagatattctgaaactgcgcctgtacgagcaacaacacggcaaatgcctgtattcgggcaaagaaatcaacttaggccgtctgaacgaaaaaggctatgtcgaaatcgaccatgccctgccgttctcgcgcacatgggacgacagtttcaacaataaagtactggtattgggcagcgaaaaccaaaacaaaggcaatcaaaccccttacgaatacttcaacggcaaagacaacagccgcgaatggcaggaatttaaagcgcgtgtcgaaaccagccgtttcccgcgcagtaaaaaacaacggattctgctgcaaaaattcgatgaagacggctttaaagaacgcaatctgaacgacacgcgctacgtcaaccgtttcctgtgtcaatttgttgccgaccgtatgcggctgacaggtaaaggcaagaaacgtgtctttgcatccaacggacaaattaccaatctgttgcgcggcttttggggattgcgcaaagtgcgtgcggaaaacgaccgccatcacgccttggacgccgtcgtcgttgcctgctcgaccgttgccatgcagcagaaaattacccgttttgtacgctataaagagatgaacgcgtttgacggtaaaaccatagacaaagaaacaggagaagtgctgcatcaaaaaacacacttcccacaaccttgggaatttttcgcacaagaagtcatgattcgcgtcttcggcaaaccggacggcaaacccgaattcgaagaagccgataccctagaaaaactgcgcacgttgcttgccgaaaaattatcatctcgccccgaagccgtacacgaatacgttacgccactgtttgtttcacgcgcgcccaatcggaagatgagcgggcaagggcatatggagaccgtcaaatccgccaaacgactggacgaaggcgtcagcgtgttgcgcgtaccgctgacacagttaaaactgaaagacttggaaaaaatggtcaatcgggagcgcgaacctaagctatacgaagcactgaaagcacggctggaagcacataaagacgatcctgccaaagcctttgccgagccgttttacaaatacgataaagcaggcaaccgcacccaacaggtaaaagccgtacgcgtagagcaagtacagaaaaccggcgtatgggtgcgcaaccataacggtattgccgacaacgcaaccatggtgcgcgtagatgtgtttgagaaaggcgacaagtattatctggtaccgatttacagttggcaggtagcgaaagggattttgccggatagggctgttgtacaaggaaaagatgaagaagattggcaacttattgatgatagtttcaactttaaattctcattacaccctaatgatttagtcgaggttataacaaaaaaagctagaatgtttggttactttgccagctgccatcgaggcacaggtaatatcaatatacgcattcatgatcttgatcataaaattggcaaaaatggaatactggaaggtatcggcgtcaaaaccgccctttcattccaaaaataccaaattgacgaactgggcaaagaaatcagaccatgccgtctgaaaaaacgcccgcctgtccgttacccatacgatgttccagattacgctgcagctccagcagcgaagaaaaagaagctggattaa

(19) United States
(12) Patent Application Publication
(10) Pub. No.: US 2024/0067992 A1
(43) Pub. Date: Feb. 29, 2024

(54) HUMAN HEMATOPOIETIC STEM CELL HAVING A MODIFIED BCL11A GENE AND METHODS OF MAKING THE CELL

(71) Applicant: Editas Medicine, Inc., Cambridge, MA (US)

(72) Inventors: Alexandra GLUCKSMANN, Lexington, MA (US); Deborah PALESTRANT, Newton, MA (US); Louis Anthony TARTAGLIA, Newton, MA (US); Jordi MATA-FINK, Sumerville, MA (US); Agnieszka Dorota CZECHOWICZ, Boston, MA (US)

(73) Assignee: Editas Medicine, Inc., Cambridge, MA (US)

(21) Appl. No.: 18/450,549

(22) Filed: Aug. 16, 2023

Related U.S. Application Data
(63) Continuation of application No. 16/938,661, filed on Jul. 24, 2020.
Application No. 16/938,661 is a continuation of application No. 15/025,222, filed on Mar. 25, 2016, now abandoned.
(60) Provisional application No. 61/883,925, filed on Sep. 27, 2013.
(60) Provisional application No. 61/898,043, filed on Oct. 31, 2013.

(51) Int. Cl. C12N 15/90; A61K 38/46; A61K 47/54; A61K 48/00; C12N 9/22; C12N 9/96; C12N 15/11; C12N 15/82; C12N 15/85

(52) U.S. Cl. CPC C12N 15/907; A61K 38/465; A61K 47/549; A61K 48/005; C12N 9/22; C12N 9/96; C12N 15/11; C12N 15/8216; C12N 15/85; C12N 15/902; C12N 2310/3513; C12N 2320/33; C12Y 301/00; Y02A 50/30

(57) ABSTRACT

Methods and compositions useful in targeting a payload to or editing target nucleic acid are disclosed herein.

### CROSS-REFERENCE TO RELATED APPLICATIONS

This application is a Continuation of U.S. application Ser. No. 16/938,661, filed Jul. 24, 2020, which is a Continuation of U.S. application Ser. No. 15/025,222, filed Mar. 25, 2016, now abandoned, which is a 371 National Stage Entry of International Application Serial No. PCT/US2014/057905, filed Sep. 26, 2014, which claims priority of U.S. Provisional Patent Application No. 61/883,925, filed on Sep. 27, 2013 and U.S. Provisional Patent Application No. 61/898,043, filed on Oct. 31, 2013, the disclosures of which are incorporated herein by reference in their entireties.

### FIELD OF THE INVENTION

The invention relates to CRISPR-related methods and components for editing of, or delivery of a payload to, a target nucleic acid sequence.

### SEQUENCE LISTING

The present application includes a Sequence Listing filed in electronic format. The Sequence Listing is entitled “4417-101US5.xml” created on Oct. 4, 2023, and is 450,560 bytes in size. The information in the electronic format of the Sequence Listing is part of the present application and is incorporated herein by reference in its entirety.

### BACKGROUND OF THE INVENTION

CRISPRs (Clustered Regularly Interspaced Short Palindromic Repeats) evolved in bacteria as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complimentary to the viral genome, mediates targeting of a Cas9 protein to the sequence in the viral genome. The Cas9 protein cleaves and thereby silences the viral target.

Recently, the CRISPR/Cas system has been adapted for genome editing in eukaryotic cells. The introduction of site-specific double strand breaks (DSBs) allows for target sequence alteration through one of two endogenous DNA repair mechanisms—either non-homologous end-joining (NHEJ) or homology-directed repair (HDR). The CRISPR/Cas system has also been used for gene regulation including transcription repression and activation without altering the target sequence. Targeted gene regulation based on the CRISPR/Cas system uses an enzymatically inactive Cas9 (also known as a catalytically dead Cas9).

### SUMMARY OF THE INVENTION

Methods and compositions disclosed herein, e.g., a Cas9 molecule complexed with a gRNA molecule, can be used to target a specific location in a target DNA. Depending on the Cas9 molecule/gRNA molecule complex used specific editing or the delivery of a payload can be effected.

In one aspect, the disclosure features a gRNA molecule comprising a targeting domain which is complementary with a target sequence from a target nucleic acid disclosed herein, e.g., a sequence from: a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In another aspect, the disclosure features a composition, e.g., pharmaceutical composition, comprising a gRNA molecule described herein.

In some embodiments, the composition further comprises a Cas9 molecule, e.g., an eaCas9 or an eiCas9 molecule. In some embodiments, said Cas9 molecule is an eaCas9 molecule. In other embodiments, said Cas9 molecule is an eiCas9 molecule.

In some embodiments, said composition comprises a payload, e.g., a payload described herein, e.g., in Section VI, e.g., in Table VI-1, VI-2, VI-3, VI-4, VI-5, VI-6, or VI-7.

In some embodiments, the payload comprises: an epigenetic modifier, e.g., a molecule that modifies DNA or chromatin; component, e.g., a molecule that modifies a histone, e.g., an epigenetic modifier described herein, e.g., in Section VI; a transcription factor, e.g., a transcription factor described herein, e.g., in Section VI; a transcriptional activator domain; an inhibitor of a transcription factor, e.g., an anti-transcription factor antibody, or other inhibitors; a small molecule; an antibody; an enzyme; an enzyme that interacts with DNA, e.g., a helicase, restriction enzyme, ligase, or polymerase; and/or a nucleic acid, e.g., an enzymatically active nucleic acid, e.g., a ribozyme, or an mRNA, siRNA, of antisense oligonucleotide. In some embodiments, the composition further comprises a Cas9 molecule, e.g., an eiCas9, molecule.

In some embodiments, said payload is coupled, e.g., covalently or noncovalently, to a Cas9 molecule, e.g., an eiCas9 molecule. In some embodiments, said payload is coupled to said Cas9 molecule by a linker. In some embodiments, said linker is or comprises a bond that is cleavable under physiological, e.g., nuclear, conditions. In some embodiments, said linker is, or comprises, a bond described herein, e.g., in Section XI. In some embodiments, said linker is, or comprises, an ester bond. In some embodiments, said payload comprises a fusion partner fused to a Cas9 molecule, e.g., an eaCas9 molecule or an eiCas9 molecule.

In some embodiments, said payload is coupled, e.g., covalently or noncovalently, to the gRNA molecule. In some embodiments, said payload is coupled to said gRNA molecule by a linker. In some embodiments, said linker is or comprises a bond that is cleavable under physiological, e.g., nuclear, conditions. In some embodiments, said linker is, or comprises, a bond described herein, e.g., in Section XI. In some embodiments, said linker is, or comprises, an ester bond.

In some embodiments, the composition comprises an eaCas9 molecule. In some embodiments, the composition comprises an eaCas9 molecule which forms a double stranded break in the target nucleic acid.

In some embodiments, the composition comprises an eaCas9 molecule which forms a single stranded break in the target nucleic acid. In some embodiments, said single stranded break is formed in the complementary strand of the target nucleic acid. In some embodiments, said single stranded break is formed in the strand which is not the complementary strand of the target nucleic acid.

In some embodiments, the composition comprises HNH-like domain cleavage activity but having no, or no significant, N-terminal RuvC-like domain cleavage activity. In some embodiments, the composition comprises N-terminal RuvC-like domain cleavage activity but having no, or no significant, HNH-like domain cleavage activity.

In some embodiments, said double stranded break is within 10, 20, 30, 40, 50, 100, 150 or 200 nucleotides of a nucleotide of the target position. In some embodiments, said single stranded break is within 10, 20, 30, 40, 50, 100, 150 or 200 nucleotides of a nucleotide of the target position.

In some embodiments, the composition further comprises a template nucleic acid, e.g., a template nucleic acid described herein, e.g., in Section IV. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the composition further comprises a second gRNA molecule, e.g., a second gRNA molecule described herein.

In some embodiments, said gRNA molecule and said second gRNA molecule mediate breaks at different sites in the target nucleic acid, e.g., flanking a target position. In some embodiments, said gRNA molecule and said second gRNA molecule are complementary to the same strand of the target. In some embodiments, said gRNA molecule and said second gRNA molecule are complementary to the different strands of the target.

In some embodiments, said Cas9 molecule mediates a double stranded break.

In some embodiments, said gRNA molecule and said second gRNA molecule are configured such that first and second break made by the Cas9 molecule flank a target position. In some embodiments, said double stranded break is within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of a target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, said Cas9 molecule mediates a single stranded break.

In some embodiments, said gRNA molecule and said second gRNA molecule are configured such that a first and second break are formed in the same strand of the nucleic acid target, e.g., in the case of transcribed sequence, the template strand or the non-template strand.

In some embodiments, said first and second break flank a target position.

In some embodiments, one of said first and second single stranded breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position. In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, said gRNA molecule and said second gRNA molecule are configured such that a first and a second breaks are formed in different strands of the target. In some embodiments, said first and second break flank a target position. In some embodiments, one of said first and second single stranded breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the composition comprises a second Cas9 molecule.

In some embodiments, one or both of said Cas9 molecule and said second Cas9 molecule are eiCas9 molecules. In some embodiments, said eiCas9 molecule is coupled to a payload by a linker and said second eiCas9 molecules is coupled to a second payload by a second linker.

In some embodiments, said payload and said second payload are the same. In some embodiments, said payload and said second payload are different. In some embodiments, said linker and said second linker are the same. In some embodiments, said linker and said second linker are different, e.g., have different release properties, e.g., different release rates.

In some embodiments, said payload and said second payload are each described herein, e.g., in Section VI, e.g., in Table VI-1, VI-2, VI-3, VI-4, VI-5, VI-6, or VI-7. In some embodiments, said payload and said second payload can interact, e.g., they are subunits of a protein.

In some embodiments, one of both of said Cas9 molecule and said second Cas9 molecule are eaCas9 molecules.

In some embodiments, said eaCas9 molecule comprises a first cleavage activity and said second eaCas9 molecule comprises a second cleavage activity. In some embodiments, said cleavage activity and said second cleavage activity are the same, e.g., both are N-terminal RuvC-like domain activity or are both HNH-like domain activity. In some embodiments, said cleavage activity and said second cleavage activity are different, e.g., one is N-terminal RuvC-like domain activity and one is HNH-like domain activity.

In some embodiments, said Cas9 molecule and said second Cas9 molecule are specific for different PAMs, e.g., one is specific for NGG and the other is specific for, e.g., NGGNG, NNAGAAW (W=A or T), or NAAR (R=A or G).

In some embodiments, said Cas9 molecule and said second Cas9 molecule both mediate double stranded breaks.

In some embodiments, said Cas9 molecule and said second Cas9 molecule are specific for different PAMs, e.g., one is specific for NGG and the other is specific for another PAM, e.g., another PAM described herein. In some embodiments, said gRNA molecule and said second gRNA molecule are configured such that first and second break flank a target position. In some embodiments, one of said first and second double stranded breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, one of said Cas9 molecule and said second Cas9 molecule mediates a double stranded break and the other mediates a single stranded break.

In some embodiments, said Cas9 molecule and said second Cas9 molecule are specific for different PAMs, e.g., one is specific for NGG and the other is specific for another PAM, e.g., another PAM described herein. In some embodiments, said gRNA molecule and said second gRNA molecule are configured such that a first and second break flank a target position. In some embodiments, said first and second break flank a target position. In some embodiments, one of said first and second breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, said Cas9 molecule and said second Cas9 molecule both mediate single stranded breaks.

In some embodiments, said Cas9 molecule and said second Cas9 molecule are specific for different PAMs, e.g., one is specific for NGG and the other is specific for another PAM, e.g., another PAM described herein. In some embodiments, said first and second break flank a target position.

In some embodiments, one of said first and second single stranded breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, said gRNA molecule, said second gRNA molecule are configured such that a first and second break are in the same strand.

In some embodiments, said Cas9 molecule and said second Cas9 molecule are specific for different PAMs, e.g., one is specific for NGG and the other is specific for another PAM, e.g., another PAM described herein. In some embodiments, said gRNA molecule, said second gRNA molecule are configured such that a first and second break flank a target position. In some embodiments, one of said first and second single stranded breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, said first and second break are on the different strands.

In some embodiments, said Cas9 molecule and said second Cas9 molecule are specific for different PAMs, e.g., one is specific for NGG and the other is specific for another PAM, e.g., another Pam described herein. In some embodiments, said gRNA molecule, said second gRNA molecule are configured such that a first and second break are on different strands.

In some embodiments, said gRNA molecule, said second gRNA molecule are configured such that a first and second break flank a target position. In some embodiments, said first and second break flank a target position.

In some embodiments, one of said first and second single stranded breaks, or both are independently, within 10, 20, 30, 40, 50, 100, 150 or 200 nucleotides of a nucleotide of the target position.

In some embodiments, the composition further comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In yet another aspect, the disclosure features a composition, e.g., a pharmaceutical composition, comprising a gRNA molecule and a second gRNA molecule described herein.

In some embodiments, the composition further comprises a nucleic acid, e.g., a DNA or mRNA, that encodes a Cas9 molecule described herein. In some embodiments, the composition further comprises a nucleic acid, e.g., a DNA or RNA, that encodes a second Cas9 molecule described herein. In some embodiments, the composition further comprises a template nucleic acid described herein.

In one aspect, the disclosure features a composition, e.g., a pharmaceutical composition, comprising, nucleic acid sequence, e.g., a DNA, that encodes one or more gRNA molecules described herein.

In some embodiments, said nucleic acid comprises a promoter operably linked to the sequence that encodes a gRNA molecule, e.g., a promoter described herein.

In some embodiments, said nucleic acid comprises a second promoter operably linked to the sequence that encodes a second gRNA molecule, e.g., a promoter described herein. In some embodiments, the promoter and second promoter are different promoters. In some embodiments, the promoter and second promoter are the same.

In some embodiments, the nucleic acid further encodes a Cas9 molecule described herein. In some embodiments, the nucleic acid further encodes a second Cas9 molecule described herein.

In some embodiments, said nucleic acid comprises a promoter operably linked to the sequence that encodes a Cas9 molecule, e.g., a promoter described herein.

In some embodiments, said nucleic acid comprises a second promoter operably linked to the sequence that encodes a second Cas9 molecule, e.g., a promoter described herein. In some embodiments, the promoter and second promoter are different promoters. In some embodiments, the promoter and second promoter are the same.

In some embodiments, the composition further comprises a template nucleic acid e.g., a template nucleic acid described herein, e.g., in Section IV.

In another aspect, the disclosure features a composition, e.g., a pharmaceutical composition, comprising nucleic acid sequence that encodes one or more of: a) a Cas9 molecule, b) a second Cas9 molecule, c) a gRNA molecule, and d) a second gRNA molecule.

In some embodiments, each of a), b), c) and d) present are encoded on the same duplex molecule.

In some embodiments, a first sequence selected from of a), b), c) and d) is encoded on a first duplex molecule and a second sequence selected from a), b), c), and d) is encoded on a second duplex molecule.

In some embodiments, said nucleic acid encodes: a) and c); a), c), and d); or a), b), c), and d).

In some embodiments, the composition further comprises a Cas9 molecule, e.g., comprising one or more of the Cas9 molecules wherein said nucleic acid does not encode a Cas9 molecule.

In some embodiments, the composition further comprises an mRNA encoding Cas9 molecule, e.g., comprising one or more mRNAs encoding one or more of the Cas9 molecules wherein said nucleic acid does not encode a Cas9 molecule.

In some embodiments, the composition further comprises a template nucleic acid e.g., a template nucleic acid described herein, e.g., in Section IV.

In yet another aspect, the disclosure features a nucleic acid described herein.

In one aspect, the disclosure features a composition comprising: a) a gRNA molecule (or combination of gRNA molecules, e.g., a gRNA molecule and a second gRNA molecule); b) an eaCas9 molecule (or combination of eaCas9 molecules, e.g., an eaCas9 molecule; and a second eaCas9 molecule); and c) optionally, a template nucleic acid e.g., a template nucleic acid described herein, e.g., in Section IV.

In another aspect, the disclosure features a composition comprising: a) a gRNA molecule (or combination of gRNA molecules, e.g., a gRNA molecule and a second gRNA molecule); b) a nucleic acid, e.g. a DNA or mRNA encoding an eaCas9 molecule (or combination of eaCas9 molecules, e.g., an eaCas9 molecule and a second eaCas9 molecule); and c) optionally, a template nucleic acid, e.g., a template nucleic acid described herein, e.g., in Section IV.

In yet another aspect, the disclosure features a composition comprising: a) a nucleic acid, e.g., a DNA, which encodes a gRNA molecule (or combination of gRNA molecules, e.g., a gRNA molecule and a second gRNA molecule); b) an eaCas9 molecule (or combination of eaCas9 molecules, e.g., an eaCas9 molecule and a second eaCas9 molecule); and c) optionally, a template nucleic acid, e.g., a template nucleic acid described herein, e.g., in Section IV.

In still another aspect, the disclosure features a composition comprising: a) nucleic acid, e.g., a DNA, which encodes a gRNA molecule or (or combination of gRNA molecules, e.g., a gRNA molecule and a second gRNA molecule); b) nucleic acid, e.g. a DNA or mRNA encoding eaCas9 molecule or (or combination of eaCas9 molecules, e.g., an eaCas9 molecule and a second eaCas9 molecule) (wherein the gRNA molecule encoding nucleic acid and the eaCas9 molecule encoding nucleic acid can be on the same or different molecules); and c) optionally, a template nucleic acid, e.g., a template nucleic acid described herein, e.g., in Section IV.

In one aspect, the disclosure features a method of altering a cell, e.g., altering the structure, e.g., sequence, of a target nucleic acid of a cell, comprising contacting said cell with:

1) a composition comprising:
a) a gRNA molecule (or combination of gRNA molecules, e.g., a gRNA molecule and a second gRNA molecule);
b) an eaCas9 molecule (or combination of eaCas9 molecules, e.g., an eaCas9 molecule; and a second eaCas9 molecule); and
c) optionally, a template nucleic acid, e.g., a template nucleic acid described herein, e.g., in Section IV;

2) a composition comprising:
a) a gRNA molecule (or combination of gRNA molecules, e.g., a gRNA molecule and a second gRNA molecule);
b) a nucleic acid, e.g. a DNA or mRNA encoding an eaCas9 molecule (or combination of eaCas9 molecules, e.g., an eaCas9 molecule and a second eaCas9 molecule); and
c) optionally, a template nucleic acid, e.g., a template nucleic acid described herein, e.g., in Section IV;

3) a composition comprising:
a) a nucleic acid, e.g., a DNA, which encodes a gRNA molecule (or combination of gRNA molecules, e.g., a gRNA molecule and a second gRNA molecule);
b) an eaCas9 molecule (or combination of eaCas9 molecules, e.g., an eaCas9 molecule and a second eaCas9 molecule); and
c) optionally, a template nucleic acid, e.g., a template nucleic acid described herein, e.g., in Section IV; or

4) a composition comprising:
a) nucleic acid, e.g., a DNA, which encodes a gRNA molecule or (or combination of gRNA molecules, e.g., a gRNA molecule and a second gRNA molecule);
b) nucleic acid, e.g. a DNA or mRNA encoding eaCas9 molecule or (or combination of eaCas9 molecules, e.g., an eaCas9 molecule and a second eaCas9 molecule), (wherein the gRNA molecule encoding nucleic acid and the eaCas9 molecule encoding nucleic acid can be on the same or different molecules); and
c) optionally, a template nucleic acid, e.g., a template nucleic acid described herein, e.g., in Section IV.

In some embodiments, a gRNA molecule or nucleic acid encoding a gRNA molecule, and an eaCas9 molecule, or nucleic acid encoding an eaCas9 molecule, are delivered in or by, one dosage form, mode of delivery, or formulation.

In some embodiments, a) a gRNA molecule or nucleic acid encoding a gRNA molecule is delivered in or by, a first dosage form, a first mode of delivery, or a first formulation; and b) an eaCas9 molecule, or nucleic acid encoding an eaCas9 molecule, is delivered in or by a second dosage form, second mode of delivery, or second formulation.

In some embodiments, the cell is an animal or plant cell. In some embodiments, the cell is a mammalian, primate, or human cell. In some embodiments, the cell is a human cell, e.g., a cell from described herein, e.g., in Section VIIA. In some embodiments, the cell is: a somatic cell, germ cell, prenatal cell, e.g., zygotic, blastocyst or embryonic, blastocyst cell, a stem cell, a mitotically competent cell, a meiotically competent cell. In some embodiments, the cell is a human cell, e.g., a cancer cell or other cell characterized by a disease or disorder.

In some embodiments, the target nucleic acid is a chromosomal nucleic acid. In some embodiments, the target nucleic acid is an organellar nucleic acid. In some embodiments, the target nucleic acid is a mitochondrial nucleic acid. In some embodiments, the target nucleic acid is a chloroplast nucleic acid.

In some embodiments, the cell is a cell of a disease causing organism, e.g., a virus, bacterium, fungus, protozoan, or parasite.

In some embodiments, the target nucleic acid is the nucleic acid of a disease causing organism, e.g., of a disease causing organism, e.g., a virus, bacterium, fungus, protozoan, or parasite.

In some embodiments, said method comprises: modulating the expression of a gene or inactivating a disease organism.

In some embodiments, said cell is a cell characterized by unwanted proliferation, e.g., a cancer cell. In some embodiments, said cell is a cell characterized by an unwanted genomic component, e.g., a viral genomic component. In some embodiments, the cell is a cell described herein, e.g., in Section IIA. In some embodiments, a control or structural sequence of at least, 2 3, 4, or 5 genes is altered.

In some embodiments, the target nucleic acid is a rearrangement, a kinase, a rearrangement that comprises a kinase, or a tumor suppressor.

In some embodiments, the method comprises cleaving a target nucleic acid within 10, 20, 30, 40, 50, 100, 150 or 200 nucleotides of a nucleotide of the target position. In some embodiments, said composition comprises a template nucleic acid.

In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII, 21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments,
a) a control region, e.g., a cis-acting or tans-acting control region, of a gene is cleaved;
b) the sequence of a control region, e.g., a cis-acting or tans-acting control region, of a gene is altered, e.g., by an alteration that modulates, e.g., increases or decreases, expression a gene under control of the control region, e.g., a control sequence is disrupted or a new control sequence is inserted;
c) the coding sequence of a gene is cleaved;
d) the sequence of a transcribed region, e.g., a coding sequence of a gene is altered, e.g., a mutation is corrected or introduced, an alteration that increases expression of or activity of the gene product is effected, e.g., a mutation is corrected; and/or
e) the sequence of a transcribed region, e.g., the coding sequence of a gene is altered, e.g., a mutation is corrected or introduced, an alteration that decreases expression of or activity of the gene product is effected, e.g., a mutation is inserted, e.g., the sequence of one or more nucleotides is altered so as to insert a stop codon.

In some embodiments, a control region or transcribed region, e.g., a coding sequence, of at least 2, 3, 4, 5, or 6 genes are altered.

In another aspect, the disclosure features a method of treating a subject, e.g., by altering the structure, e.g., altering the sequence, of a target nucleic acid, comprising administering to the subject, an effective amount of:

1) a composition comprising:
a) a gRNA molecule (or combination of gRNA molecules, e.g., a gRNA molecule and a second gRNA molecule);
b) an eaCas9 molecule (or combination of eaCas9 molecules, e.g., an eaCas9 molecule; and a second eaCas9 molecule); and
c) optionally, a template nucleic acid, e.g., a template nucleic acid described herein, e.g., in Section IV;

2) a composition comprising:
a) a gRNA molecule (or combination of gRNA molecules, e.g., a gRNA molecule and a second gRNA molecule);
b) a nucleic acid, e.g. a DNA or mRNA encoding an eaCas9 molecule (or combination of eaCas9 molecules, e.g., an eaCas9 molecule and a second eaCas9 molecule); and
c) optionally, a template nucleic acid, e.g., a template nucleic acid described herein, e.g., in Section IV;

3) a composition comprising:
a) a nucleic acid, e.g., a DNA, which encodes a gRNA molecule (or combination of gRNA molecules, e.g., a gRNA molecule and a second gRNA molecule);
b) an eaCas9 molecule (or combination of eaCas9 molecules, e.g., an eaCas9 molecule and a second eaCas9 molecule); and
c) optionally, a template nucleic acid, e.g., a template nucleic acid described herein, e.g., in Section IV; and/or

4) a composition comprising:
a) nucleic acid, e.g., a DNA, which encodes a gRNA molecule or (or combination of gRNA molecules, e.g., a gRNA molecule and a second gRNA molecule);
b) nucleic acid, e.g. a DNA or mRNA encoding eaCas9 molecule or (or combination of eaCas9 molecules, e.g., an eaCas9 molecule and a second eaCas9 molecule), (wherein the gRNA molecule encoding nucleic acid and the eaCas9 molecule encoding nucleic acid can be on the same or different molecules); and
c) optionally, a template nucleic acid, e.g., a template nucleic acid described herein, e.g., in Section IV.

In some embodiments, a gRNA molecule or nucleic acid encoding a gRNA molecule, and an eaCas9 molecule, or nucleic acid encoding an eaCas9 molecule, are delivered in or by one dosage form, mode of delivery, or formulation.

In some embodiments, a gRNA molecule or nucleic acid encoding a gRNA molecule is delivered in or by a first dosage form, in a first mode of delivery, or first formulation; and an eaCas9 molecule, or nucleic acid encoding an eaCas9 molecule, is delivered in or by a second dosage form, second mode of delivery, or second formulation.

In some embodiments, the subject is an animal or plant. In some embodiments, the subject is a mammalian, primate, or human.

In some embodiments, the target nucleic acid is the nucleic acid of a human cell, e.g., a cell described herein, e.g., in Section VIIA. In some embodiments, the target nucleic acid is the nucleic acid of: a somatic cell, germ cell, prenatal cell, e.g., zygotic, blastocyst or embryonic, blastocyst cell, a stem cell, a mitotically competent cell, a meiotically competent cell.

In some embodiments, the target nucleic acid is a chromosomal nucleic acid. In some embodiments, the target nucleic acid is an organellar nucleic acid. In some embodiments, the nucleic acid is a mitochondrial nucleic acid. In some embodiments, the nucleic acid is a chloroplast nucleic acid.

In some embodiments, the target nucleic acid is the nucleic acid of a disease causing organism, e.g., of a disease causing organism, e.g., a virus, bacterium, fungus, protozoan, or parasite. In some embodiments, said method comprises modulating expression of a gene or inactivating a disease organism.

In some embodiments, the target nucleic acid is the nucleic acid of a cell characterized by unwanted proliferation, e.g., a cancer cell. In some embodiments, said target nucleic acid comprises an unwanted genomic component, e.g., a viral genomic component. In some embodiments, a control or structural sequence of at least, 2 3, 4, or 5 genes is altered. In some embodiments, the target nucleic acid is a rearrangement, a kinase, a rearrangement that comprises a kinase, or a tumor suppressor.

In some embodiments, the method comprises cleaving a target nucleic acid within 10, 20, 30, 40, 50, 100, 150 or 200 nucleotides of a nucleotide of the target position.

In some embodiments, said composition comprises a template nucleic acid. In some embodiments, the template nucleic acid comprises a nucleotide that corresponds to a nucleotide of the target position.

In some embodiments, said template nucleic acid comprises a nucleotide that corresponds to a nucleotide of the target position from a sequence of: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length from a sequence in: a gene, or a gene from a pathway, described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

In some embodiments, the template nucleic acid is or comprises a fragment of 10 to 500, 10 to 400, 10 to 300, 10 to 200 nucleotides in length, which differs at at least 1 nucleotide, but not more than 5, 10, 20 or 30% of its nucleotides, from a corresponding sequence in:

In some embodiments,
a) a control region, e.g., a cis-acting or trans-acting control region, of a gene is cleaved;
b) the sequence of a control region, e.g., a cis-acting or trans-acting control region, of a gene is altered, e.g., by an alteration that modulates, e.g., increases or decreases, expression a gene under control of the control region, e.g., a control sequence is disrupted or a new control sequence is inserted;
c) the coding sequence of a gene is cleaved;
d) the sequence of a transcribed region, e.g., a coding sequence of a gene is altered, e.g., a mutation is corrected or introduced, an alteration that increases expression of or activity of the gene product is effected, e.g., a mutation is corrected;
e) the non-coding sequence of a gene or an intergenic region between genes is cleaved; and/or
f) the sequence of a transcribed region, e.g., the coding sequence of a gene is altered, e.g., a mutation is corrected or introduced, an alteration that decreases expression of or activity of the gene product is effected, e.g., a mutation is inserted, e.g., the sequence of one or more nucleotides is altered so as to insert a stop codon.

In some embodiments, a control region or transcribed region, e.g., a coding sequence, of at least 2, 3, 4, 5, or 6 genes are altered.

In one aspect, the disclosure features a composition comprising: a) a gRNA molecule (or combination of gRNA molecules, e.g., a gRNA molecule and a second gRNA molecule); b) a Cas9 molecule, e.g., an eiCas9 molecule (or combination of Cas9 molecules, e.g., an eiCas9 molecule and a second eiCas9 molecule); and c) a payload coupled, covalently or non-covalently, to a complex of the gRNA molecule and the Cas9 molecule, e.g., coupled to the Cas9 molecule or the gRNA molecule.

In another aspect, the disclosure features a composition comprising: a) a gRNA molecule (or combination of gRNA molecules, e.g., a gRNA molecule and a second gRNA molecule); b) a nucleic acid, e.g. a DNA or mRNA encoding a Cas9 molecule, e.g., an eiCas9 molecule (or combination of Cas9 molecules, e.g., an eiCas9 molecule and a second eiCas9 molecule); and c) a payload which is: coupled, covalently or non-covalently, the gRNA molecule; or a fusion partner with the Cas9 molecule.

In yet another aspect, the disclosure features a composition comprising: a) a nucleic acid, e.g., a DNA, which encodes a gRNA molecule (or combination of gRNA molecules, e.g., a gRNA molecule and a second gRNA molecule); b) a Cas9 molecule, e.g., an eiCas9 molecule (or combination of Cas9 molecules, e.g., an eiCas9 molecule and a second eiCas9 molecule); and c) a payload which is coupled, covalently or non-covalently, to the Cas9 molecule.

In still another aspect, the disclosure features a composition comprising: a) nucleic acid, e.g., a DNA, which encodes a gRNA molecule or (or combination of gRNA molecules, e.g., a gRNA molecule and a second gRNA molecule); b) nucleic acid, e.g. a DNA or mRNA, encoding a Cas9 molecule, e.g., an eiCas9 molecule (or combination of Cas9 molecules, e.g., an eiCas9 molecule and a second eiCas9 molecule) (wherein the gRNA molecule encoding nucleic acid and the eaCas9 molecule encoding nucleic acid can be on the same or different molecules); and c) a payload which is a fusion partner with the Cas9 molecule.

In one aspect, the disclosure features a method of delivering a payload to a cell, e.g., by targeting a payload to target nucleic acid, comprising contacting said cell with:

1) a composition comprising:
a) a gRNA molecule (or combination of gRNA molecules, e.g., a gRNA molecule and a second gRNA molecule);
b) a Cas9 molecule, e.g., an eiCas9 molecule (or combination of Cas9 molecules, e.g., an eiCas9 molecule and a second eiCas9 molecule); and
c) a payload coupled, covalently or non-covalently, to a complex of the gRNA molecule and the Cas9 molecule, e.g., coupled to the Cas9 molecule or the gRNA molecule;

2) a composition comprising:
a) a gRNA molecule (or combination of gRNA molecules, e.g., a gRNA molecule and a second gRNA molecule);
b) a nucleic acid, e.g. a DNA or mRNA encoding a Cas9 molecule, e.g., an eiCas9 molecule (or combination of Cas9 molecules, e.g., an eiCas9 molecule and a second eiCas9 molecule); and
c) a payload which is: coupled, covalently or non-covalently, the gRNA molecule; or a fusion partner with the Cas9 molecule;

3) a composition comprising:
a) a nucleic acid, e.g., a DNA, which encodes a gRNA molecule (or combination of gRNA molecules, e.g., a gRNA molecule and a second gRNA molecule);
b) a Cas9 molecule, e.g., an eiCas9 molecule (or combination of Cas9 molecules, e.g., an eiCas9 molecule and a second eiCas9 molecule); and
c) a payload which is coupled, covalently or non-covalently, to the Cas9 molecule; and/or

4) a composition comprising:
a) nucleic acid, e.g., a DNA, which encodes a gRNA molecule or (or combination of gRNA molecules, e.g., a gRNA molecule and a second gRNA molecule);
b) nucleic acid, e.g. a DNA or mRNA, encoding a Cas9 molecule, e.g., an eiCas9 molecule (or combination of Cas9 molecules, e.g., an eiCas9 molecule and a second eiCas9 molecule) (wherein the gRNA molecule encoding nucleic acid and the eaCas9 molecule encoding nucleic acid can be on the same or different molecules); and
c) a payload which is a fusion partner with the Cas9 molecule.

In some embodiments, a gRNA molecule or nucleic acid encoding a gRNA molecule, and an eaCas9 molecule, or nucleic acid encoding an eaCas9 molecule, are delivered in or by one dosage form, mode of delivery, or formulation.

In some embodiments, a gRNA molecule or nucleic acid encoding a gRNA molecule is delivered in or by a first dosage form, first mode of delivery, or first formulation; and a Cas9 molecule, or nucleic acid encoding a Cas9 molecule, is delivered in or by a second dosage form, second mode of delivery, or second formulation.

In some embodiments, the cell is an animal or plant cell. In some embodiments, the cell is a mammalian, primate, or human cell. In some embodiments, the cell is a human cell, e.g., a human cell described herein, e.g., in Section VIIA. In some embodiments, the cell is: a somatic cell, germ cell, prenatal cell, e.g., zygotic, blastocyst or embryonic, blastocyst cell, a stem cell, a mitotically competent cell, a meiotically competent cell. In some embodiments, the cell is a human cell, e.g., a cancer cell, a cell comprising an unwanted genetic element, e.g., all or part of a viral genome.

In some embodiments, the gRNA mediates targeting of a chromosomal nucleic acid. In some embodiments, the gRNA mediates targeting of a selected genomic signature. In some embodiments, the gRNA mediates targeting of an organellar nucleic acid. In some embodiments, the gRNA mediates targeting of a mitochondrial nucleic acid. In some embodiments, the gRNA mediates targeting of a chloroplast nucleic acid.

In some embodiments, the cell is a cell of a disease causing organism, e.g., a virus, bacterium, fungus, protozoan, or parasite.

In some embodiments, the gRNA mediates targeting of the nucleic acid of a disease causing organism, e.g., of a disease causing organism, e.g., a virus, bacterium, fungus, protozoan, or parasite.

In some embodiments, the payload comprises a payload described herein, e.g., in Section VI.

In some embodiments, said cell is a cell characterized by unwanted proliferation, e.g., a cancer cell. In some embodiments, said cell is characterized by an unwanted genomic component, e.g., a viral genomic component.

In some embodiments, a control or structural sequence of at least, 2 3, 4, or 5 genes is altered.

In some embodiments, the gRNA targets a selected genomic signature, e.g., a mutation, e.g., a germline or acquired somatic mutation. In some embodiments, the gRNA targets a rearrangement, a kinase, a rearrangement that comprises a kinase, or tumor suppressor. In some embodiments, the gRNA targets a cancer cell, e.g., a cancer cell disclosed herein, e.g., in Section VIIA. In some embodiments, the gRNA targets a cell which has been infected with a virus.

In another aspect, the disclosure features a method of treating a subject, e.g., by targeting a payload to target nucleic acid, comprising administering to the subject, an effective amount of:

1) a composition comprising:
a) a gRNA molecule (or combination of gRNA molecules, e.g., a gRNA molecule and a second gRNA molecule);
b) a Cas9 molecule, e.g., an eiCas9 molecule (or combination of Cas9 molecules, e.g., an eiCas9 molecule and a second eiCas9 molecule); and
c) a payload coupled, covalently or non-covalently, to a complex of the gRNA molecule and the Cas9 molecule, e.g., coupled to the Cas9 molecule;

2) a composition comprising:
a) a gRNA molecule (or combination of gRNA molecules, e.g., a gRNA molecule and a second gRNA molecule);
b) a nucleic acid, e.g. a DNA or mRNA encoding a Cas9 molecule, e.g., an eiCas9 molecule (or combination of Cas9 molecules, e.g., an eiCas9 molecule and a second eiCas9 molecule); and
c) a payload which is:
   coupled, covalently or non-covalently, the gRNA molecule; or
   is a fusion partner with the Cas9 molecule;

3) a composition comprising:
a) a nucleic acid, e.g., a DNA, which encodes a gRNA molecule (or combination of gRNA molecules, e.g., a gRNA molecule and a second gRNA molecule);
b) a Cas9 molecule, e.g., an eiCas9 molecule (or combination of Cas9 molecules, e.g., an eiCas9 molecule and a second eiCas9 molecule); and
c) a payload which is coupled, covalently or non-covalently, to the Cas9 molecule; and/or

4) a composition comprising:
a) a nucleic acid, e.g., a DNA, which encodes a gRNA molecule or (or combination of gRNA molecules, e.g., a gRNA molecule and a second gRNA molecule);
b) a nucleic acid, e.g. a DNA or mRNA, encoding a Cas9 molecule, e.g., an eiCas9 molecule (or combination of Cas9 molecules, e.g., an eiCas9 molecule and a second eiCas9 molecule), (wherein the gRNA molecule encoding nucleic acid and the eaCas9 molecule encoding nucleic acid can be on the same or different molecules); and
c) a payload which is a fusion partner with the Cas9 molecule.

In some embodiments, a gRNA molecule or nucleic acid encoding a gRNA molecule, and an eaCas9 molecule, or nucleic acid encoding an eaCas9 molecule, are delivered in or by one dosage form, mode of delivery, or formulation.

In some embodiments, a gRNA molecule or nucleic acid encoding a gRNA molecule is delivered in or by a first dosage, mode of delivery form or formulation; and a Cas9 molecule, or nucleic acid encoding a Cas9 molecule, is delivered in or by a second dosage form, mode of delivery, or formulation.

In some embodiments, the subject is an animal or plant cell. In some embodiments, the subject is a mammalian, primate, or human cell.

In some embodiments, the gRNA mediates targeting of a human cell, e.g., a human cell described herein, e.g., in Section VIIA. In some embodiments, the gRNA mediates targeting of: a somatic cell, germ cell, prenatal cell, e.g., zygotic, blastocyst or embryonic, blastocyst cell, a stem cell, a mitotically competent cell, a meiotically competent cell. In some embodiments, the gRNA mediates targeting of a cancer cell or a cell comprising an unwanted genomic element, e.g., all or part of a viral genome. In some embodiments, the gRNA mediates targeting of a chromosomal nucleic acid. In some embodiments, the gRNA mediates targeting of a selected genomic signature. In some embodiments, the gRNA mediates targeting of an organellar nucleic acid. In some embodiments, the gRNA mediates targeting of a mitochondrial nucleic acid. In some embodiments, the gRNA mediates targeting of a chloroplast nucleic acid. In some embodiments, the gRNA mediates targeting of the nucleic acid of a disease causing organism, e.g., of a disease causing organism, e.g., a virus, bacterium, fungus, protozoan, or parasite. In some embodiments, the gRNA targets a cell characterized by unwanted proliferation, e.g., a cancer cell, e.g., a cancer cell from Section VIIA, e.g., from Table VII-11. In some embodiments, the gRNA targets a cell characterized by an unwanted genomic component, e.g., a viral genomic component.

In some embodiments, a control element, e.g., a promoter or enhancer, is targeted. In some embodiments, the gRNA targets a rearrangement, a kinase, a rearrangement that comprises a kinase, or a tumor suppressor. In some embodiments, the gRNA targets a selected genomic signature, e.g., a mutation, e.g., a germline or acquired somatic mutation.

In some embodiments, the gRNA targets a cancer cell. In some embodiments, the gRNA targets a cell which has been infected with a virus.

In some embodiments, at least one eaCas9 molecule and a payload are administered. In some embodiments, the payload comprises a payload described herein, e.g., in Section VI.

In one aspect, the disclosure features a reaction mixture comprising a composition described herein and a cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Headings, including numeric and alphabetical headings and subheadings, are for organization and presentation and are not intended to be limiting.

Other features and advantages of the invention will be apparent from the detailed description, drawings, and from the claims.

### BRIEF DESCRIPTION OF THE DRAWING

The Figures described below, that together make up the Drawing, are for illustration purposes only, not for limitation.

FIGS. 1A-1G are representations of several exemplary gRNAs.

FIG. 1A depicts a modular gRNA molecule derived in part (or modeled on a sequence in part) from Streptococcus pyogenes (S. pyogenes) as a duplexed structure (SEQ ID NOS 42 and 43, respectively, in order of appearance);

FIG. 1B depicts a unimolecular (or chimeric) gRNA molecule derived in part from S. pyogenes as a duplexed structure (SEQ ID NO: 44);

FIG. 1C depicts a unimolecular gRNA molecule derived in part from S. pyogenes as a duplexed structure (SEQ ID NO: 45);

FIG. 1D depicts a unimolecular gRNA molecule derived in part from S. pyogenes as a duplexed structure (SEQ ID NO: 46);

FIG. 1E depicts a unimolecular gRNA molecule derived in part from S. pyogenes as a duplexed structure (SEQ ID NO: 47);

FIG. 1F depicts a modular gRNA molecule derived in part from Streptococcus thermophilus (S. thermophilus) as a duplexed structure (SEQ ID NOS 48 and 49, respectively, in order of appearance);

FIG. 1G depicts an alignment of modular gRNA molecules of S. pyogenes and S. thermophilus (SEQ ID NOS 50-53, respectively, in order of appearance).

FIG. 2 depicts an alignment of Cas9 sequences from Chylinski et al., RNA BIOL. 2013; 10(5): 726-737. The N-terminal RuvC-like domain is boxed and indicated with a “Y”. The other two RuvC-like domains are boxed and indicated with a “B”. The HNH-like domain is boxed and indicated by a “G”. Sm: S. mutans (SEQ ID NO: 1); Sp: S. pyogenes (SEQ ID NO: 2); St: S. thermophilus (SEQ ID NO: 3); Li: L. innocua (SEQ ID NO: 4). Motif: this is a motif based on the four sequences: residues conserved in all four sequences are indicated by single letter amino acid abbreviation; “*” indicates any amino acid found in the corresponding position of any of the four sequences; and “-” indicates any amino acid, e.g., any of the 20 naturally occurring amino acids.

FIG. 3A shows an alignment of the N-terminal RuvC-like domain from the Cas9 molecules disclosed in Chylinski et al. (SEQ ID NOS 54-103, respectively, in order of appearance). The last line of FIG. 3A identifies 3 highly conserved residues.

FIG. 3B shows an alignment of the N-terminal RuvC-like domain from the Cas9 molecules disclosed in Chylinski et al. with sequence outliers removed (SEQ ID NOS 104-177, respectively, in order of appearance). The last line of FIG. 3B identifies 4 highly conserved residues.

FIG. 4A shows an alignment of the HNH-like domain from the Cas9 molecules disclosed in Chylinski et al. (SEQ ID NOS 178-252, respectively, in order of appearance). The last line of FIG. 4A identifies conserved residues.

FIG. 4B shows an alignment of the HNH-like domain from the Cas9 molecules disclosed in Chylinski et al. with sequence outliers removed (SEQ ID NOS 253-302, respectively, in order of appearance). The last line of FIG. 4B identifies 3 highly conserved residues.

FIG. 5 depicts an alignment of Cas9 sequences from S. pyogenes and Neisseria meningitidis (N. meningitidis). The N-terminal RuvC-like domain is boxed and indicated with a “Y”. The other two RuvC-like domains are boxed and indicated with a “B”. The HNH-like domain is boxed and indicated with a “G”. Sp: S. pyogenes; Nm: N. meningitidis. Motif: this is a motif based on the two sequences: residues conserved in both sequences are indicated by a single amino acid designation; “*” indicates any amino acid found in the corresponding position of any of the two sequences; “-” indicates any amino acid, e.g., any of the 20 naturally occurring amino acids, and “-” indicates any amino acid, e.g., any of the 20 naturally occurring amino acids, or absent.

FIG. 6 shows a nucleic acid sequence encoding Cas9 of N. meningitidis (SEQ ID NO: 303). Sequence indicated by an “R” is an SV40 NLS; sequence indicated as “G” is an HA tag; sequence indicated by an “O” is a synthetic NLS sequence. The remaining (unmarked) sequence is the open reading frame (ORF).

### DEFINITIONS

“Domain”, as used herein, is used to describe segments of a protein or nucleic acid. Unless otherwise indicated, a domain is not required to have any specific functional property.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein, in some embodiments, amino acid or nucleic acid “identity” is equivalent to amino acid or nucleic acid “homology”). The percent identity between the two sequences is a function of the number of identical positions shared by the sequences.

“Modulator”, as used herein, refers to an entity, e.g., a drug, that can alter the activity (e.g., enzymatic activity, transcriptional activity, or translational activity), amount, distribution, or structure of a subject molecule or genetic sequence. In an embodiment, modulation comprises cleavage, e.g., breaking of a covalent or non-covalent bond, or the forming of a covalent or non-covalent bond, e.g., the attachment of a moiety, to the subject molecule. In an embodiment, a modulator alters the, three dimensional, secondary, tertiary, or quaternary structure, of a subject molecule. A modulator can increase, decrease, initiate, or eliminate a subject activity.

“Large molecule”, as used herein, refers to a molecule having a molecular weight of at least 2, 3, 5, 10, 20, 30, 40, 50, 60, 70, 80, 90, or 100 kD. Large molecules include proteins, polypeptides, nucleic acids, biologics, and carbohydrates.

“Polypeptide”, as used herein, refers to a polymer of amino acids having less than 100 amino acid residues. In an embodiment, it has less than 50, 20, or 10 amino acid residues.

“Reference molecule”, e.g., a reference Cas9 molecule or reference gRNA, as used herein, refers to a molecule to which a subject molecule, e.g., a subject Cas9 molecule of subject gRNA molecule, e.g., a modified or candidate Cas9 molecule is compared. For example, a Cas9 molecule can be characterized as having no more than 10% of the nuclease activity of a reference Cas9 molecule. Examples of reference Cas9 molecules include naturally occurring unmodified Cas9 molecules, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology with the Cas9 molecule to which it is being compared. In an embodiment, the reference Cas9 molecule is a sequence, e.g., a naturally occurring or known sequence, which is the parental form on which a change, e.g., a mutation has been made.

“Replacement”, or “replaced”, as used herein with reference to a modification of a molecule does not require a process limitation but merely indicates that the replacement entity is present.

“Small molecule”, as used herein, refers to a compound having a molecular weight less than about 2 kD, e.g., less than about 2 kD, less than about 1.5 kD, less than about 1 kD, or less than about 0.75 kD.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human. In other embodiments, the subject is poultry.

“Treat”, “treating” and “treatment”, as used herein, mean the treatment of a disease in a mammal, e.g., in a human, including (a) inhibiting the disease, i.e., arresting or preventing its development; (b) relieving the disease, i.e., causing regression of the disease state; or (c) curing the disease.

“X” as used herein in the context of an amino acid sequence, refers to any amino acid (e.g., any of the twenty natural amino acids) unless otherwise specified.

### DETAILED DESCRIPTION

### I. gRNA Molecules

A gRNA molecule, as that term is used herein, refers to a nucleic acid that promotes the specific targeting or homing of a gRNA molecule/Cas9 molecule complex to a target nucleic acid. gRNA molecules can be unimolecular (having a single RNA molecule), sometimes referred to herein as “chimeric” gRNAs, or modular (comprising more than one, and typically two, separate RNA molecules). A gRNA molecule comprises a number of domains. The gRNA molecule domains are described in more detail below.

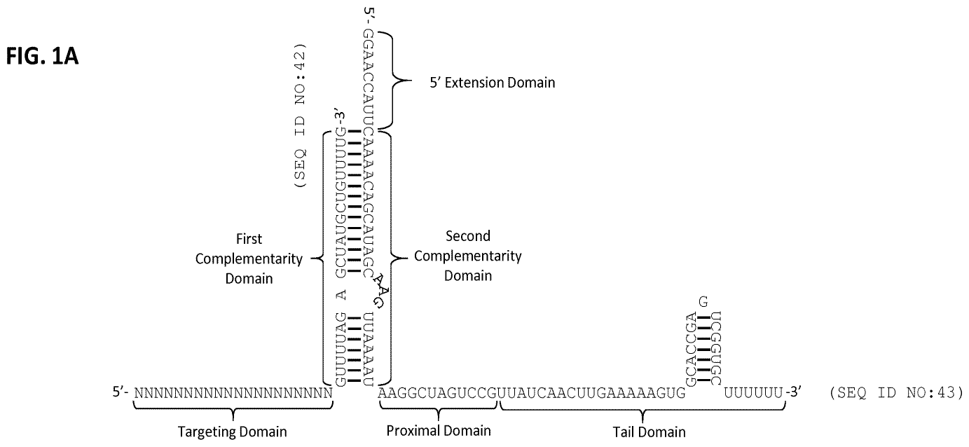

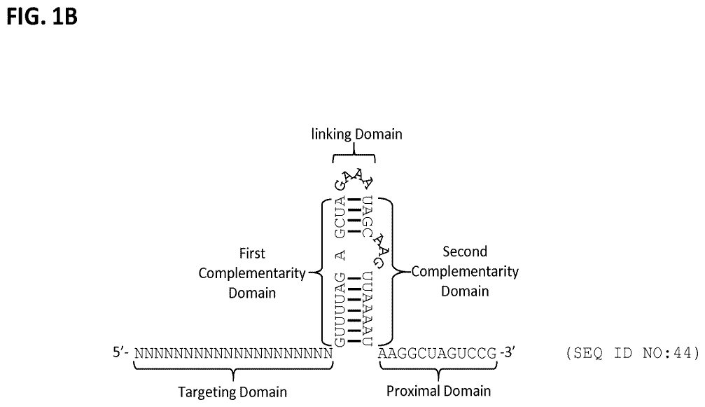

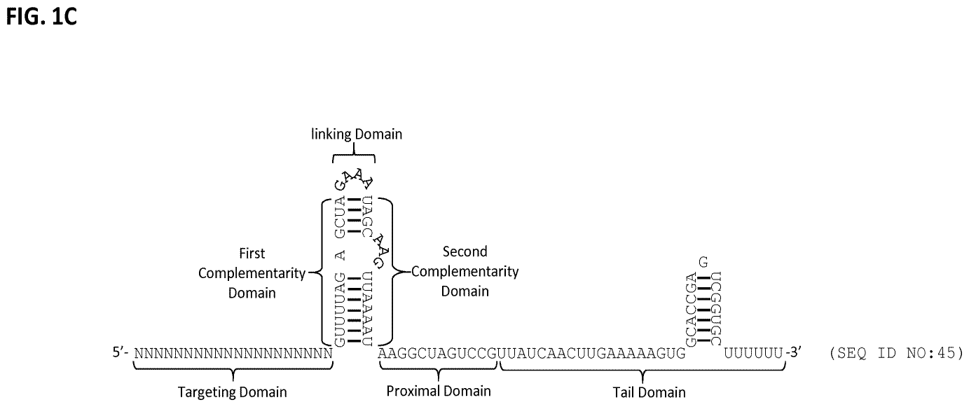

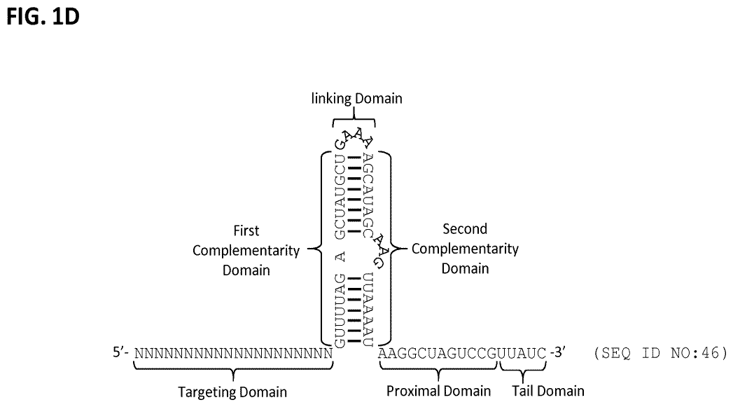

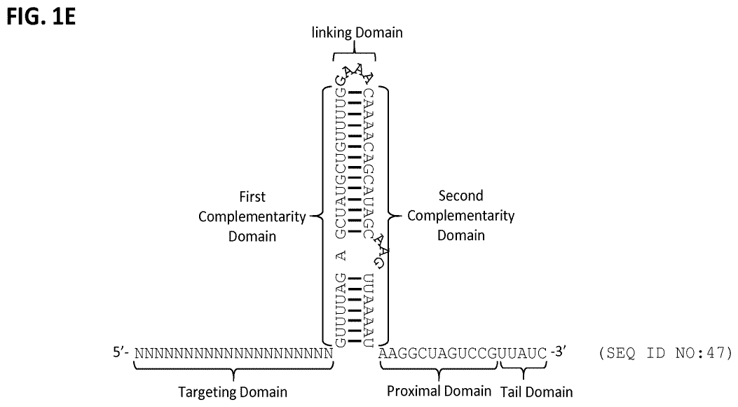

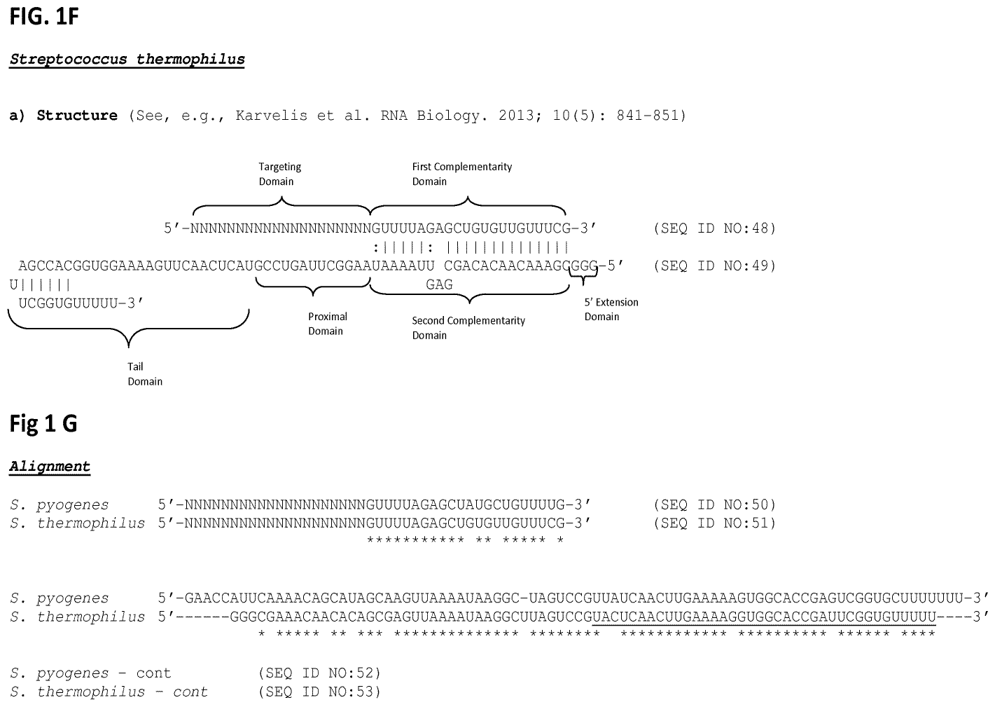

Several exemplary gRNA structures, with domains indicated thereon, are provided in FIG. 1. While not wishing to be bound by theory with regard to the three dimensional form, or intra- or inter-strand interactions of an active form of a gRNA, regions of high complementarity are sometimes shown as duplexes in FIG. 1 and other depictions provided herein.

In an embodiment, a unimolecular, or chimeric, gRNA comprises, preferably from 5′ to 3′:
a targeting domain (which is complementary to a target nucleic acid);
a first complementarity domain;
a linking domain;
a second complementarity domain (which is complementary to the first complementarity domain);
a proximal domain; and
optionally, a tail domain.

In an embodiment, a modular gRNA comprises:
a first strand comprising, preferably from 5′ to 3′;
   a targeting domain (which is complementary with a target sequence from a target nucleic acid disclosed herein, e.g., a sequence from: a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII); and
   a first complementarity domain; and
      a second strand, comprising, preferably from 5′ to 3′:
   optionally, a 5′ extension domain;
   a second complementarity domain; and
   a proximal domain; and
   optionally, a tail domain.

The domains are discussed briefly below:

1) The Targeting Domain:

FIGS. 1A-1G provide examples of the placement of targeting domains.

The targeting domain comprises a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In an embodiment, the target domain itself comprises, in the 5′ to 3′ direction, an optional secondary domain, and a core domain. In an embodiment, the core domain is fully complementary with the target sequence. In an embodiment, the targeting domain is 5 to 50, e.g., 10 to 40, e.g., 10 to 30, e.g., 15 to 30, e.g., 15 to 25 nucleotides in length. In an embodiment, the targeting domain is 15, 16, 17, 18, 19, 20, 21, 22, 23, 24 or 25 nucleotides in length. The strand of the target nucleic acid with which the targeting domain is complementary is referred to herein as the complementary strand. Some or all of the nucleotides of the domain can have a modification, e.g., modification found in Section X herein.

In an embodiment, the targeting domain is 16 nucleotides in length.

In an embodiment, the targeting domain is 17 nucleotides in length.

In an embodiment, the targeting domain is 18 nucleotides in length.

In an embodiment, the targeting domain is 19 nucleotides in length.

In an embodiment, the targeting domain is 20 nucleotides in length.

In an embodiment, the targeting domain is 21 nucleotides in length.

In an embodiment, the targeting domain is 22 nucleotides in length.

In an embodiment, the targeting domain is 23 nucleotides in length.

In an embodiment, the targeting domain is 24 nucleotides in length.

In an embodiment, the targeting domain is 25 nucleotides in length.

Targeting domains are discussed in more detail below.

2) The First Complementarity Domain:

FIGS. 1A-IG provides examples of first complementarity domains.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain is 5 to 25 nucleotides in length. In an embodiment, the first complementary domain is 7 to 25 nucleotides in length. In an embodiment, the first complementary domain is 7 to 22 nucleotides in length. In an embodiment, the first complementary domain is 7 to 18 nucleotides in length. In an embodiment, the first complementary domain is 7 to 15 nucleotides in length. In an embodiment, the first complementary domain is 5, 6, 7, 8, 9, 10, 11, 12, 13, 14, 15, 16, 17, 18, 19, 20, 21, 22, 23, 24, or 25 nucleotides in length.

In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4-9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4-22, 4-18, or 4 to 10, or 3, 4, 5, 6, 7, 8, 9, 10, 11, 12, 13, 14, 15, 16, 17, 18, 19, 20, 21, 22, 23, 24, or 25, nucleotides in length.

The first complementarity domain can share homology with, or be derived from, a naturally occurring first complementarity domain. In an embodiment, it has at least 50% homology with a first complementarity domain disclosed herein, e.g., an S. pyogenes, or S. thermophilus, first complementarity domain.

Some or all of the nucleotides of the domain can have a modification, e.g., modification found in Section X herein.

First complementarity domains are discussed in more detail below.

3) The Linking Domain

FIGS. 1B-1E provide examples of linking domains.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain covalently couples the first and second complementarity domains, see, e.g., FIGS. 1B-1E. In an embodiment, the linking domain is, or comprises, a covalent bond interposed between the first complementarity domain and the second complementarity domain. Typically, the linking domain comprises one or more, e.g., 2, 3, 4, 5, 6, 7, 8, 9, or 10 nucleotides.

In modular gRNA molecules the two molecules can be associated by virtue of the hybridization of the complementarity domains, see e.g., FIG. 1A.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length.

In an embodiment, a linking domain is 2, 3, 4, 5, 6, 7, 8, 9, 10, 15, 20, or 25 or more nucleotides in length. In an embodiment, a linking domain is 2 to 50, 2 to 40, 2 to 30, 2 to 20, 2 to 10, or 2 to 5 nucleotides in length. In an embodiment, a linking domain shares homology with, or is derived from, a naturally occurring sequence, e.g., the sequence of a tracrRNA that is 5′ to the second complementarity domain. In an embodiment, the linking domain has at least 50% homology with a linking domain disclosed herein.

Some or all of the nucleotides of the domain can have a modification, e.g., modification found in Section X herein.

Linking domains are discussed in more detail below.

4) The 5′ Extension Domain

In an embodiment, a modular gRNA can comprise additional sequence, 5′ to the second complementarity domain, referred to herein as the 5′ extension domain, see, e.g., FIG. 1A. In an embodiment, the 5′ extension domain is, 2-10, 2-9, 2-8, 2-7, 2-6, 2-5, 2-4 nucleotides in length. In an embodiment, the 5′ extension domain is 2, 3, 4, 5, 6, 7, 8, 9, or 10 or more nucleotides in length.

5) The Second Complementarity Domain:

FIGS. 1A-IF provide examples of second complementarity domains.

The second complementarity domain is complementary with the first complementarity domain, and in an embodiment, has sufficient complementarity to the second complementarity domain to form a duplexed region under at least some physiological conditions. In an embodiment, e.g., as shown in FIG. 1A or FIG. 1B, the second complementarity domain can include sequence that lacks complementarity with the first complementarity domain, e.g., sequence that loops out from the duplexed region.

In an embodiment, the second complementarity domain is 5 to 27 nucleotides in length. In an embodiment, it is longer than the first complementarity region.

In an embodiment, the second complementary domain is 7 to 27 nucleotides in length. In an embodiment, the second complementary domain is 7 to 25 nucleotides in length. In an embodiment, the second complementary domain is 7 to 20 nucleotides in length. In an embodiment, the second complementary domain is 7 to 17 nucleotides in length. In an embodiment, the complementary domain is 5, 6, 7, 8, 9, 10, 11, 12, 13, 14, 15, 16, 17, 18, 19, 20, 21, 22, 23, 24 or 25 nucleotides in length.

In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length.

In an embodiment, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In an embodiment, it has at least 50% homology with a second complementarity domain disclosed herein, e.g., an S. pyogenes, or S. thermophilus, first complementarity domain.

Some or all of the nucleotides of the domain can have a modification, e.g., modification found in Section X herein.

6) A Proximal Domain:

FIGS. 1A-IF provide examples of proximal domains.

In an embodiment, the proximal domain is 5 to 20 nucleotides in length. In an embodiment, the proximal domain can share homology with or be derived from a naturally occurring proximal domain. In an embodiment, it has at least 50% homology with a proximal domain disclosed herein, e.g., an S. pyogenes, or S. thermophilus, proximal domain.

Some or all of the nucleotides of the domain can have a modification, e.g., modification found in Section X herein.

7) A Tail Domain:

FIG. 1A and FIGS. 1C-1F provide examples of tail domains.

As can be seen by inspection of the tail domains in FIG. 1A and FIGS. 1C-1F, a broad spectrum of tail domains are suitable for use in gRNA molecules. In an embodiment, the tail domain is 0 (absent), 1, 2, 3, 4, 5, 6, 7, 8, 9, or 10 nucleotides in length. In an embodiment, the tail domain nucleotides are from or share homology with sequence from the 5′ end of a naturally occurring tail domain, see e.g., FIG. 1D or FIG. 1E. In an embodiment, the tail domain includes sequences that are complementary to each other and which, under at least some physiological conditions, form a duplexed region.

In an embodiment, the tail domain is absent or is 1 to 50 nucleotides in length. In an embodiment, the tail domain can share homology with or be derived from a naturally occurring proximal tail domain. In an embodiment, it has at least 50% homology with a tail domain disclosed herein, e.g., an S. pyogenes, or S. thermophilus, tail domain.

Some or all of the nucleotides of the domain can have a modification, e.g., modification found in Section X herein.

In an embodiment, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

The domains of gRNA molecules are described in more detail below.

The Targeting Domain

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. Guidance on the selection of targeting domains can be found, e.g., in Fu Y et al., NAT BIOTECHNOL 2014 (doi: 10.1038/nbt.2808) and Sternberg S H et al., NATURE 2014 (doi: 10.1038/nature13011).

In an embodiment, the targeting domain is 16, 17, 18, 19, 20, 21, 22, 23, 24 or 25 nucleotides in length.

In an embodiment, the targeting domain comprises 16, 17, 18, 19, 20, 21, 22, 23, 24 or 25 nucleotides in length.

In an embodiment, the targeting domain is 16 nucleotides in length.

In an embodiment, the targeting domain is 17 nucleotides in length.

In an embodiment, the targeting domain is 18 nucleotides in length.

In an embodiment, the targeting domain is 19 nucleotides in length.

In an embodiment, the targeting domain is 20 nucleotides in length.

In an embodiment, the targeting domain is 21 nucleotides in length.

In an embodiment, the targeting domain is 22 nucleotides in length.

In an embodiment, the targeting domain is 23 nucleotides in length.

In an embodiment, the targeting domain is 24 nucleotides in length.

In an embodiment, the targeting domain is 25 nucleotides in length.

In an embodiment, the targeting domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the targeting domain is 20+/−5 nucleotides in length.

In an embodiment, the targeting domain is 20+/−10, 30+/−10, 40+/−10, 50+/−10, 60+/−10, 70+/−10, 80+/−10, 90+/−10, or 100+/−10 nucleotides, in length.

In an embodiment, the targeting domain is 30+/−10 nucleotides in length.

In an embodiment, the targeting domain is 10 to 100, 10 to 90, 10 to 80, 10 to 70, 10 to 60, 10 to 50, 10 to 40, 10 to 30, 10 to 20 or 10 to 15 nucleotides in length. In other embodiments, the targeting domain is 20 to 100, 20 to 90, 20 to 80, 20 to 70, 20 to 60, 20 to 50, 20 to 40, 20 to 30, or 20 to 25 nucleotides in length.

Typically the targeting domain has full complementarity with the target sequence. In some embodiments the targeting domain has or includes 1, 2, 3, 4, 5, 6, 7 or 8 nucleotides that are not complementary with the corresponding nucleotide of the targeting domain.

In an embodiment, the target domain includes 1, 2, 3, 4 or 5 nucleotides that are complementary with the corresponding nucleotide of the targeting domain within 5 nucleotides of its 5′ end. In an embodiment, the target domain includes 1, 2, 3, 4 or 5 nucleotides that are complementary with the corresponding nucleotide of the targeting domain within 5 nucleotides of its 3′ end.

In an embodiment, the target domain includes 1, 2, 3, or 4 nucleotides that are not complementary with the corresponding nucleotide of the targeting domain within 5 nucleotides of its 5′ end. In an embodiment, the target domain includes 1, 2, 3, or 4 nucleotides that are not complementary with the corresponding nucleotide of the targeting domain within 5 nucleotides of its 3′ end.

In an embodiment, the degree of complementarity, together with other properties of the gRNA, is sufficient to allow targeting of a Cas9 molecule to the target nucleic acid.

In some embodiments, the targeting domain comprises two consecutive nucleotides that are not complementary to the target domain (“non-complementary nucleotides”), e.g., two consecutive noncomplementary nucleotides that are within 5 nucleotides of the 5′ end of the targeting domain, within 5 nucleotides of the 3′ end of the targeting domain, or more than 5 nucleotides away from one or both ends of the targeting domain.

In an embodiment, no two consecutive nucleotides within 5 nucleotides of the 5′ end of the targeting domain, within 5 nucleotides of the 3′ end of the targeting domain, or within a region that is more than 5 nucleotides away from one or both ends of the targeting domain, are not complementary to the targeting domain.

In an embodiment, there are no noncomplementary nucleotides within 5 nucleotides of the 5′ end of the targeting domain, within 5 nucleotides of the 3′ end of the targeting domain, or within a region that is more than 5 nucleotides away from one or both ends of the targeting domain.

In an embodiment, the targeting domain nucleotides do not comprise modifications, e.g., modifications of the type provided in Section X. However, in an embodiment, the targeting domain comprises one or more modifications, e.g., modifications that it render it less susceptible to degradation or more bio-compatible, e.g., less immunogenic. By way of example, the backbone of the targeting domain can be modified with a phosphorothioate, or other modification from Section X. In an embodiment, a nucleotide of the targeting domain can comprise a 2′ modification (e.g., a modification at the 2′ position on ribose), e.g., a 2′ acetylation, e.g., a 2′ methylation, or other modification from Section X.

In some embodiments, the targeting domain includes 1, 2, 3, 4, 5, 6, 7 or 8 or more modifications. In an embodiment, the targeting domain includes 1, 2, 3, or 4 modifications within 5 nucleotides of its 5′ end. In an embodiment, the targeting domain comprises as many as 1, 2, 3, or 4 modifications within 5 nucleotides of its 3′ end.

In some embodiments, the targeting domain comprises modifications at two consecutive nucleotides, e.g., two consecutive nucleotides that are within 5 nucleotides of the 5′ end of the targeting domain, within 5 nucleotides of the 3′ end of the targeting domain, or more than 5 nucleotides away from one or both ends of the targeting domain.

In an embodiment, no two consecutive nucleotides are modified within 5 nucleotides of the 5′ end of the targeting domain, within 5 nucleotides of the 3′ end of the targeting domain, or within a region that is more than 5 nucleotides away from one or both ends of the targeting domain. In an embodiment, no nucleotide is modified within 5 nucleotides of the 5′ end of the targeting domain, within 5 nucleotides of the 3′ end of the targeting domain, or within a region that is more than 5 nucleotides away from one or both ends of the targeting domain.

Modifications in the targeting domain can be selected so as to not interfere with targeting efficacy, which can be evaluated by testing a candidate modification in the system described in Section III. gRNA's having a candidate targeting domain having a selected length, sequence, degree of complementarity, or degree of modification, can be evaluated in a system in Section III. The candidate targeting domain can be placed, either alone, or with one or more other candidate changes in a gRNA molecule/Cas9 molecule system known to be functional with a selected target and evaluated.

In some embodiments, all of the modified nucleotides are complementary to and capable of hybridizing to corresponding nucleotides present in the target domain. In other embodiments, 1, 2, 3, 4, 5, 6, 7 or 8 or more modified nucleotides are not complementary to or capable of hybridizing to corresponding nucleotides present in the target domain.

In an embodiment, the targeting domain comprises, preferably in the 5′→3′ direction: a secondary domain and a core domain. These domains are discussed in more detail below.

The Core Domain and Secondary Domain of the Targeting Domain

The “core domain” of the targeting domain is complementary to the “core domain target” on the target nucleic acid. In an embodiment, the core domain comprises about 8 to about 13 nucleotides from the 3′ end of the targeting domain (e.g., the most 3′ 8 to 13 nucleotides of the targeting domain).

In an embodiment, the core domain is 6+/−2, 7+/−2, 8+/−2, 9+/−2, 10+/−2, 11+/−2, 12+/−2, 13+/−2, 14+/−2, 15+/−2, or 16+−2 nucleotides in length.

In an embodiment, the core domain is 10+/−2 nucleotides in length.

In an embodiment, the core domain is 10+/−4 nucleotides in length.

In an embodiment, the core domain is 6, 7, 8, 9, 10, 11, 12, 13, 14, 15, or 16 nucleotides in length.

In an embodiment, the core domain is 8 to 13, e.g., 8 to 12, 8 to 11, 8 to 10, 8 to 9, 9 to 13, 9 to 12, 9 to 11, or 9 to 10 nucleotides in length.

In an embodiment, the core domain is 6 to 16, e.g., 6 to 15, 6 to 14, 6 to 13, 7 to 14, 7 to 13, 7 to 12, 7 to 11, 7 to 10, 8 to 14, 8 to 13, 8 to 12, 8 to 11, 8 to 10, or 8 to 9 nucleotides in length.

The core domain is complementary with the core domain target. Typically the core domain has exact complementarity with the core domain target. In some embodiments, the core domain can have 1, 2, 3, 4 or 5 nucleotides that are not complementary with the corresponding nucleotide of the core domain. In an embodiment, the degree of complementarity, together with other properties of the gRNA, is sufficient to allow targeting of a Cas9 molecule to the target nucleic acid.

The “secondary domain” of the targeting domain of the gRNA is complementary to the “secondary domain target” of the target nucleic acid.

In an embodiment, the secondary domain is positioned 5′ to the core domain.

In an embodiment, the secondary domain is absent or optional.

In an embodiment, if the targeting domain is 25 nucleotides in length and the core domain (counted from the 3′ end of the targeting domain) is 8 to 13 nucleotides in length, the secondary domain is 12 to 17 nucleotides in length.

In an embodiment, if the targeting domain is 24 nucleotides in length and the core domain (counted from the 3′ end of the targeting domain) is 8 to 13 nucleotides in length, the secondary domain is 11 to 16 nucleotides in length.

In an embodiment, if the targeting domain is 23 nucleotides in length and the core domain (counted from the 3′ end of the targeting domain) is 8 to 13 nucleotides in length, the secondary domain is 10 to 15 nucleotides in length.

In an embodiment, if the targeting domain is 22 nucleotides in length and the core domain (counted from the 3′ end of the targeting domain) is 8 to 13 nucleotides in length, the secondary domain is 9 to 14 nucleotides in length.

In an embodiment, if the targeting domain is 21 nucleotides in length and the core domain (counted from the 3′ end of the targeting domain) is 8 to 13 nucleotides in length, the secondary domain is 8 to 13 nucleotides in length.

In an embodiment, if the targeting domain is 20 nucleotides in length and the core domain (counted from the 3′ end of the targeting domain) is 8 to 13 nucleotides in length, the secondary domain is 7 to 12 nucleotides in length.

In an embodiment, if the targeting domain is 19 nucleotides in length and the core domain (counted from the 3′ end of the targeting domain) is 8 to 13 nucleotides in length, the secondary domain is 6 to 11 nucleotides in length.

In an embodiment, if the targeting domain is 18 nucleotides in length and the core domain (counted from the 3′ end of the targeting domain) is 8 to 13 nucleotides in length, the secondary domain is 5 to 10 nucleotides in length.

In an embodiment, if the targeting domain is 17 nucleotides in length and the core domain (counted from the 3′ end of the targeting domain) is 8 to 13 nucleotides in length, the secondary domain is 4 to 9 nucleotides in length.

In an embodiment, if the targeting domain is 16 nucleotides in length and the core domain (counted from the 3′ end of the targeting domain) is 8 to 13 nucleotides in length, the secondary domain is 3 to 8 nucleotides in length.

In an embodiment, the secondary domain is 0, 1, 2, 3, 4, 5, 6, 7, 8, 9, 10, 11, 12, 13, 14 or 15 nucleotides in length.

The secondary domain is complementary with the secondary domain target. Typically the secondary domain has exact complementarity with the secondary domain target. In some embodiments the secondary domain can have 1, 2, 3, 4 or 5 nucleotides that are not complementary with the corresponding nucleotide of the secondary domain. In an embodiment, the degree of complementarity, together with other properties of the gRNA, is sufficient to allow targeting of a Cas9 molecule to the target nucleic acid.

In an embodiment, the core domain nucleotides do not comprise modifications, e.g., modifications of the type provided in Section X. However, in an embodiment, the core domain comprises one or more modifications, e.g., modifications that it render it less susceptible to degradation or more bio-compatible, e.g., less immunogenic. By way of example, the backbone of the core domain can be modified with a phosphorothioate, or other modification from Section X. In an embodiment, a nucleotide of the core domain can comprise a 2′ modification (e.g., a modification at the 2′ position on ribose), e.g., a 2′-acetylation, e.g., a 2′ methylation, or other modification from Section X. Typically, a core domain will contain no more than 1, 2, or 3 modifications.

Modifications in the core domain can be selected to not interfere with targeting efficacy, which can be evaluated by testing a candidate modification in the system described in Section III. gRNA's having a candidate core domain having a selected length, sequence, degree of complementarity, or degree of modification, can be evaluated in the system described at Section III. The candidate core domain can be placed, either alone, or with one or more other candidate changes in a gRNA molecule/Cas9 molecule system known to be functional with a selected target and evaluated.

In an embodiment, the secondary domain nucleotides do not comprise modifications, e.g., modifications of the type provided in Section X. However, in an embodiment, the secondary domain comprises one or more modifications, e.g., modifications that render it less susceptible to degradation or more bio-compatible, e.g., less immunogenic. By way of example, the backbone of the secondary domain can be modified with a phosphorothioate, or other modification from Section X. In an embodiment, a nucleotide of the secondary domain can comprise a 2′ modification (e.g., a modification at the 2′ position on ribose), e.g., a 2′-acetylation, e.g., a 2′ methylation, or other modification from Section X. Typically, a secondary domain will contain no more than 1, 2, or 3 modifications.

Modifications in the secondary domain can be selected to not interfere with targeting efficacy, which can be evaluated by testing a candidate modification in the system described in Section III. gRNA's having a candidate secondary domain having a selected length, sequence, degree of complementarity, or degree of modification, can be evaluated in the system described at Section III. The candidate secondary domain can be placed, either alone, or with one or more other candidate changes in a gRNA molecule/Cas9 molecule system known to be functional with a selected target and evaluated.

In an embodiment, (1) the degree of complementarity between the core domain and its target, and (2) the degree of complementarity between the secondary domain and its target, may differ. In an embodiment, (1) may be greater than (2). In an embodiment, (1) may be less than (2). In an embodiment, (1) and (2) may be the same, e.g., each may be completely complementary with its target.

In an embodiment, (1) the number of modifications (e.g., modifications from Section X) of the nucleotides of the core domain and (2) the number of modification (e.g., modifications from Section X) of the nucleotides of the secondary domain, may differ. In an embodiment, (1) may be less than (2). In an embodiment, (1) may be greater than (2). In an embodiment, (1) and (2) may be the same, e.g., each may be free of modifications.

The First and Second Complementarity Domains

The first complementarity domain is complementary with the second complementarity domain.

Typically the first domain does not have exact complementarity with the second complementarity domain target. In some embodiments, the first complementarity domain can have 1, 2, 3, 4 or 5 nucleotides that are not complementary with the corresponding nucleotide of the second complementarity domain. In an embodiment, 1, 2, 3, 4, 5 or 6, e.g., 3 nucleotides, will not pair in the duplex, and, e.g., form a non-duplexed or looped-out region. In an embodiment, an unpaired, or loop-out, region, e.g., a loop-out of 3 nucleotides, is present on the second complementarity domain. In an embodiment, the unpaired region begins 1, 2, 3, 4, 5, or 6, e.g., 4, nucleotides from the 5′ end of the second complementarity domain.

In an embodiment, the degree of complementarity, together with other properties of the gRNA, is sufficient to allow targeting of a Cas9 molecule to the target nucleic acid.

In an embodiment, the first and second complementarity domains are:
independently, 6+/−2, 7+/−2, 8+/−2, 9+/−2, 10+/−2, 11+/−2, 12+/−2, 13+/−2, 14+/−2, 15+/−2, 16+/−2, 17+/−2, 18+/−2, 19+/−2, or 20+/−2, 21+/−2, 22+/−2, 23+/−2, or 24+/−2 nucleotides in length;
independently, 6, 7, 8, 9, 10, 11, 12, 13, 14, 14, 16, 17, 18, 19, 20, 21, 22, 23, 24, 25, or 26 nucleotides in length; or
independently, 5 to 24, 5 to 23, 5 to 22, 5 to 21, 5 to 20, 7 to 18, 9 to 16, or 10 to 14 nucleotides in length.

In an embodiment, the second complementarity domain is longer than the first complementarity domain, e.g., 2, 3, 4, 5, or 6, e.g., 6, nucleotides longer.

In an embodiment, the first and second complementary domains, independently, do not comprise modifications, e.g., modifications of the type provided in Section X.

In an embodiment, the first and second complementary domains, independently, comprise one or more modifications, e.g., modifications that the render the domain less susceptible to degradation or more bio-compatible, e.g., less immunogenic. By way of example, the backbone of the domain can be modified with a phosphorothioate, or other modification from Section X. In an embodiment, a nucleotide of the domain can comprise a 2′ modification (e.g., a modification at the 2′ position on ribose), e.g., a 2′-acetylation, e.g., a 2′ methylation, or other modification from Section X.

In an embodiment, the first and second complementary domains, independently, include 1, 2, 3, 4, 5, 6, 7 or 8 or more modifications. In an embodiment, the first and second complementary domains, independently, include 1, 2, 3, or 4 modifications within 5 nucleotides of its 5′ end. In an embodiment, the first and second complementary domains, independently, include as many as 1, 2, 3, or 4 modifications within 5 nucleotides of its 3′ end.

In an embodiment, the first and second complementary domains, independently, include modifications at two consecutive nucleotides, e.g., two consecutive nucleotides that are within 5 nucleotides of the 5′ end of the domain, within 5 nucleotides of the 3′ end of the domain, or more than 5 nucleotides away from one or both ends of the domain. In an embodiment, the first and second complementary domains, independently, include no two consecutive nucleotides that are modified, within 5 nucleotides of the 5′ end of the domain, within 5 nucleotides of the 3′ end of the domain, or within a region that is more than 5 nucleotides away from one or both ends of the domain. In an embodiment, the first and second complementary domains, independently, include no nucleotide that is modified within 5 nucleotides of the 5′ end of the domain, within 5 nucleotides of the 3′ end of the domain, or within a region that is more than 5 nucleotides away from one or both ends of the domain.

Modifications in a complementarity domain can be selected to not interfere with targeting efficacy, which can be evaluated by testing a candidate modification in the system described in Section III. gRNA's having a candidate complementarity domain having a selected length, sequence, degree of complementarity, or degree of modification, can be evaluated in the system described in Section III. The candidate complementarity domain can be placed, either alone, or with one or more other candidate changes in a gRNA molecule/Cas9 molecule system known to be functional with a selected target and evaluated.

In an embodiment, the first complementarity domain has at least 60, 70, 80, 85%, 90%, or 95% homology with, or differs by no more than 1, 2, 3, 4, 5, or 6 nucleotides from, a reference first complementarity domain, e.g., a naturally occurring, e.g., an S. pyogenes, or S. thermophilus, first complementarity domain, or a first complementarity domain described herein, e.g., from FIGS. 1A-1F.

In an embodiment, the second complementarity domain has at least 60, 70, 80, 85%, 90%, or 95% homology with, or differs by no more than 1, 2, 3, 4, 5, or 6 nucleotides from, a reference second complementarity domain, e.g., a naturally occurring, e.g., an S. pyogenes, or S. thermophilus, second complementarity domain, or a second complementarity domain described herein, e.g., from FIGS. 1A-1F.

The duplexed region formed by first and second complementarity domains is typically 6, 7, 8, 9, 10, 11, 12, 13, 14, 15, 16, 17, 18, 19, 20, 21 or 22 base pairs in length (excluding any looped out or unpaired nucleotides).

In some embodiments, the first and second complementarity domains, when duplexed, comprise 11 paired nucleotides, for example, in the gRNA sequence (one paired strand underlined, one bolded):

(SEQ ID NO: 5)
NNNNNNNNNNNNNNNNNNNNGUUUUAGAGCUAGAAAUAGCAAG
UUAAAAUAAGGCUAGUCCGUUAUCAACUUGAAAAAGUGGCACCG
AGUCGGUGC.

In some embodiments, the first and second complementarity domains, when duplexed, comprise 15 paired nucleotides, for example in the gRNA sequence (one paired strand underlined, one bolded):

(SEQ ID NO: 27)
NNNNNNNNNNNNNNNNNNNNGUUUUAGAGCUAUGCUGAAAAGC
AUAGCAAGUUAAAAUAAGGCUAGUCCGUUAUCAACUUGAAAAA
GUGGCACCGAGUCGGUGC.

In some embodiments the first and second complementarity domains, when duplexed, comprise 16 paired nucleotides, for example in the gRNA sequence (one paired strand underlined, one bolded):

(SEQ ID NO: 28)
NNNNNNNNNNNNNNNNNNNNGUUUUAGAGCUAUGCUGGAAACA
GCAUAGCAAGUUAAAAUAAGGCUAGUCCGUUAUCAACUUGAAA
AAGUGGCACCGAGUCGGUGC.

In some embodiments the first and second complementarity domains, when duplexed, comprise 21 paired nucleotides, for example in the gRNA sequence (one paired strand underlined, one bolded):

(SEQ ID NO: 29)
NNNNNNNNNNNNNNNNNNNNGUUUUAGAGCUAUGCUGUUUUGG
AAACAAAACAGCAUAGCAAGUUAAAAUAAGGCUAGUCCGUUAU
CAACUUGAAAAAGUGGCACCGAGUCGGUGC.

In some embodiments, nucleotides are exchanged to remove poly-U tracts, for example in the gRNA sequences (exchanged nucleotides underlined):

(SEQ ID NO: 30)
NNNNNNNNNNNNNNNNNNNNGUAUUAGAGCUAGAAAUAGCAAG
UUAAUAUAAGGCUAGUCCGUUAUCAACUUGAAAAAGUGGCACCG
AGUCGGUGC;
(SEQ ID NO: 31)
NNNNNNNNNNNNNNNNNNNNGUUUAAGAGCUAGAAAUAGCAAG
UUUAAAUAAGGCUAGUCCGUUAUCAACUUGAAAAAGUGGCACCG
AGUCGGUGC;
and
(SEQ ID NO: 32)
NNNNNNNNNNNNNNNNNNNNGUAUUAGAGCUAUGCUGUAUUGG
AAACAAUACAGCAUAGCAAGUUAAUAUAAGGCUAGUCCGUUAUC
AACUUGAAAAAGUGGCACCGAGUCGGUGC.

The 5′ Extension Domain

In an embodiment, a modular gRNA can comprise additional sequence, 5′ to the second complementarity domain. In an embodiment, the 5′ extension domain is 2 to 10, 2 to 9, 2 to 8, 2 to 7, 2 to 6, 2 to 5, or 2 to 4 nucleotides in length. In an embodiment, the 5′ extension domain is 2, 3, 4, 5, 6, 7, 8, 9, or 10 or more nucleotides in length.

In an embodiment, the 5′ extension domain nucleotides do not comprise modifications, e.g., modifications of the type provided in Section X. However, in an embodiment, the 5′ extension domain comprises one or more modifications, e.g., modifications that it render it less susceptible to degradation or more bio-compatible, e.g., less immunogenic. By way of example, the backbone of the 5′ extension domain can be modified with a phosphorothioate, or other modification from Section X. In an embodiment, a nucleotide of the 5′ extension domain can comprise a 2′ modification (e.g., a modification at the 2′ position on ribose), e.g., a 2′-acetylation, e.g., a 2′ methylation, or other modification from Section X.

In some embodiments, the 5′ extension domain can comprise as many as 1, 2, 3, 4, 5, 6, 7 or 8 modifications. In an embodiment, the 5′ extension domain comprises as many as 1, 2, 3, or 4 modifications within 5 nucleotides of its 5′ end, e.g., in a modular gRNA molecule. In an embodiment, the 5′ extension domain comprises as many as 1, 2, 3, or 4 modifications within 5 nucleotides of its 3′ end, e.g., in a modular gRNA molecule.

In some embodiments, the 5′ extension domain comprises modifications at two consecutive nucleotides, e.g., two consecutive nucleotides that are within 5 nucleotides of the 5′ end of the 5′ extension domain, within 5 nucleotides of the 3′ end of the 5′ extension domain, or more than 5 nucleotides away from one or both ends of the 5′ extension domain. In an embodiment, no two consecutive nucleotides are modified within 5 nucleotides of the 5′ end of the 5′ extension domain, within 5 nucleotides of the 3′ end of the 5′ extension domain, or within a region that is more than 5 nucleotides away from one or both ends of the 5′ extension domain. In an embodiment, no nucleotide is modified within 5 nucleotides of the 5′ end of the 5′ extension domain, within 5 nucleotides of the 3′ end of the 5′ extension domain, or within a region that is more than 5 nucleotides away from one or both ends of the 5′ extension domain.

Modifications in the 5′ extension domain can be selected to not interfere with gRNA molecule efficacy, which can be evaluated by testing a candidate modification in the system described in Section III. gRNAs having a candidate 5′ extension domain having a selected length, sequence, degree of complementarity, or degree of modification, can be evaluated in the system described at Section III. The candidate 5′ extension domain can be placed, either alone, or with one or more other candidate changes in a gRNA molecule/Cas9 molecule system known to be functional with a selected target and evaluated.

In an embodiment, the 5′ extension domain has at least 60, 70, 80, 85, 90 or 95% homology with, or differs by no more than 1, 2, 3, 4, 5, or 6 nucleotides from, a reference 5′ extension domain, e.g., a naturally occurring, e.g., an S. pyogenes, or S. thermophilus, 5′ extension domain, or a 5′ extension domain described herein, e.g., from FIG. 1A and FIG. 1F.

The Linking Domain

In a unimolecular gRNA molecule the linking domain is disposed between the first and second complementarity domains. In a modular gRNA molecule, the two molecules are associated with one another by the complementarity domains.

In an embodiment, the linking domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the linking domain is 20+/−10, 30+/−10, 40+/−10, 50+/−10, 60+/−10, 70+/−10, 80+/−10, 90+/−10, or 100+/−10 nucleotides, in length.

In an embodiment, the linking domain is 10 to 100, 10 to 90, 10 to 80, 10 to 70, 10 to 60, 10 to 50, 10 to 40, 10 to 30, 10 to 20 or 10 to 15 nucleotides in length. In other embodiments, the targeting domain is 20 to 100, 20 to 90, 20 to 80, 20 to 70, 20 to 60, 20 to 50, 20 to 40, 20 to 30, or 20 to 25 nucleotides in length.

In an embodiment, the linking domain is 1, 2, 3, 4, 5, 6, 7, 8, 9, 10, 11, 12, 13, 14, 15, 16 17, 18, 19, or 20 nucleotides in length.

In an embodiment, the linking domain is a covalent bond.

In an embodiment, the linking domain comprises a duplexed region, typically adjacent to or within 1, 2, or 3 nucleotides of the 3′ end of the first complementarity domain and/or the 5-end of the second complementarity domain. In an embodiment, the duplexed region can be 20+/−10, 30+/−10, 40, +/−10 or 50+/−10 base pairs in length. In an embodiment, the duplexed region can be 10+/−5, 15+/−5, 20+/−5, or 30+/−5 base pairs in length. In an embodiment, the duplexed region can be 1, 2, 3, 4, 5, 6, 7, 8, 9, 10, 11, 12, 13, 14, or 15 base pairs in length.

Typically the sequences forming the duplexed region have exact complementarity with one another, though in some embodiments as many as 1, 2, 3, 4, 5, 6, 7 or 8 nucleotides are not complementary with the corresponding nucleotides.

In an embodiment, the linking domain nucleotides do not comprise modifications, e.g., modifications of the type provided in Section X. However, in an embodiment the linking domain comprises one or more modifications, e.g., modifications that it render it less susceptible to degradation or more bio-compatible, e.g., less immunogenic. By way of example, the backbone of the linking domain can be modified with a phosphorothioate, or other modification from Section X. In an embodiment, a nucleotide of the linking domain can comprise a 2′ modification (e.g., a modification at the 2′ position on ribose), e.g., a 2′-acetylation, e.g., a 2′ methylation, or other modification from Section X.

In some embodiments, the linking domain can comprise as many as 1, 2, 3, 4, 5, 6, 7 or 8 modifications.

Modifications in a linking domain can be selected to not interfere with targeting efficacy, which can be evaluated by testing a candidate modification in the system described in Section III. gRNA's having a candidate linking domain having a selected length, sequence, degree of complementarity, or degree of modification, can be evaluated a system described in Section III. A candidate linking domain can be placed, either alone, or with one or more other candidate changes in a gRNA molecule/Cas9 molecule system known to be functional with a selected target and evaluated.

In an embodiment, the linking domain has at least 60, 70, 80, 85, 90 or 95% homology with, or differs by no more than 1, 2, 3, 4, 5, or 6 nucleotides from, a reference linking domain, e.g., a linking domain described herein, e.g., from FIGS. 1B-1E.

The Proximal Domain

In an embodiment, the proximal domain is 6+/−2, 7+/−2, 8+/−2, 9+/−2, 10+/−2, 11+/−2, 12+/−2, 13+/−2, 14+/−2, 14+/−2, 16+/−2, 17+/−2, 18+/−2, 19+/−2, or 20+/−2 nucleotides in length.

In an embodiment, the proximal domain is 6, 7, 8, 9, 10, 11, 12, 13, 14, 14, 16, 17, 18, 19, or 20 nucleotides in length.

In an embodiment, the proximal domain is 5 to 20, 7, to 18, 9 to 16, or 10 to 14 nucleotides in length.

In an embodiment, the proximal domain nucleotides do not comprise modifications, e.g., modifications of the type provided in Section X. However, in an embodiment, the proximal domain comprises one or more modifications, e.g., modifications that it render it less susceptible to degradation or more bio-compatible, e.g., less immunogenic. By way of example, the backbone of the proximal domain can be modified with a phosphorothioate, or other modification from Section X. In an embodiment, a nucleotide of the proximal domain can comprise a 2′ modification (e.g., a modification at the 2′ position on ribose), e.g., a 2′-acetylation, e.g., a 2′ methylation, or other modification from Section X.

In some embodiments, the proximal domain can comprise as many as 1, 2, 3, 4, 5, 6, 7 or 8 modifications. In an embodiment, the proximal domain comprises as many as 1, 2, 3, or 4 modifications within 5 nucleotides of its 5′ end, e.g., in a modular gRNA molecule. In an embodiment, the target domain comprises as many as 1, 2, 3, or 4 modifications within 5 nucleotides of its 3′ end, e.g., in a modular gRNA molecule.

In some embodiments, the proximal domain comprises modifications at two consecutive nucleotides, e.g., two consecutive nucleotides that are within 5 nucleotides of the 5′ end of the proximal domain, within 5 nucleotides of the 3′ end of the proximal domain, or more than 5 nucleotides away from one or both ends of the proximal domain. In an embodiment, no two consecutive nucleotides are modified within 5 nucleotides of the 5′ end of the proximal domain, within 5 nucleotides of the 3′ end of the proximal domain, or within a region that is more than 5 nucleotides away from one or both ends of the proximal domain. In an embodiment, no nucleotide is modified within 5 nucleotides of the 5′ end of the proximal domain, within 5 nucleotides of the 3′ end of the proximal domain, or within a region that is more than 5 nucleotides away from one or both ends of the proximal domain.

Modifications in the proximal domain can be selected to not interfere with gRNA molecule efficacy, which can be evaluated by testing a candidate modification in the system described in Section III. gRNA's having a candidate proximal domain having a selected length, sequence, degree of complementarity, or degree of modification, can be evaluated in the system described at Section III. The candidate proximal domain can be placed, either alone, or with one or more other candidate changes in a gRNA molecule/Cas9 molecule system known to be functional with a selected target and evaluated.

In an embodiment, the proximal domain has at least 60%, 70%, 80%, 85%, 90%, or 95% homology with, or differs by no more than 1, 2, 3, 4, 5, or 6 nucleotides from, a reference proximal domain, e.g., a naturally occurring, e.g., an S. pyogenes, or S. thermophilus, proximal domain, or a proximal domain described herein, e.g., from FIGS. 1A-1F.

The Tail Domain

In an embodiment, the tail domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the tail domain is 20+/−5 nucleotides in length.

In an embodiment, the tail domain is 20+/−10, 30+/−10, 40+/−10, 50+/−10, 60+/−10, 70+/−10, 80+/−10, 90+/−10, or 100+/−10 nucleotides, in length.

In an embodiment, the tail domain is 25+/−10 nucleotides in length.

In an embodiment, the tail domain is 10 to 100, 10 to 90, 10 to 80, 10 to 70, 10 to 60, 10 to 50, 10 to 40, 10 to 30, 10 to 20 or 10 to 15 nucleotides in length.

In other embodiments, the tail domain is 20 to 100, 20 to 90, 20 to 80, 20 to 70, 20 to 60, 20 to 50, 20 to 40, 20 to 30, or 20 to 25 nucleotides in length.

In an embodiment, the tail domain is 1 to 20, 1 to 1, 1 to 10, or 1 to 5 nucleotides in length.

In an embodiment, the tail domain nucleotides do not comprise modifications, e.g., modifications of the type provided in Section X. However, in an embodiment the tail domain comprises one or more modifications, e.g., modifications that it render it less susceptible to degradation or more bio-compatible, e.g., less immunogenic. By way of example, the backbone of the tail domain can be modified with a phosphorothioate, or other modification from Section X. In an embodiment, a nucleotide of the tail domain can comprise a 2′ modification (e.g., a modification at the 2′ position on ribose), e.g., a 2′-acetylation, e.g., a 2′ methylation, or other modification from Section X.

In some embodiments, the tail domain can have as many as 1, 2, 3, 4, 5, 6, 7 or 8 modifications. In an embodiment, the target domain comprises as many as 1, 2, 3, or 4 modifications within 5 nucleotides of its 5′ end. In an embodiment, the target domain comprises as many as 1, 2, 3, or 4 modifications within 5 nucleotides of its 3′ end.

In an embodiment, the tail domain comprises a tail duplex domain, which can form a tail duplexed region. In an embodiment, the tail duplexed region can be 3, 4, 5, 6, 7, 8, 9, 10, 11, or 12 base pairs in length. In an embodiment, a further single stranded domain, exists 3′ to the tail duplexed domain. In an embodiment, this domain is 3, 4, 5, 6, 7, 8, 9, or 10 nucleotides in length. In an embodiment, it is 4 to 6 nucleotides in length.

In an embodiment, the tail domain has at least 60, 70, 80, or 90% homology with, or differs by no more than 1, 2, 3, 4, 5, or 6 nucleotides from, a reference tail domain, e.g., a naturally occurring, e.g., an S. pyogenes, or S. thermophilus, tail domain, or a tail domain described herein, e.g., from FIG. 1A and FIGS. 1C-1F.

In an embodiment, the proximal and tail domain, taken together comprise the following sequences:

(SEQ ID NO: 33)
AAGGCUAGUCCGUUAUCAACUUGAAAAAGUGGCACCGAGUCGGU
GCU;
(SEQ ID NO: 34)
AAGGCUAGUCCGUUAUCAACUUGAAAAAGUGGCACCGAGUCGGU
GGUGC;
(SEQ ID NO: 35)
AAGGCUAGUCCGUUAUCAACUUGAAAAAGUGGCACCGAGUCGGU
GCGGAUC;
(SEQ ID NO: 36)
AAGGCUAGUCCGUUAUCAACUUGAAAAAGUG;
(SEQ ID NO: 37)
AAGGCUAGUCCGUUAUCA;
or
(SEQ ID NO: 38)
AAGGCUAGUCCG.

In an embodiment, the tail domain comprises the 3′ sequence UUUUUU, e.g., if a U6 promoter is used for transcription.

In an embodiment, the tail domain comprises the 3′ sequence UUUU, e.g., if an H1 promoter is used for transcription.

In an embodiment, tail domain comprises variable numbers of 3′ U's depending, e.g., on the termination signal of the pol-III promoter used.

In an embodiment, the tail domain comprises variable 3′ sequence derived from the DNA template if a T7 promoter is used.

In an embodiment, the tail domain comprises variable 3′ sequence derived from the DNA template, e.g., if in vitro transcription is used to generate the RNA molecule.

In an embodiment, the tail domain comprises variable 3′ sequence derived from the DNA template, e.g, if a pol-II promoter is used to drive transcription.

Modifications in the tail domain can be selected to not interfere with targeting efficacy, which can be evaluated by testing a candidate modification in the system described in Section III. gRNA's having a candidate tail domain having a selected length, sequence, degree of complementarity, or degree of modification, can be evaluated in the system described in Section III. The candidate tail domain can be placed, either alone, or with one or more other candidate changes in a gRNA molecule/Cas9 molecule system known to be functional with a selected target and evaluated.

In some embodiments, the tail domain comprises modifications at two consecutive nucleotides, e.g., two consecutive nucleotides that are within 5 nucleotides of the 5′ end of the tail domain, within 5 nucleotides of the 3′ end of the tail domain, or more than 5 nucleotides away from one or both ends of the tail domain. In an embodiment, no two consecutive nucleotides are modified within 5 nucleotides of the 5′ end of the tail domain, within 5 nucleotides of the 3′ end of the tail domain, or within a region that is more than 5 nucleotides away from one or both ends of the tail domain. In an embodiment, no nucleotide is modified within 5 nucleotides of the 5′ end of the tail domain, within 5 nucleotides of the 3′ end of the tail domain, or within a region that is more than 5 nucleotides away from one or both ends of the tail domain.

In an embodiment a gRNA has the following structure:

5′ [targeting domain]-[first complementarity domain]-[linking domain]-[second complementarity domain]-[proximal domain]-[tail domain]-3′
wherein,
the targeting domain comprises a core domain and optionally a secondary domain, and is 10 to 50 nucleotides in length;
the first complementarity domain is 5 to 25 nucleotides in length and, in an embodiment has
   at least 50, 60, 70, 80, 85, 90, or 95% homology with a reference first
   complementarity domain disclosed herein;
   the linking domain is 1 to 5 nucleotides in length;
the proximal domain is 5 to 20 nucleotides in length and, in an embodiment has at least 50, 60, 70, 80, 85, 90 or 95% homology with a reference proximal domain disclosed herein;
and
the tail domain is absent or a nucleotide sequence is 1 to 50 nucleotides in length and, in an embodiment has at least 50, 60, 70, 80, 85, 90 or 95% homology with a reference tail domain disclosed herein.

Exemplary Chimeric gRNAs

In an embodiment, a unimolecular, or chimeric, gRNA comprises, preferably from 5′ to 3′:
a targeting domain (which is complementary to a target nucleic acid);
a first complementarity domain;
a linking domain;
a second complementarity domain (which is complementary to the first complementarity domain);
a proximal domain; and
a tail domain,
wherein,
(a) the proximal and tail domain, when taken together, comprise at least 15, 18, 20, 25, 30, 31, 35, 40, 45, 49, 50, or 53 nucleotides;
(b) there are at least 15, 18, 20, 25, 30, 31, 35, 40, 45, 49, 50, or 53 nucleotides 3′ to the last nucleotide of the second complementarity domain; or
(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein.

In an embodiment, the proximal and tail domain, when taken together, comprise at least 15, 18, 20, 25, 30, 31, 35, 40, 45, 49, 50, or 53 nucleotides.

In an embodiment, there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of 16, 17, 18, 19, 20, 21, 22, 23, 24 or 25 nucleotides (e.g., 16, 17, 18, 19, 20, 21, 22, 23, 24 or 25 consecutive nucleotides) having complementarity with the target domain, e.g., the targeting domain is 16, 17, 18, 19, 20, 21, 22, 23, 24 or 25 nucleotides in length.

In an embodiment, the targeting domain has, or consists of, 16 nucleotides (e.g., 16 consecutive nucleotides) having complementarity with the target domain, e.g., the targeting domain is 16 nucleotides in length.

In an embodiment, the targeting domain has, or consists of, 17 nucleotides (e.g., 17 consecutive nucleotides) having complementarity with the target domain, e.g., the targeting domain is 17 nucleotides in length.

In an embodiment, the targeting domain has, or consists of, 18 nucleotides (e.g., 18 consecutive nucleotides) having complementarity with the target domain, e.g., the targeting domain is 18 nucleotides in length.

In an embodiment, the targeting domain has, or consists of, 19 nucleotides (e.g., 19 consecutive nucleotides) having complementarity with the target domain, e.g., the targeting domain is 19 nucleotides in length.

In an embodiment, the targeting domain has, or consists of, 20 nucleotides (e.g., 20 consecutive nucleotides) having complementarity with the target domain, e.g., the targeting domain is 20 nucleotides in length.

In an embodiment, the targeting domain has, or consists of, 21 nucleotides (e.g., 21 consecutive nucleotides) having complementarity with the target domain, e.g., the targeting domain is 21 nucleotides in length.

In an embodiment, the targeting domain has, or consists of, 22 nucleotides (e.g., 22 consecutive nucleotides) having complementarity with the target domain, e.g., the targeting domain is 22 nucleotides in length.

In an embodiment, the targeting domain has, or consists of, 23 nucleotides (e.g., 23 consecutive nucleotides) having complementarity with the target domain, e.g., the targeting domain is 23 nucleotides in length.

In an embodiment, the targeting domain has, or consists of, 24 nucleotides (e.g., 24 consecutive nucleotides) having complementarity with the target domain, e.g., the targeting domain is 24 nucleotides in length.

In an embodiment, the targeting domain has, or consists of, 25 nucleotides (e.g., 25 consecutive nucleotides) having complementarity with the target domain, e.g., the targeting domain is 25 nucleotides in length.

In an embodiment, the targeting domain has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

Exemplary Modular gRNAs

In an embodiment, a modular gRNA comprises:
a first strand comprising, preferably from 5′ to 3′;
   a targeting domain;
   a first complementarity domain; and
   a second strand, comprising, preferably from 5′ to 3′:
   optionally a 5′ extension domain;
   a second complementarity domain;
   a proximal domain; and
   a tail domain,
wherein:
(a) the proximal and tail domain, when taken together, comprise at least 15, 18, 20, 25, 30, 31, 35, 40, 45, 49, 50, or 53 nucleotides;
(b) there are at least 15, 18, 20, 25, 30, 31, 35, 40, 45, 49, 50, or 53 nucleotides 3′ to the last nucleotide of the second complementarity domain; or
(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein.

In an embodiment, the proximal and tail domain, when taken together, comprise at least 15, 18, 20, 25, 30, 31, 35, 40, 45, 49, 50, or 53 nucleotides.

In an embodiment, there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 16, 17, 18, 19, 20, 21, 22, 23, 24 or 25 nucleotides (e.g., 16, 17, 18, 19, 20, 21, 22, 23, 24 or 25 consecutive nucleotides) having complementarity with the target domain, e.g., the targeting domain is 16, 17, 18, 19, 20, 21, 22, 23, 24 or 25 nucleotides in length.

In an embodiment, the targeting domain has, or consists of, 16 nucleotides (e.g., 16 consecutive nucleotides) having complementarity with the target domain, e.g., the targeting domain is 16 nucleotides in length.

In an embodiment, the targeting domain has, or consists of, 17 nucleotides (e.g., 17 consecutive nucleotides) having complementarity with the target domain, e.g., the targeting domain is 17 nucleotides in length.

In an embodiment, the targeting domain has, or consists of, 18 nucleotides (e.g., 18 consecutive nucleotides) having complementarity with the target domain, e.g., the targeting domain is 18 nucleotides in length.

In an embodiment, the targeting domain has, or consists of, 19 nucleotides (e.g., 19 consecutive nucleotides) having complementarity with the target domain, e.g., the targeting domain is 19 nucleotides in length.

In an embodiment, the targeting domain has, or consists of, 20 nucleotides (e.g., 20 consecutive nucleotides) having complementarity with the target domain, e.g., the targeting domain is 20 nucleotides in length.

In an embodiment, the targeting domain has, or consists of, 21 nucleotides (e.g., 21 consecutive nucleotides) having complementarity with the target domain, e.g., the targeting domain is 21 nucleotides in length.

In an embodiment, the targeting domain has, or consists of, 22 nucleotides (e.g., 22 consecutive nucleotides) having complementarity with the target domain, e.g., the targeting domain is 22 nucleotides in length.

In an embodiment, the targeting domain has, or consists of, 23 nucleotides (e.g., 23 consecutive nucleotides) having complementarity with the target domain, e.g., the targeting domain is 23 nucleotides in length.

In an embodiment, the targeting domain has, or consists of, 24 nucleotides (e.g., 24 consecutive nucleotides) having complementarity with the target domain, e.g., the targeting domain is 24 nucleotides in length.

In an embodiment, the targeting domain has, or consists of, 25 nucleotides (e.g., 25 consecutive nucleotides) having complementarity with the target domain, e.g., the targeting domain is 5 nucleotides in length.

In an embodiment, the targeting domain has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In an embodiment, the targeting domain has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

Methods for Designing gRNAs

Methods for designing gRNAs are described herein, including methods for selecting, designing and validating target domains. Exemplary targeting domains are also provided herein. Targeting Domains discussed herein can be incorporated into the gRNAs described herein.

Methods for selection and validation of target sequences as well as off-target analyses are described, e.g., in Mali et al., 2013 SCIENCE 339(6121): 823-826; Hsu et al., 2013 NAT BIOTECHNOL, 31(9): 827-32; Fu et al., 2014 NAT BIOTECHNOL, doi: 10.1038/nbt.2808. PubMed PMID: 24463574; Heigwer et al., 2014 NAT METHODS 11(2):122-3. doi: 10.1038/nmeth.2812. PubMed PMID: 24481216; Bae et al., 2014 BIOINFORMATICS PubMed PMID: 24463181; Xiao A et al., 2014 BIOINFORMATICS PubMed PMID: 24389662.

For example, a software tool can be used to optimize the choice of gRNA within a user's target sequence, e.g., to minimize total off-target activity across the genome. Off target activity may be other than cleavage. For each possible gRNA choice e.g., using S. pyogenes Cas9, the tool can identify all off-target sequences (e.g., preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate gRNA molecules can be evaluated by art-known methods or as described in Section IV herein.

### II. Cas9 Molecules

Cas9 molecules of a variety of species can be used in the methods and compositions described herein. While the S. pyogenes and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes and S. thermophilus Cas9 molecules, Cas9 molecules from the other species can replace them, e.g., Staphylococcus aureus and Neisseria meningitidis Cas9 molecules. Additional Cas9 species include: Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

A Cas9 molecule, as that term is used herein, refers to a molecule that can interact with a gRNA molecule and, in concert with the gRNA molecule, localize (e.g., target or home) to a site which comprises a target domain and PAM sequence.

In an embodiment, the Cas9 molecule is capable of cleaving a target nucleic acid molecule. A Cas9 molecule that is capable of cleaving a target nucleic acid molecule is referred to herein as an eaCas9 (an enzymatically active Cas9) molecule. In an embodiment, an eaCas9 molecule, comprises one or more of the following activities:
a nickase activity, i.e., the ability to cleave a single strand, e.g., the non-complementary strand or the complementary strand, of a nucleic acid molecule;
a double stranded nuclease activity, i.e., the ability to cleave both strands of a double stranded nucleic acid and create a double stranded break, which in an embodiment is the presence of two nickase activities;
an endonuclease activity;
an exonuclease activity; and
a helicase activity, i.e., the ability to unwind the helical structure of a double stranded nucleic acid.

In an embodiment, an enzymatically active Cas9 or an eaCas9 molecule cleaves both DNA strands and results in a double stranded break. In an embodiment, an eaCas9 molecule cleaves only one strand, e.g., the strand to which the gRNA hybridizes to, or the strand complementary to the strand the gRNA hybridizes with. In an embodiment, an eaCas9 molecule comprises cleavage activity associated with an HNH-like domain. In an embodiment, an eaCas9 molecule comprises cleavage activity associated with an N-terminal RuvC-like domain. In an embodiment, an eaCas9 molecule comprises cleavage activity associated with an HNH-like domain and cleavage activity associated with an N-terminal RuvC-like domain. In an embodiment, an eaCas9 molecule comprises an active, or cleavage competent, HNH-like domain and an inactive, or cleavage incompetent, N-terminal RuvC-like domain. In an embodiment, an eaCas9 molecule comprises an inactive, or cleavage incompetent, HNH-like domain and an active, or cleavage competent, N-terminal RuvC-like domain.

In an embodiment, the ability of an eaCas9 molecule to interact with and cleave a target nucleic acid is PAM sequence dependent. A PAM sequence is a sequence in the target nucleic acid. In an embodiment, cleavage of the target nucleic acid occurs upstream from the PAM sequence. EaCas9 molecules from different bacterial species can recognize different sequence motifs (e.g., PAM sequences). In an embodiment, an eaCas9 molecule of S. pyogenes recognizes the sequence motif NGG and directs cleavage of a target nucleic acid sequence 1 to 10, e.g., 3 to 5, base pairs upstream from that sequence. See, e.g., Mali et al., SCIENCE 2013; 339(6121): 823-826. In an embodiment, an eaCas9 molecule of S. thermophilus recognizes the sequence motif NGGNG and NNAGAAW (W=A or T) and directs cleavage of a core target nucleic acid sequence 1 to 10, e.g., 3 to 5, base pairs upstream from these sequences. See, e.g., Horvath et al., SCIENCE 2010; 327(5962):167-170, and Deveau et al., J BACTERIOL 2008; 190(4): 1390-1400. In an embodiment, an eaCas9 molecule of S. mutans recognizes the sequence motif NGG or NAAR (R=A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400. In an embodiment, an eaCas9 molecule of S. aureus recognizes the sequence motif NNGRR (R=A or G) and directs cleavage of a target nucleic acid sequence 1 to 10, e.g., 3 to 5, base pairs upstream from that sequence. In an embodiment, an eaCas9 molecule of N. meningitidis recognizes the sequence motif NNNNGATT and directs cleavage of a target nucleic acid sequence 1 to 10, e.g., 3 to 5, base pairs upstream from that sequence. See, e.g., Hou et al., PNAS EARLY EDITION 2013, 1-6. The ability of a Cas9 molecule to recognize a PAM sequence can be determined, e.g., using a transformation assay described in Jinek et al., SCIENCE 2012, 337:816.

Some Cas9 molecules have the ability to interact with a gRNA molecule, and in conjunction with the gRNA molecule home (e.g., targeted or localized) to a core target domain, but are incapable of cleaving the target nucleic acid, or incapable of cleaving at efficient rates. Cas9 molecules having no, or no substantial, cleavage activity are referred to herein as an eiCas9 (an enzymatically inactive Cas9) molecule. For example, an eiCas9 molecule can lack cleavage activity or have substantially less, e.g., less than 20, 10, 5, 1 or 0.1% of the cleavage activity of a reference Cas9 molecule, as measured by an assay described herein.

Exemplary naturally occurring Cas9 molecules are described in Chylinski et al., RNA Biology 2013; 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408). Additional exemplary Cas9 molecules are a Cas9 molecule of Neisseria meningitidis (Hou et al. PNAS Early Edition 2013, 1-6) and a S. aureus Cas9 molecule.

In an embodiment, a Cas9 molecule, e.g., an eaCas9 molecule or eiCas9 molecule, comprises an amino acid sequence:
having 60%, 65%, 70%, 75%, 80%, 85%, 90%, 95%, 96%, 97%, 98%, or 99% homology with;
differs at no more than, 2, 5, 10, 15, 20, 30, or 40% of the amino acid residues when compared with;
differs by at least 1, 2, 5, 10 or 20 amino acids but by no more than 100, 80, 70, 60, 50, 40 or 30 amino acids from; or is identical to;
   any Cas9 molecule sequence described herein or a naturally occurring Cas9 molecule sequence, e.g., a Cas9 molecule from a species listed herein or described in Chylinski et al., RNA Biology 2013, 10:5, 727-737; Hou et al. PNAS Early Edition 2013, 1-6. In an embodiment, the Cas9 molecule comprises one or more of the following activities: a nickase activity; a double stranded cleavage activity (e.g., an endonuclease and/or exonuclease activity); a helicase activity; or the ability, together with a gRNA molecule, to localize to a target nucleic acid.

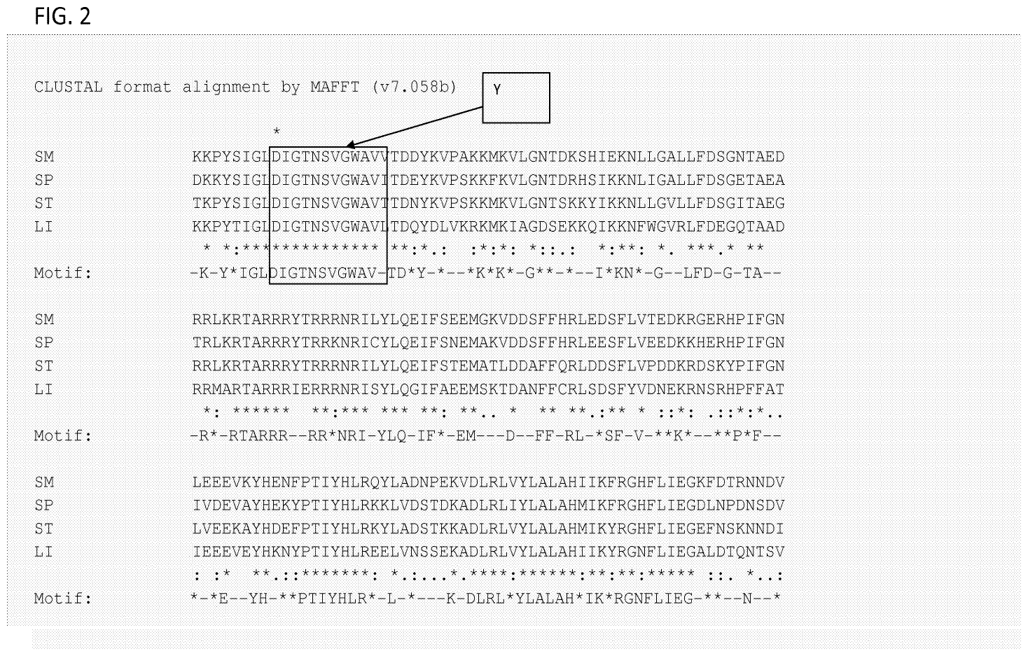

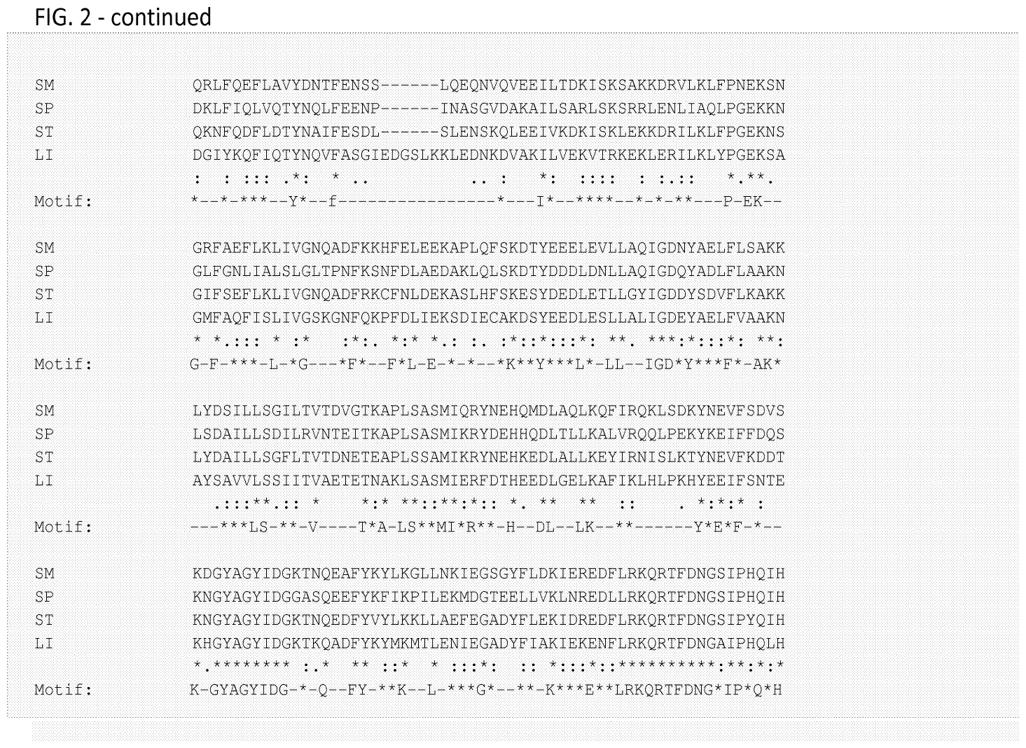

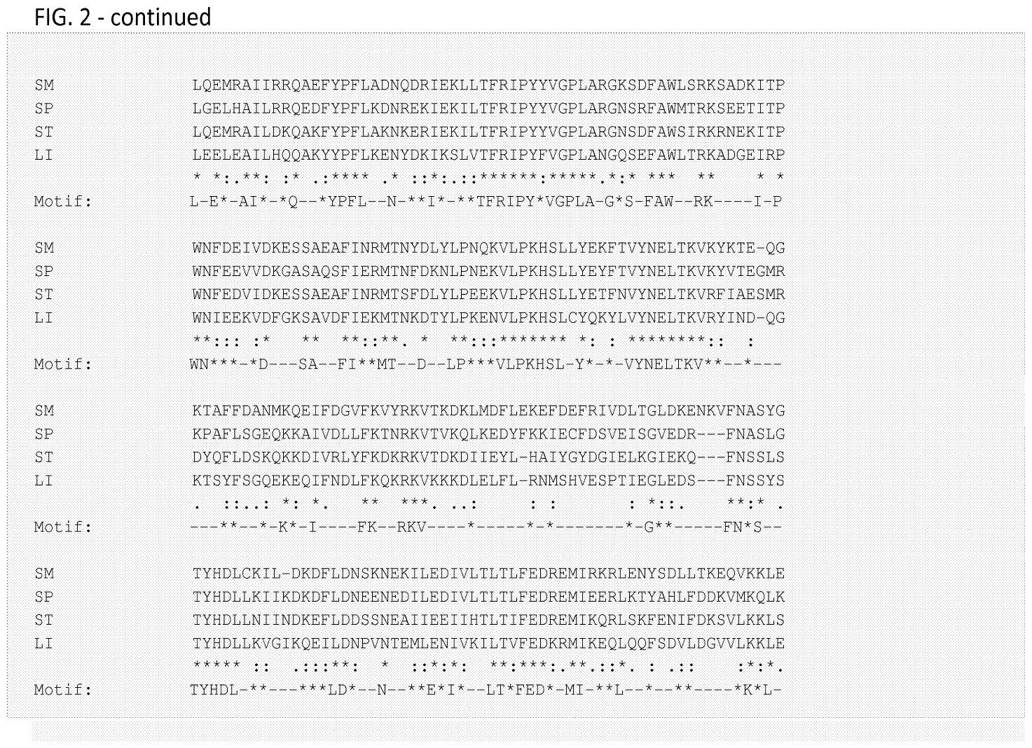

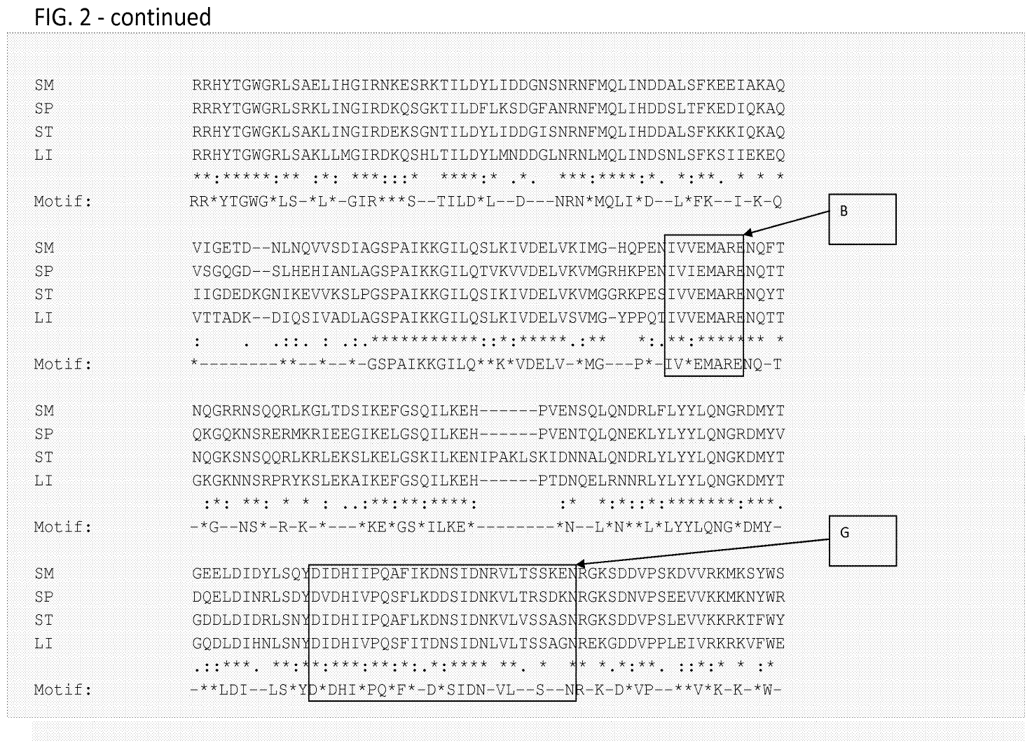

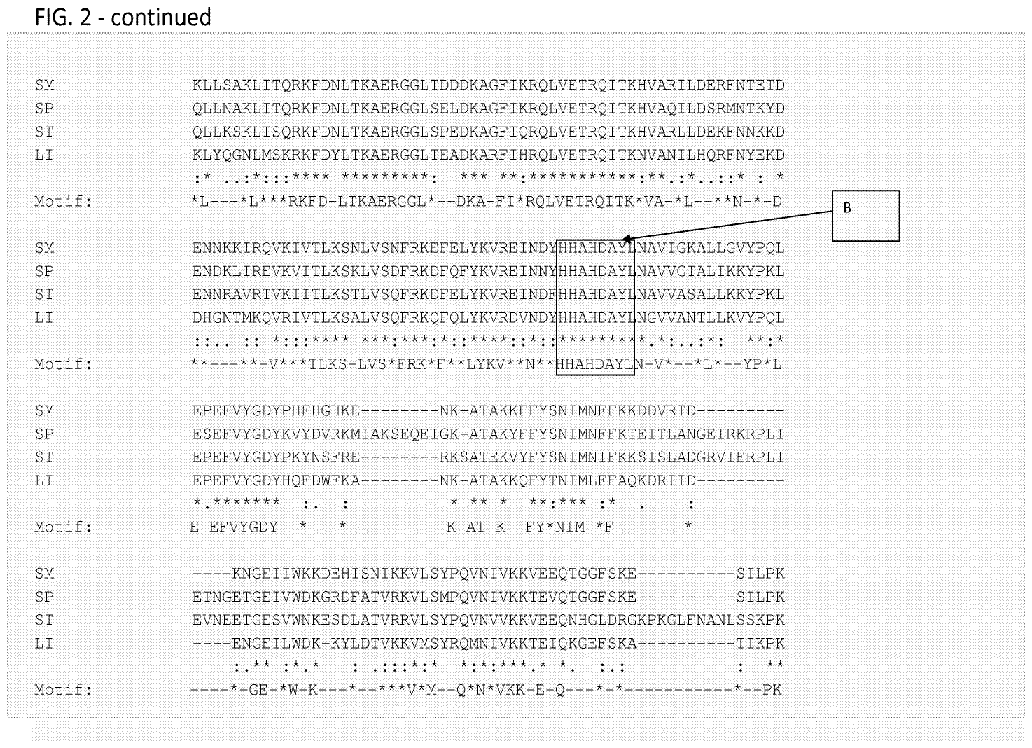

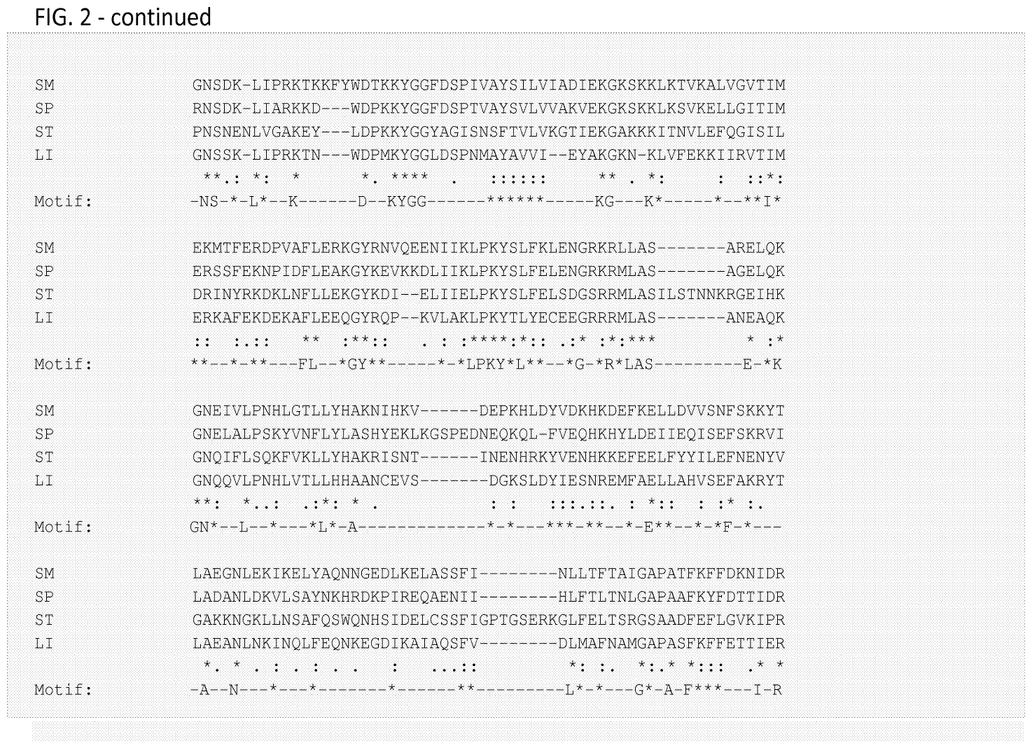

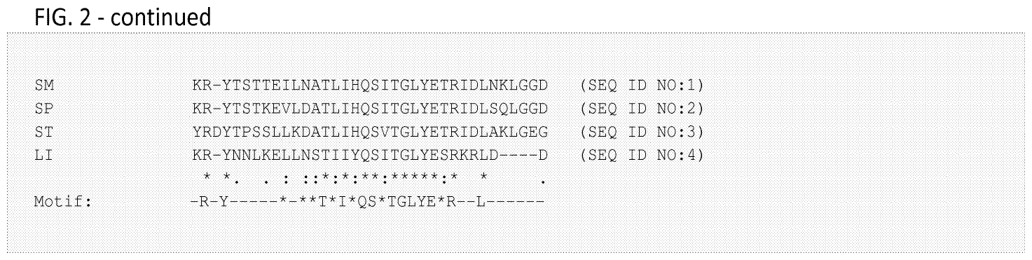

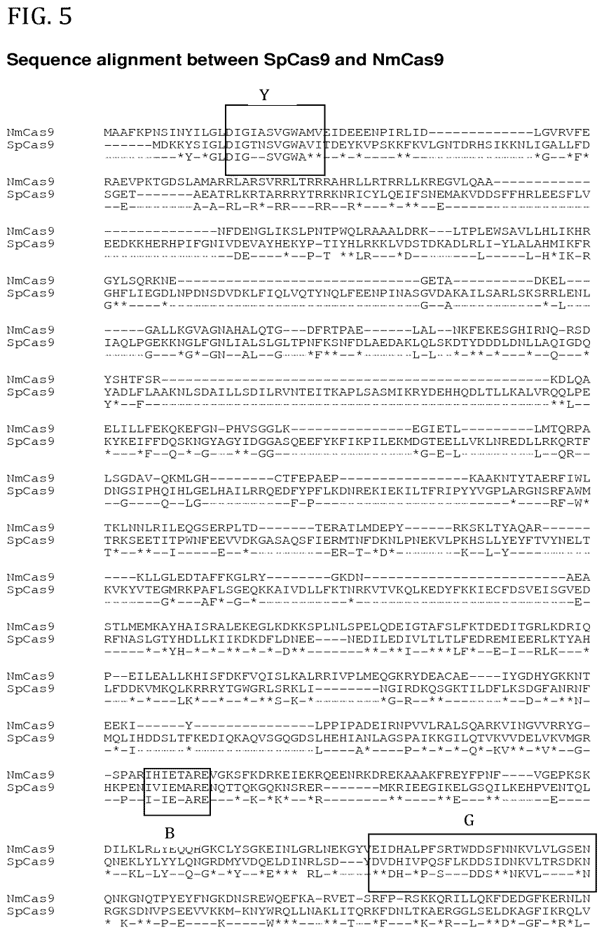

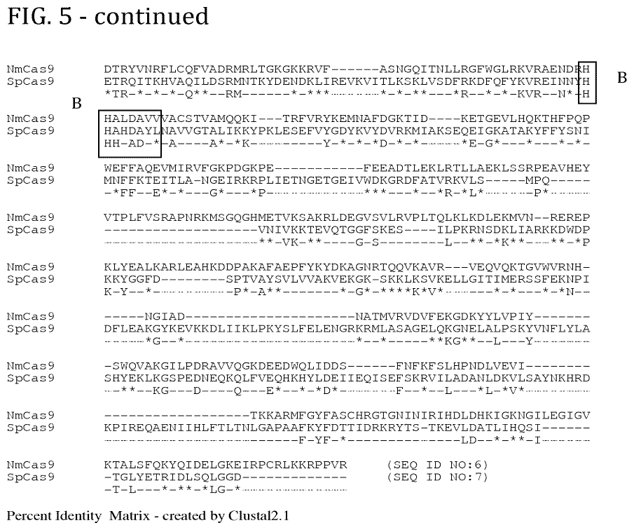

In an embodiment, a Cas9 molecule comprises the amino acid sequence of the consensus sequence of FIG. 2, wherein “*” indicates any amino acid found in the corresponding position in the amino acid sequence of a Cas9 molecule of S. pyogenes, S. thermophilus, S. mutans and L. innocua, and “-” indicates any amino acid. In an embodiment, a Cas9 molecule differs from the sequence of the consensus sequence disclosed in FIG. 2 by at least 1, but no more than 2, 3, 4, 5, 6, 7, 8, 9, or 10 amino acid residues. In an embodiment, a Cas9 molecule comprises the amino acid sequence of SEQ ID NO:7 of FIG. 5, wherein “*” indicates any amino acid found in the corresponding position in the amino acid sequence of a Cas9 molecule of S. pyogenes, or N. meningitidis, “-” indicates any amino acid, and “-” indicates any amino acid or absent. In an embodiment, a Cas9 molecule differs from the sequence of SEQ ID NO:6 or 7 by at least 1, but no more than 2, 3, 4, 5, 6, 7, 8, 9, or 10 amino acid residues.

A comparison of the sequence of a number of Cas9 molecules indicate that certain regions are conserved. These are identified below as:
region 1 (residues 1 to 180, or in the case of region 1′ residues 120 to 180)
region 2 (residues 360 to 480);
region 3 (residues 660 to 720);
region 4 (residues 817 to 900); and
region 5 (residues 900 to 960).

In an embodiment, a Cas9 molecule comprises regions 1-5, together with sufficient additional Cas9 molecule sequence to provide a biologically active molecule, e.g., a Cas9 molecule having at least one activity described herein. In an embodiment, each of regions 1-6, independently, have, 50%, 60%, 70%, 80%, 85%, 90%, 95%, 96%, 97%, 98% or 99% homology with the corresponding residues of a Cas9 molecule described herein, e.g., a sequence from FIG. 2 or from FIG. 5.

In an embodiment, a Cas9 molecule, e.g., an eaCas9 molecule or eiCas9 molecule, comprises an amino acid sequence referred to as region 1:
having 50%, 60%, 70%, 80%, 85%, 90%, 95%, 96%, 97%, 98% or 99% homology with amino acids 1-180 (the numbering is according to the motif sequence in FIG. 2; 52% of residues in the four Cas9 sequences in FIG. 2 are conserved) of the amino acid sequence of Cas9 of S. pyogenes;
differs by at least 1, 2, 5, 10 or 20 amino acids but by no more than 90, 80, 70, 60, 50, 40 or 30 amino acids from amino acids 1-180 of the amino acid sequence of Cas9 of S. pyogenes, S. thermophilus, S. mutans, L. innocua, N. meningitidis, or S. aureus; or
is identical to 1-180 of the amino acid sequence of Cas9 of S. pyogenes, S. thermophilus, S. mutans or, L. innocua, N. meningitidis, or S. aureus.

In an embodiment, a Cas9 molecule, e.g., an eaCas9 molecule or eiCas9 molecule, comprises an amino acid sequence referred to as region 1′:
having 55%, 60%, 65%, 70%, 75%, 80%, 85%, 90%, 95%, 96%, 97%, 98% or 99% homology with amino acids 120-180 (55% of residues in the four Cas9 sequences in FIG. 2 are conserved) of the amino acid sequence of Cas9 of S. pyogenes, S. thermophilus, S. mutans or, L. innocua, N. meningitidis, or S. aureus;
differs by at least 1, 2, or 5 amino acids but by no more than 35, 30, 25, 20 or 10 amino acids from amino acids 120-180 of the amino acid sequence of Cas9 of S. pyogenes, S. thermophilus, S. mutans or, L. innocua, N. meningitidis, or S. aureus; or
is identical to 120-180 of the amino acid sequence of Cas9 of S. pyogenes, S. thermophilus, S. mutans or, L. innocua, N. meningitidis, or S. aureus.

In an embodiment, a Cas9 molecule, e.g., an eaCas9 molecule or eiCas9 molecule, comprises an amino acid sequence referred to as region 2:
having 50%, 55%, 60%, 65%, 70%, 75%, 80%, 85%, 90%, 95%, 96%, 97%, 98% or 99% homology with amino acids 360-480 (52% of residues in the four Cas9 sequences in FIG. 2 are conserved) of the amino acid sequence of Cas9 of S. pyogenes, S. thermophilus, S. mutans or, L. innocua, N. meningitidis, or S. aureus;
differs by at least 1, 2, or 5 amino acids but by no more than 35, 30, 25, 20 or 10 amino acids from amino acids 360-480 of the amino acid sequence of Cas9 of S. pyogenes, S. thermophilus, S. mutans or, L. innocua, N. meningitidis, or S. aureus; or
is identical to 360-480 of the amino acid sequence of Cas9 of S. pyogenes, S. thermophilus, S. mutans or, L. innocua, N. meningitidis, or S. aureus.

In an embodiment, a Cas9 molecule, e.g., an eaCas9 molecule or eiCas9 molecule, comprises an amino acid sequence referred to as region 3:
having 55%, 60%, 65%, 70%, 75%, 80%, 85%, 90%, 95%, 96%, 97%, 98% or 99% homology with amino acids 660-720 (56% of residues in the four Cas9 sequences in FIG. 2 are conserved) of the amino acid sequence of Cas9 of S. pyogenes, S. thermophilus, S. mutans or, L. innocua, N. meningitidis, or S. aureus;
differs by at least 1, 2, or 5 amino acids but by no more than 35, 30, 25, 20 or 10 amino acids from amino acids 660-720 of the amino acid sequence of Cas9 of S. pyogenes, S. thermophilus, S. mutans or, L. innocua, N. meningitidis, or S. aureus; or
is identical to 660-720 of the amino acid sequence of Cas9 of S. pyogenes, S. thermophilus, S. mutans or, L. innocua, N. meningitidis, or S. aureus.

In an embodiment, a Cas9 molecule, e.g., an eaCas9 molecule or eiCas9 molecule, comprises an amino acid sequence referred to as region 4:
having 50%, 55%, 60%, 65%, 70%, 75%, 80%, 85%, 90%, 95%, 96%, 97%, 98% or 99% homology with amino acids 817-900 (55% of residues in the four Cas9 sequences in FIG. 2 are conserved) of the amino acid sequence of Cas9 of S. pyogenes, S. thermophilus, S. mutans or, L. innocua, N. meningitidis, or S. aureus;
differs by at least 1, 2, or 5 amino acids but by no more than 35, 30, 25, 20 or 10 amino acids from amino acids 817-900 of the amino acid sequence of Cas9 of S. pyogenes, S. thermophilus, S. mutans or, L. innocua, N. meningitidis, or S. aureus; or
is identical to 817-900 of the amino acid sequence of Cas9 of S. pyogenes, S. thermophilus, S. mutans or, L. innocua, N. meningitidis, or S. aureus.

In an embodiment, a Cas9 molecule, e.g., an eaCas9 molecule or eiCas9 molecule, comprises an amino acid sequence referred to as region 5:
having 50%, 55%, 60%, 65%, 70%, 75%, 80%, 85%, 90%, 95%, 96%, 97%, 98% or 99% homology with amino acids 900-960 (60% of residues in the four Cas9 sequences in FIG. 2 are conserved) of the amino acid sequence of Cas9 of S. pyogenes, S. thermophilus, S. mutans or, L. innocua, N. meningitidis, or S. aureus;
differs by at least 1, 2, or 5 amino acids but by no more than 35, 30, 25, 20 or 10 amino acids from amino acids 900-960 of the amino acid sequence of Cas9 of S. pyogenes, S. thermophilus, S. mutans or, L. innocua, N. meningitidis, or S. aureus; or
is identical to 900-960 of the amino acid sequence of Cas9 of S. pyogenes, S. thermophilus, S. mutans or, L. innocua, N. meningitidis, or S. aureus.

A RuvC-Like Domain and an HNH-Like Domain

In an embodiment, a Cas9 molecule comprises an HNH-like domain and an RuvC-like domain. In an embodiment, cleavage activity is dependent on a RuvC-like domain and an HNH-like domain. A Cas9 molecule, e.g., an eaCas9 or eiCas9 molecule, can comprise one or more of the following domains: a RuvC-like domain and an HNH-like domain. In an embodiment, a cas9 molecule is an eaCas9 molecule and the eaCas9 molecule comprises a RuvC-like domain, e.g., a RuvC-like domain described below, and/or an HNH-like domain, e.g., an HNH-like domain described below. In an embodiment, a Cas9 molecule is an eiCas9 molecule comprising one or more difference in an RuvC-like domain and/or in an HNH-like domain as compared to a reference Cas9 molecule, and the eiCas9 molecule does not cleave a nucleic acid, or cleaves with significantly less efficiency than does wildype, e.g., when compared with wild type in a cleavage assay, e.g., as described herein, cuts with less than 50, 25, 10, or 1% of the a reference Cas9 molecule, as measured by an assay described herein.

RuvC-Like Domains

In an embodiment, a RuvC-like domain cleaves, a single strand, e.g., the non-complementary strand of the target nucleic acid molecule. A Cas9 molecule can include more than one RuvC-like domain (e.g., one, two, three or more RuvC-like domains). In an embodiment, an RuvC-like domain is at least 5, 6, 7, 8 amino acids in length but not more than 20, 19, 18, 17, 16 or 15 amino acids in length. In an embodiment, the cas9 molecule comprises an N-terminal RuvC-like domain of about 10 to 20 amino acids, e.g., about 15 amino acids in length.

N-Terminal RuvC-Like Domains

Some naturally occurring Cas9 molecules comprise more than one RuvC-like domain, with cleavage being dependent on the N-terminal RuvC-like domain. Accordingly, Cas9 molecules can comprise an N-terminal RuvC-like domain. Exemplary N-terminal RuvC-like domains are described below.

In an embodiment, an eaCas9 molecule comprises an N-terminal RuvC-like domain comprising an amino acid sequence of formula I:

(SEQ ID NO: 8)
D-X1-G-X2-X3-X4-X5-G-X6-X7-X8-X9,

wherein,
X1 is selected from I, V, M, L and T (e.g., selected from I, V, and L);
X2 is selected from T, I, V, 5, N, Y, E and L (e.g., selected from T, V, and I);
X3 is selected from N, S, G, A, D, T, R, M and F (e.g., A or N);
X4 is selected from S, Y, N and F (e.g., S);
X5 is selected from V, I, L, C, T and F (e.g., selected from V, I and L);
X6 is selected from W, F, V, Y, S and L (e.g., W);
X7 is selected from A, S, C, V and G (e.g., selected from A and S);
X8 is selected from V, I, L, A, M and H (e.g., selected from V, I, M and L); and
X9 is selected from any amino acid or is absent (e.g., selected from T, V, I, L, Δ, F, S, A, Y, M and R, or, e.g., selected from T, V, I, L and Δ).

In an embodiment, the N-terminal RuvC-like domain differs from a sequence of SEQ ID NO:8, by as many as 1 but no more than 2, 3, 4, or 5 residues.

In embodiment the N-terminal RuvC-like domain is cleavage competent.

In embodiment the N-terminal RuvC-like domain is cleavage incompetent.

In an embodiment, an eaCas9 molecule comprises an N-terminal RuvC-like domain comprising an amino acid sequence of formula II:

(SEQ ID NO: 9)
D-X1-G-X2-X3-S-X5-G-X6-X7-X8-X9,

wherein
X1 is selected from I, V, M, L and T (e.g., selected from I, V, and L);
X2 is selected from T, I, V, S, N, Y, E and L (e.g., selected from T, V, and I);
X3 is selected from N, S, G, A, D, T, R, M and F (e.g., A or N);
X5 is selected from V, I, L, C, T and F (e.g., selected from V, I and L);
X6 is selected from W, F, V, Y, S and L (e.g., W);
X7 is selected from A, S, C, V and G (e.g., selected from A and S);
X8 is selected from V, I, L, A, M and H (e.g., selected from V, I, M and L); and
X9 is selected from any amino acid or is absent (e.g., selected from T, V, I, L, Δ, F, S, A, Y, M and R or selected from e.g., T, V, I, L and Δ).

In an embodiment, the N-terminal RuvC-like domain differs from a sequence of SEQ ID NO:9 by as many as 1, but no more than 2, 3, 4, or 5 residues.

In an embodiment, the N-terminal RuvC-like domain comprises an amino acid sequence of formula III:

(SEQ ID NO: 10)
D-I-G-X2-X3-S-V-G-W-A-X8-X9,

wherein
X2 is selected from T, I, V, S, N, Y, E and L (e.g., selected from T, V, and I);
X3 is selected from N, S, G, A, D, T, R, M and F (e.g., A or N);
X8 is selected from V, I, L, A, M and H (e.g., selected from V, I, M and L); and
X9 is selected from any amino acid or is absent (e.g., selected from T, V, I, L, Δ, F, S, A, Y, M and R or selected from e.g., T, V, I, L and A).

In an embodiment, the N-terminal RuvC-like domain differs from a sequence of SEQ ID NO:10 by as many as 1, but no more than, 2, 3, 4, or 5 residues.

In an embodiment, the N-terminal RuvC-like domain comprises an amino acid sequence of formula III:

(SEQ ID NO: 11)
D-I-G-T-N-S-V-G-W-A-V-X,

wherein
X is a non-polar alkyl amino acid or a hydroxyl amino acid, e.g., X is selected from V, I, L and T (e.g., the eaCas9 molecule can comprise an N-terminal RuvC-like domain shown in FIG. 2 (depicted as “Y”)).

In an embodiment, the N-terminal RuvC-like domain differs from a sequence of SEQ ID NO:11 by as many as 1 but no more than, 2, 3, 4, or 5 residues.

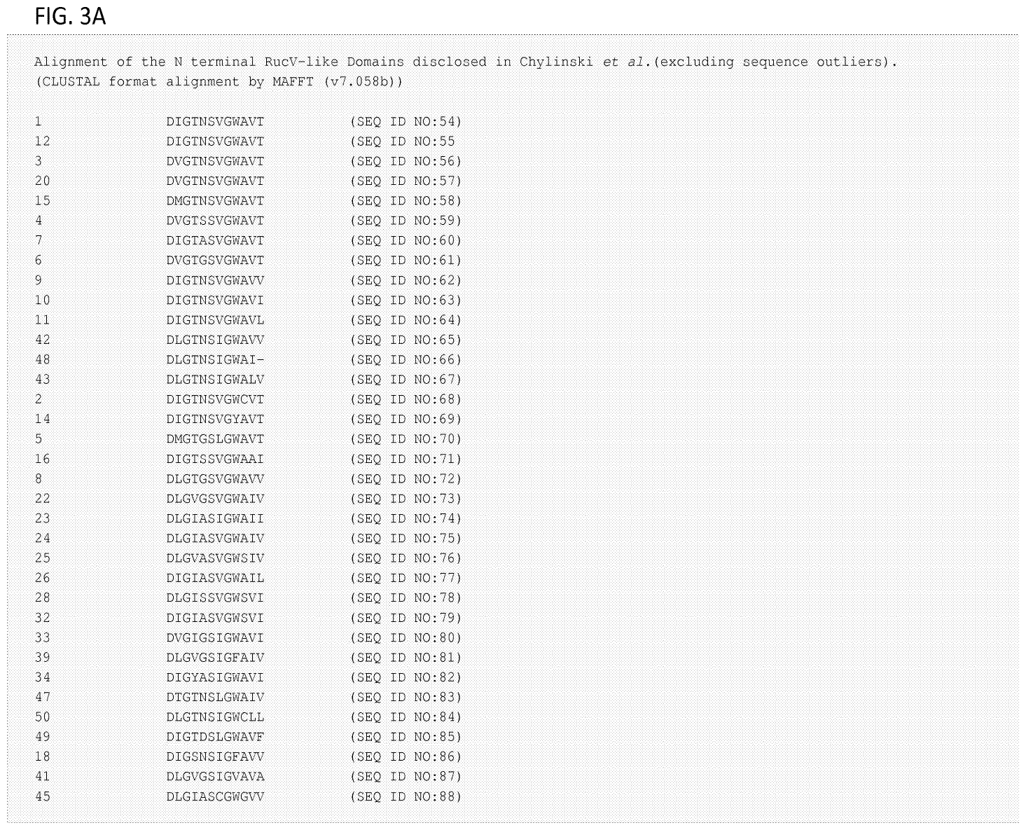

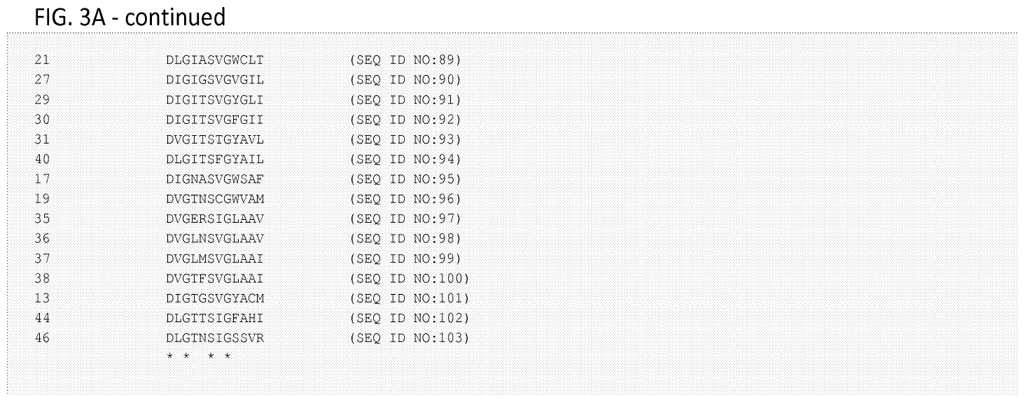

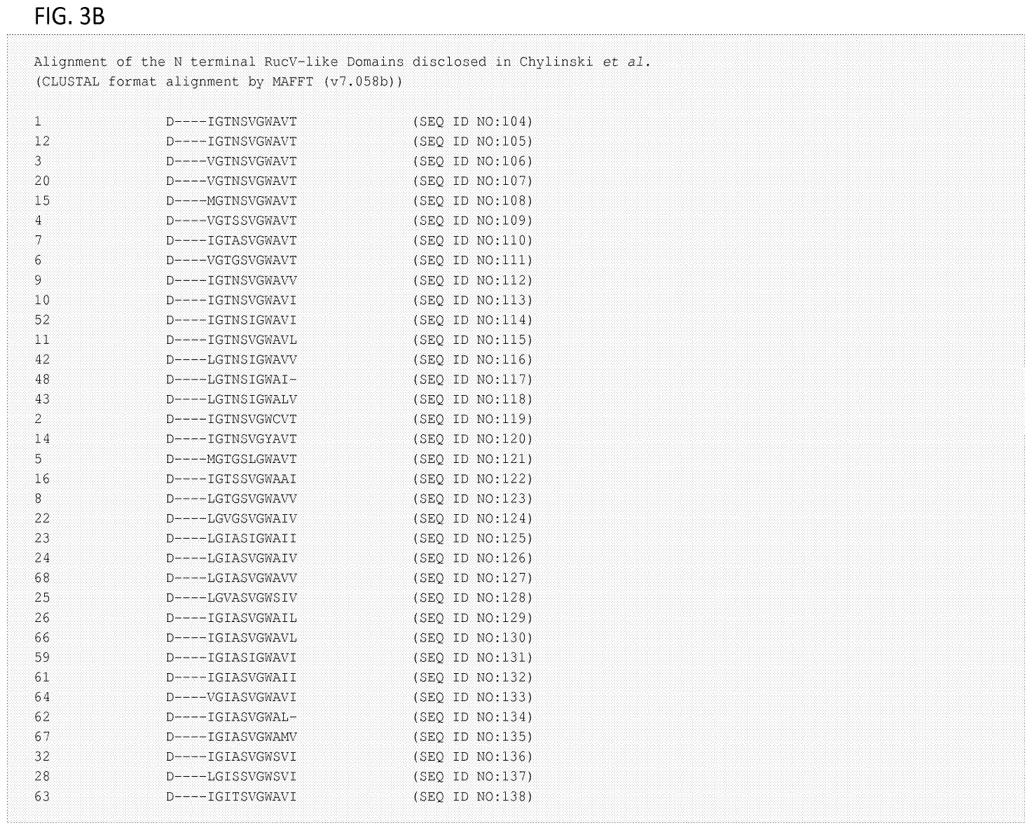

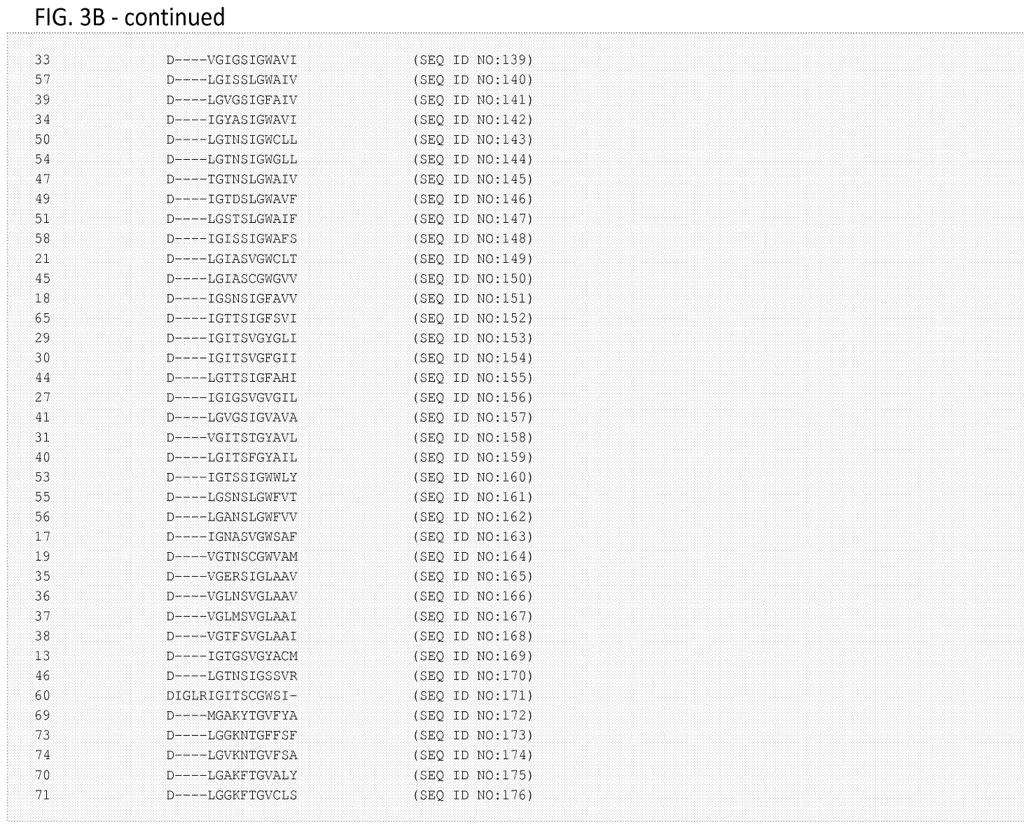

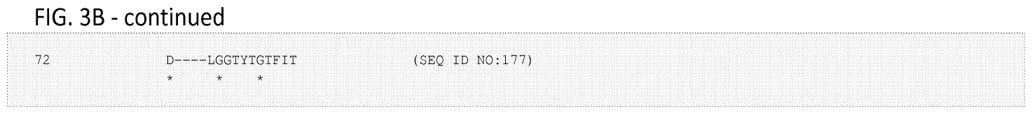

In an embodiment, the N-terminal RuvC-like domain differs from a sequence of an N-terminal RuvC-like domain disclosed herein, e.g., in FIG. 3A or FIG. 5, as many as 1, but no more than 2, 3, 4, or 5 residues. In an embodiment, 1, 2, or all 3 of the highly conserved residues identified in FIG. 3A or FIG. 5 are present.

In an embodiment, the N-terminal RuvC-like domain differs from a sequence of an N-terminal RuvC-like domain disclosed herein, e.g., in FIG. 3B, as many as 1, but no more than 2, 3, 4, or 5 residues. In an embodiment, 1, 2, 3 or all 4 of the highly conserved residues identified in FIG. 3B are present.

Additional RuvC-Like Domains

In addition to the N-terminal RuvC-like domain, a Cas9 molecule, e.g., an eaCas9 molecule, can comprise one or more additional RuvC-like domains. In an embodiment, a Cas9 molecule can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

An additional RuvC-like domain can comprise an amino acid sequence:
I-X1-X2-E-X3-A-R-E (SEQ ID NO:12), wherein
X1 is V or H,
X2 is I, L or V (e.g., I or V); and
X3 is M or T.

In an embodiment, the additional RuvC-like domain comprises the amino acid sequence:
I-V-X2-E-M-A-R-E (SEQ ID NO:13), wherein
X2 is I, L or V (e.g., I or V) (e.g., the eaCas9 molecule can comprise an additional RuvC-like domain shown in FIG. 2 or FIG. 5 (depicted as “B”)).

An additional RuvC-like domain can comprise an amino acid sequence:
H-H-A-X1-D-A-X2-X3 (SEQ ID NO:14), wherein
X1 is H or L;
X2 is R or V; and
X3 is E or V.

In an embodiment, the additional RuvC-like domain comprises the amino acid sequence: H-H-A-H-D-A-Y-L (SEQ ID NO:15).

In an embodiment, the additional RuvC-like domain differs from a sequence of SEQ ID NO:13, 15, 12 or 14 by as many as 1, but no more than 2, 3, 4, or 5 residues.

In some embodiments, the sequence flanking the N-terminal RuvC-like domain is a sequences of formula V:

(SEQ ID NO: 16)
K-X1′-Y-X2′-X3′-X4′-Z-T-D-X9′-Y,

wherein
X1′ is selected from K and P,
X2′ is selected from V, L, I, and F (e.g., V, I and L);
X3′ is selected from G, A and S (e.g., G),
X4′ is selected from L, I, V and F (e.g., L);
X9′ is selected from D, E, N and Q; and
Z is an N-terminal RuvC-like domain, e.g., as described above.

HNH-Like Domains

In an embodiment, an HNH-like domain cleaves a single stranded complementary domain, e.g., a complementary strand of a double stranded nucleic acid molecule. In an embodiment, an HNH-like domain is at least 15, 20, 25 amino acids in length but not more than 40, 35 or 30 amino acids in length, e.g., 20 to 35 amino acids in length, e.g., 25 to 30 amino acids in length. Exemplary HNH-like domains are described below.

In an embodiment, an eaCas9 molecule comprises an HNH-like domain having an amino acid sequence of formula VI:
X1-X2-X3-H-X4-X5-P-X6-X7-X8-X9-X10-X11-X12-X13-X14-X15-N-X16-X17-X18-X19-X20-X21-X22-X23-N(SEQ ID NO:17), wherein
X1 is selected from D, E, Q and N (e.g., D and E);
X2 is selected from L, I, R, Q, V, M and K;
X3 is selected from D and E;
X4 is selected from I, V, T, A and L (e.g., A, I and V);
X5 is selected from V, Y, I, L, F and W (e.g., V, I and L);
X6 is selected from Q, H, R, K, Y, I, L, F and W;
X7 is selected from S, A, D, T and K (e.g., S and A);
X8 is selected from F, L, V, K, Y, M, I, R, A, E, D and Q (e.g., F);
X9 is selected from L, R, T, I, V, S, C, Y, K, F and G;
X10 is selected from K, Q, Y, T, F, L, W, M, A, E, G, and S;
X11 is selected from D, S, N, R, L and T (e.g., D);
X12 is selected from D, N and S;
X13 is selected from S, A, T, G and R (e.g., S);
X14 is selected from I, L, F, S, R, Y, Q, W, D, K and H (e.g., I, L and F);
X15 is selected from D, S, I, N, E, A, H, F, L, Q, M, G, Y and V;
X16 is selected from K, L, R, M, T and F (e.g., L, R and K);
X17 is selected from V, L, I, A and T;
X18 is selected from L, I, V and A (e.g., L and I);
X19 is selected from T, V, C, E, S and A (e.g., T and V);
X20 is selected from R, F, T, W, E, L, N, C, K, V, S, Q, I, Y, H and A;
X21 is selected from S, P, R, K, N, A, H, Q, G and L;
X22 is selected from D, G, T, N, S, K, A, I, E, L, Q, R and Y; and
X23 is selected from K, V, A, E, Y, I, C, L, S, T, G, K, M, D and F.

In an embodiment, a HNH-like domain differs from a sequence of SEQ ID NO:17 by at least 1, but no more than, 2, 3, 4, or 5 residues.

In an embodiment, the HNH-like domain is cleavage competent.

In an embodiment, the HNH-like domain is cleavage incompetent.

In an embodiment, an eaCas9 molecule comprises an HNH-like domain comprising an amino acid sequence of formula VII:

(SEQ ID NO: 18)
X1-X2-X3-H-X4-X5-P-X6-S-X8-X9-X10-D-D-S-X14-X15-N-
K-V-L-X19-X20-X21-X22-X23-N,

wherein
X1 is selected from D and E;
X2 is selected from L, I, R, Q, V, M and K;
X3 is selected from D and E;
X4 is selected from I, V, T, A and L (e.g., A, I and V);
X5 is selected from V, Y, I, L, F and W (e.g., V, I and L);
X6 is selected from Q, H, R, K, Y, I, L, F and W;
X8 is selected from F, L, V, K, Y, M, I, R, A, E, D and Q (e.g., F);
X9 is selected from L, R, T, I, V, S, C, Y, K, F and G;
X10 is selected from K, Q, Y, T, F, L, W, M, A, E, G, and S;
X14 is selected from I, L, F, S, R, Y, Q, W, D, K and H (e.g., I, L and F);
X15 is selected from D, S, I, N, E, A, H, F, L, Q, M, G, Y and V;
X19 is selected from T, V, C, E, S and A (e.g., T and V);
X20 is selected from R, F, T, W, E, L, N, C, K, V, S, Q, I, Y, H and A;
X21 is selected from S, P, R, K, N, A, H, Q, G and L;
X22 is selected from D, G, T, N, S, K, A, I, E, L, Q, R and Y; and
X23 is selected from K, V, A, E, Y, I, C, L, S, T, G, K, M, D and F.

In an embodiment, the HNH-like domain differs from a sequence of SEQ ID NO:18 by 1, 2, 3, 4, or 5 residues.

In an embodiment, an eaCas9 molecule comprises an HNH-like domain comprising an amino acid sequence of formula VII:

(SEQ ID NO: 19)
X1-V-X3-H-I-V-P-X6-S-X8-X9-X10-D-D-S-X14-X15-N-K-V-
L-T-X20-X21-X22-X23-N,

wherein
X1 is selected from D and E;
X3 is selected from D and E;
X6 is selected from Q, H, R, K, Y, I, L and W;
X8 is selected from F, L, V, K, Y, M, I, R, A, E, D and Q (e.g., F);
X9 is selected from L, R, T, I, V, S, C, Y, K, F and G;
X10 is selected from K, Q, Y, T, F, L, W, M, A, E, G, and S;
X14 is selected from I, L, F, S, R, Y, Q, W, D, K and H (e.g., I, L and F);
X15 is selected from D, S, I, N, E, A, H, F, L, Q, M, G, Y and V;
X20 is selected from R, F, T, W, E, L, N, C, K, V, S, Q, I, Y, H and A;
X21 is selected from S, P, R, K, N, A, H, Q, G and L;
X22 is selected from D, G, T, N, S, K, A, I, E, L, Q, R and Y; and
X23 is selected from K, V, A, E, Y, I, C, L, S, T, G, K, M, D and F.

In an embodiment, the HNH-like domain differs from a sequence of SEQ ID NO:19 by 1, 2, 3, 4, or 5 residues.

In an embodiment, an eaCas9 molecule comprises an HNH-like domain having an amino acid sequence of formula VIII:

(SEQ ID NO: 20)
D-X2-D-H-I-X5-P-Q-X7-F-X9-X10-D-X12-S-I-D-N-X16-V-
L-X19-X20-S-X22-X23-N,

wherein
X2 is selected from I and V;
X5 is selected from I and V;
X7 is selected from A and S;
X9 is selected from I and L;
X10 is selected from K and T;
X12 is selected from D and N;
X16 is selected from R, K and L; X19 is selected from T and V;
X20 is selected from S and R;
X22 is selected from K, D and A; and
X23 is selected from E, K, G and N (e.g., the eaCas9 molecule can comprise an HNH-like domain as described herein).

In an embodiment, the HNH-like domain differs from a sequence of SEQ ID NO:20 by as many as 1, but no more than 2, 3, 4, or 5 residues.

In an embodiment, an eaCas9 molecule comprises the amino acid sequence of formula IX:

(SEQ ID NO: 21)
L-Y-Y-L-Q-N-G-X1′-D-M-Y-X2′-X3′-X4′-X5′-L-D-I—X6′-
X7′-L-S-X8′-Y-Z-N-R-X9′-K-X10′-D-X11′-V-P,

wherein
X1′ is selected from K and R;
X2′ is selected from V and T;
X3′ is selected from G and D;
X4′ is selected from E, Q and D;
X5′ is selected from E and D;
X6′ is selected from D, N and H;
X7′ is selected from Y, R and N;
X8′ is selected from Q, D and N; X9′ is selected from G and E;
X10′ is selected from S and G;
X11′ is selected from D and N; and
Z is an HNH-like domain, e.g., as described above.

In an embodiment, the eaCas9 molecule comprises an amino acid sequence that differs from a sequence of SEQ ID NO:21 by as many as 1, but no more than 2, 3, 4, or 5 residues.

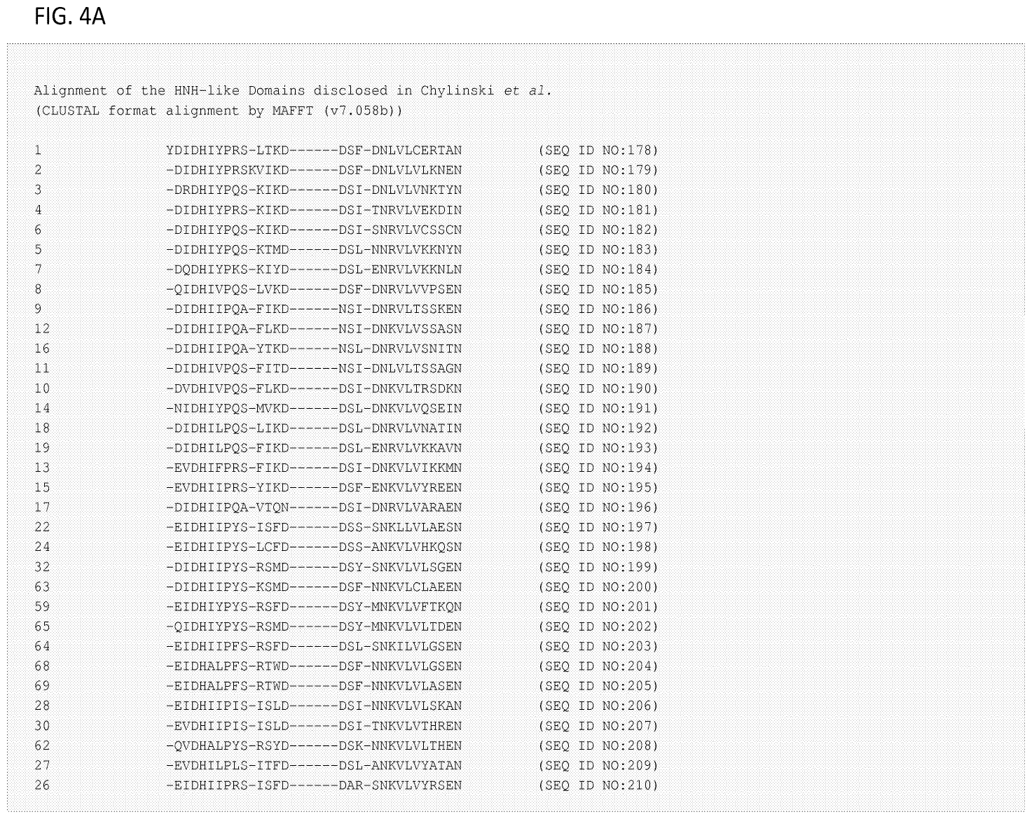

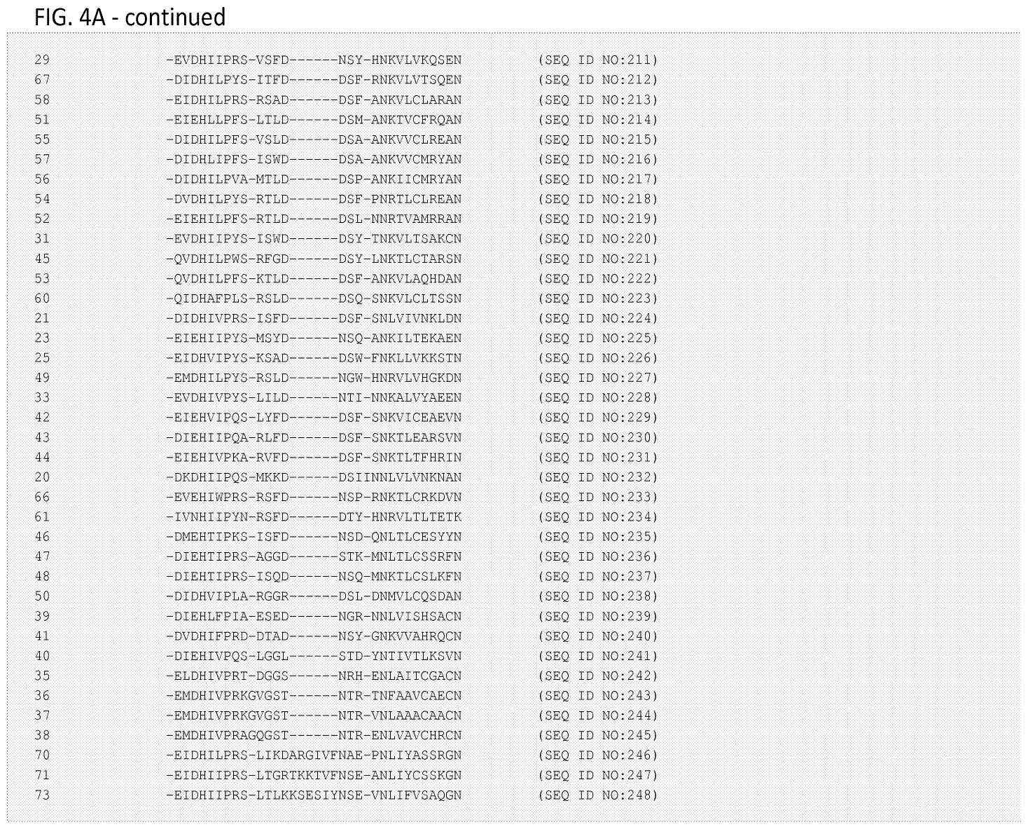

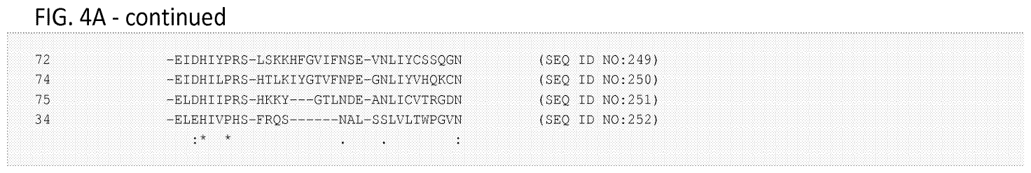

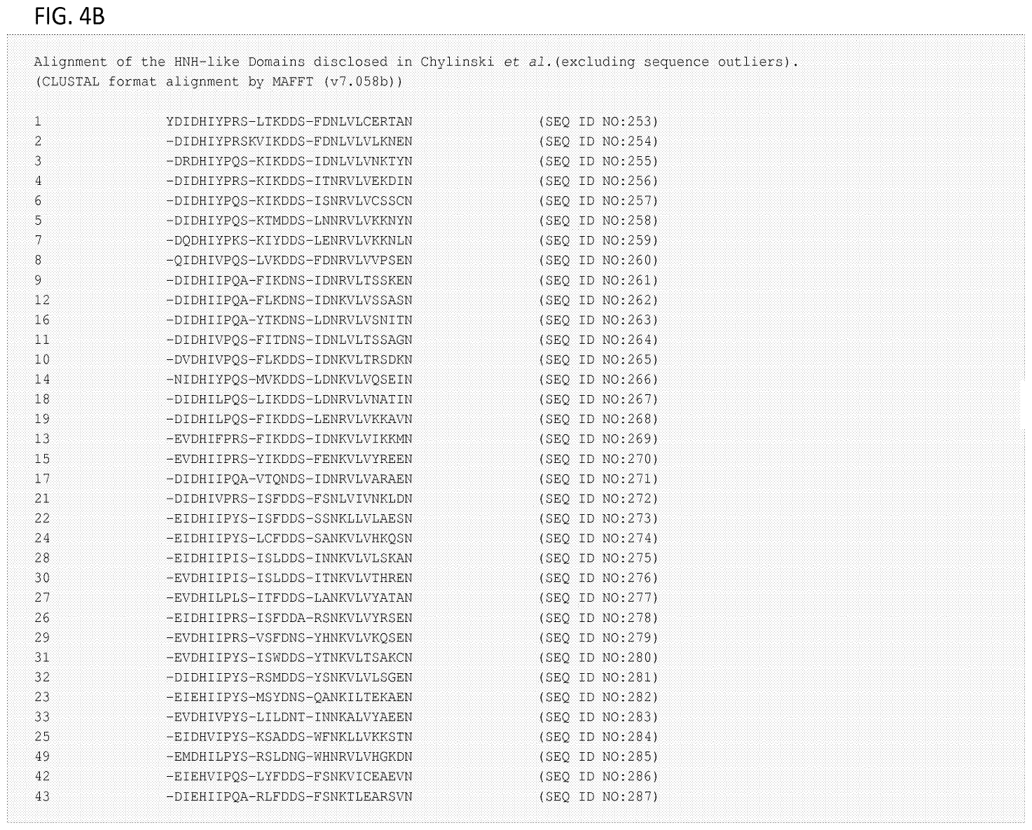

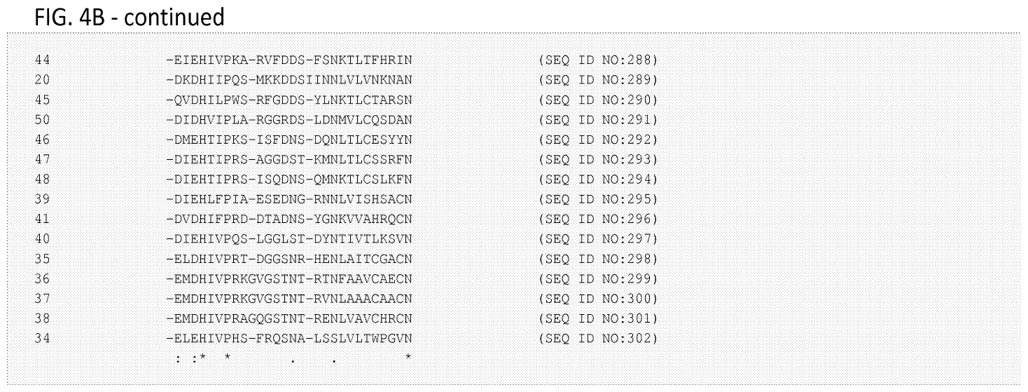

In an embodiment, the HNH-like domain differs from a sequence of an HNH-like domain disclosed herein, e.g., in FIG. 4A or FIG. 5, as many as 1, but no more than 2, 3, 4, or 5 residues.

In an embodiment, the HNH-like domain differs from a sequence of an HNH-like domain disclosed herein, e.g., in FIG. 4B, by as many as 1, but no more than 2, 3, 4, or 5 residues. In an embodiment, 1, 2, all 3 of the highly conserved residues identified in FIG. 4B are present.

Altered Cas9 Molecules

Naturally occurring Cas9 molecules possess a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In an embodiment, a Cas9 molecules can include all or a subset of these properties. In typical embodiments, Cas9 molecules have the ability to interact with a gRNA molecule and, in concert with the gRNA molecule, localize to a site in a nucleic acid. Other activities, e.g., PAM specificity, cleavage activity, or helicase activity can vary more widely in Cas9 molecules.

Cas9 molecules with desired properties can be made in a number of ways, e.g., by alteration of a parental, e.g., naturally occurring Cas9 molecules to provide an altered Cas9 molecule having a desired property. For example, one or more mutations or differences relative to a parental Cas9 molecule can be introduced. Such mutations and differences comprise: substitutions (e.g., conservative substitutions or substitutions of non-essential amino acids); insertions; or deletions. In an embodiment, a Cas9 molecule can comprises one or more mutations or differences, e.g., at least 1, 2, 3, 4, 5, 10, 15, 20, 30, 40 or 50 mutations but less than 200, 100, or 80 mutations relative to a reference Cas9 molecule.

In an embodiment, a mutation or mutations do not have a substantial effect on a Cas9 activity, e.g. a Cas9 activity described herein. In an embodiment, a mutation or mutations have a substantial effect on a Cas9 activity, e.g. a Cas9 activity described herein. In an embodiment, exemplary activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in an N-terminal RuvC-like domain. In some embodiments, a mutation(s) is present in an HNH-like domain. In some embodiments, mutations are present in both an N-terminal RuvC-like domain and an HNH-like domain.

Whether or not a particular sequence, e.g., a substitution, may affect one or more activity, such as targeting activity, cleavage activity, etc, can be evaluated or predicted, e.g., by evaluating whether the mutation is conservative or by the method described in Section III. In an embodiment, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In an embodiment, the altered Cas9 molecule is an eaCas9 molecule comprising the fixed amino acid residues of S. pyogenes shown in the consensus sequence disclosed in FIG. 2, and has one or more amino acids that differ from the amino acid sequence of S. pyogenes (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIG. 2 or SEQ ID NO:7. In an embodiment, the altered Cas9 molecule is an eiCas9 molecule wherein one or more of the fixed amino acid residues of S. pyogenes shown in the consensus sequence disclosed in FIG. 2 (e.g., 2, 3, 5, 10, 15, 20, 30, 50, 70, 80, 90, 100, 200 amino acid residues) is mutated.

In an embodiment, the altered Cas9 molecule comprises a sequence in which:
the sequence corresponding to the fixed sequence of the consensus sequence disclosed in FIG. 2 differs at no more than 1, 2, 3, 4, 5, 10, 15, or 20% of the fixed residues in the consensus sequence disclosed in FIG. 2;
the sequence corresponding to the residues identified by “*” in the consensus sequence disclosed in FIG. 2 differ at no more than 1, 2, 3, 4, 5, 10, 15, 20, 25, 30, 35, or 40% of the “*” residues from the corresponding sequence of naturally occurring Cas9 molecule, e.g., an S. pyogenes Cas9 molecule; and,
the sequence corresponding to the residues identified by “-” in the consensus sequence disclosed in FIG. 2 differ at no more than 5, 10, 15, 20, 25, 30, 35, 40, 45, 55, or 60% of the “-” residues from the corresponding sequence of naturally occurring Cas9 molecule, e.g., an S. pyogenes Cas9 molecule.

In an embodiment, the altered Cas9 molecule is an eaCas9 molecule comprising the fixed amino acid residues of S. thermophilus shown in the consensus sequence disclosed in FIG. 2, and has one or more amino acids that differ from the amino acid sequence of S. thermophilus (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIG. 2. In an embodiment, the altered Cas9 molecule is an eiCas9 molecule wherein one or more of the fixed amino acid residues of S. thermophilus shown in the consensus sequence disclosed in FIG. 2 (e.g., 2, 3, 5, 10, 15, 20, 30, 50, 70, 80, 90, 100, 200 amino acid residues) is mutated.

In an embodiment the altered Cas9 molecule comprises a sequence in which:
the sequence corresponding to the fixed sequence of the consensus sequence disclosed in FIG. 2 differs at no more than 1, 2, 3, 4, 5, 10, 15, or 20% of the fixed residues in the consensus sequence disclosed in FIG. 2;
the sequence corresponding to the residues identified by “*” in the consensus sequence disclosed in FIG. 2 differ at no more than 1, 2, 3, 4, 5, 10, 15, 20, 25, 30, 35, or 40% of the “*” residues from the corresponding sequence of naturally occurring Cas9 molecule, e.g., an S. thermophilus Cas9 molecule; and,
the sequence corresponding to the residues identified by “-” in the consensus sequence disclosed in FIG. 2 differ at no more than 5, 10, 15, 20, 25, 30, 35, 40, 45, 55, or 60% of the “-” residues from the corresponding sequence of naturally occurring Cas9 molecule, e.g., an S. thermophilus Cas9 molecule.

In an embodiment, the altered Cas9 molecule is an eaCas9 molecule comprising the fixed amino acid residues of S. mutans shown in the consensus sequence disclosed in FIG. 2, and has one or more amino acids that differ from the amino acid sequence of S. mutans (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIG. 2. In an embodiment, the altered Cas9 molecule is an eiCas9 molecule wherein one or more of the fixed amino acid residues of S. mutans shown in the consensus sequence disclosed in FIG. 2 (e.g., 2, 3, 5, 10, 15, 20, 30, 50, 70, 80, 90, 100, 200 amino acid residues) is mutated.

In an embodiment the altered Cas9 molecule comprises a sequence in which:
the sequence corresponding to the fixed sequence of the consensus sequence disclosed in FIG. 2 differs at no more than 1, 2, 3, 4, 5, 10, 15, or 20% of the fixed residues in the consensus sequence disclosed in FIG. 2;
the sequence corresponding to the residues identified by “*” in the consensus sequence disclosed in FIG. 2 differ at no more than 1, 2, 3, 4, 5, 10, 15, 20, 25, 30, 35, or 40% of the “*” residues from the corresponding sequence of naturally occurring Cas9 molecule, e.g., an S. mutans Cas9 molecule; and,
the sequence corresponding to the residues identified by “-” in the consensus sequence disclosed in FIG. 2 differ at no more than 5, 10, 15, 20, 25, 30, 35, 40, 45, 55, or 60% of the “-” residues from the corresponding sequence of naturally occurring Cas9 molecule, e.g., an S. mutans Cas9 molecule.

In an embodiment, the altered Cas9 molecule is an eaCas9 molecule comprising the fixed amino acid residues of L. innocula shown in the consensus sequence disclosed in FIG. 2, and has one or more amino acids that differ from the amino acid sequence of L. innocula (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIG. 2. In an embodiment, the altered Cas9 molecule is an eiCas9 molecule wherein one or more of the fixed amino acid residues of L. innocula shown in the consensus sequence disclosed in FIG. 2 (e.g., 2, 3, 5, 10, 15, 20, 30, 50, 70, 80, 90, 100, 200 amino acid residues) is mutated.

In an embodiment the altered Cas9 molecule comprises a sequence in which:
the sequence corresponding to the fixed sequence of the consensus sequence disclosed in FIG. 2 differs at no more than 1, 2, 3, 4, 5, 10, 15, or 20% of the fixed residues in the consensus sequence disclosed in FIG. 2;
the sequence corresponding to the residues identified by “*” in the consensus sequence disclosed in FIG. 2 differ at no more than 1, 2, 3, 4, 5, 10, 15, 20, 25, 30, 35, or 40% of the “*” residues from the corresponding sequence of naturally occurring Cas9 molecule, e.g., an L. innocula Cas9 molecule; and,
the sequence corresponding to the residues identified by “-” in the consensus sequence disclosed in FIG. 2 differ at no more than 5, 10, 15, 20, 25, 30, 35, 40, 45, 55, or 60% of the “-” residues from the corresponding sequence of naturally occurring Cas9 molecule, e.g., an L. innocula Cas9 molecule.

In an embodiment, the altered Cas9 molecule, e.g., an eaCas9 molecule or an eiCas9 molecule, can be a fusion, e.g., of two of more different Cas9 molecules, e.g., of two or more naturally occurring Cas9 molecules of different species. For example, a fragment of a naturally occurring Cas9 molecule of one species can be fused to a fragment of a Cas9 molecule of a second species. As an example, a fragment of Cas9 of S. pyogenes comprising an N-terminal RuvC-like domain can be fused to a fragment of Cas9 of a species other than S. pyogenes (e.g., S. thermophilus) comprising an HNH-like domain.

Cas9 Molecules with Altered PAM Recognition or No PAM Recognition

Naturally occurring Cas9 molecules can recognize specific PAM sequences, for example the PAM recognition sequences described above for S. pyogenes, S. thermophilus, S. mutans, S. aureus and N. meningitidis.

In an embodiment, a Cas9 molecule has the same PAM specificities as a naturally occurring Cas9 molecule. In other embodiments, a Cas9 molecule has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule recognizes to decrease off target sites and/or improve specificity; or eliminate a PAM recognition requirement. In an embodiment, a Cas9 molecule can be altered, e.g., to increase length of PAM recognition sequence and/or improve Cas9 specificity to high level of identity to decrease off target sites and increase specificity. In an embodiment, the length of the PAM recognition sequence is at least 4, 5, 6, 7, 8, 9, 10 or 15 amino acids in length. Cas9 molecules that recognize different PAM sequences and/or have reduced off-target activity can be generated using directed evolution. Exemplary methods and systems that can be used for directed evolution of Cas9 molecules are described, e.g., in Esvelt et al., Nature 2011, 472(7344): 499-503. Candidate Cas9 molecules can be evaluated, e.g., by methods described in Section III.

Non-Cleaving and Modified-Cleavage Cas9 Molecules

In an embodiment, a Cas9 molecule comprises a cleavage property that differs from naturally occurring Cas9 molecules, e.g., that differs from the naturally occurring Cas9 molecule having the closest homology. For example, a Cas9 molecule can differ from naturally occurring Cas9 molecules, e.g., a Cas9 molecule of S. pyogenes, as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

Modified Cleavage eaCas9 Molecules

In an embodiment, an eaCas9 molecule comprises one or more of the following activities: cleavage activity associated with an N-terminal RuvC-like domain; cleavage activity associated with an HNH-like domain; cleavage activity associated with an HNH domain and cleavage activity associated with an N-terminal RuvC-like domain.

In an embodiment an eaCas9 molecule comprises an active, or cleavage competent, HNH-like domain (e.g., an HNH-like domain described herein, e.g., SEQ ID NO:17, SEQ ID NO:18, SEQ ID NO:19, SEQ ID NO:20 or SEQ ID NO:21) and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIG. 2 or an aspartic acid at position 10 of SEQ ID NO:7, e.g., can be substituted with an alanine. In an embodiment, the eaCas9 differs from wild type in the N-terminal RuvC-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

In an embodiment, an eaCas9 molecule comprises an inactive, or cleavage incompetent, HNH domain and an active, or cleavage competent, N-terminal RuvC-like domain (e.g., an HNH-like domain described herein, e.g., SEQ ID NO:8, SEQ ID NO:9, SEQ ID NO:10, SEQ ID NO:11, SEQ ID NO:12, SEQ ID NO:13, SEQ ID NO:14 or SEQ ID NO:15). Exemplary inactive, or cleavage incompetent HNH-like domains can have a mutation at one or more of: a histidine in an HNH-like domain, e.g., a histidine at position 856 of the consensus sequence disclosed in FIG. 2, e.g., can be substituted with an alanine; and one or more asparagines in an HNH-like domain, e.g., an asparagine at position 870 of the consensus sequence disclosed in FIG. 2 and/or at position 879 of the consensus sequence disclosed in FIG. 2, e.g., can be substituted with an alanine. In an embodiment, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

Non-Cleaving eiCas9 Molecules

In an embodiment, the altered Cas9 molecule is an eiCas9 molecule which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. thermophilus, S. aureus or N. meningitidis. In an embodiment, the reference Cas9 molecule is the naturally occurring Cas9 molecule having the closest sequence identity or homology. In an embodiment, the eiCas9 molecule lacks substantial cleavage activity associated with an N-terminal RuvC-like domain and cleavage activity associated with an HNH-like domain.

In an embodiment, an eiCas9 molecule comprises an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIG. 2 or an aspartic acid at position 10 of SEQ ID NO:7, e.g., can be substituted with an alanine.

In an embodiment an eiCas9 molecule comprises an inactive, or cleavage incompetent, HNH domain (e.g., an HNH-like domain described herein, e.g., SEQ ID NO:8, SEQ ID NO:9, SEQ ID NO:10, SEQ ID NO:11, SEQ ID NO:12, SEQ ID NO:13, SEQ ID NO:14 or SEQ ID NO:15). Exemplary inactive, or cleavage incompetent HNH-like domains can have a mutation at one or more of: a histidine in an HNH-like domain, e.g., a histidine at position 856 of the consensus sequence disclosed in FIG. 2, e.g., can be substituted with an alanine; and one or more asparagines in an HNH-like domain, e.g., an asparagine at position 870 of the consensus sequence disclosed in FIG. 2 and/or at position 879 of the consensus sequence disclosed in FIG. 2, e.g., can be substituted with an alanine.

A catalytically inactive Cas9 molecule may be fused with a transcription repressor. An eiCas9 fusion protein complexes with a gRNA and localizes to a DNA sequence specified by gRNA's targeting domain, but, unlike an eaCas9, it will not cleave the target DNA. Fusion of an effector domain, such as a transcriptional repression domain, to an eiCas9 enables recruitment of the effector to any DNA site specified by the gRNA. Site specific targeting of an eiCas9 or an eiCas9 fusion protein to a promoter region of a gene can block RNA polymerase binding to the promoter region, a transcription factor (e.g., a transcription activator) and/or a transcriptional enhancer to inhibit transcription activation. Alternatively, site specific targeting of an eiCas9-fusion to a transcription repressor to a promoter region of a gene can be used to decrease transcription activation.

Transcription repressors or transcription repressor domains that may be fused to an eiCas9 molecule can include Kruppel associated box (KRAB or SKD), the Mad mSIN3 interaction domain (SID) or the ERF repressor domain (ERD).

In another embodiment, an eiCas9 molecule may be fused with a protein that modifies chromatin. For example, an eiCas9 molecule may be fused to heterochromatin protein 1 (HP1), a histone lysine methyltransferase (e.g., SUV39H1, SUV39H2, G9A, ESET/SETDB1, Pr-SET7/8, SUV4-20H1, RIZ1), a histone lysine demethylates (e.g., LSD1/BHC110, SpLsd1/Sw, 1/Saf110, Su(var)3-3, JMJD2A/JHDM3A, JMJD2B, JMJD2C/GASC1, JMJD2D, Rph1, JARID1A/RBP2, JAR1D1B/PLU-1, JAR1D1C/SMCX, JARID1D/SMCY, Lid, Jhn2, Jmj2), a histone lysine deacetylases (e.g., HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11) and a DNA methylases (DNMT1, DNMT2a/DMNT3b, MET1). An eiCas9-chromatin modifying molecule fusion protein can be used to alter chromatin status to reduce expression a target gene.

The heterologous sequence (e.g., the transcription repressor domain) may be fused to the N- or C-terminus of the eiCas9 protein. In an alternative embodiment, the heterologous sequence (e.g., the transcription repressor domain) may be fused to an internal portion (i.e., a portion other than the N-terminus or C-terminus) of the eiCas9 protein.

The ability of a Cas9 molecule/gRNA molecule complex to bind to and cleave a target nucleic acid can be evaluated, e.g., by the methods described herein in Section III. The activity of a Cas9 molecule, either an eaCas9 or a eiCas9, alone or in a complex with a gRNA molecule may also be evaluated by methods well-known in the art, including, gene expression assays and chromatin-based assays, e.g., chromatin immunoprecipitation (ChiP) and chromatin in vivo assay (CiA).

Nucleic Acids Encoding Cas9 Molecules

Nucleic acids encoding the Cas9 molecules, e.g., an eaCas9 molecule or an eiCas9 molecule are provided herein.

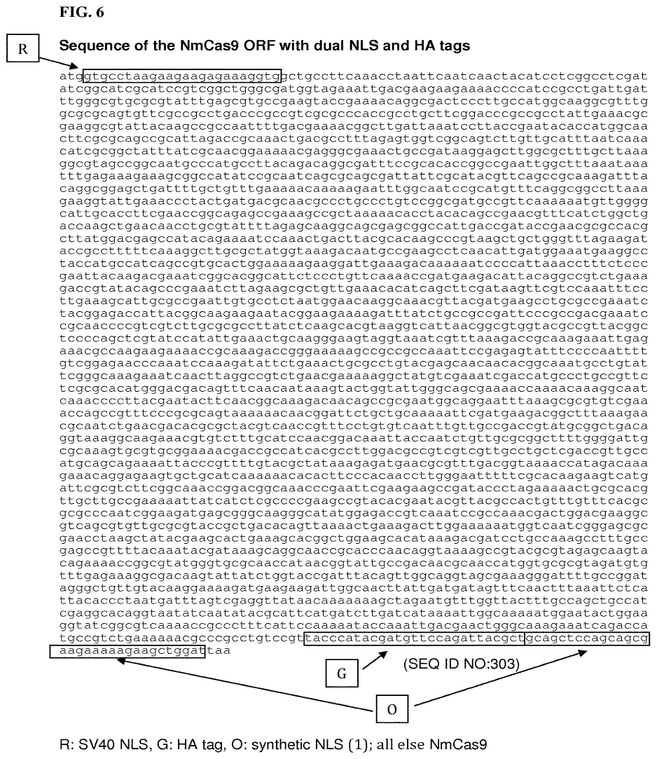

Exemplary nucleic acids encoding Cas9 molecules are described in Cong et al., SCIENCE 2013, 399(6121):819-823; Wang et al., CELL 2013, 153(4):910-918; Mali et al., SCIENCE 2013, 399(6121):823-826; Jinek et al., SCIENCE 2012, 337(6096):816-821. Another exemplary nucleic acid encoding a Cas9 molecule of N. meningitidis is shown in FIG. 6.

In an embodiment, a nucleic acid encoding a Cas9 molecule can be a synthetic nucleic acid sequence. For example, the synthetic nucleic acid molecule can be chemically modified, e.g., as described in Section X. In an embodiment, the Cas9 mRNA has one or more of, e.g., all of the following properties: it is capped, polyadenylated, substituted with 5-methylcytidine and/or pseudouridine.

In addition or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule may comprise a nuclear localization sequence (NLS). Nuclear localization sequences are known in the art.

Provided below is an exemplary codon optimized nucleic acid sequence encoding a Cas9 molecule of S. pyogenes.

(SEQ ID NO: 22)
ATGGATAAAA AGTACAGCAT CGGGCTGGAC ATCGGTACAA
ACTCAGTGGG GTGGGCCGTG ATTACGGACG AGTACAAGGT
ACCCTCCAAA AAATTTAAAG TGCTGGGTAA CACGGACAGA
CACTCTATAA AGAAAAATCT TATTGGAGCC TTGCTGTTCG
ACTCAGGCGA GACAGCCGAA GCCACAAGGT TGAAGCGGAC
CGCCAGGAGG CGGTATACCA GGAGAAAGAA CCGCATATGC
TACCTGCAAG AAATCTTCAG TAACGAGATG GCAAAGGTTG
ACGATAGCTT TTTCCATCGC CTGGAAGAAT CCTTTCTTGT
TGAGGAAGAC AAGAAGCACG AACGGCACCC CATCTTTGGC
AATATTGTCG ACGAAGTGGC ATATCACGAA AAGTACCCGA
CTATCTACCA CCTCAGGAAG AAGCTGGTGG ACTCTACCGA
TAAGGCGGAC CTCAGACTTA TTTATTTGGC ACTCGCCCAC
ATGATTAAAT TTAGAGGACA TTTCTTGATC GAGGGCGACC
TGAACCCGGA CAACAGTGAC GTCGATAAGC TGTTCATCCA
ACTTGTGCAG ACCTACAATC AACTGTTCGA AGAAAACCCT
ATAAATGCTT CAGGAGTCGA CGCTAAAGCA ATCCTGTCCG
CGCGCCTCTC AAAATCTAGA AGACTTGAGA ATCTGATTGC
TCAGTTGCCC GGGGAAAAGA AAAATGGATT GTTTGGCAAC
CTGATCGCCC TCAGTCTCGG ACTGACCCCA AATTTCAAAA
GTAACTTCGA CCTGGCCGAA GACGCTAAGC TCCAGCTGTC
CAAGGACACA TACGATGACG ACCTCGACAA TCTGCTGGCC
CAGATTGGGG ATCAGTACGC CGATCTCTTT TTGGCAGCAA
AGAACCTGTC CGACGCCATC CTGTTGAGCG ATATCTTGAG
AGTGAACACC GAAATTACTA AAGCACCCCT TAGCGCATCT
ATGATCAAGC GGTACGACGA GCATCATCAG GATCTGACCC
TGCTGAAGGC TCTTGTGAGG CAACAGCTCC CCGAAAAATA
CAAGGAAATC TTCTTTGACC AGAGCAAAAA CGGCTACGCT
GGCTATATAG ATGGTGGGGC CAGTCAGGAG GAATTCTATA
AATTCATCAA GCCCATTCTC GAGAAAATGG ACGGCACAGA
GGAGTTGCTG GTCAAACTTA ACAGGGAGGA CCTGCTGCGG
AAGCAGCGGA CCTTTGACAA CGGGTCTATC CCCCACCAGA
TTCATCTGGG CGAACTGCAC GCAATCCTGA GGAGGCAGGA
GGATTTTTAT CCTTTTCTTA AAGATAACCG CGAGAAAATA
GAAAAGATTC TTACATTCAG GATCCCGTAC TACGTGGGAC
CTCTCGCCCG GGGCAATTCA CGGTTTGCCT GGATGACAAG
GAAGTCAGAG GAGACTATTA CACCTTGGAA CTTCGAAGAA
GTGGTGGACA AGGGTGCATC TGCCCAGTCT TTCATCGAGC
GGATGACAAA TTTTGACAAG AACCTCCCTA ATGAGAAGGT
GCTGCCCAAA CATTCTCTGC TCTACGAGTA CTTTACCGTC
TACAATGAAC TGACTAAAGT CAAGTACGTC ACCGAGGGAA
TGAGGAAGCC GGCATTCCTT AGTGGAGAAC AGAAGAAGGC
GATTGTAGAC CTGTTGTTCA AGACCAACAG GAAGGTGACT
GTGAAGCAAC TTAAAGAAGA CTACTTTAAG AAGATCGAAT
GTTTTGACAG TGTGGAAATT TCAGGGGTTG AAGACCGCTT
CAATGCGTCA TTGGGGACTT ACCATGATCT TCTCAAGATC
ATAAAGGACA AAGACTTCCT GGACAACGAA GAAAATGAGG
ATATTCTCGA AGACATCGTC CTCACCCTGA CCCTGTTCGA
AGACAGGGAA ATGATAGAAG AGCGCTTGAA AACCTATGCC
CACCTCTTCG ACGATAAAGT TATGAAGCAG CTGAAGCGCA
GGAGATACAC AGGATGGGGA AGATTGTCAA GGAAGCTGAT
CAATGGAATT AGGGATAAAC AGAGTGGCAA GACCATACTG
GATTTCCTCA AATCTGATGG CTTCGCCAAT AGGAACTTCA
TGCAACTGAT TCACGATGAC TCTCTTACCT TCAAGGAGGA
CATTCAAAAG GCTCAGGTGA GCGGGCAGGG AGACTCCCTT
CATGAACACA TCGCGAATTT GGCAGGTTCC CCCGCTATTA
AAAAGGGCAT CCTTCAAACT GTCAAGGTGG TGGATGAATT
GGTCAAGGTA ATGGGCAGAC ATAAGCCAGA AAATATTGTG
ATCGAGATGG CCCGCGAAAA CCAGACCACA CAGAAGGGCC
AGAAAAATAG TAGAGAGCGG ATGAAGAGGA TCGAGGAGGG
CATCAAAGAG CTGGGATCTC AGATTCTCAA AGAACACCCC
GTAGAAAACA CACAGCTGCA GAACGAAAAA TTGTACTTGT
ACTATCTGCA GAACGGCAGA GACATGTACG TCGACCAAGA
ACTTGATATT AATAGACTGT CCGACTATGA CGTAGACCAT
ATCGTGCCCC AGTCCTTCCT GAAGGACGAC TCCATTGATA
ACAAAGTCTT GACAAGAAGC GACAAGAACA GGGGTAAAAG
TGATAATGTG CCTAGCGAGG AGGTGGTGAA AAAAATGAAG
AACTACTGGC GACAGCTGCT TAATGCAAAG CTCATTACAC
AACGGAAGTT CGATAATCTG ACGAAAGCAG AGAGAGGTGG
CTTGTCTGAG TTGGACAAGG CAGGGTTTAT TAAGCGGCAG
CTGGTGGAAA CTAGGCAGAT CACAAAGCAC GTGGCGCAGA
TTTTGGACAG CCGGATGAAC ACAAAATACG ACGAAAATGA
TAAACTGATA CGAGAGGTCA AAGTTATCAC GCTGAAAAGC
AAGCTGGTGT CCGATTTTCG GAAAGACTTC CAGTTCTACA
AAGTTCGCGA GATTAATAAC TACCATCATG CTCACGATGC
GTACCTGAAC GCTGTTGTCG GGACCGCCTT GATAAAGAAG
TACCCAAAGC TGGAATCCGA GTTCGTATAC GGGGATTACA
AAGTGTACGA TGTGAGGAAA ATGATAGCCA AGTCCGAGCA
GGAGATTGGA AAGGCCACAG CTAAGTACTT CTTTTATTCT
AACATCATGA ATTTTTTTAA GACGGAAATT ACCCTGGCCA
ACGGAGAGAT CAGAAAGCGG CCCCTTATAG AGACAAATGG
TGAAACAGGT GAAATCGTCT GGGATAAGGG CAGGGATTTC
GCTACTGTGA GGAAGGTGCT GAGTATGCCA CAGGTAAATA
TCGTGAAAAA AACCGAAGTA CAGACCGGAG GATTTTCCAA
GGAAAGCATT TTGCCTAAAA GAAACTCAGA CAAGCTCATC
GCCCGCAAGA AAGATTGGGA CCCTAAGAAA TACGGGGGAT
TTGACTCACC CACCGTAGCC TATTCTGTGC TGGTGGTAGC
TAAGGTGGAA AAAGGAAAGT CTAAGAAGCT GAAGTCCGTG
AAGGAACTCT TGGGAATCAC TATCATGGAA AGATCATCCT
TTGAAAAGAA CCCTATCGAT TTCCTGGAGG CTAAGGGTTA
CAAGGAGGTC AAGAAAGACC TCATCATTAA ACTGCCAAAA
TACTCTCTCT TCGAGCTGGA AAATGGCAGG AAGAGAATGT
TGGCCAGCGC CGGAGAGCTG CAAAAGGGAA ACGAGCTTGC
TCTGCCCTCC AAATATGTTA ATTTTCTCTA TCTCGCTTCC
CACTATGAAA AGCTGAAAGG GTCTCCCGAA GATAACGAGC
AGAAGCAGCT GTTCGTCGAA CAGCACAAGC ACTATCTGGA
TGAAATAATC GAACAAATAA GCGAGTTCAG CAAAAGGGTT
ATCCTGGCGG ATGCTAATTT GGACAAAGTA CTGTCTGCTT
ATAACAAGCA CCGGGATAAG CCTATTAGGG AACAAGCCGA
GAATATAATT CACCTCTTTA CACTCACGAA TCTCGGAGCC
CCCGCCGCCT TCAAATACTT TGATACGACT ATCGACCGGA
AACGGTATAC CAGTACCAAA GAGGTCCTCG ATGCCACCCT
CATCCACCAG TCAATTACTG GCCTGTACGA AACACGGATC
GACCTCTCTC AACTGGGCGG CGACTAG

Provided below is the corresponding amino acid sequence of a S. pyogenes Cas9 molecule.

(SEQ ID NO: 23)
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKRYTSTKEVLDATLIHQSITGLYETRIDLSQLGGD*

Provided below is an exemplary codon optimized nucleic acid sequence encoding a Cas9 molecule of N. meningitidis.

(SEQ ID NO: 24)
ATGGCCGCCTTCAAGCCCAACCCCATCAACTACATCCTGGGCCTGGACATC
GGCATCGCCAGCGTGGGCTGGGCCATGGTGGAGATCGACGAGGACGAGAAC
CCCATCTGCCTGATCGACCTGGGTGTGCGCGTGTTCGAGCGCGCTGAGGTG
CCCAAGACTGGTGACAGTCTGGCTATGGCTCGCCGGCTTGCTCGCTCTGTT
CGGCGCCTTACTCGCCGGCGCGCTCACCGCCTTCTGCGCGCTCGCCGCCTG
CTGAAGCGCGAGGGTGTGCTGCAGGCTGCCGACTTCGACGAGAACGGCCTG
ATCAAGAGCCTGCCCAACACTCCTTGGCAGCTGCGCGCTGCCGCTCTGGAC
CGCAAGCTGACTCCTCTGGAGTGGAGCGCCGTGCTGCTGCACCTGATCAAG
CACCGCGGCTACCTGAGCCAGCGCAAGAACGAGGGCGAGACCGCCGACAAG
GAGCTGGGTGCTCTGCTGAAGGGCGTGGCCGACAACGCCCACGCCCTGCAG
ACTGGTGACTTCCGCACTCCTGCTGAGCTGGCCCTGAACAAGTTCGAGAAG
GAGAGCGGCCACATCCGCAACCAGCGCGGCGACTACAGCCACACCTTCAGC
CGCAAGGACCTGCAGGCCGAGCTGATCCTGCTGTTCGAGAAGCAGAAGGAG
TTCGGCAACCCCCACGTGAGCGGCGGCCTGAAGGAGGGCATCGAGACCCTG
CTGATGACCCAGCGCCCCGCCCTGAGCGGCGACGCCGTGCAGAAGATGCTG
GGCCACTGCACCTTCGAGCCAGCCGAGCCCAAGGCCGCCAAGAACACCTAC
ACCGCCGAGCGCTTCATCTGGCTGACCAAGCTGAACAACCTGCGCATCCTG
GAGCAGGGCAGCGAGCGCCCCCTGACCGACACCGAGCGCGCCACCCTGATG
GACGAGCCCTACCGCAAGAGCAAGCTGACCTACGCCCAGGCCCGCAAGCTG
CTGGGTCTGGAGGACACCGCCTTCTTCAAGGGCCTGCGCTACGGCAAGGAC
AACGCCGAGGCCAGCACCCTGATGGAGATGAAGGCCTACCACGCCATCAGC
CGCGCCCTGGAGAAGGAGGGCCTGAAGGACAAGAAGAGTCCTCTGAACCTG
AGCCCCGAGCTGCAGGACGAGATCGGCACCGCCTTCAGCCTGTTCAAGACC
GACGAGGACATCACCGGCCGCCTGAAGGACCGCATCCAGCCCGAGATCCTG
GAGGCCCTGCTGAAGCACATCAGCTTCGACAAGTTCGTGCAGATCAGCCTG
AAGGCCCTGCGCCGCATCGTGCCCCTGATGGAGCAGGGCAAGCGCTACGAC
GAGGCCTGCGCCGAGATCTACGGCGACCACTACGGCAAGAAGAACACCGAG
GAGAAGATCTACCTGCCTCCTATCCCCGCCGACGAGATCCGCAACCCCGTG
GTGCTGCGCGCCCTGAGCCAGGCCCGCAAGGTGATCAACGGCGTGGTGCGC
CGCTACGGCAGCCCCGCCCGCATCCACATCGAGACCGCCCGCGAGGTGGGC
AAGAGCTTCAAGGACCGCAAGGAGATCGAGAAGCGCCAGGAGGAGAACCGC
AAGGACCGCGAGAAGGCCGCCGCCAAGTTCCGCGAGTACTTCCCCAACTTC
GTGGGCGAGCCCAAGAGCAAGGACATCCTGAAGCTGCGCCTGTACGAGCAG
CAGCACGGCAAGTGCCTGTACAGCGGCAAGGAGATCAACCTGGGCCGCCTG
AACGAGAAGGGCTACGTGGAGATCGACCACGCCCTGCCCTTCAGCCGCACC
TGGGACGACAGCTTCAACAACAAGGTGCTGGTGCTGGGCAGCGAGAACCAG
AACAAGGGCAACCAGACCCCCTACGAGTACTTCAACGGCAAGGACAACAGC
CGCGAGTGGCAGGAGTTCAAGGCCCGCGTGGAGACCAGCCGCTTCCCCCGC
AGCAAGAAGCAGCGCATCCTGCTGCAGAAGTTCGACGAGGACGGCTTCAAG
GAGCGCAACCTGAACGACACCCGCTACGTGAACCGCTTCCTGTGCCAGTTC
GTGGCCGACCGCATGCGCCTGACCGGCAAGGGCAAGAAGCGCGTGTTCGCC
AGCAACGGCCAGATCACCAACCTGCTGCGCGGCTTCTGGGGCCTGCGCAAG
GTGCGCGCCGAGAACGACCGCCACCACGCCCTGGACGCCGTGGTGGTGGCC
TGCAGCACCGTGGCCATGCAGCAGAAGATCACCCGCTTCGTGCGCTACAAG
GAGATGAACGCCTTCGACGGTAAAACCATCGACAAGGAGACCGGCGAGGTG
CTGCACCAGAAGACCCACTTCCCCCAGCCCTGGGAGTTCTTCGCCCAGGAG
GTGATGATCCGCGTGTTCGGCAAGCCCGACGGCAAGCCCGAGTTCGAGGAG
GCCGACACCCCCGAGAAGCTGCGCACCCTGCTGGCCGAGAAGCTGAGCAGC
CGCCCTGAGGCCGTGCACGAGTACGTGACTCCTCTGTTCGTGAGCCGCGCC
CCCAACCGCAAGATGAGCGGTCAGGGTCACATGGAGACCGTGAAGAGCGCC
AAGCGCCTGGACGAGGGCGTGAGCGTGCTGCGCGTGCCCCTGACCCAGCTG
AAGCTGAAGGACCTGGAGAAGATGGTGAACCGCGAGCGCGAGCCCAAGCTG
TACGAGGCCCTGAAGGCCCGCCTGGAGGCCCACAAGGACGACCCCGCCAAG
GCCTTCGCCGAGCCCTTCTACAAGTACGACAAGGCCGGCAACCGCACCCAG
CAGGTGAAGGCCGTGCGCGTGGAGCAGGTGCAGAAGACCGGCGTGTGGGTG
CGCAACCACAACGGCATCGCCGACAACGCCACCATGGTGCGCGTGGACGTG
TTCGAGAAGGGCGACAAGTACTACCTGGTGCCCATCTACAGCTGGCAGGTG
GCCAAGGGCATCCTGCCCGACCGCGCCGTGGTGCAGGGCAAGGACGAGGAG
GACTGGCAGCTGATCGACGACAGCTTCAACTTCAAGTTCAGCCTGCACCCC
AACGACCTGGTGGAGGTGATCACCAAGAAGGCCCGCATGTTCGGCTACTTC
GCCAGCTGCCACCGCGGCACCGGCAACATCAACATCCGCATCCACGACCTG
GACCACAAGATCGGCAAGAACGGCATCCTGGAGGGCATCGGCGTGAAGACC
GCCCTGAGCTTCCAGAAGTACCAGATCGACGAGCTGGGCAAGGAGATCCGC
CCCTGCCGCCTGAAGAAGCGCCCTCCTGTGCGCTAA

Provided below is the corresponding amino acid sequence of a N. meningitidis Cas9 molecule.

(SEQ ID NO: 25)
MAAFKPNPINYILGLDIGIASVGWAMVEIDEDENPICLIDLGVRVFERAEV
PKTGDSLAMARRLARSVRRLTRRRAHRLLRARRLLKREGVLQAADFDENGL
IKSLPNTPWQLRAAALDRKLTPLEWSAVLLHLIKHRGYLSQRKNEGETADK
ELGALLKGVADNAHALQTGDFRTPAELALNKFEKESGHIRNQRGDYSHTFS
RKDLQAELILLFEKQKEFGNPHVSGGLKEGIETLLMTQRPALSGDAVQKML
GHCTFEPAEPKAAKNTYTAERFIWLTKLNNLRILEQGSERPLTDTERATLM
DEPYRKSKLTYAQARKLLGLEDTAFFKGLRYGKDNAEASTLMEMKAYHAIS
RALEKEGLKDKKSPLNLSPELQDEIGTAFSLFKTDEDITGRLKDRIQPEIL
EALLKHISFDKFVQISLKALRRIVPLMEQGKRYDEACAEIYGDHYGKKNTE
EKIYLPPIPADEIRNPVVLRALSQARKVINGVVRRYGSPARIHIETAREVG
KSFKDRKEIEKRQEENRKDREKAAAKFREYFPNFVGEPKSKDILKLRLYEQ
QHGKCLYSGKEINLGRLNEKGYVEIDHALPFSRTWDDSFNNKVLVLGSENQ
NKGNQTPYEYFNGKDNSREWQEFKARVETSRFPRSKKQRILLQKFDEDGFK
ERNLNDTRYVNRFLCQFVADRMRLTGKGKKRVFASNGQITNLLRGFWGLRK
VRAENDRHHALDAVVVACSTVAMQQKITRFVRYKEMNAFDGKTIDKETGEV
LHQKTHFPQPWEFFAQEVMIRVFGKPDGKPEFEEADTPEKLRTLLAEKLSS
RPEAVHEYVTPLFVSRAPNRKMSGQGHMETVKSAKRLDEGVSVLRVPLTQL
KLKDLEKMVNREREPKLYEALKARLEAHKDDPAKAFAEPFYKYDKAGNRTQ
QVKAVRVEQVQKTGVWVRNHNGIADNATMVRVDVFEKGDKYYLVPIYSWQV
AKGILPDRAVVQGKDEEDWQLIDDSFNFKFSLHPNDLVEVITKKARMFGYF
ASCHRGTGNINIRIHDLDHKIGKNGILEGIGVKTALSFQKYQIDELGKEIR
PCRLKKRPPVR*

Provided below is an amino acid sequence of a S. aureus Cas9 molecule.

(SEQ ID NO: 26)
MKRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLSE
EEFSAALLHLAKRRGVHNVNEVEEDTGNELSTKEQISRNSKALEEKYVAEL
QLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADL
YNALNDLNNLVITRDENEKLEYYEKFQIIENVFKQKKKPTLKQIAKEILVN
EEDIKGYRVTSTGKPEFTNLKVYHDIKDITARKEIIENAELLDQIAKILTI
YQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIK
VINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPR
SVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSY
FRVNNLDVKVKSINGGFTSFLRRKWKFKKERNKGYKHHAEDALIIANADFI
FKEWKKLDKAKKVMENQMFEEKQAESMPEIETEQEYKEIFITPHQIKHIKD
FKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYL
DNGVYKFVTVKNLDVIKKENYYEVNSKCYEEAKKLKKISNQAEFIASFYNN
DLIKINGELYRVIGVNNDLLNRIEVNMIDITYREYLENMNDKRPPRIIKTI
ASKTQSIKKYSTDILGNLYEVKSKKHPQIIKKG*

If any of the above Cas9 sequences are fused with a peptide or polypeptide at the C-terminus (e.g., an eiCas9 fused with a transcription repressor at the C-terminus), it is understood that the stop codon will be removed.

Other Cas Molecules

Various types of Cas molecules can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLOS COMPUTATIONAL BIOLOGY 2005, 1(6): e60 and Makarova et al., NATURE REVIEW MICROBIOLOGY 2011, 9:467-477, the contents of both references are incorporated herein by reference in their entirety. Exemplary Cas molecules (and Cas systems) are also shown in Table II-1.

TABLE II-1
Cas Systems

| | | | Structure of | Families (and | |
|---|---|---|---|---|---|
| | System | | encoded protein | superfamily) of | |
| Gene | type or | Name from | (PDB | encoded | |
| name‡ | subtype | Haft et al.§ | accessions)¶ | protein#** | Representatives |
| cas1 | Type I | cas1 | 3GOD, 3LFX and | COG1518 | SERP2463, SPy1047 |
| | Type II | | 2YZS | | and ygbT |
| | Type III | | | | |
| cas2 | Type I | cas2 | 2IVY, 2I8E and | COG1343 and | SERP2462, SPy1048, |
| | Type II | | 3EXC | COG3512 | SPy1723 (N-terminal |
| | Type III | | | | domain) and ygbF |
| cas3′ | Type I^{‡‡} | cas3 | NA | COG1203 | APE1232 and ygcB |
| cas3″ | Subtype | NA | NA | COG2254 | APE1231 and BH0336 |
| | I-A | | | | |
| | Subtype | | | | |
| | I-B | | | | |
| cas4 | Subtype | cas4 and csa1 | NA | COG1468 | APE1239 and BH0340 |
| | I-A | | | | |
| | Subtype | | | | |
| | I-B | | | | |
| | Subtype | | | | |
| | I-C | | | | |
| | Subtype | | | | |
| | I-D | | | | |
| | Subtype | | | | |
| | II-B | | | | |
| cas5 | Subtype | cas5a, cas5d, | 3KG4 | COG1688 | APE1234, BH0337, |
| | I-A | cas5e, cas5h, | | (RAMP) | devS and ygcI |
| | Subtype | cas5p, cas5t | | | |
| | I-B | and cmx5 | | | |
| | Subtype | | | | |
| | I-C | | | | |
| | Subtype | | | | |
| | I-E | | | | |
| cas6 | Subtype | cas6 and cmx6 | 3I4H | COG1583 and | PF1131 and slr7014 |
| | I-A | | | COG5551 | |
| | Subtype | | | (RAMP) | |
| | I-B | | | | |
| | Subtype | | | | |
| | I-D | | | | |
| | Subtype | | | | |
| | III-A | | | | |
| | Subtype | | | | |
| | III-B | | | | |
| cas6e | Subtype | cse3 | 1WJ9 | (RAMP) | ygcH |
| | I-E | | | | |
| cas6f | Subtype | csy4 | 2XLJ | (RAMP) | y1727 |
| | I-F | | | | |
| cas7 | Subtype | csa2, csd2, | NA | COG1857 and | devR and ygcJ |
| | I-A | cse4, csh2, csp1 | | COG3649 | |
| | Subtype | and cst2 | | (RAMP) | |
| | I-B | | | | |
| | Subtype | | | | |
| | I-C | | | | |
| | Subtype | | | | |
| | I-E | | | | |
| cas8a1 | Subtype | cmx1, cst1, | NA | BH0338-like | LA3191^{§§} and |
| | I-A^{‡‡} | csx8, csx13 and | | | PG2018^{§§} |
| | | CXXC-CXXC | | | |
| cas8a2 | Subtype | csa4 and csx9 | NA | PH0918 | AF0070, AF1873, |
| | I-A^{‡‡} | | | | MJ0385, PF0637, |
| | | | | | PH0918 and SSO1401 |
| cas8b | Subtype | csh1 and | NA | BH0338-like | MTH1090 and |
| | I-B^{‡‡} | TM1802 | | | TM1802 |
| cas8c | Subtype | csd1 and csp2 | NA | BH0338-like | BH0338 |
| | I-C^{‡‡} | | | | |
| cas9 | Type II^{‡‡} | csn1 and csx12 | NA | COG3513 | FTN_0757 and |
| | | | | | SPy1046 |
| cas10 | Type III^{‡‡} | cmr2, csm1 and | NA | COG1353 | MTH326, Rv2823c^{§§} |
| | | csx11 | | | and TM1794^{§§} |
| cas10d | Subtype | csc3 | NA | COG1353 | slr7011 |
| | I-D^{‡‡} | | | | |
| csy1 | Subtype | csy1 | NA | y1724-like | y1724 |
| | I-F^{‡‡} | | | | |
| csy2 | Subtype | csy2 | NA | (RAMP) | y1725 |
| | I-F | | | | |
| csy3 | Subtype | csy3 | NA | (RAMP) | y1726 |
| | I-F | | | | |
| cse1 | Subtype | cse1 | NA | YgcL-like | ygcL |
| | I-E^{‡‡} | | | | |
| cse2 | Subtype | cse2 | 2ZCA | YgcK-like | ygcK |
| | I-E | | | | |
| csc1 | Subtype | csc1 | NA | alr1563-like | alr1563 |
| | I-D | | | (RAMP) | |
| csc2 | Subtype | csc1 and csc2 | NA | COG1337 | slr7012 |
| | I-D | | | (RAMP) | |
| csa5 | Subtype | csa5 | NA | AF1870 | AF1870, MJ0380, |
| | I-A | | | | PF0643 and SSO1398 |
| csn2 | Subtype | csn2 | NA | SPy1049-like | SPy1049 |
| | II-A | | | | |
| csm2 | Subtype | csm2 | NA | COG1421 | MTH1081 and |
| | III-A^{‡‡} | | | | SERP2460 |
| csm3 | Subtype | csc2 and csm3 | NA | COG1337 | MTH1080 and |
| | III-A | | | (RAMP) | SERP2459 |
| csm4 | Subtype | csm4 | NA | COG1567 | MTH1079 and |
| | III-A | | | (RAMP) | SERP2458 |
| csm5 | Subtype | csm5 | NA | COG1332 | MTH1078 and |
| | III-A | | | (RAMP) | SERP2457 |
| csm6 | Subtype | APE2256 and | 2WTE | COG1517 | APE2256 and |
| | III-A | csm6 | | | SSO1445 |
| cmr1 | Subtype | cmr1 | NA | COG1367 | PF1130 |
| | III-B | | | (RAMP) | |
| cmr3 | Subtype | cmr3 | NA | COG1769 | PF1128 |
| | III-B | | | (RAMP) | |
| cmr4 | Subtype | cmr4 | NA | COG1336 | PF1126 |
| | III-B | | | (RAMP) | |
| cmr5 | Subtype | cmr5 | 2ZOP and 2OEB | COG3337 | MTH324 and PF1125 |
| | III-B^{‡‡} | | | | |
| cmr6 | Subtype | cmr6 | NA | COG1604 | PF1124 |
| | III-B | | | (RAMP) | |
| csb1 | Subtype | GSU0053 | NA | (RAMP) | Balac_1306 and |
| | I-U | | | | GSU0053 |
| csb2 | Subtype | NA | NA | (RAMP) | Balac_1305 and |
| | I-U^{§§} | | | | GSU0054 |
| csb3 | Subtype | NA | NA | (RAMP) | Balac_1303^{§§} |
| | I-U | | | | |
| csx17 | Subtype | NA | NA | NA | Btus_2683 |
| | I-U | | | | |
| csx14 | Subtype | NA | NA | NA | GSU0052 |
| | I-U | | | | |
| csx10 | Subtype | csx10 | NA | (RAMP) | Caur_2274 |
| | I-U | | | | |
| csx16 | Subtype | VVA1548 | NA | NA | VVA1548 |
| | III-U | | | | |
| csaX | Subtype | csaX | NA | NA | SSO1438 |
| | III-U | | | | |
| csx3 | Subtype | csx3 | NA | NA | AF1864 |
| | III-U | | | | |
| csx1 | Subtype | csa3, csx1, | 1XMX and 2I71 | COG1517 and | MJ1666, NE0113, |
| | III-U | csx2, DXTHG, | | COG4006 | PF1127 and TM1812 |
| | | NE0113 and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

### III. Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule are described, e.g., in Jinek et al., SCIENCE 2012; 337(6096):816-821.

Binding and Cleavage Assay: Testing the Endonuclease Activity of Cas9 Molecule

The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and ˜3-6 pmol (˜20-40 mCi) [γ-32P]-ATP in 1× T4 polynucleotide kinase reaction buffer at 37° C. for 30 min, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl2, 1 mM DTT, 5% glycerol) in a total volume of 9 μl. Reactions are initiated by the addition of 1 μl target DNA (10 nM) and incubated for 1 h at 37° C. Reactions are quenched by the addition of 20 μl of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging. The resulting cleavage products indicate that whether the complementary strand, the non-complementary strand, or both, are cleaved.

One or both of these assays can be used to evaluate the suitability of a candidate gRNA molecule or candidate Cas9 molecule.

Binding Assay: Testing the Binding of Cas9 Molecule to Target DNA

Exemplary methods for evaluating the binding of Cas9 molecule to target DNA are described, e.g., in Jinek et al., SCIENCE 2012; 337(6096):816-821.

For example, in an electrophoretic mobility shift assay, target DNA duplexes are formed by mixing of each strand (10 nmol) in deionized water, heating to 95° C. for 3 min and slow cooling to room temperature. All DNAs are purified on 8% native gels containing 1×TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-32P]-ATP using T4 polynucleotide kinase for 30 min at 37° C. Polynucleotide kinase is heat denatured at 65° C. for 20 min, and unincorporated radiolabel is removed using a column. Binding assays are performed in buffer containing 20 mM HEPES pH 7.5, 100 mM KCl, 5 mM MgCl₂, 1 mM DTT and 10% glycerol in a total volume of 10 l. Cas9 protein molecule is programmed with equimolar amounts of pre-annealed gRNA molecule and titrated from 100 pM to 1 μM. Radiolabeled DNA is added to a final concentration of 20 pM. Samples are incubated for 1 h at 37° C. and resolved at 4° C. on an 8% native polyacrylamide gel containing 1×TBE and 5 mM MgCl₂. Gels are dried and DNA visualized by phosphorimaging.

### IV. Template Nucleic Acids (Genome Editing Approaches)

The terms “template nucleic acid” and “swap nucleic acid” are used interchangeably and have identical meaning in this document and its priority documents.

Mutations in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII, may be corrected using one of the approaches discussed herein. In an embodiment, a mutation in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII, is corrected by homology directed repair (HDR) using a template nucleic acid (see Section IV.1). In an embodiment, a mutation in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII, is corrected by Non-Homologous End Joining (NHEJ) repair using a template nucleic acid (see Section IV.2).

### IV.1 HDR Repair and Template Nucleic Acids

As described herein, nuclease-induced homology directed repair (HDR) can be used to alter a target sequence and correct (e.g., repair or edit) a mutation in the genome. While not wishing to be bound by theory, it is believed that alteration of the target sequence occurs by homology-directed repair (HDR) with a donor template or template nucleic acid. For example, the donor template or the template nucleic acid provides for alteration of the target sequence. It is contemplated that a plasmid donor can be used as a template for homologous recombination. It is further contemplated that a single stranded donor template can be used as a template for alteration of the target sequence by alternate methods of homology directed repair (e.g., single strand annealing) between the target sequence and the donor template. Donor template-effected alteration of a target sequence depends on cleavage by a Cas9 molecule. Cleavage by Cas9 can comprise a double strand break or two single strand breaks.

In an embodiment, a mutation can be corrected by either a single double-strand break or two single strand breaks. In an embodiment, a mutation can be corrected by (1) a single double-strand break, (2) two single strand breaks, (3) two double stranded breaks with a break occurring on each side of the target sequence, (4) one double stranded breaks and two single strand breaks with the double strand break and two single strand breaks occurring on each side of the target sequence or (5) four single stranded breaks with a pair of single stranded breaks occurring on each side of the target sequence.

Double Strand Break Mediated Correction

In an embodiment, double strand cleavage is effected by a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

Single Strand Break Mediated Correction

In other embodiments, two single strand breaks, or nicks, are effected by a Cas9 molecule having nickase activity, e.g., cleavage activity associated with an HNH-like domain or cleavage activity associated with an N-terminal RuvC-like domain. Such embodiments require two gRNAs, one for placement of each single strand break. In an embodiment, the Cas9 molecule having nickase activity cleaves the strand to which the gRNA hybridizes, but not the strand that is complementary to the strand to which the gRNA hybridizes. In an embodiment, the Cas9 molecule having nickase activity does not cleave the strand to which the gRNA hybridizes, but rather cleaves the strand that is complementary to the strand to which the gRNA hybridizes.

In an embodiment, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (e.g., the complementary strand, which does not have the NGG PAM on it). In other embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA).

In an embodiment, in which a nickase and two gRNAs are used to position two single strand nicks, one nick is on the + strand and one nick is on the − strand of the target nucleic acid. The PAMs are outwardly facing. The gRNAs can be selected such that the gRNAs are separated by, from about 0-50, 0-100, or 0-200 nucleotides. In an embodiment, there is no overlap between the target sequence that is complementary to the targeting domains of the two gRNAs. In an embodiment, the gRNAs do not overlap and are separated by as much as 50, 100, or 200 nucleotides. In an embodiment, the use of two gRNAs can increase specificity, e.g., by decreasing off-target binding (Ran et al., CELL 2013).

In an embodiment, a single nick can be used to induce HDR. It is contemplated herein that a single nick can be used to increase the ratio of HR to NHEJ at a given cleavage site.

Placement of the Double Strand Break or a Single Strand Break Relative to Target Position

The double strand break or single strand break in one of the strands should be sufficiently close to target position such that correction occurs. In an embodiment, the distance is not more than 50, 100, 200, 300, 350 or 400 nucleotides. While not wishing to be bound by theory, it is believed that the break should be sufficiently close to target position such that the break is within the region that is subject to exonuclease-mediated removal during end resection. If the distance between the target position and a break is too great, the mutation may not be included in the end resection and, therefore, may not be corrected, as donor sequence may only be used to correct sequence within the end resection region.

In an embodiment, in which a gRNA (unimolecular (or chimeric) or modular gRNA) and Cas9 nuclease induce a double strand break for the purpose of inducing HDR-mediated correction, the cleavage site is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position. In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In an embodiment, in which two gRNAs (independently, unimolecular (or chimeric) or modular gRNA) complexing with Cas9 nickases induce two single strand breaks for the purpose of inducing HDR-mediated correction, the closer nick is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position and the two nicks will ideally be within 25-55 bp of each other (e.g., 25 to 50, 25 to 45, 25 to 40, 25 to 35, 25 to 30, 30 to 55, 30 to 50, 30 to 45, 30 to 40, 30 to 35, 35 to 55, 35 to 50, 35 to 45, 35 to 40, 40 to 55, 40 to 50, 40 to 45 bp) and no more than 100 bp away from each other (e.g., no more than 90, 80, 70, 60, 50, 40, 30, 20, 10 or 5 bp away from each other). In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In one embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position (e.g., the first gRNA is used to target upstream (i.e., 5′) of the target position and the second gRNA is used to target downstream (i.e., 3′) of the target position). In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position (e.g., the first gRNA is used to target upstream (i.e., 5′) of the target position and the second gRNA is used to target downstream (i.e., 3′) of the target position). The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

In one embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position (e.g., the first gRNA is used to target upstream (i.e., 5′) of the mutation in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII and the second gRNA is used to target downstream (i.e., 3′) of the mutation in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII). In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position (e.g., the first gRNA is used to target upstream (i.e., 5′) of the mutation in a gene or pathway described herein, and the second gRNA is used to target downstream (i.e., 3′) of the mutation in a gene or pathway described herein). The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

Length of the Homology Arms

The homology arm should extend at least as far as the region in which end resection may occur, e.g., in order to allow the resected single stranded overhang to find a complementary region within the donor template. The overall length could be limited by parameters such as plasmid size or viral packaging limits. In an embodiment, a homology arm does not extend into repeated elements, e.g., ALU repeats, LINE repeats.

Exemplary homology arm lengths include a least 50, 100, 250, 500, 750 or 1000 nucleotides.

Target position, as used herein, refers to a site on a target nucleic acid (e.g., the chromosome) that is modified by a Cas9 molecule-dependent process. For example, the target position can be a modified Cas9 molecule cleavage of the target nucleic acid and template nucleic acid directed modification, e.g., correction, of the target position. In an embodiment, a target position can be a site between two nucleotides, e.g., adjacent nucleotides, on the target nucleic acid into which one or more nucleotides is added. The target position may comprise one or more nucleotides that are altered, e.g., corrected, by a template nucleic acid. In an embodiment, the target position is within a target sequence (e.g., the sequence to which the gRNA binds). In an embodiment, a target position is upstream or downstream of a target sequence (e.g., the sequence to which the gRNA binds).

A template nucleic acid, as that term is used herein, refers to a nucleic acid sequence which can be used in conjunction with a Cas9 molecule and a gRNA molecule to alter the structure of a target position. In an embodiment, the target nucleic acid is modified to have some or all of the sequence of the template nucleic acid, typically at or near cleavage site(s). In an embodiment, the template nucleic acid is single stranded. In an alternate embodiment, the template nucleic acid is double stranded. In an embodiment, the template nucleic acid is DNA, e.g., double stranded DNA. In an alternate embodiment, the template nucleic acid is single stranded DNA.

In an embodiment, the template nucleic acid alters the structure of the target position by participating in a homology directed repair event. In an embodiment, the template nucleic acid alters the sequence of the target position. In an embodiment, the template nucleic acid results in the incorporation of a modified, or non-naturally occurring base into the target nucleic acid.

Typically, the template sequence undergoes a breakage mediated or catalyzed recombination with the target sequence. In an embodiment, the template nucleic acid includes sequence that corresponds to a site on the target sequence that is cleaved by an eaCas9 mediated cleavage event. In an embodiment, the template nucleic acid includes sequence that corresponds to both, a first site on the target sequence that is cleaved in a first Cas9 mediated event, and a second site on the target sequence that is cleaved in a second Cas9 mediated event.

In an embodiment, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation.

In other embodiments, the template nucleic acid can include sequence which results in an alteration in a non-coding sequence, e.g., an alteration in an exon or in a 5′ or 3′ non-translated or non-transcribed region. Such alterations include
an alteration in a control element, e.g., a promoter, enhancer,
and an alteration in a cis-acting or trans-acting control element.

A template nucleic acid having homology with a target position in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII, can be used to alter the structure of a target sequence. The template sequence can be used to alter an unwanted structure, e.g., an unwanted or mutant nucleotide.

The template nucleic acid can include sequence which, when integrated, results in: decreasing the activity of a positive control element;
increasing the activity of a positive control element;
decreasing the activity of a negative control element;
increasing the activity of a negative control element;
decreasing the expression of a gene;
increasing the expression of a gene;
increasing resistance to a disorder or disease;
increasing resistance to viral entry;
correcting a mutation or altering an unwanted amino acid residue
conferring, increasing, abolishing or decreasing a biological property of a gene product, e.g., increasing the enzymatic activity of an enzyme, or increasing the ability of a gene product to interact with another molecule.

The template nucleic acid can include sequence which results in:
a change in sequence of 1, 2, 3, 4, 5, 6, 7, 8, 9, 10, 11, 12 or more nucleotides of the target sequence.

In an embodiment, the template nucleic acid is 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length.

In an embodiment, the template nucleic acid is 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length.

In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A template nucleic acid comprises the following components:

[5′ homology arm]-[replacement sequence]-[3′ homology arm].

The homology arms provide for recombination into the chromosome, thus replacing the undesired element, e.g., a mutation or signature, with the replacement sequence. In an embodiment, the homology arms flank the most distal cleavage sites.

In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence.

In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

It is contemplated herein that one or both homology arms may be shortened to avoid including certain sequence repeat elements, e.g., Alu repeats, LINE elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

It is contemplated herein that template nucleic acids for correcting a mutation may designed for use as a single-stranded oligonucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made.

In an embodiment, an ssODN may be used to correct a mutation in a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII.

### IV.2 NHEJ Approaches for Gene Targeting

As described herein, nuclease-induced non-homologous end-joining (NHEJ) can be used to target gene-specific knockouts. Nuclease-induced NHEJ can also be used to remove (e.g., delete) sequence in a gene of interest.

While not wishing to be bound by theory, it is believed that, in an embodiment, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein.

The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

Placement of Double Strand or Single Strand Breaks Relative to the Target Position

In an embodiment, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In an embodiment, the cleavage site is between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In an embodiment, the gRNAs are configured to position cuts at the same position, or within a few nucleotides of one another, on different strands, essentially mimicking a double strand break. In an embodiment, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In an embodiment, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position (e.g., of a gene or pathway described herein, e.g., in Section VIIB, e.g., in Table VII-13, VII-14, VII-15, VII-16, VII-17, VII-18, VII-19, VII-20, VII-21, VII-22, VII-23, VII-24, VII-25, IX-1, IX-1A, IX-2, IX-3, XIV-1, or Section VIII) to remove the nucleic acid sequence between the two cuts (e.g., the region between the two breaks is deleted). In one embodiment, two gRNAs, e.g., independently, unimolecular (or chimeric) or modular gRNA, are configured to position a double-strand break on both sides of a target position (e.g., the first gRNA is used to target upstream (i.e., 5′) of the mutation in a gene or pathway described herein, and the second gRNA is used to target downstream (i.e., 3′) of the mutation in a gene or pathway described herein). In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of a target position (e.g., the first gRNA is used to target upstream (i.e., 5′) of the mutation in a gene or pathway described herein, and the second gRNA is used to target downstream (i.e., 3′) of the mutation in a gene or pathway described herein). In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position (e.g., the first gRNA is used to target upstream (i.e., 5′) of the mutation in a gene or pathway described herein, and the second gRNA is used to target downstream (i.e., 3′) of the mutation in a gene or pathway described herein). The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

### IV.3 Targeted Knockdown

Unlike CRISPR/Cas-mediated gene knockout, which permanently eliminates expression by mutating the gene at the DNA level, CRISPR/Cas knockdown allows for temporary reduction of gene expression through the use of artificial transcription factors. Mutating key residues in both DNA cleavage domains of the Cas9 protein (e.g. the D10A and H840A mutations) results in the generation of a catalytically inactive Cas9 (eiCas9 which is also known as dead Cas9 or dCas9). A catalytically inactive Cas9 complexes with a gRNA and localizes to the DNA sequence specified by that gRNA's targeting domain, however, it does not cleave the target DNA. Fusion of the dCas9 to an effector domain, e.g., a transcription repression domain, enables recruitment of the effector to any DNA site specified by the gRNA. While it has been show that the eiCas9 itself can block transcription when recruited to early regions in the coding sequence, more robust repression can be achieved by fusing a transcriptional repression domain (for example KRAB, SID or ERD) to the Cas9 and recruiting it to the promoter region of a gene. It is likely that targeting DNAseI hypersensitive regions of the promoter may yield more efficient gene repression or activation because these regions are more likely to be accessible to the Cas9 protein and are also more likely to harbor sites for endogenous transcription factors. Especially for gene repression, it is contemplated herein that blocking the binding site of an endogenous transcription factor would aid in downregulating gene expression. In another embodiment, an eiCas9 can be fused to a chromatin modifying protein. Altering chromatin status can result in decreased expression of the target gene.

In an embodiment, a gRNA molecule can be targeted to a known transcription response elements (e.g., promoters, enhancers, etc.), a known upstream activating sequences (UAS), and/or sequences of unknown or known function that are suspected of being able to control expression of the target DNA.

CRISPR/Cas-mediated gene knockdown can be used to reduce expression of an unwanted allele or transcript. Contemplated herein are scenarios wherein permanent destruction of the gene is not ideal. In these scenarios, site-specific repression may be used to temporarily reduce or eliminate expression. It is also contemplated herein that the off-target effects of a Cas-repressor may be less severe than those of a Cas-nuclease as a nuclease can cleave any DNA sequence and cause mutations whereas a Cas-repressor may only have an effect if it targets the promoter region of an actively transcribed gene. However, while nuclease-mediated knockout is permanent, repression may only persist as long as the Cas-repressor is present in the cells. Once the repressor is no longer present, it is likely that endogenous transcription factors and gene regulatory elements would restore expression to its natural state.

### IV.4 Examples of gRNAs in Genome Editing Methods

gRNA molecules as described herein can be used with Cas9 molecules that generate a double strand break or a single strand break to alter the sequence of a target nucleic acid, e.g., a target position or target genetic signature. gRNA molecules useful in these methods are described below.

In an embodiment, the gRNA, e.g., a chimeric gRNA, is configured such that it comprises one or more of the following properties;
a) it can position, e.g., when targeting a Cas9 molecule that makes double strand breaks, a double strand break (i) within 50, 100, 150 or 200 nucleotides of a target position, or (ii) sufficiently close that the target position is within the region of end resection;
b) it has a targeting domain of at least 17 nucleotides, e.g., a targeting domain of (i) 17, (ii) 18, or (iii) 20 nucleotides; and
c)
   (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
   (ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
   (iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5, 6, 7, 8, 9 or 10 nucleotides therefrom;
   iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, or a sequence that differs by no more than 1, 2, 3, 4, 5, 6, 7, 8, 9 or 10 nucleotides therefrom; or
   (v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In an embodiment, the gRNA is configured such that it comprises properties: a and b(i).

In an embodiment, the gRNA is configured such that it comprises properties: a and b(ii).

In an embodiment, the gRNA is configured such that it comprises properties: a and b(iii).

In an embodiment, the gRNA is configured such that it comprises properties: a and c.

In an embodiment, the gRNA is configured such that in comprises properties: a, b, and c.

In an embodiment, the gRNA is configured such that in comprises properties: a(i), b(i), and c(i).

In an embodiment, the gRNA is configured such that in comprises properties: a(i), b(i), and c(ii).

In an embodiment, the gRNA is configured such that in comprises properties: a(i), b(iii), and c(i).

In an embodiment, the gRNA is configured such that in comprises properties: a(i), b(iii), and c(ii).

In an embodiment, the gRNA, e.g., a chimeric gRNA, is configured such that it comprises one or more of the following properties;
a) it can position, e.g., when targeting a Cas9 molecule that makes single strand breaks, a single strand break (i) within 50, 100, 150 or 200 nucleotides of a target position, or (ii) sufficiently close that the target position is within the region of end resection;
b) it has a targeting domain of at least 17 nucleotides, e.g., a targeting domain of (i) 17, (ii) 18, or (iii) 20 nucleotides; and
c)
   (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5, 6, 7, 8, 9 or 10 nucleotides therefrom;
   (ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5, 6, 7, 8, 9 or 10 nucleotides therefrom;
   (iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
   iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, a sequence that differs by no more than 1, 2, 3, 4, 5, 6, 7, 8, 9 or 10 nucleotides therefrom; or
   (v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In an embodiment, the gRNA is configured such that it comprises properties: a and b(i).

In an embodiment, the gRNA is configured such that it comprises properties: a and b(ii).

In an embodiment, the gRNA is configured such that it comprises properties: a and b(iii).

In an embodiment, the gRNA is configured such that it comprises properties: a and c.

In an embodiment, the gRNA is configured such that in comprises properties: a, b, and c.

In an embodiment, the gRNA is configured such that in comprises properties: a(i), b(i), and c(i).

In an embodiment, the gRNA is configured such that in comprises properties: a(i), b(i), and c(ii).

In an embodiment, the gRNA is configured such that in comprises properties: a(i), b(iii), and c(i).

In an embodiment, the gRNA is configured such that in comprises properties: a(i), b(iii), and c(ii).

In an embodiment, the gRNA is used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNA is used with a Cas9 nickase molecule having RuvC activity, e.g., a Cas9 molecule having the HNH activity inactivated, e.g., a Cas9 molecule having a mutation at H840, e.g., a H840A.

In an embodiment, a pair of gRNAs, e.g., a pair of chimeric gRNAs, comprising a first and a second gRNA, is configured such that they comprises one or more of the following properties;
a) one or both of the gRNAs can position, e.g., when targeting a Cas9 molecule that makes single strand breaks, a single strand break within (i) 50, 100, 150 or 200 nucleotides of a target position, or (ii) sufficiently close that the target position is within the region of end resection;
b) one or both have a targeting domain of at least 17 nucleotides, e.g., a targeting domain of (i) 17 or (ii) 18 nucleotides;
c) for one or both:
   (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5, 6, 7, 8, 9 or 10 nucleotides therefrom;
   (ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5, 6, 7, 8, 9 or 10 nucleotides therefrom;
   (iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
   iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, or a sequence that differs by no more than 1, 2, 3, 4, 5, 6, 7, 8, 9 or 10 nucleotides therefrom; or
   (v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain;
d) the gRNAs are configured such that, when hybridized to target nucleic acid, they are separated by 0-50, 0-100, 0-200, at least 10, at least 20, at least 30 or at least 50 nucleotides;
e) the breaks made by the first gRNA and second gRNA are on different strands; and
f) the PAMs are facing outwards.

In an embodiment, one or both of the gRNAs is configured such that it comprises properties: a and b(i).

In an embodiment, one or both of the gRNAs is configured such that it comprises properties: a and b(ii).

In an embodiment, one or both of the gRNAs is configured such that it comprises properties: a and b(iii).

In an embodiment, one or both of the gRNAs configured such that it comprises properties: a and c.

In an embodiment, one or both of the gRNAs is configured such that in comprises properties: a, b, and c.

In an embodiment, one or both of the gRNAs is configured such that in comprises properties: a(i), b(i), and c(i).

In an embodiment, one or both of the gRNAs is configured such that in comprises properties: a(i), b(i), and c(ii).

In an embodiment, one or both of the gRNAs is configured such that in comprises properties: a(i), b(i), c, and d.

In an embodiment, one or both of the gRNAs is configured such that in comprises properties: a(i), b(i), c, and e.

In an embodiment, one or both of the gRNAs is configured such that in comprises properties: a(i), b(i), c, d, and e.

In an embodiment, one or both of the gRNAs is configured such that in comprises properties: a(i), b(iii), and c(i).

In an embodiment, one or both of the gRNAs is configured such that in comprises properties: a(i), b(iii), and c(ii).

In an embodiment, one or both of the gRNAs is configured such that in comprises properties: a(i), b(iii), c, and d.

In an embodiment, one or both of the gRNAs is configured such that in comprises properties: a(i), b(iii), c, and e.

In an embodiment, one or both of the gRNAs is configured such that in comprises properties: a(i), b(iii), c, d, and e.

In an embodiment, the gRNAs are used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNAs are used with a Cas9 nickase molecule having RuvC activity, e.g., a Cas9 molecule having the HNH activity inactivated, e.g., a Cas9 molecule having a mutation at H840, e.g., a H840A.

### V. Constructs/Components

The components, e.g., a Cas9 molecule or gRNA molecule, or both, can be delivered, formulated, or administered in a variety of forms, see, e.g., Table V-1a and Table V-1b. When a component is delivered encoded in DNA the DNA will typically include a control region, e.g., comprising a promoter, to effect expression. Useful promoters for Cas9 molecule sequences include CMV, EF-1a, MSCV, PGK, CAG control promoters. Useful promoters for gRNAs include H1, EF-1a and U6 promoters. Promoters with similar or dissimilar strengths can be selected to tune the expression of components. Sequences encoding a Cas9 molecule can comprise a nuclear localization signal (NLS), e.g., an SV40 NLS. In an embodiment, a promoter for a Cas9 molecule or a gRNA molecule can be, independently, inducible, tissue specific, or cell specific.

Table V-1a and Table V-1b provide examples of how the components can be formulated, delivered, or administered.

TABLE V-1a
Element

| Cas9 | gRNA | Template | |
|---|---|---|---|
| Mole- | mole- | Nucleic | |
| cule(s) | cule(s) | Acid | Comments |
| DNA | DNA | DNA | In this embodiment a Cas9 molecule, |
| | | | typically an eaCas9 molecule, and a |
| | | | gRNA are transcribed from DNA. In |
| | | | this embodiment they are encoded on |
| | | | separate molecules. |
| DNA | | DNA | In this embodiment a Cas9 molecule, |
| | | | typically an eaCas9 molecule, and a |
| | | | gRNA are transcribed from DNA, here |
| | | | from a single molecule. |
| DNA | RNA | DNA | In this embodiment a Cas9 molecule, |
| | | | typically an eaCas9 molecule, is |
| | | | transcribed from DNA. A gRNA is |
| | | | provided as RNA. In an embodiment, |
| | | | the gRNA comprises one or more |
| | | | modifications, e.g., as described in |
| | | | Section X. |
| mRNA | RNA | DNA | In this embodiment a Cas9 molecule, |
| | | | typically an eaCas9 molecule, is |
| | | | transcribed from DNA. A gRNA is |
| | | | provided as RNA. In an embodiment, |
| | | | the gRNA comprises one or more |
| | | | modifications, e.g., as described in |
| | | | Section X. In an embodiment, the mRNA |
| | | | comprises one or more modifications, |
| | | | e.g., as described in Section X. |
| Protein | DNA | DNA | In this embodiment a Cas9 molecule, |
| | | | typically an eaCas9 molecule, is |
| | | | provided as a protein. A gRNA is |
| | | | transcribed from DNA. |
| Protein | RNA | DNA | In this embodiment an eaCas9 molecule |
| | | | is provided as a protein. A gRNA is |
| | | | provided as RNA. In an embodiment, |
| | | | the gRNA comprises one or more |
| | | | modifications, e.g., as described in |
| | | | Section X. |

TABLE V-1b
Element

| Cas9 | gRNA | | |
|---|---|---|---|
| Mole- | mole- | | |
| cule(s) | cule(s) | Payload | Comments |
| DNA | DNA | Yes | In this embodiment a Cas9 molecule, |
| | | | typically an eiCas9 molecule, and a |
| | | | gRNA are transcribed from DNA. Here |
| | | | they are provided on separate |
| | | | molecules. |
| DNA | | Yes | Similar to above, but in this embodiment |
| | | | a Cas9 molecule, typically an eiCas9 |
| | | | molecule, and a gRNA are transcribed |
| | | | from a single molecule. |
| DNA | RNA | Yes | In this embodiment a Cas9 molecule, |
| | | | typically an eiCas9 molecule, is |
| | | | transcribed from DNA. A gRNA is |
| | | | provided as RNA. In an embodiment, |
| | | | the gRNA comprises one or more |
| | | | modifications, e.g., as described in |
| | | | Section X. |
| mRNA | RNA | Yes | In this embodiment a Cas9 molecule, |
| | | | typically an eiCas9 molecule, is |
| | | | provided as encoded in mRNA. A gRNA |
| | | | is provided as RNA. In an embodiment, |
| | | | the gRNA comprises one or more |
| | | | modifications, e.g., as described |
| | | | in Section X. In an embodiment, the |
| | | | mRNA comprises one or more modifica- |
| | | | tions, e.g., as described in section X. |
| Protein | DNA | Yes | In this embodiment a Cas9 molecule, |
| | | | typically an eiCas9 molecule, is provided |
| | | | as a protein. A gRNA is provided encoded |
| | | | in DNA. |
| Protein | RNA | Yes | In this embodiment a Cas9 molecule, |
| | | | typically an eiCas9 molecule, is provided |
| | | | as a protein. A gRNA is provided as RNA. |
| | | | In an embodiment, the gRNA comprises one |
| | | | or more modifications, e.g., as described |
| | | | in Section X. |

DNA-Based Delivery of a Cas9 Molecule and or a gRNA Molecule

DNA encoding Cas9 molecules (e.g., eaCas9 molecules or eiCas9 molecules) and/or gRNA molecules, can be administered to subjects or delivered into cells by art-known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof.

In some embodiments, the Cas9- and/or gRNA-encoding DNA is delivered by a vector (e.g., viral vector/virus or plasmid).

A vector can comprise a sequence that encodes a Cas9 molecule and/or a gRNA molecule. A vector can also comprise a sequence encoding a signal peptide (e.g., for nuclear localization, nucleolar localization, mitochondrial localization), fused, e.g., to a Cas9 molecule sequence. For example, a vector can comprise a nuclear localization sequence (e.g., from SV40) fused to the sequence encoding the Cas9 molecule.

One or more regulatory/control elements, e.g., a promoter, an enhancer, an intron, a polyadenylation signal, a Kozak consensus sequence, internal ribosome entry sites (IRES), a 2A sequence, and a splice acceptor or donor can be included in the vectors. In some embodiments, the promoter is recognized by RNA polymerase II (e.g., a CMV promoter). In other embodiments, the promoter is recognized by RNA polymerase III (e.g., a U6 promoter). In some embodiments, the promoter is a regulated promoter (e.g., inducible promoter). In other embodiments, the promoter is a constitutive promoter. In some embodiments, the promoter is a tissue specific promoter. In some embodiments, the promoter is a viral promoter. In other embodiments, the promoter is a non-viral promoter.

In some embodiments, the vector or delivery vehicle is a viral vector (e.g., for generation of recombinant viruses). In some embodiments, the virus is a DNA virus (e.g., dsDNA or ssDNA virus). In other embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in human. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the Cas9 molecule and/or the gRNA molecule. In other embodiments, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the Cas9- and/or gRNA-encoding DNA is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the Cas9- and/or gRNA-encoding DNA is delivered by a recombinant lentivirus. For example, the lentivirus is replication-defective, e.g., does not comprise one or more genes required for viral replication.

In some embodiments, the Cas9- and/or gRNA-encoding DNA is delivered by a recombinant adenovirus. In some embodiments, the adenovirus is engineered to have reduced immunity in human.

In some embodiments, the Cas9- and/or gRNA-encoding DNA is delivered by a recombinant AAV. In some embodiments, the AAV can incorporate its genome into that of a host cell, e.g., a target cell as described herein. In some embodiments, the AAV is a self-complementary adeno-associated virus (scAAV), e.g., a scAAV that packages both strands which anneal together to form double stranded DNA. AAV serotypes that may be used in the disclosed methods include, e.g., AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods.

In some embodiments, the Cas9- and/or gRNA-encoding DNA is delivered by a hybrid virus, e.g., a hybrid of one or more of the viruses described herein.

A Packaging cell is used to form a virus particle that is capable of infecting a host or target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In an embodiment, the viral vector has the ability of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In an embodiment, the viral vector achieves cell type specific expression. For example, a tissue-specific promoter can be constructed to restrict expression of the transgene (Cas 9 and gRNA) in only the target cell. The specificity of the vector can also be mediated by microRNA-dependent control of transgene expression. In an embodiment, the viral vector has increased efficiency of fusion of the viral vector and a target cell membrane. For example, a fusion protein such as fusion-competent hemagglutinin (HA) can be incorporated to increase viral uptake into cells. In an embodiment, the viral vector has the ability of nuclear localization. For example, a virus that requires the breakdown of the cell wall (during cell division) and therefore will not infect a non-diving cell can be altered to incorporate a nuclear localization peptide in the matrix protein of the virus thereby enabling the transduction of non-proliferating cells.

In some embodiments, the Cas9- and/or gRNA-encoding DNA is delivered by a non-vector based method (e.g., using naked DNA or DNA complexes). For example, the DNA can be delivered, e.g., by organically modified silica or silicate (Ormosil), electroporation, gene gun, sonoporation, magnetofection, lipid-mediated transfection, dendrimers, inorganic nanoparticles, calcium phosphates, or a combination thereof.

In some embodiments, the Cas9- and/or gRNA-encoding DNA is delivered by a combination of a vector and a non-vector based method. For example, a virosome comprises a liposome combined with an inactivated virus (e.g., HIV or influenza virus), which can result in more efficient gene transfer, e.g., in a respiratory epithelial cell than either a viral or a liposomal method alone.

In an embodiment, the delivery vehicle is a non-viral vector. In an embodiment, the non-viral vector is an inorganic nanoparticle (e.g., attached to the payload to the surface of the nanoparticle). Exemplary inorganic nanoparticles include, e.g., magnetic nanoparticles (e.g., Fe₃MnO₂), or silica. The outer surface of the nanoparticle can be conjugated with a positively charged polymer (e.g., polyethylenimine, polylysine, polyserine) which allows for attachment (e.g., conjugation or entrapment) of payload. In an embodiment, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids for gene transfer are shown in Table XII-2.

Exemplary polymers for gene transfer are shown below in Table XII-3.

In an embodiment, the vehicle has targeting modifications to increase target cell update of nanoparticles and liposomes, e.g., cell specific antigens, monoclonal antibodies, single chain antibodies, aptamers, polymers, sugars, and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimuli-cleavable polymer is used, e.g., for release in a cellular compartment. For example, disulfide-based cationic polymers that are cleaved in the reducing cellular environment can be used.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In an embodiment, the vehicle is a genetically modified bacteriophage (e.g., engineered phages having large packaging capacity, less immunogenic, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovesicle (30-100 nm) of endocytic origin (e.g., can be produced from various cell types and can therefore be taken up by cells without the need of for targeting ligands).

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the components of the Cas system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In an embodiment, the nucleic acid molecule is delivered by a different means than one or more of the components of the Cas system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component and/or the gRNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

Delivery of RNA Encoding a Cas9 Molecule

RNA encoding Cas9 molecules (e.g., eaCas9 molecules, eiCas9 molecules or eiCas9 fusion proteins) and/or gRNA molecules, can be delivered into cells, e.g., target cells described herein, by art-known methods or as described herein. For example, Cas9-encoding and/or gRNA-encoding RNA can be delivered, e.g., by microinjection, electroporation, lipid-mediated transfection, peptide-mediated delivery, or a combination thereof.

Delivery Cas9 Molecule Protein

Cas9 molecules (e.g., eaCas9 molecules, eiCas9 molecules or eiCas9 fusion proteins) can be delivered into cells by art-known methods or as described herein. For example, Cas9 protein molecules can be delivered, e.g., by microinjection, electroporation, lipid-mediated transfection, peptide-mediated delivery, or a combination thereof. Delivery can be accompanied by DNA encoding a gRNA or by a gRNA.

Route of Administration

Systemic modes of administration include oral and parenteral routes. Parenteral routes include, by way of example, intravenous, intraarterial, intraosseous, intramuscular, intradermal, subcutaneous, intranasal and intraperitoneal routes. Components administered systemically may be modified or formulated to target the components to the eye.

Local modes of administration include, by way of example, intrathecal, intracerebroventricular, intraparenchymal (e.g., localized intraparenchymal delivery to the striatum (e.g., into the caudate or into the putamen)), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum or substantia nigra intraocular, intraorbital, subconjunctival, intravitreal, subretinal or transscleral routes. In an embodiment, significantly smaller amounts of the components (compared with systemic approaches) may exert an effect when administered locally (for example, intraparenchymal or intravitreal) compared to when administered systemically (for example, intravenously). Local modes of administration can reduce or eliminate the incidence of potentially toxic side effects that may occur when therapeutically effective amounts of a component are administered systemically.

In an embodiment, components described herein are delivered by intraparenchymal injection into discrete regions of the brain, including, e.g., regions comprising medium spiny neurons, or regions comprising cortical neurons. Injections may be made directly into more than one region of the brain.

In an embodiment, components described herein are delivered by subretinally, e.g., by subretinal injection. Subretinal injections may be made directly into the macular, e.g., submacular injection.

In an embodiment, components described herein are delivered by intravitreal injection. Intravitreal injection has a relatively low risk of retinal detachment risk. In an embodiment, a nanoparticle or viral vector, e.g., AAV vector, e.g., an AAV2 vector, e.g., a modified AAV2 vector, is delivered intravitreally.

In an embodiment, a nanoparticle or viral vector, e.g., AAV vector, delivery is via intraparenchymal injection.

Methods for administration of agents to the eye are known in the medical arts and can be used to administer components described herein. Exemplary methods include intraocular injection (e.g., retrobulbar, subretinal, submacular, intravitreal and intrachoridal), iontophoresis, eye drops, and intraocular implantation (e.g., intravitreal, sub-Tenons and sub-conjunctival).

Administration may be provided as a periodic bolus (for example, subretinally, intravenously or intravitreally) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag). Components may be administered locally, for example, by continuous release from a sustained release drug delivery device immobilized to an inner wall of the eye or via targeted transscleral controlled release into the choroid (see, for example, PCT/US00/00207, PCT/US02/14279, Ambati et al. (2000) INVEST. OPHTHALMOL. VIS. SCI.41:1181-1185, and Ambati et al. (2000) INVEST. OPHTHALMOL. VIS. SCI.41:1186-1191). A variety of devices suitable for administering components locally to the inside of the eye are known in the art. See, for example, U.S. Pat. Nos. 6,251,090, 6,299,895, 6,416,777, 6,413,540, and PCT/US00/28187.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used for intraocular injection. Typically the microspheres are composed of a polymer of lactic acid and glycolic acid, which are structured to form hollow spheres. The spheres can be approximately 15-30 microns in diameter and can be loaded with components described herein.

Bi-Modal or Differential Delivery of Components

Separate delivery of the components of a Cas system, e.g., the Cas9 molecule component and the gRNA molecule component, and more particularly, delivery of the components by differing modes, can enhance performance, e.g., by improving tissue specificity and safety.

In an embodiment, the Cas9 molecule and the gRNA molecule are delivered by different modes, or as sometimes referred to herein as differential modes. Different or differential modes, as used herein, refer modes of delivery that confer different pharmacodynamic or pharmacokinetic properties on the subject component molecule, e.g., a Cas9 molecule, gRNA molecule, or template nucleic acid. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component.

### VI. Payloads

Cas9 molecules, typically eiCas9 molecules and gRNA molecules, e.g., an eiCas9 molecule/gRNA molecule complex, can be used to deliver a wide variety of payloads. In an embodiment, the payload is delivered to target nucleic acids or to chromatin, or other components, near or associated with a target nucleic acid.

While not wishing to be bound by theory, it is believed that the sequence specificity of the gRNA molecule of an eiCas9 molecule/gRNA molecule complex contributes to a specific interaction with the target sequence, thereby effecting the delivery of a payload associated with, e.g., covalently or noncovalently coupled to, the Cas9 molecule/gRNA molecule complex.

In an embodiment, the payload is covalently or non-covalently coupled to a Cas9, e.g., an eiCas9 molecule. In an embodiment, the payload is covalently or non-covalently coupled to a gRNA molecule. In an embodiment, the payload is linked to a Cas9 molecule, or gRNA molecule, by a linker, e.g., a linker which comprises a bond cleavable under physiological conditions. In other embodiments the bond is not cleavable or is only poorly cleavable, under physiological conditions. In an embodiment, “covalently coupled” means as part of a fusion protein containing a Cas9 molecule.

Delivery of Multiple Payloads

In an embodiment, a first payload molecule is delivered by a first Cas9 molecule and a second payload molecule is delivered by a second Cas9 molecule. In an embodiment, the first and second payloads are the same. In an embodiment, first and second Cas9 molecules are the same, e.g. are from the same species, have the same PAM, and/or have the same sequence. In an embodiment, first and second Cas9 molecules are different, e.g. are from different species, have the different PAMs, and/or have different sequences. Examples of configurations are provided in Table VI-1. Typically the Cas9 molecules of Table VI-1 are eiCas9 molecules. In other embodiments a Cas9 molecule is selected such that payload delivery and cleavage are both effected. In an embodiment, multiple payloads, e.g., two payloads, is delivered with a single Cas9 molecule.

TABLE VI-1
Configurations for delivery of payloads by more
than one Cas9 molecule/gRNA molecule complex

| | | | Sec- | |
|---|---|---|---|---|
| | Second | First | ond | |
| First Cas9 | Cas9 | Pay- | Pay- | |
| molecule | molecule | load | load | Comments |
| C1 | C1 | P1 | P1 | In this embodiment, both Cas9 |
| | | | | molecules are the same, as are |
| | | | | both payloads. In an embodi- |
| | | | | ment, the first and second |
| | | | | Cas9 molecule are guided by |
| | | | | different gRNA molecules. |
| C1 | C1 | P1 | P2 | In this embodiment, both Cas9 |
| | | | | molecules are the same but each |
| | | | | delivers a different Payloads. |
| | | | | In an embodiment, the first and |
| | | | | second Cas9 molecule are |
| | | | | guided by different gRNA |
| | | | | molecules. |
| C1 | C2 | P1 | P1 | In this embodiment, the Cas9 |
| | | | | molecules are different but each |
| | | | | delivers the same payload. In an |
| | | | | embodiment, the first and |
| | | | | second Cas9 molecule are |
| | | | | guided by different gRNA |
| | | | | molecules. |
| C1 | C2 | P1 | P2 | In this embodiment, the Cas9 |
| | | | | molecules are different as are the |
| | | | | payloads. In an embodiment, the |
| | | | | first and second Cas9 molecule |
| | | | | are guided by different gRNA |
| | | | | molecules. |

In an embodiment, two different drugs are delivered. In an embodiment, a first payload, e.g., a drug, coupled by a first linker to a first Cas9 molecule and a second payload, e.g., a drug, coupled by a second linker to a second Cas9 molecule are delivered. In an embodiment, the first and second payloads are the same, and, in an embodiment, are coupled to the respective Cas9 molecule by different linkers, e.g., having different release kinetics. In an embodiment, the first and second payloads are different, and, in an embodiment, are coupled to the respective Cas9 molecule by the same linker. In an embodiment, the first and second payload interact. E.g., the first and second payloads form a complex, e.g., a dimeric or multimeric complex, e.g., a dimeric protein. In an embodiment, the first payload can activate the second payload, e.g., the first payload can modify, e.g., cleave or phosphorylate, the second payload. In an embodiment the first payload interacts with the second payload to modify, e.g., increase or decrease, an activity of the second payload.

A payload can be delivered in vitro, ex vivo, or in vivo.

Classes of Payloads

A payload can comprise a large molecule or biologics (e.g., antibody molecules), a fusion protein, an amino acid sequence fused, as a fusion partner, to a Cas9 molecule, e.g., an eiCas9 molecule, an enzyme, a small molecules (e.g., HDAC and other chromatin modifiers/inhibitors, exon skipping molecules, transcription inhibitors), a microsatellite extension inhibitor, a carbohydrate, and DNA degraders (e.g., in an infectious disease or “foreign” DNA setting), a nucleic acid, e.g., a DNA, RNA, mRNA, siRNA, RNAi, or an antisense oligonucleotide.

Table VI-2 provides exemplary classes of payloads.

TABLE VI-2
Exemplary Classes of Payloads

Large Molecules
Small Molecules
Polymers
Biologics
Proteins and polypeptides, e.g., antibodies, enzymes, structural peptides,
ligands, receptors, fusion proteins, fusion partners (as a fusion protein with
a Cas9, e.g., and eiCas9)
Carbohydrates
HDAC and other chromatin modifiers/inhibitors
Exon skipping molecules,
Transcription inhibitors
Microsatellite extension inhibitors
Entities that degrade DNA

Large Molecules

In an embodiment a payload comprises a polymer, e.g., a biological polymer, e.g., a protein, nucleic acid, or carbohydrate.

In an embodiment the payload comprises a protein, biologic, or other large molecule (i.e., a molecule having a molecular weight of at least, 3, 5, 10, 20, 30, 40, 50, 60, 70, 80, 90, or 100 kD). In an embodiment a payload comprises a polymer, e.g., a biological polymer, e.g., a protein, nucleic acid, or carbohydrate. The polymer can be a naturally occurring or non-naturally occurring polymer. In an embodiment, the payload is a natural product. For example, the natural product can be a large molecule or a small molecule.

Polypeptides, Proteins

In an embodiment the payload comprises a protein or polypeptide, e.g., a protein or polypeptide covalently or non-covalently coupled to a Cas9 molecule.

In an embodiment, the protein or polypeptide is dimeric or multimeric, and each subunit is delivered by a Cas9 molecule. In an embodiment, a first protein and second protein are delivered by one or more Cas9 molecules, e.g., each by a separate Cas9 molecule or both by the same Cas9 molecule.

In an embodiment, the protein or polypeptide is linked to a Cas9 molecule by a linker, e.g., a linker which comprises a bond cleavable under physiological conditions. In an embodiment, a linker is a linker from Section XI herein. In an embodiment, the bond is not cleavable under physiological conditions.

Specific Binding Ligands, Antibodies

In an embodiment the payload comprises a ligand, e.g., a protein, having specific affinity for a counter ligand. In an embodiment, the ligand can be a receptor (or the ligand for a receptor), or an antibody.

In an embodiment a payload comprises an antibody molecule. Exemplary antibody molecules include, e.g., proteins or polypeptides that include at least one immunoglobulin variable domain. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and domain antibodies (dAb) fragments (de Wildt et al., Eur J Immunol. 1996; 26(3):629-639)). For example, antigen-binding fragments of antibodies can include, e.g., (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. An antibody can have the structural features of IgA, IgG, IgE, IgD, IgM (as well as subtypes thereof). Antibodies may be from any source, but primate (human and non-human primate) and primatized are preferred. In some embodiments, the antibody is a human antibody or humanized antibody.

In an embodiment, the antibody molecule is a single-domain antibody (e.g., an sdAb, e.g., a nanobody), e.g., an antibody fragment consisting of a single monomeric variable antibody domain. In an embodiment, the molecular weight of the single-domain antibody is about 12-15 kDa. For example, the single-domain antibody can be engineered from heavy-chain antibodies found in camelids (e.g., VHH fragments). Cartilaginous fishes also have heavy-chain antibodies (IgNAR, ‘immunoglobulin new antigen receptor’), from which single-domain antibodies called VNAR fragments can be obtained. An alternative approach is to split the dimeric variable domains from common immunoglobulin G (IgG), e.g., from humans or mice, into monomers. Single-domain antibodies derived from either heavy or light chain can be obtained to bind specifically to target epitopes. For example, a single-domain antibody can be a peptide chain of about 110 amino acids long, comprising one variable domain (VH) of a heavy-chain antibody, or of a common IgG.

Single-domain antibodies can have similar affinity to antigens as whole antibodies. They can also be more heat-resistant and/or stable towards detergents and high concentrations of urea. Those, e.g., derived from camelid and fish antibodies can be less lipophilic and more soluble in water, owing to their complementarity determining region 3 (CDR3), which forms an extended loop covering the lipophilic site that normally binds to a light chain. In an embodiment, the single-domain antibody does not show complement system triggered cytotoxicity, e.g., because they lack an Fc region. Single-domain antibodies, e.g., camelid and fish derived sdAbs, can bind to hidden antigens that may not be accessible to whole antibodies, for example to the active sites of enzymes. This property can result from their extended CDR3 loop, which is able to penetrate such sites.

A single-domain antibody can be obtained by immunization of, e.g., dromedaries, camels, llamas, alpacas or sharks with the desired antigen and subsequent isolation of the mRNA coding for heavy-chain antibodies. By reverse transcription and polymerase chain reaction, a gene library of single-domain antibodies containing several million clones is produced. Screening techniques like phage display and ribosome display help to identify the clones binding the antigen.

A different method uses gene libraries from animals that have not been immunized beforehand. Such naïve libraries usually contain only antibodies with low affinity to the desired antigen, making it necessary to apply affinity maturation by random mutagenesis as an additional step.

When the most potent clones have been identified, their DNA sequence can be optimized, for example to improve their stability towards enzymes. Another goal is humanization to prevent immunological reactions of the human organism against the antibody. Humanization is unproblematic because of the homology between, e.g., camelid VHH and human VH fragments. The final step is the translation of the optimized single-domain antibody in E. coli, Saccharomyces cerevisiae or other suitable organisms.

Alternatively, single-domain antibodies can be made from common murine or human IgG with four chains. The process is similar, comprising gene libraries from immunized or naïve donors and display techniques for identification of the most specific antigens. Monomerization is usually accomplished by replacing lipophilic by hydrophilic amino acids. If affinity can be retained, the single-domain antibodies can likewise be produced in E. coli, S. cerevisiae or other organisms.

In an embodiment, a payload comprises a transcription activator protein or domain, e.g., a VP16 protein or domain, or a transcription repressor protein or domain.

Fusion Proteins and Fusion Partners

In an embodiment the payload comprises a fusion protein. Exemplary fusion proteins include a first and second fusion partner, which can possess different functional properties or which can be derived from different proteins. In an embodiment, the fusion protein can comprise a first fusion partner that binds a nucleic acid and a second fusion partner that that comprises an enzymatic activity or that promotes or inhibits gene expression. In an embodiment, the payload itself is a fusion protein. In an embodiment, the payload is fused to a Cas9 molecule.

For example, the fusion protein can contain a segment that adds stability and/or deliverability to the fused protein. In some embodiments, the fusion protein can be a protein described herein (e.g., a receptor) fused to an immunoglobulin fragment (e.g., Fc fragment), transferring, or a plasma protein, e.g., albumin. The fusion protein can also contain a segment that adds toxicity to the fused protein (e.g. conveyed by toxins, enzymes or cytokines). Fusion proteins can also be used to enable delivery and/or targeting routes (e.g., by HIV-1 TAT protein). Other examples include, e.g., fusions that allow for mutivalency, such as streptavidin fusions, or fusions of two active components (e.g., with or without a cleavable linker in between).

In an embodiment, the protein or polypeptide is a fusion partner with a Cas9 molecule, e.g., an eiCas9 molecule.

In an embodiment, a payload comprises fusion partner with a Cas9 molecule comprising a transcription activator protein or domain, e.g., a VP16 protein or domain, or a transcription repressor protein or domain.

Enzymes

In an embodiment a payload comprises an enzyme. Exemplary enzymes include, e.g., oxidoreductases (e.g., catalyze oxidation/reduction reactions), transferases (e.g., transfer a functional group (e.g. a methyl or phosphate group)), hydrolases (e.g., catalyze the hydrolysis of various bonds), lyases (e.g., cleave various bonds by means other than hydrolysis and oxidation), isomerases (catalyze isomerization changes within a single molecule), and ligases (e.g., join two molecules with covalent bonds). In an embodiment an enzymes mediates or is associated with one or more functions in the cell nucleus, e.g., DNA synthesis, transcription, epigenetic modification of DNA and histones, RNA post-transcriptional modification, cell cycle control, DNA damage repair, or genomic instability.

Small Molecules

In an embodiment a payload comprises a small molecule compounds.

In an embodiment a small molecule is a regulator of a biological process. For example, a small molecule can bind to a second molecule, e.g., biopolymer, e.g., a carbohydrate, protein, polypeptide, or a nucleic acid, and in an embodiment, alter one or more of the structure, distribution, activity, or function of the second molecule. In some embodiments, the size of the small molecule is on the order of 10⁻⁹ m. In some embodiments, the molecular weight of the small molecule is, e.g., between 200 amu and 500 amu, between 300 amu and 700 amu, between 500 amu and 700 amu, between 700 amu and 900 amu, or between 500 amu and 900 amu.

Exemplary small molecules include histone deacetylase (HDAC) inhibitors (e.g., suberoylanilide hydroxamic acid (SAHA), or romidepsin), histone methyltransferase inhibitors (DNA methyltransferase inhibitors (e.g., azacitidine (or 5-azacitidine), decitabine (or 5-aza-2′-deoxycytidine), or DNA replication inhibitors. Small molecules can also include, e.g., small nucleic acid molecules (1-4 bases depending upon the base, e.g., that would be under 2 kD) and peptides.

Exemplary classes of small molecules that may be used as payloads include, but are not limited to, 5-alpha Reductase Inhibitor, 5-alpha Reductase Inhibitors, 5-Lipoxygenase Inhibitor, 5-Lipoxygenase Inhibitors, Acetyl Aldehyde Dehydrogenase Inhibitors, Acetylcholine Release Inhibitor, Acetylcholine Release Inhibitors, Acetylcholine Releasing Agent, Acidifying Activity, Actinomycin, Actively Acquired Immunity, Adenosine Deaminase, Adenosine Receptor Agonist, Adenosine Receptor Agonists, Adenovirus Vaccines, Adrenal Steroid Synthesis Inhibitor, Adrenal Steroid Synthesis Inhibitors, Adrenergic Agonists, Adrenergic alpha-Agonists, Adrenergic alpha-Antagonists, Adrenergic alpha2-Agonists, Adrenergic beta-Agonists, Adrenergic beta-Antagonists, Adrenergic beta1-Antagonists, Adrenergic beta2-Agonists, Adrenergic beta2-Antagonists, Adrenergic beta3-Agonists, Adrenergic Receptor Agonist, Adrenocorticotropic Hormone, Adrenocorticotropic Hormone, Aldehyde Dehydrogenase Inhibitor, Aldosterone Antagonist, Aldosterone Antagonists, Alkylating Activity, Alkylating Drug, Allergens, Allogeneic Cord Blood Hematopoietic Progenitor Cell Therapy, Allogeneic Cultured Cell Scaffold, Allylamine Antifungal, Allylamine, alpha Glucosidase Inhibitors, alpha-Adrenergic Agonist, alpha-Adrenergic Blocker, alpha-Glucosidase Inhibitor, alpha-Glucosidases, Aluminum Complex, Alveolar Surface Tension Reduction, Amide Local Anesthetic, Amides, Amino Acid Hypertonic Solution, Amino Acid, Amino Acids, Aminoglycoside Antibacterial, Aminoglycosides, Aminoketone, Aminosalicylate, Aminosalicylic Acids, Ammonium Ion Binding Activity, AMPA Receptor Antagonists, Amphenicol-class Antibacterial, Amphenicols, Amphetamine Anorectic, Amphetamines, Amylin Agonists, Amylin Analog, Androgen Receptor Agonists, Androgen Receptor Antagonists, Androgen Receptor Inhibitor, Androgen, Androstanes, Angiotensin 2 Receptor Antagonists, Angiotensin 2 Receptor Blocker, Angiotensin 2 Type 1 Receptor Antagonists, Angiotensin Converting Enzyme Inhibitor, Angiotensin-converting Enzyme Inhibitors, Ant Venoms, Anthracycline Topoisomerase Inhibitor, Anthracyclines, Anti-anginal, Anti-coagulant, Anti-epileptic Agent, Anti-IgE, Anti-inhibitor Coagulant Complex, Antiarrhythmic, Antibodies, Monoclonal, Antibody-Surface Protein Interactions, Anticholinergic, Antidiarrheal, Antidiuretic Hormone Antagonists, Antidote for Acetaminophen Overdose, Antidote, Antiemetic, Antifibrinolytic Agent, Antigen Neutralization, Antigens, Bacterial, Antigens, Dermatophagoides, Antigens, Fungal, Antihelminthic, Antihistamine, Antimalarial, Antimetabolite Immunosuppressant, Antimetabolite, Antimycobacterial, Antiparasitic, Antiprotozoal, Antirheumatic Agent, Antiseptic, Antitoxins, Antivenin, Antivenins, Appetite Suppression, Aptamers, Nucleotide, Aromatase Inhibitor, Aromatase Inhibitors, Aromatic Amino Acid Decarboxylation Inhibitor, Arteriolar Vasodilation, Arteriolar Vasodilator, Asparaginase, Asparagine-specific Enzyme, Atypical Antipsychotic, Autologous Cellular Immunotherapy, Autologous Cultured Cell, Autonomic Ganglionic Blocker, Azole Antifungal, Azoles, B Lymphocyte Stimulator-directed Antibody Interactions, B Lymphocyte Stimulator-specific Inhibitor, Bacterial Neurotoxin Neutralization, Bacterial Proteins, Barbiturate, Barbiturates, BCG Vaccine, Bee Venoms, Benzodiazepine Antagonist, Benzodiazepine, Benzodiazepines, Benzothiazole, Benzothiazoles, Benzylamine Antifungal, Benzylamines, beta Lactamase Inhibitor, beta Lactamase Inhibitors, beta-Adrenergic Agonist, beta-Adrenergic Blocker, beta2-Adrenergic Agonist, beta3-Adrenergic Agonist, Biguanide, Biguanides, Bile Acid Sequestrant, Bile Acid, Bile Acids and Salts, Bile-acid Binding Activity, Bismuth, Bismuth, Bisphosphonate, Blood Coagulation Factor, Blood Coagulation Factors, Blood Viscosity Reducer, Bovine Intestinal Adenosine Deaminase, Bradykinin B2 Receptor Antagonist, Bradykinin B2 Receptor Antagonists, Calcineurin Inhibitor Immunosuppressant, Calcineurin Inhibitors, Calcitonin, Calcitonin, Calcium Channel Antagonists, Calcium Channel Blocker, Calcium Chelating Activity, Calcium, Calcium, Calcium-sensing Receptor Agonist, Calculi Dissolution Agent, Cannabinoid, Cannabinoids, Carbamoyl Phosphate Synthetase 1 Activator, Carbamoyl Phosphate Synthetase 1 Activators, Carbapenems, Carbon Radioisotopes, Carbonic Anhydrase Inhibitor, Carbonic Anhydrase Inhibitors, Cardiac Glycoside, Cardiac Glycosides, Carnitine Analog, Carnitine, Caseins, Catechol O-Methyltransferase Inhibitors, Catechol-O-Methyltransferase Inhibitor, Catecholamine Synthesis Inhibitor, Catecholamine Synthesis Inhibitors, Catecholamine, Catecholamine-depleting Sympatholytic, Catecholamines, Cations, Divalent, CCR5 Co-receptor Antagonist, CD20-directed Antibody Interactions, CD20-directed Cytolytic Antibody, CD20-directed Radiotherapeutic Antibody, CD25-directed Cytotoxin, CD3 Blocker Immunosuppressant, CD3 Receptor Antagonists, CD3-directed Antibody Interactions, CD30-directed Antibody Interactions, CD30-directed Immunoconjugate, CD52-directed Antibody Interactions, CD52-directed Cytolytic Antibody, CD80-directed Antibody Interactions, CD86-directed Antibody Interactions, Cell Death Inducer, Cell-mediated Immunity, Cells, Allogeneic, Cells, Cultured, Allogeneic, Cells, Cultured, Autologous, Cells, Epidermal, Central alpha-2 Adrenergic Agonist, Central Nervous System Depressant, Central Nervous System Depression, Central Nervous System Stimulant, Central Nervous System Stimulation, Centrally-mediated Muscle Relaxation, Cephalosporin Antibacterial, Cephalosporins, Chemokine Co-receptor 5 Antagonists, Chloride Channel Activation Potentiators, Chloride Channel Activator, Chloride Channel Activators, Cholecalciferol, Cholecystokinin Analog, Cholecystokinin, Cholinergic Agonists, Cholinergic Antagonists, Cholinergic Muscarinic Agonist, Cholinergic Muscarinic Agonists, Cholinergic Muscarinic Antagonist, Cholinergic Muscarinic Antagonists, Cholinergic Nicotinic Agonist, Cholinergic Receptor Agonist, Cholinesterase Inhibitor, Cholinesterase Inhibitors, Cholinesterase Reactivator, Cholinesterase Reactivators, Chondrocytes, Collagen, Collagen-specific Enzyme, Collagenases, Competitive Opioid Antagonists, Complement Inhibitor, Complement Inhibitors, Contrast Agent for Ultrasound Imaging, Copper Absorption Inhibitor, Copper, Copper-containing Intrauterine Device, Corticosteroid Hormone Receptor Agonists, Corticosteroid, CTLA-4-directed Antibody Interactions, CTLA-4-directed Blocking Antibody, Cyclooxygenase Inhibitors, Cysteine Depleting Agent, Cystic Fibrosis Transmembrane Conductance Regulator Potentiator, Cystine Disulfide Reduction, Cytochrome P450 1A2 Inhibitors, Cytochrome P450 2B6 Inducers, Cytochrome P450 2C19 Inducers, Cytochrome P450 2C19 Inhibitors, Cytochrome P450 2C8 Inducers, Cytochrome P450 2C8 Inhibitors, Cytochrome P450 2C9 Inducers, Cytochrome P450 2C9 Inhibitors, Cytochrome P450 2D6 Inhibitor, Cytochrome P450 2D6 Inhibitors, Cytochrome P450 3A Inducers, Cytochrome P450 3A Inhibitors, Cytochrome P450 3A4 Inducers, Cytochrome P450 3A4 Inhibitors, Cytochrome P450 3A5 Inhibitors, Cytomegalovirus Nucleoside Analog DNA Polymerase Inhibitor, Cytoprotective Agent, Dander, Decarboxylase Inhibitor, Decarboxylase Inhibitors, Decreased Autonomic Ganglionic Activity, Decreased B Lymphocyte Activation, Decreased Blood Pressure, Decreased Cell Wall Integrity, Decreased Cell Wall Synthesis & Repair, Decreased Central Nervous System Disorganized Electrical Activity, Decreased Central Nervous System Organized Electrical Activity, Decreased Cholesterol Absorption, Decreased Coagulation Factor Activity, Decreased Copper Ion Absorption, Decreased Cytokine Activity, Decreased DNA Replication, Decreased Embryonic Implantation, Decreased Fibrinolysis, Decreased GnRH Secretion, Decreased Histamine Release, Decreased IgE Activity, Decreased Immunologic Activity, Decreased Immunologically Active Molecule Activity, Decreased Leukotriene Production, Decreased Mitosis, Decreased Parasympathetic Acetylcholine Activity, Decreased Platelet Aggregation, Decreased Platelet Production, Decreased Prostaglandin Production, Decreased Protein Synthesis, Decreased Renal K+ Excretion, Decreased Respiratory Secretion Viscosity, Decreased RNA Replication, Decreased Sebaceous Gland Activity, Decreased Sperm Motility, Decreased Striated Muscle Contraction, Decreased Striated Muscle Tone, Decreased Sympathetic Activity, Decreased Tracheobronchial Stretch Receptor Activity, Decreased Vascular Permeability, Demulcent Activity, Demulcent, Deoxyribonuclease I, Deoxyuridine, Depigmenting Activity, Depigmenting Agent, Depolarizing Neuromuscular Blocker, Diagnostic Dye, Dietary Cholesterol Absorption Inhibitor, Dietary Proteins, Digestive/GI System Activity Alteration, Digoxin Binding Activity, Dihydrofolate Reductase Inhibitor Antibacterial, Dihydrofolate Reductase Inhibitor Antimalarial, Dihydrofolate Reductase Inhibitors, Dihydroorotate Dehydrogenase Inhibitors, Dihydropyridine Calcium Channel Blocker, Dihydropyridines, Dipeptidase Inhibitors, Dipeptidyl Peptidase 4 Inhibitor, Dipeptidyl Peptidase 4 Inhibitors, Diphosphonates, Diphtheria Toxin, Direct Thrombin Inhibitor, DNA Polymerase Inhibitors, DOPA Decarboxylase Inhibitors, Dopamine Agonists, Dopamine D2 Antagonists, Dopamine Uptake Inhibitors, Dopamine-2 Receptor Antagonist, Dopaminergic Agonist, Dyes, Echinocandin Antifungal, Egg Proteins, Dietary, Emesis Suppression, Endogenous Antigen Neutralization, Endoglycosidase, Endothelin Receptor Antagonist, Endothelin Receptor Antagonists, Enzyme Precursors, Epidermal Growth Factor Receptor Antagonist, Ergocalciferols, Ergolines, Ergot Alkaloids, Ergot Derivative, Ergot-derived Dopamine Receptor Agonist, Ergotamine Derivative, Ergotamines, Erythropoiesis-stimulating Agent, Erythropoietin, Ester Local Anesthetic, Esters, Estradiol Congeners, Estradiol, Estrogen Agonist/Antagonist, Estrogen Receptor Agonists, Estrogen Receptor Antagonist, Estrogen Receptor Antagonists, Estrogen, Estrogens, Conjugated (USP), Factor VIII Activator, Factor VIII, Factor Xa Inhibitor, Factor Xa Inhibitors, Fatty Acids, Omega-3, Feathers, Fibroblast Growth Factor 7, Fibroblasts, Fish Proteins, Dietary, Folate Analog Metabolic Inhibitor, Folate Analog, Folic Acid Metabolism Inhibitors, Folic Acid, Food Additives, Free Radical Scavenging Activity, Fruit Proteins, Full Opioid Agonists, Fungal Proteins, Fur, Fusion Protein Inhibitors, GABA A Agonists, GABA B Agonists, Gadolinium-based Contrast Agent, gamma-Aminobutyric Acid A Receptor Agonist, gamma-Aminobutyric Acid-ergic Agonist, General Anesthesia, General Anesthetic, Genitourinary Arterial Vasodilation, GI Motility Alteration, Glinide, GLP-1 Receptor Agonist, Glucagon-Like Peptide 1, Glucagon-like Peptide-1 (GLP-1) Agonists, Glucosylceramidase, Glucosylceramide Synthase Inhibitor, Glucosylceramide Synthase Inhibitors, Glycerol, Glycopeptide Antibacterial, Glycopeptides, Glycosaminoglycan, Glycosaminoglycans, Glycoside Hydrolases, Gonadotropin Releasing Hormone Antagonist, Gonadotropin Releasing Hormone Receptor Agonist, Gonadotropin Releasing Hormone Receptor Agonists, Gonadotropin Releasing Hormone Receptor Antagonists, Gonadotropin, Gonadotropins, Grain Proteins, Granulocyte Colony-Stimulating Factor, Granulocyte-Macrophage Colony-Stimulating Factor, Growth Hormone Receptor Antagonist, Growth Hormone Receptor Antagonists, Growth Hormone Releasing Factor Analog, Guanylate Cyclase Activators, Guanylate Cyclase Stimulators, Guanylate Cyclase-C Agonist, HEALTHCARE/PHARMACEUTICAL INDUSTRY MENU, Home, News, DailyMed Announcements, Get RSS News & Updates, Search, Advanced Search, Browse Drug Classes, Labels Archives, Tablet/Capsule ID Tool, FDA Guidances & Information, NLM SPL Resources, Download Data, All Drug Labels, All Index Files, All Mapping Files, SPL Image Guidelines, Presentations & Articles, Application Development Support, Resources, Web Services, Mapping Files, Help, SWITCH TO CONSUMER/PATIENT MENU, HCV NS3/4A Protease Inhibitors, Hedgehog Pathway Inhibitor, Helicobacter pylori Diagnostic, Hematologic Activity Alteration, Hematopoietic Stem Cell Mobilizer, Hematopoietic Stem Cells, Heparin Binding Activity, Heparin Reversal Agent, Heparin, Heparin, Low-Molecular-Weight, Hepatitis B Virus Nucleoside Analog Reverse Transcriptase Inhibitor, Hepatitis C Virus NS3/4A Protease Inhibitor, HER1 Antagonists, HER2 Receptor Antagonist, HER2/Neu/cerbB2 Antagonists, Herpes Simplex Virus Nucleoside Analog DNA Polymerase Inhibitor, Herpes Zoster Virus Nucleoside Analog DNA Polymerase Inhibitor, Herpesvirus Nucleoside Analog DNA Polymerase Inhibitor, Histamine H1 Receptor Antagonists, Histamine H2 Receptor Antagonists, Histamine Receptor Antagonists, Histamine-1 Receptor Antagonist, Histamine-1 Receptor Inhibitor, Histamine-2 Receptor Antagonist, Histone Deacetylase Inhibitor, Histone Deacetylase Inhibitors, HIV Integrase Inhibitors, HIV Protease Inhibitors, HMG-CoA Reductase Inhibitor, House Dust, Human alpha-1 Proteinase Inhibitor, Human Antihemophilic Factor, Human Blood Coagulation Factor, Human C1 Esterase Inhibitor, Human Immunodeficiency Virus 1 Fusion Inhibitor, Human Immunodeficiency Virus 1 Non-Nucleoside Analog Reverse Transcriptase Inhibitor, Human Immunodeficiency Virus Integrase Strand Transfer Inhibitor, Human Immunodeficiency Virus Nucleoside Analog Reverse Transcriptase Inhibitor, Human Immunoglobulin G, Human Immunoglobulin, Human Platelet-derived Growth Factor, Human Serum Albumin, Hydrolytic Lysosomal Glucocerebroside-specific Enzyme, Hydrolytic Lysosomal Glycogen-specific Enzyme, Hydrolytic Lysosomal Glycosaminoglycan-specific Enzyme, Hydrolytic Lysosomal Neutral Glycosphingolipid-specific Enzyme, Hydroxymethylglutaryl-CoA Reductase Inhibitors, Hydroxyphenyl-Pyruvate Dioxygenase Inhibitor, Hydroxyphenylpyruvate Dioxygenase Inhibitors, IgE-directed Antibody Interactions, Immunoconjugates, Immunoglobulin G, Immunoglobulins, Inactivated Salmonella typhi Vaccine, Increased Acetylcholine Activity, Increased Blood Pressure, Increased Calcium-sensing Receptor Sensitivity, Increased Cellular Death, Increased Coagulation Activity, Increased Coagulation Factor Activity, Increased Coagulation Factor IX Activity, Increased Coagulation Factor VIII Activity, Increased Coagulation Factor VIII Concentration, Increased Coagulation Factor X Activity, Increased Cytokine Activity, Increased Cytokine Production, Increased Diuresis at Loop of Henle, Increased Diuresis, Increased Dopamine Activity, Increased Epithelial Proliferation, Increased Erythroid Cell Production, Increased Fibrin Polymerization Activity, Increased GHRH Activity, Increased Glutathione Concentration, Increased Hematopoietic Stem Cell Mobilization, Increased Histamine Release, Increased IgG Production, Increased Immunologically Active Molecule Activity, Increased Intravascular Volume, Increased Large Intestinal Motility, Increased Lymphocyte Activation, Increased Lymphocyte Cell Production, Increased Macrophage Proliferation, Increased Medullary Respiratory Drive, Increased Megakaryocyte Maturation, Increased Myeloid Cell Production, Increased Norepinephrine Activity, Increased Oncotic Pressure, Increased Platelet Aggregation, Increased Platelet Production, Increased Prostaglandin Activity, Increased Prothrombin Activity, Increased Sympathetic Activity, Increased T Lymphocyte Activation, Increased T Lymphocyte Destruction, Increased Thrombolysis, Increased Uterine Smooth Muscle Contraction or Tone, Influenza A M2 Protein Inhibitor, Inhalation Diagnostic Agent, Inhibit Ovum Fertilization, Insect Proteins, Insulin Analog, Insulin, Insulin, Integrin Receptor Antagonist, Integrin Receptor Antagonists, Interferon Alfa-2a, Interferon Alfa-2b, Interferon alpha, Interferon gamma, Interferon Inducers, Interferon-alpha, Interferon-beta, Interferon-gamma, Interleukin 1 Receptor Antagonists, Interleukin 2 Receptor Antagonists, Interleukin 2 Receptor-directed Antibody Interactions, Interleukin 6 Receptor Antagonists, Interleukin-1 Receptor Antagonist, Interleukin-2 Receptor Blocking Antibody, Interleukin-2, Interleukin-6 Receptor Antagonist, Intestinal Lipase Inhibitor, Iodine, Iron Chelating Activity, Iron Chelator, Iron, Irrigation, Kallikrein Inhibitors, Keratinocytes, Ketolide Antibacterial, Ketolides, Kinase Inhibitor, 1-Thyroxine, 1-Triiodothyronine, Lead Chelating Activity, Lead Chelator, Leukocyte Growth Factor, Leukotriene Receptor Antagonist, Leukotriene Receptor Antagonists, Lincosamide Antibacterial, Lincosamides, Lipase Inhibitors, Lipid-based Polyene Antifungal, Lipopeptide Antibacterial, Lipopeptides, Live Attenuated Bacillus Calmette-Guerin Immunotherapy, Live Attenuated Bacillus Calmette-Guerin Vaccine, Live Attenuated Mumps Virus Vaccine, Live Human Adenovirus Type 4 Vaccine, Live Human Adenovirus Type 7 Vaccine, Live Rotavirus Vaccine, Local Anesthesia, Local Anesthetic, Loop Diuretic, Low Molecular Weight Heparin, Lymphocyte Function Alteration, Lymphocyte Growth Factor, M2 Protein Inhibitors, Macrolide Antibacterial, Macrolide Antimicrobial, Macrolide, Macrolides, Magnesium Ion Exchange Activity, Magnetic Resonance Contrast Activity, Mast Cell Stabilizer, Meat Proteins, Megakaryocyte Growth Factor, Melanin Synthesis Inhibitor, Melanin Synthesis Inhibitors, Melatonin Receptor Agonist, Melatonin Receptor Agonists, Metal Chelating Activity, Metal Chelator, Methylated Sulfonamide Antibacterial, Methylated Sulfonamides, Methylating Activity, Methylating Agent, Methylxanthine, Microtubule Inhibition, Microtubule Inhibitor, Milk Proteins, Monoamine Oxidase Inhibitor, Monoamine Oxidase Inhibitors, Monoamine Oxidase Type B Inhibitor, Monoamine Oxidase-B Inhibitors, Monobactam Antibacterial, Monobactams, Mood Stabilizer, mTOR Inhibitor Immunosuppressant, mTOR Inhibitors, Mucocutaneous Epithelial Cell Growth Factor, Mucolytic, Mumps Vaccine, Muscle Relaxant, N-Calcium Channel Receptor Antagonists, N-methyl-D-aspartate Receptor Antagonist, N-substituted Glycines, N-type Calcium Channel Antagonist, Natriuretic Peptide, Natriuretic Peptides, Neuraminidase Inhibitor, Neuraminidase Inhibitors, Neurokinin 1 Antagonists, Neuromuscular Depolarizing Blockade, Neuromuscular Nondepolarizing Blockade, Nicotine, Nicotinic Acid, Nicotinic Acids, Nitrate Vasodilator, Nitrates, Nitrofuran Antibacterial, Nitrofurans, Nitrogen Binding Agent, Nitrogen Mustard Compounds, Nitroimidazole Antimicrobial, Nitroimidazoles, NMDA Receptor Antagonists, Non-narcotic Antitussive, Non-Nucleoside Analog, Non-Nucleoside Reverse Transcriptase Inhibitors, Non-Standardized Animal Dander Allergenic Extract, Non-Standardized Animal Hair Allergenic Extract, Non-Standardized Animal Skin Allergenic Extract, Non-Standardized Bacterial Allergenic Extract, Non-Standardized Chemical Allergen, Non-Standardized Feather Allergenic Extract, Non-Standardized Food Allergenic Extract, Non-Standardized Fungal Allergenic Extract, Non-Standardized House Dust Allergenic Extract, Non-Standardized Insect Allergenic Extract, Non-Standardized Insect Venom Allergenic Extract, Non-Standardized Plant Allergenic Extract, Non-Standardized Plant Fiber Allergenic Extract, Non-Standardized Pollen Allergenic Extract, Noncompetitive AMPA Glutamate Receptor Antagonist, Nondepolarizing Neuromuscular Blocker, Nonergot Dopamine Agonist, Nonsteroidal Anti-inflammatory Compounds, Nonsteroidal Anti-inflammatory Drug, Norepinephrine Reuptake Inhibitor, Norepinephrine Uptake Inhibitors, Nucleic Acid Synthesis Inhibitors, Nucleoside Analog Antifungal, Nucleoside Analog Antiviral, Nucleoside Analog, Nucleoside Metabolic Inhibitor, Nucleoside Reverse Transcriptase Inhibitors, Nut Proteins, Oligonucleotides, Omega-3 Fatty Acid, Opioid Agonist, Opioid Agonist/Antagonist, Opioid Agonists, Opioid Antagonist, Opioid Antagonists, Organic Anion Transporting Polypeptide 1B1 Inhibitors, Organic Anion Transporting Polypeptide 1B3 Inhibitors, Organic Anion Transporting Polypeptide 2B1 Inhibitors, Organic Cation Transporter 2 Inhibitors, Organometallic Compounds, Osmotic Activity, Osmotic Diuretic, Osmotic Laxative, Oxazolidinone Antibacterial, Oxazolidinones, Oxytocic, Oxytocin, P-Glycoprotein Inhibitors, P-Glycoprotein Interactions, P2Y12 Platelet Inhibitor, P2Y12 Receptor Antagonists, Paramagnetic Contrast Agent, Parathyroid Hormone Analog, Parathyroid Hormone, Parenteral Iron Replacement, Partial Cholinergic Nicotinic Agonist, Partial Cholinergic Nicotinic Agonists, Partial Opioid Agonist, Partial Opioid Agonist/Antagonist, Partial Opioid Agonists, Passively Acquired Immunity, Pediculicide, peginterferon alfa-2a, peginterferon alfa-2b, Penem Antibacterial, Penicillin-class Antibacterial, Penicillins, Peripheral Blood Mononuclear Cells, Peroxisome Proliferator Receptor alpha Agonist, Peroxisome Proliferator Receptor gamma Agonist, Peroxisome Proliferator-activated Receptor Activity, Peroxisome Proliferator-activated Receptor alpha Agonists, Phenothiazine, Phenothiazines, Phenylalanine Hydroxylase Activator, Phenylalanine Hydroxylase Activators, Phosphate Binder, Phosphate Chelating Activity, Phosphodiesterase 3 Inhibitor, Phosphodiesterase 3 Inhibitors, Phosphodiesterase 5 Inhibitor, Phosphodiesterase 5 Inhibitors, Photoabsorption, Photoactivated Radical Generator, Photoenhancer, Photosensitizing Activity, Plant Proteins, Plasma Volume Expander, Platelet Aggregation Inhibitor, Platelet-Derived Growth Factor, Platelet-reducing Agent, Platinum-based Drug, Platinum-containing Compounds, Pleuromutilin Antibacterial, pleuromutilin, Pollen, Polyene Antifungal, Polyene Antimicrobial, Polyenes, Polymyxin-class Antibacterial, Polymyxins, Porphyrin Precursor, Porphyrinogens, Positron Emitting Activity, Potassium Channel Antagonists, Potassium Channel Opener, Potassium Channel Openers, Potassium Compounds, Potassium Salt, Potassium-sparing Diuretic, Poultry Proteins, PPAR alpha, PPAR gamma, Progestational Hormone Receptor Antagonists, Progesterone Congeners, Progesterone, Progesterone, Progestin Antagonist, Progestin, Progestin-containing Intrauterine Device, Prostacycline Vasodilator, Prostacycline, Prostaglandin Analog, Prostaglandin E1 Agonist, Prostaglandin E1 Analog, Prostaglandin Receptor Agonists, Prostaglandins E, Synthetic, Prostaglandins I, Prostaglandins, Protease Inhibitor, Proteasome Inhibitor, Proteasome Inhibitors, Protein C, Protein Kinase Inhibitors, Protein Synthesis Inhibitors, Proton Pump Inhibitor, Proton Pump Inhibitors, Provitamin D2 Compound, Psoralen, Psoralens, Purine Antimetabolite, Purines, Pyrethrins, Pyrethroid, Pyrimidine Synthesis Inhibitor, Pyrophosphate Analog DNA Polymerase Inhibitor, Pyrophosphate Analog, Quaternary Ammonium Compounds, Quinolone Antimicrobial, Quinolones, Radioactive Diagnostic Agent, Radioactive Therapeutic Agent, Radioactive Tracers, Radiographic Contrast Agent, Radiopharmaceutical Activity, RANK Ligand Blocking Activity, RANK Ligand Inhibitor, Receptor Tyrosine Kinase Inhibitors, Recombinant Antithrombin, Recombinant Fusion Proteins, Recombinant Human Deoxyribonuclease 1, Recombinant Human Growth Hormone, Recombinant Human Growth Hormones, Recombinant Human Interferon beta, Recombinant Proteins, Reducing and Complexing Thiol, Reduction Activity, Renal Dehydropeptidase Inhibitor, Renin Inhibitor, Renin Inhibitors, Respiratory Stimulant, Respiratory Syncytial Virus Anti-F Protein Monoclonal Antibody, Retinoid, Retinoids, Reversed Anticoagulation Activity, Rifamycin Antibacterial, Rifamycin Antimycobacterial, Rifamycins, RNA Synthetase Inhibitor Antibacterial, RNA Synthetase Inhibitors, Rotavirus Vaccines, Salivary Proteins and Peptides, Sclerosing Activity, Sclerosing Agent, Seed Storage Proteins, Selective Estrogen Receptor Modulators, Selective T Cell Costimulation Blocker, Selective T Cell Costimulation Modulator, Serotonin 1b Receptor Agonists, Serotonin 1d Receptor Agonists, Serotonin 2c Receptor Agonists, Serotonin 3 Receptor Antagonists, Serotonin 4 Receptor Antagonists, Serotonin and Norepinephrine Reuptake Inhibitor, Serotonin Reuptake Inhibitor, Serotonin Uptake Inhibitors, Serotonin-1b and Serotonin-1d Receptor Agonist, Serotonin-2c Receptor Agonist, Serotonin-3 Receptor Antagonist, Serotonin-4 Receptor Antagonist, Serum Albumin, Shellfish Proteins, Sigma-1 Agonist, Sigma-1 Receptor Agonists, Silk, Skeletal Muscle Relaxant, Skin Barrier Activity, Skin Test Antigen, Smoothened Receptor Antagonists, Sodium-Glucose Cotransporter 2 Inhibitor, Sodium-Glucose Transporter 2 Inhibitors, Soluble Guanylate Cyclase Stimulator, Somatostatin Analog, Somatostatin Receptor Agonists, Sphingosine 1-phosphate Receptor Modulator, Sphingosine 1-Phosphate Receptor Modulators, Standardized Animal Hair Allergenic Extract, Standardized Animal Skin Allergenic Extract, Standardized Chemical Allergen, Standardized Insect Allergenic Extract, Standardized Insect Venom Allergenic Extract, Standardized Pollen Allergenic Extract, Starch, Stimulant Laxative, Stimulation Large Intestine Fluid/Electrolyte Secretion, Streptogramin Antibacterial, Streptogramins, Substance P/Neurokinin-1 Receptor Antagonist, Sucrose-specific Enzyme, Sulfonamide Antibacterial, Sulfonamide Antimicrobial, Sulfonamides, Sulfone, Sulfones, Sulfonylurea Compounds, Sulfonylurea, Surfactant Activity, Surfactant, Sympathomimetic Amine Anorectic, Sympathomimetic-like Agent, T Lymphocyte Costimulation Activity Blockade, Tetracycline-class Antibacterial, Tetracycline-class Antimicrobial, Tetracycline-class Drug, Tetracyclines, Thalidomide Analog, Thiazide Diuretic, Thiazide-like Diuretic, Thiazides, Thiazolidinedione, Thiazolidinediones, Thrombin Inhibitors, Thrombolytic Agent, Thrombopoiesis Stimulating Agent, Thrombopoietin Receptor Agonists, Thrombopoietin Receptor Interactions, Thrombopoietin, Thyroid Hormone Synthesis Inhibitor, Thyroid Hormone Synthesis Inhibitors, Thyroid Stimulating Hormone, Thyrotropin, Thyroxine, Tissue Scaffolds, Topoisomerase Inhibitor, Topoisomerase Inhibitors, Transglutaminases, Tricyclic Antidepressant, Triiodothyronine, Trypsin Inhibitors, Tuberculosis Skin Test, Tumor Necrosis Factor alpha Receptor Blocking Activity, Tumor Necrosis Factor Blocker, Tumor Necrosis Factor Receptor Blocking Activity, Typical Antipsychotic, Ultrasound Contrast Activity, Uncompetitive N-methyl-D-aspartate Receptor Antagonist, Uncompetitive NMDA Receptor Antagonists, Unfractionated Heparin, Urate Oxidase, Urea, Urease Inhibitor, Urease Inhibitors, Uric Acid-specific Enzyme, Vaccines, Attenuated, Vaccines, Inactivated, Vaccines, Live, Unattenuated, Vaccines, Typhoid, Vascular Endothelial Growth Factor Receptor Inhibitors, Vascular Endothelial Growth Factor-directed Antibody Interactions, Vascular Endothelial Growth Factor-directed Antibody, Vascular Sclerosing Activity, Vasodilation, Vasodilator, Vasopressin Analog, Vasopressin Antagonist, Vasopressins, Vegetable Proteins, Venom Neutralization, Venous Vasodilation, Vi polysaccharide vaccine, typhoid, Vinca Alkaloid, Vinca Alkaloids, Virus Neutralization, Virus-specific Hyperimmune Globulins, Vitamin A, Vitamin A, Vitamin B 12, Vitamin B Complex Compounds, Vitamin B Complex Member, Vitamin B12, Vitamin D Analog, Vitamin D, Vitamin D, Vitamin D2 Analog, Vitamin D3 Analog, Vitamin K Antagonist, Vitamin K Inhibitors, Vitamin K, Vitamin K, von Willebrand Factor, Warfarin Reversal Agent, Wasp Venoms, X-Ray Contrast Activity, Xanthine Oxidase Inhibitor, Xanthine Oxidase Inhibitors, Xanthines, or combinations thereof.

Microsatellite Extension Inhibitors

In an embodiment a payload comprises a microsatellite extension inhibitor. In an embodiment, the microsatellite extension inhibitor is a DNA mismatch repair protein. Exemplary DNA mismatch repair proteins that can be delivered by the molecules and methods described herein include, e.g., MSH2, MSH3, MSH6, MLH1, MLH3, PMS1, PMS2.

Signal Generators, Radionuclides, Reporter Molecules, Diagnostic Probes

In an embodiment a payload comprises a molecule that generates a signal. Such payloads are useful, e.g., in research, therapeutic (e.g., cancer therapy) and diagnostic applications. In an embodiment, the signal comprises: an electromagnetic emission, e.g., in the infrared, visible, or ultraviolet range; a particle, e.g., a product of radioactive decay, e.g., an alpha, beta, or gamma particle; a detectable substrate, e.g., a colored substrate; a reaction product, e.g., the product of an enzymatic reaction; or a ligand detectable by a specific binding agent, e.g., an antibody; or a dye. In an embodiment the signal comprises a fluorescent emission, e.g., by a fluorescent protein. Exemplary fluorescent proteins include, Blue/UV Proteins (e.g., TagBFP, mTagBFP, Azurite, EBFP2, mKalamal, Sirius, Sapphire, T-Sapphire), Cyan Proteins (e.g., ECFP, Cerulean, SCFP3A, mTurquoise, mTurquoise2, monomeric Midoriishi-Cyan, TagCFP, mTFP1), Green Proteins (e.g., EGFP, Emerald, Superfolder GFP, Monomeric Azami Green, TagGFP2, mUKG, mWasabi, Clover, mNeonGreen), Yellow Proteins (e.g., EYFP, Citrine, Venus, SYFP2, TagYFP), Orange Proteins (e.g., Monomeric Kusabira-Orange, mKOx, mKO2, mOrange, mOrange2), Red Proteins (mRaspberry, mCherry, mStrawberry, mTangerine, tdTomato, TagRFP, TagRFP-T, mApple, mRuby, mRuby2), Far-Red Proteins (e.g., mPlum, HcRed-Tandem, mKate2, mNeptune, NirFP, TagRFP657, IFP1.4, iRFP), Long Stokes Shift Proteins (e.g., mKeima Red, LSS-mKate1, LSS-mKate2, mBeRFP), Photoactivatible Proteins (e.g., PA-GFP, PAmCherryl, PATagRFP), Photoconvertible Proteins (e.g., Kaede (green), Kaede (red), KikGR1 (green), KikGR1 (red), PS-CFP2, mEos2 (green), mEos2 (red), mEos3.2 (green), mEos3.2 (red), PSmOrange), Photoswitchable Proteins (e.g., Dronpa).

In an embodiment, a signal producing moiety is provided as the fusion partner of a Cas9 molecule, e.g., an eiCas9 molecule.

Signal generators or reporters, useful, e.g., for labelingr polypeptides include, but are not limited to, the following: radioisotopes or radionuclides (e.g., indium (¹¹¹In), iodine (¹³¹I or ¹²⁵I) yttrium (⁹⁰Y), lutetium (¹⁷⁷Lu), actinium (²²⁵Ac), bismuth (²¹²Bi or ²¹³Bi), sulfur (³⁵S), carbon (¹⁴C), tritium (³H), rhodium (¹⁸⁸Rh), technetium (⁹⁹mTc), praseodymium, or phosphorous (³²P) or a positron-emitting radionuclide, e.g., carbon-11 (¹¹C), potassium-40 (⁴⁰K), nitrogen-13 (¹³N), oxygen-15 (¹⁵O), fluorine-18 (¹⁸F), and iodine-121 (¹²¹I)), fluorescent labels (e.g., FITC, rhodamine, lanthanide phosphors), enzymatic labels (e.g., horseradish peroxidase, beta-galactosidase, luciferase, alkaline phosphatase), chemiluminescent, biotinyl groups (which can be detected by a marked avidin, e.g., a molecule containing a streptavidin moiety and a fluorescent marker or an enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

In an embodiment, a payload comprises a radionuclide. The radionuclide can be incorporated into the gRNA molecule, the Cas9 molecule, or into a payload molecule. Exemplary radionuclides include, e.g., beta emitters, alpha emitters or gamma emitters. In an embodiment the radionuclide is iodine, e.g., ¹³¹I or ¹²⁵I, yttrium, e.g., ⁹⁰Y, lutetium, e.g., ¹⁷⁷Lu, Actinium, e.g., ²²⁵Ac, bismuth, e.g., ²¹²Bi or ²¹³Bi), sulfur, e.g., ³⁵S), carbon, e.g., ¹⁴C, tritium, ³H), rhodium, e.g., ¹⁸⁸Rh, technetium, e.g., ⁹⁹Tc, praseodymium, or phosphorous, e.g., ³²P.

Modulators of DNA and Chromatin Structure

In an embodiment a payload comprises an endogenous or exogenous modulator of DNA structure. A modulator, as is typical of payloads, can be delivered in vitro, ex vivo, or in vivo.

In an embodiment, the payload comprises a modulator of an epigenetic state or characteristic of DNA. In an embodiment an epigenetic state or characteristic can be altered to treat a disorder, or to influence the developmental or other state of a cell.

In an embodiment, the epigenetic state or characteristic comprises DNA methylation. For example, the payloads described herein can modulate the addition of methyl groups to DNA, e.g., to convert cytosine to 5-methylcytosine, e.g., at CpG sites.

Aberrant DNA methylation patterns (e.g., hypermethylation and hypomethylation compared to normal tissue) are associated with various diseases and conditions, e.g., cancer. The modulators described herein can be used to reactivate transcriptionally silenced genes or to inhibit transcriptionally hyperactive genes, e.g., to treat diseases, e.g., cancer.

DNA methylation can affect gene transcription. Genes with high levels of 5-methylcytosine, e.g., in their promoter region, can be transcriptionally less active or silent. Thus, methods described herein can be used to target and suppress transcriptional activity, e.g., of genes described herein.

In some embodiments, the modulator promotes maintenance of DNA methylation. For example, the modulators can have DNA methyltransferase (DNMT) activity or modulate DNMT activity, e.g., to maintain DNA methylation or reduce passive DNA demethylation, e.g., after DNA replication.

In some embodiments, the modulator promotes de novo DNA methylation. For example, the modulators described herein can have de novo DNA methyltransferase (DNMT) (e.g., DNMT3a, DNMT3b, DNMT3L) activity or modulate de novo DNMT (e.g., DNMT3a, DNMT3b, DNMT3L) activity, e.g., to produce DNA methylation patterns, e.g., early in development.

Epigenetic changes in DNA (e.g., methylation), can be evaluated by art-known methods or as described herein. Exemplary methods for detecting DNA methylation include, e.g., Methylation-Specific PCR (MSP), whole genome bisulfite sequencing (BS-Seq), HELP (HpaII tiny fragment Enrichment by Ligation-mediated PCR) assay, ChIP-on-chip assays, restriction landmark genomic scanning, Methylated DNA immunoprecipitation (MeDIP), pyrosequencing of bisulfite treated DNA, molecular break light assay for DNA adenine methyltransferase activity, methyl sensitive Southern Blotting, separation of native DNA into methylated and unmethylated fractions using MethylCpG Binding Proteins (MBPs) and fusion proteins containing just the Methyl Binding Domain (MBD).

In an embodiment, the modulator cleaves DNA. For example, a modulator can catalyze the hydrolytic cleavage of phosphodiester linkages in the DNA backbone. In some embodiments, the modulator (e.g., DNase I) cleaves DNA preferentially at phosphodiester linkages adjacent to a pyrimidine nucleotide, yielding 5′-phosphate-terminated polynucleotides with a free hydroxyl group on position 3′. In other embodiments, the modulator (e.g., DNase II) hydrolyzes deoxyribonucleotide linkages in DNA, yielding products with 3′-phosphates. In some embodiments, the modulator comprises endodeoxyribonuclease activity. In other embodiments, the modulator comprises exodeoxyribonuclease activity (e.g., having 3′ to 5′ or 5′ to 3′ exodeoxyribonuclease activity). In some embodiments, the modulator recognizes a specific DNA sequence (e.g., a restriction enzyme). In other embodiments, the modulator does not cleave DNA in a sequence-specific manner. A modulator can cleave single-stranded DNA (e.g., having nickase activity), double-stranded DNA, or both.

In an embodiment, modulator affects, e.g., alters or preserves, tertiary or quaternary DNA structure. For example, the modulators described herein can modulate tertiary structure, e.g., handedness (right or left), length of the helix turn, number of base pairs per turn, and/or difference in size between the major and minor grooves. In some embodiments, the modulator mediates the formation of B-DNA, A-DNA, and/or Z-DNA. The modulators described herein can also modulate quaternary structure, e.g., the interaction of DNA with other molecules (DNA or non-DNA molecules, e.g., histones), e.g., in the form of chromatin. In some embodiments, the modulator that mediate or modify tertiary or quaternary DNA structure comprises DNA helicases activity or modulates DNA helicase activity.

In some embodiments, the modulator promotes or inhibits DNA damage response and/or repair. For example, a modulator can promote one or more DNA damage response and repair mechanisms, e.g., direct reversal, base excision repair (BER), nucleotide excision repair (NER) (e.g., global genomic repair (GG-NER), transcription-coupled repair (TC-NER)), mismatch repair (MMR), non-homologous end joining (NHEJ), microhomology-mediated end joining (MMEJ), homologous recombination, and/or translesion synthesis (TLS). In some embodiments, a modulator promotes the step of damage recognition. In other embodiments, a modulator promotes the step of DNA repair.

Aberrant DNA damage repair is associated with various diseases and conditions, e.g., aging, hereditary DNA repair disorders, and cancer. For example, DNA repair gene mutations that can increase cancer risk include, e.g., BRCA1 and BRCA2 (e.g., involved in homologous recombination repair (HRR) of double-strand breaks and daughter strand gaps, e.g., in breast and ovarian cancer); ATM (e.g., different mutations reduce HRR, single strand annealing (SSA), NHEJ or homology-directed DSBR (HDR), e.g., in leukemia, lymphoma, and breast cancer), NBS (e.g., involved in NHEJ, e.g., in lymphoid malignancies); MRE11 (e.g., involved in HRR, e.g., in breast cancer); BLM (e.g., involved in HRR, e.g., in leukemia, lymphoma, colon, breast, skin, auditory canal, tongue, esophagus, stomach, tonsil, larynx, lung, and uterus cancer); WRN (e.g., involved in HRR, NHEJ, long-patch BER, e.g., in soft tissue sarcomas, colorectal, skin, thyroid, and pancreatic cancer); RECQ4 (RECQL4) (e.g., involved in HRR, e.g., causing Rothmund-Thomson syndrome (RTS), RAPADILINO syndrome or Baller Gerold syndrome, cutaneous carcinomas, including basal cell carcinoma, squamous cell carcinoma, and Bowen's disease); FANCA, FANCB, FANCC, FANCD1, FANCD2, FANCE, FANCF, FANCG, FANCI, FANCJ, FANCL, FANCM, and FANCN (e.g., involved in HRR and TLS, e.g., in leukemia, liver tumors, solid tumors in many locations), XPC and XPE(DDB2) (e.g., involved in NER(GGR type), e.g., in skin cancer (melanoma and non-melanoma)); XPA, XPB, XPD, XPF, and XPG (e.g., involved in NER (both GGR type and TCR type), e.g., in skin cancer (melanoma and non-melanoma) and central nervous system); XPV(POLH) (e.g., involved in TLS, e.g., in skin cancer (melanoma and non-melanoma)); hMSH2, hMSH6, hMLH1, and hPMS2 (involved in MMR, e.g., in colorectal, endometrial and ovarian cancer); MUTYH (e.g., involved in BER of A mispaired with 80H-dG, as well as mispairs with G, FapydG and C, e.g., in colon cancer)

Modulators can be used to treat a disease or condition associated with aberrant DNA damage repair, e.g., by modulating one or more DNA damage repair mechanisms described herein.

In some embodiments, the modulator is selected from, or modulates, one or more proteins involved in direct reversal, e.g., methyl guanine methyl transferase (MGMT).

In some embodiments, the modulator is selected from, or modulates, one or more proteins involved in BER, e.g., DNA glycosylase, AP endonuclease, DNA polymerase, DNA ligase.

In some embodiments, the modulator is selected from, or modulates, one or more proteins involved in GG-NER, e.g., XPC, HR23b, CAK, TFIIH, XPA, RPA, XPG, XPF, ERCC1, TFIIH, PCNA, RFC, ADN Pol, and Ligase I.

In some embodiments, the modulator is selected from, or modulates, one or more proteins involved in TC-NER, e.g., CSB, XPA, RPA, XPG, XPF, ERCC1, CSA-CNS, TFIIH, CAK, PCNA, RFC, Ligase I, and RNA Polymerase II.

In some embodiments, the modulator is selected from, or modulates, one or more DNA mismatch repair proteins.

In some embodiments, the modulator is selected from, or modulates, one or more proteins involved in NHEJ, e.g., Ku70/80, DNA-PKcs, DNA Ligase IV, XRCC4, XLF, Artemis, DNA polymerase mu, DNA polymerase lambda, PNKP, Aprataxin, and APLF.

In some embodiments, the modulator is selected from, or modulates, one or more proteins involved in homologous recombination, e.g., as described herein.

In some embodiments, the modulator is selected from, or modulates, one or more proteins involved in TLS, e.g., DNA polymerase eta, iota, kappa, zeta, and PCNA.

In an embodiment, a modulator can modulate global response to DNA damage, e.g., DNA damage checkpoints and/or transcriptional responses to DNA damage. For example, DNA damage checkpoints can occur at the GUS and G2/M boundaries. An intra-S checkpoint can also exist. Checkpoint activation can be modulated by two master kinases, ATM and ATR. ATM can respond to DNA double-strand breaks and disruptions in chromatin structure and ATR can respond to stalled replication forks. These kinases can phosphorylate downstream targets in a signal transduction cascade, e.g., leading to cell cycle arrest. A class of checkpoint mediator proteins (e.g., BRCA1, MDC1, and 53BP1), which transmit the checkpoint activation signal to downstream proteins, can be modulated. Exemplary downstream proteins that can be modulated include, e.g., p53, p21, and cyclin/cyclin-dependent kinase complexes.

In some embodiments, the modulator modulates nuclear DNA damage response and repair. In other embodiments, the modulator modulates mitochondrial DNA damage response and repair.

In some embodiments, the modulator promotes or inhibits DNA replication. For example, a modulator can promote or inhibit one or more stages of DNA replication, e.g., initiation (e.g., assembly of pre-replicative complex and/or initiation complex), elongation (e.g., formation of replication fork), and termination (e.g., formation of replication fork barrier). In some embodiments, the modulator is selected from, or modulates, one or more proteins involved in initiation, e.g., the origin recognition complex (ORC), CDC6, CDT1, minichromosome maintenance proteins (e.g., MCM2, MCM3, MCM4, MCM5, MCM6, MCM7, and MCM10), CDC45, CDK, DDK, CDC101, CDC102, CDC103, and CDC105. In some embodiments, the modulator is selected from, or modulates, one or more proteins involved in elongation, e.g., DNA helicases, DNA polymerase, PCNA, CDC45-MCM-GINS helicase complex, and Replication Factor C complex.

In some embodiments, the modulator is selected, from or modulates, one or more proteins involved in termination, e.g., type II topoisomerase and telomerase. In some embodiments, the modulator is selected from, or modulates, one or more replication checkpoint proteins, e.g., ATM, ATR, ATRIP, TOPBP1, RAD9, HUS1, Rad1, and CHK1.

In some embodiments, the payload comprises a modulator of nuclear DNA replication. In other embodiments, the modulator promotes or inhibits mitochondrial DNA replication.

Defects in DNA replication can be associated with various diseases and conditions, e.g., cancer and neurological diseases (e.g., Alzheimer's disease). Defects in mitochondrial DNA replication can also be associated with diseases and conditions, e.g., mtDNA depletion syndromes (e.g., Alpers or early infantile hepatocerebral syndromes) and mtDNA deletion disorders (e.g., progressive external ophthalmoplegia (PEO), ataxia-neuropathy, or mitochondrial neurogastrointestinal encephalomyopathy (MNGIE)). A modulator can be used to treat a disease or condition associated with aberrant DNA replication, e.g., by modulating DNA replication as described herein.

Exemplary endogenous or exogenous modulators of DNA structure are described herein, e.g., in Table VI-3.

TABLE VI-3

| | |
|---|---|
| DNA2 | DNA replication helicase/nuclease 2 |
| DNAAF1 | dynein, axonemal, assembly factor 1 |
| DNAAF2 | dynein, axonemal, assembly factor 2 |
| DNAAF3 | dynein, axonemal, assembly factor 3 |
| DNAH1 | dynein, axonemal, heavy chain 1 |
| DNAH2 | dynein, axonemal, heavy chain 2 |
| DNAH3 | dynein, axonemal, heavy chain 3 |
| DNAH5 | dynein, axonemal, heavy chain 5 |
| DNAH6 | dynein, axonemal, heavy chain 6 |
| DNAH7 | dynein, axonemal, heavy chain 7 |
| DNAH8 | dynein, axonemal, heavy chain 8 |
| DNAH9 | dynein, axonemal, heavy chain 9 |
| DNAH10 | dynein, axonemal, heavy chain 10 |
| DNAH10OS | dynein, axonemal, heavy chain 10 opposite strand |
| DNAH11 | dynein, axonemal, heavy chain 11 |
| DNAH12 | dynein, axonemal, heavy chain 12 |
| DNAH14 | dynein, axonemal, heavy chain 14 |
| DNAH17 | dynein, axonemal, heavy chain 17 |
| DNAH17-AS1 | DNAH17 antisense RNA 1 |
| DNAI1 | dynein, axonemal, intermediate chain 1 |
| DNAI2 | dynein, axonemal, intermediate chain 2 |
| DNAJB8-AS1 | DNAJB8 antisense RNA 1 |
| DNAJC3-AS1 | DNAJC3 antisense RNA 1 (head to head) |
| DNAJC9-AS1 | DNAJC9 antisense RNA 1 |
| DNAJC25- | DNAJC25-GNG10 readthrough |
| GNG10 | |
| DNAJC27- | DNAJC27 antisense RNA 1 |
| AS1 | |
| DNAL1 | dynein, axonemal, light chain 1 |
| DNAL4 | dynein, axonemal, light chain 4 |
| DNALI1 | dynein, axonemal, light intermediate chain 1 |
| DNASE1 | deoxyribonuclease I |
| DNASE1L1 | deoxyribonuclease I-like 1 |
| DNASE1L2 | deoxyribonuclease I-like 2 |
| DNASE1L3 | deoxyribonuclease I-like 3 |
| DNASE2 | deoxyribonuclease II, lysosomal |
| DNASE2B | deoxyribonuclease II beta |
| CD226 | CD226 molecule |
| FAM120A | family with sequence similarity 120A |
| GAK | cyclin G associated kinase |
| GCFC2 | GC-rich sequence DNA-binding factor 2 |
| MCM10 | minichromosome maintenance complex component 10 |
| PRKDC | protein kinase, DNA-activated, catalytic polypeptide |
| SACS | spastic ataxia of Charlevoix-Saguenay (sacsin) |
| SCNN1D | sodium channel, non-voltage-gated 1, delta subunit |
| SPATS2L | spermatogenesis associated, serine-rich 2-like |
| MT7SDNA | mitochondrially encoded 7S DNA |
| DCLRE1A | DNA cross-link repair 1A |
| DCLRE1B | DNA cross-link repair 1B |
| DCLRE1C | DNA cross-link repair 1C |
| DDIT3 | DNA-damage-inducible transcript 3 |
| DDIT4 | DNA-damage-inducible transcript 4 |
| DDIT4L | DNA-damage-inducible transcript 4-like |
| DFFA | DNA fragmentation factor, 45 kDa, alpha polypeptide |
| DFFB | DNA fragmentation factor, 40 kDa, beta polypeptide |
| | (caspase-activated DNase) |
| DMAP1 | DNA methyltransferase 1 associated protein 1 |
| DMC1 | DNA meiotic recombinase 1 |
| DNMT1 | DNA (cytosine-5-)-methyltransferase 1 |
| DNMT3A | DNA (cytosine-5-)-methyltransferase 3 alpha |
| DNMT3B | DNA (cytosine-5-)-methyltransferase 3 beta |
| DNMT3L | DNA (cytosine-5-)-methyltransferase 3-like |
| DNTT | DNA nucleotidylexotransferase |
| DRAM1 | DNA-damage regulated autophagy modulator 1 |
| DRAM2 | DNA-damage regulated autophagy modulator 2 |
| DSCC1 | DNA replication and sister chromatid cohesion 1 |
| ZBP1 | Z-DNA binding protein 1 |
| SON | SON DNA binding protein |
| TARDBP | TAR DNA binding protein |
| BMF | Bcl2 modifying factor |
| CENPBD1 | CENPB DNA-binding domains containing 1 |
| UNG | uracil-DNA glycosylase |
| PDRG1 | p53 and DNA-damage regulated 1 |
| TDG | thymine-DNA glycosylase |
| TDP1 | tyrosyl-DNA phosphodiesterase 1 |
| TDP2 | tyrosyl-DNA phosphodiesterase 2 |
| AHDC1 | AT hook, DNA binding motif, containing 1 |
| GMNN | geminin, DNA replication inhibitor |
| PRIM1 | primase, DNA, polypeptide 1 (49 kDa) |
| PRIM2 | primase, DNA, polypeptide 2 (58 kDa) |
| HELB | helicase (DNA) B |
| LIG1 | ligase I, DNA, ATP-dependent |
| SUMF1 | sulfatase modifying factor 1 |
| SUMF2 | sulfatase modifying factor 2 |
| LIG4 | ligase IV, DNA, ATP-dependent |
| LIG3 | ligase III, DNA, ATP-dependent |
| MDC1 | mediator of DNA-damage checkpoint 1 |
| MMS22L | MMS22-like, DNA repair protein |
| POLA1 | polymerase (DNA directed), alpha 1, catalytic subunit |
| POLA2 | polymerase (DNA directed), alpha 2, accessory subunit |
| POLB | polymerase (DNA directed), beta |
| POLD1 | polymerase (DNA directed), delta 1, catalytic subunit |
| POLD2 | polymerase (DNA directed), delta 2, accessory subunit |
| POLD3 | polymerase (DNA-directed), delta 3, accessory subunit |
| POLD4 | polymerase (DNA-directed), delta 4, accessory subunit |
| POLDIP2 | polymerase (DNA-directed), delta interacting protein 2 |
| POLDIP3 | polymerase (DNA-directed), delta interacting protein 3 |
| POLE | polymerase (DNA directed), epsilon, catalytic subunit |
| POLE2 | polymerase (DNA directed), epsilon 2, accessory subunit |
| POLE3 | polymerase (DNA directed), epsilon 3, accessory subunit |
| POLE4 | polymerase (DNA-directed), epsilon 4, accessory subunit |
| POLG | polymerase (DNA directed), gamma |
| POLG2 | polymerase (DNA directed), gamma 2, accessory subunit |
| POLH | polymerase (DNA directed), eta |
| POLI | polymerase (DNA directed) iota |
| POLK | polymerase (DNA directed) kappa |
| POLL | polymerase (DNA directed), lambda |
| POLM | polymerase (DNA directed), mu |
| POLN | polymerase (DNA directed) nu |
| POLQ | polymerase (DNA directed), theta |
| ID1 | inhibitor of DNA binding 1, dominant negative helix- |
| | loop-helix protein |
| ID2 | inhibitor of DNA binding 2, dominant negative helix- |
| | loop-helix protein |
| ID3 | inhibitor of DNA binding 3, dominant negative helix- |
| | loop-helix protein |
| ID4 | inhibitor of DNA binding 4, dominant negative helix- |
| | loop-helix protein |
| OGGI | 8-oxoguanine DNA glycosylase |
| MSANTD1 | Myb/SANT-like DNA-binding domain containing 1 |
| MSANTD2 | Myb/SANT-like DNA-binding domain containing 2 |
| MSANTD3 | Myb/SANT-like DNA-binding domain containing 3 |
| MSANTD4 | Myb/SANT-like DNA-binding domain containing 4 with |
| | coiled-coils |
| PIF1 | PIF1 5′-to-3′ DNA helicase |
| TONSL | tonsoku-like, DNA repair protein |
| MPG | N-methylpurine-DNA glycosylase |
| TOP1 | topoisomerase (DNA) I |
| TOP1MT | topoisomerase (DNA) I, mitochondrial |
| TOP2A | topoisomerase (DNA) II alpha 170 kDa |
| TOP2B | topoisomerase (DNA) II beta 180 kDa |
| TOP3A | topoisomerase (DNA) III alpha |
| TOP3B | topoisomerase (DNA) III beta |
| TOPBP1 | topoisomerase (DNA) II binding protein 1 |
| DDB1 | damage-specific DNA binding protein 1, 127 kDa |
| DDB2 | damage-specific DNA binding protein 2, 48 kDa |
| SSBP1 | single-stranded DNA binding protein 1, mitochondrial |
| SSBP2 | single-stranded DNA binding protein 2 |
| SSBP3 | single stranded DNA binding protein 3 |
| SSBP4 | single stranded DNA binding protein 4 |
| GADD45A | growth arrest and DNA-damage-inducible, alpha |
| GADD45B | growth arrest and DNA-damage-inducible, beta |
| GADD45G | growth arrest and DNA-damage-inducible, gamma |
| GADD45GIP1 | growth arrest and DNA-damage-inducible, gamma |
| | interacting protein 1 |
| MGMT | O-6-methylguanine-DNA methyltransferase |
| REV1 | REV1, polymerase (DNA directed) |
| RECQL | RecQ protein-like (DNA helicase Q1-like) |
| CCDC6 | coiled-coil domain containing 6 |
| KLRK1 | killer cell lectin-like receptor subfamily K, member 1 |
| N6AMT1 | N-6 adenine-specific DNA methyltransferase 1 (putative) |
| N6AMT2 | N-6 adenine-specific DNA methyltransferase 2 (putative) |
| POLR2A | polymerase (RNA) II (DNA directed) polypeptide A, |
| | 220 kDa |
| POLR2B | polymerase (RNA) II (DNA directed) polypeptide B, |
| | 140 kDa |
| POLR2C | polymerase (RNA) II (DNA directed) polypeptide C, |
| | 33 kDa |
| POLR2D | polymerase (RNA) II (DNA directed) polypeptide D |
| POLR2E | polymerase (RNA) II (DNA directed) polypeptide E, |
| | 25 kDa |
| POLR2F | polymerase (RNA) II (DNA directed) polypeptide F |
| POLR2G | polymerase (RNA) II (DNA directed) polypeptide G |
| POLR2H | polymerase (RNA) II (DNA directed) polypeptide H |
| POLR2I | polymerase (RNA) II (DNA directed) polypeptide I, |
| | 14.5 kDa |
| POLR2J | polymerase (RNA) II (DNA directed) polypeptide J, |
| | 13.3 kDa |
| POLR2J2 | polymerase (RNA) II (DNA directed) polypeptide J2 |
| POLR2J3 | polymerase (RNA) II (DNA directed) polypeptide J3 |
| POLR2K | polymerase (RNA) II (DNA directed) polypeptide K, |
| | 7.0 kDa |
| POLR2L | polymerase (RNA) II (DNA directed) polypeptide L, |
| | 7.6 kDa |
| POLR2M | polymerase (RNA) II (DNA directed) polypeptide M |
| TRDMT1 | tRNA aspartic acid methyltransferase 1 |
| CHD1 | chromodomain helicase DNA binding protein 1 |
| CHD1L | chromodomain helicase DNA binding protein 1-like |
| CHD2 | chromodomain helicase DNA binding protein 2 |
| CHD3 | chromodomain helicase DNA binding protein 3 |
| CHD4 | chromodomain helicase DNA binding protein 4 |
| CHD5 | chromodomain helicase DNA binding protein 5 |
| CHD6 | chromodomain helicase DNA binding protein 6 |
| CHD7 | chromodomain helicase DNA binding protein 7 |
| CHD8 | chromodomain helicase DNA binding protein 8 |
| CHD9 | chromodomain helicase DNA binding protein 9 |
| KLLN | killin, p53-regulated DNA replication inhibitor |
| POLR3A | polymerase (RNA) III (DNA directed) polypeptide A, |
| | 155 kDa |
| POLR3B | polymerase (RNA) III (DNA directed) polypeptide B |
| POLR3C | polymerase (RNA) III (DNA directed) polypeptide C |
| | (62 kD) |
| POLR3D | polymerase (RNA) III (DNA directed) polypeptide D, |
| | 44 kDa |
| POLR3E | polymerase (RNA) III (DNA directed) polypeptide E |
| | (80 kD) |
| POLR3F | polymerase (RNA) III (DNA directed) polypeptide F, |
| | 39 kDa |
| POLR3G | polymerase (RNA) III (DNA directed) polypeptide G |
| | (32 kD) |
| POLR3GL | polymerase (RNA) III (DNA directed) polypeptide G |
| | (32 kD)-like |
| POLR3H | polymerase (RNA) III (DNA directed) polypeptide H |
| | (22.9 kD) |
| POLR3K | polymerase (RNA) III (DNA directed) polypeptide K, |
| | 12.3 kDa |
| WDHD1 | WD repeat and HMG-box DNA binding protein 1 |
| PGAP1 | post-GPI attachment to proteins 1 |
| PGAP2 | post-GPI attachment to proteins 2 |
| PGAP3 | post-GPI attachment to proteins 3 |
| REV3L | REV3-like, polymerase (DNA directed), zeta, catalytic |
| | subunit |
| CDT1 | chromatin licensing and DNA replication factor 1 |
| PANDAR | promoter of CDKN1A antisense DNA damage activated |
| | RNA |
| APEX1 | APEX nuclease (multifunctional DNA repair enzyme) 1 |
| CHMP1A | charged multivesicular body protein 1A |
| CHMP1B | charged multivesicular body protein 1B |
| CHMP2A | charged multivesicular body protein 2A |
| CHMP2B | charged multivesicular body protein 2B |
| CHMP4A | charged multivesicular body protein 4A |
| CHMP4B | charged multivesicular body protein 4B |
| CHMP4C | charged multivesicular body protein 4C |
| CHMP5 | charged multivesicular body protein 5 |
| CHMP6 | charged multivesicular body protein 6 |
| POLRMT | polymerase (RNA) mitochondrial (DNA directed) |
| SPIDR | scaffolding protein involved in DNA repair |
| MCIDAS | multiciliate differentiation and DNA synthesis associated |
| | cell cycle protein |
| PAPD7 | PAP associated domain containing 7 |
| RFX8 | RFX family member 8, lacking RFX DNA binding |
| | domain |
| DEK | DEK oncogene |
| NUB1 | negative regulator of ubiquitin-like proteins 1 |
| PAXBP1 | PAX3 and PAX7 binding protein 1 |
| RAMP1 | receptor (G protein-coupled) activity modifying protein 1 |
| RAMP2 | receptor (G protein-coupled) activity modifying protein 2 |
| RAMP3 | receptor (G protein-coupled) activity modifying protein 3 |
| RC3H2 | ring finger and CCCH-type domains 2 |
| ARHGAP35 | Rho GTPase activating protein 35 |
| SMUG1 | single-strand-selective monofunctional uracil-DNA |
| | glycosylase 1 |
| CXXC1 | CXXC finger protein 1 |
| FAM50A | family with sequence similarity 50, member A |
| FANCG | Fanconi anemia, complementation group G |
| GLI3 | GLI family zinc finger 3 |
| GTF2H5 | general transcription factor IIH, polypeptide 5 |
| LAGE3 | L antigen family, member 3 |
| MYCNOS | MYCN opposite strand/antisense RNA |
| NFRKB | nuclear factor related to kappaB binding protein |
| RAD51D | RAD51 paralog D |
| RFX2 | regulatory factor X, 2 (influences HLA class II |
| | expression) |
| RFXANK | regulatory factor X-associated ankyrin-containing |
| | protein |
| RRP1 | ribosomal RNA processing 1 |
| SPRTN | SprT-like N-terminal domain |
| XRCC4 | X-ray repair complementing defective repair in Chinese |
| | hamster cells 4 |
| CDK11A | cyclin-dependent kinase 11A |
| CDK11B | cyclin-dependent kinase 11B |
| LURAP1L | leucine rich adaptor protein 1-like |
| MAD2L2 | MAD2 mitotic arrest deficient-like 2 (yeast) |
| PRDM2 | PR domain containing 2, with ZNF domain |
| NABP2 | nucleic acid binding protein 2 |
| NABP1 | nucleic acid binding protein 1 |
| PPP1R15A | protein phosphatase 1, regulatory subunit 15A |
| TATDN1 | TatD DNase domain containing 1 |
| TATDN2 | TatD DNase domain containing 2 |
| TATDN3 | TatD DNase domain containing 3 |
| CEBPB | CCAAT/enhancer binding protein (C/EBP), beta |
| INIP | INTS3 and NABP interacting protein |
| INTS3 | integrator complex subunit 3 |
| SDIM1 | stress responsive DNAJB4 interacting membrane protein |
| | 1 |
| DHX9 | DEAH (Asp-Glu-Ala-His) (SEQ ID NO: 39) box helicase |
| | 9 |
| SATB1 | SATB homeobox 1 |
| FEN1 | flap structure-specific endonuclease 1 |
| HCST | hematopoietic cell signal transducer |
| TYROBP | TYRO protein tyrosine kinase binding protein |
| AFA | ankyloblepharon filiforme adnatum |
| C9orf169 | chromosome 9 open reading frame 169 |
| TSPO2 | translocator protein 2 |
| TCIRG1 | T-cell, immune regulator 1, ATPase, H+ transporting, |
| | lysosomal V0 subunit A3 |
| C1orf61 | chromosome 1 open reading frame 61 |
| HLA-DOA | major histocompatibility complex, class II, DO alpha |
| SPINK13 | serine peptidase inhibitor, Kazal type 13 (putative) |

In some embodiments, the payload comprises a modulator of an epigenetic state or characteristic of a component of chromatin, e.g., a chromatin associated protein, e.g., a histone. For example, the epigenetic state or characteristic can comprise histone acetylation, deacetylation, methylation (e.g., mono, di, or tri-methylation), demethylation, phosphorylation, dephosphorylation, ubiquitination (e.g., mono or polyubiquitination), deubiquitination, sumoylation, ADP-ribosylation, deamination, or a combination thereof.

In some embodiments, the modulator is selected from, or modulates, one or more histone modifying enzymes. In an embodiment, the histone modifying enzyme is a histone methyltransferase (HMT). In some embodiments, the histone modifying enzyme is a histone demethyltransferase (HDMT). In some embodiments, the histone modification enzyme is a histone acetyltransferase (HAT). In some embodiments, the histone modifying enzyme is a histone deacetylase (HDAC). In some embodiments, the histone modification enzyme is a kinase. In some embodiments, the histone modifying enzyme is a phosphatase. In some embodiments, the histone modifying enzyme is ubiquitin-activating enzymes (E1s), ubiquitin-conjugating enzymes (E2s), or ubiquitin ligases (E3s). In some embodiments, the histone modifying enzyme is a deubiquitinating (DUB) enzyme.

In some embodiments, histone modifications involved in regulation of gene transcription are modulated. For example, mono-methylation of H3K4, H3K9, H3K27, H3K79, H4K20, H2BK5, di-methylation of H3K79, tri-methylation of H3K4, H3K79, H3K36, and acetylation of H3K9, H3K14, H3K27, can be associated with transcription activation. As another example, di-methylation of H3K9, H3K27, and tri-methylation of H3K9, H3K27, H3K79, H2BK5 can be associated with transcription repression. In some embodiments, the modulator modulates trimethylation of H3 lysine 4 (H3K4Me3) and/or trimethylation of H3 lysine 36 (H3K36Me3), e.g., in active genes. In other embodiments, the modulator modulates trimethylation of H3 lysine 27 (H3K27Me3), di- and tri-methylation of H3 lysine 9 (H3K9Me2/3), and/or trimethylation of H4 lysine 20 (H4K20Me3), e.g., in repressed genes. In some embodiments, the modulator modulates both activating (e.g., H3K4Me3) and repressing (e.g., H3K27Me3) marks, e.g., in stem cells.

In some embodiments, histone modifications involved in DNA damage response and repair are modulated. For example, the modulators described herein can modulate phosphorylation of H2AX at Serine 139 and/or acetylation of H3 lysine 56 (H3K56Ac).

Aberrant histone modifications are associated with various diseases and conditions, e.g., cancer, cardiovascular disease, and neurodegenerative disorder. The modulators described herein can be used to treat a disease or condition described herein, e.g., by modulating one or more histone modifications, as described herein.

Epigenetic changes in histones can be evaluated by art-known methods or as described herein. Exemplary methods for detecting histone modifications include, e.g., chromatin immunoprecipitation (ChIP) using antibodies against modified histones, e.g., followed by quantitative PCR.

Exemplary endogenous or exogenous modulators of chromatin structure are described herein, e.g., in Table VI-4.

TABLE VI-4

| Approved | | | |
|---|---|---|---|
| Symbol | Approved Name | Synonyms | Ref Seq IDs |
| SUV39H1 | suppressor of variegation 3-9 | KMT1A | NM_003173 |
| | homolog 1 (Drosophila) | | |
| SUV39H2 | suppressor of variegation 3-9 | FLJ23414, KMT1B | NM_024670 |
| | homolog 2 (Drosophila) | | |
| EHMT2 | euchromatic histone-lysine N- | G9A, Em: AF134726.3, | NM_006709 |
| | methyltransferase 2 | NG36/G9a, KMT1C | |
| EHMT1 | euchromatic histone-lysine N- | Eu-HMTase1, | NM_024757 |
| | methyltransferase 1 | FLJ12879, KIAA1876, | |
| | | bA188C12.1, KMT1D | |
| SETDB1 | SET domain, bifurcated 1 | KG1T, KIAA0067, | |
| | | ESET, KMT1E, | |
| | | TDRD21 | |
| SETDB2 | SET domain, bifurcated 2 | CLLD8, CLLL8, | NM_031915 |
| | | KMT1F | |
| KMT2A | lysine (K)-specific methyltransferase | TRX1, HRX, ALL-1, | NM_005933 |
| | 2A | HTRX1, CXXC7, | |
| | | MLL1A | |
| KMT2B | lysine (K)-specific methyltransferase | KIAA0304, MLL2, | NM_014727 |
| | 2B | TRX2, HRX2, WBP7, | |
| | | MLL1B, MLL4 | |
| KMT2C | lysine (K)-specific methyltransferase | KIAA1506, HALR | |
| | 2C | | |
| KMT2D | lysine (K)-specific methyltransferase | ALR, MLL4, | |
| | 2D | CAGL114 | |
| KMT2E | lysine (K)-specific methyltransferase | HDCMC04P | |
| | 2E | | |
| SETD1A | SET domain containing 1A | KIAA0339, Set1, | NM_014712 |
| | | KMT2F | |
| SETD1B | SET domain containing 1B | KIAA1076, Set1B, | XM_037523 |
| | | KMT2G | |
| ASH1L | ash1 (absent, small, or homeotic)-like | huASH1, ASH1, | NM_018489 |
| | (Drosophila) | ASH1L1, KMT2H | |
| SETD2 | SET domain containing 2 | HYPB, HIF-1, | NM_014159 |
| | | KIAA1732, FLJ23184, | |
| | | KMT3A | |
| NSD1 | nuclear receptor binding SET domain | ARA267, FLJ22263, | NM_172349 |
| | protein 1 | KMT3B | |
| SMYD2 | SET and MYND domain containing | HSKM-B, ZMYND14, | NM_020197 |
| | 2 | KMT3C | |
| SMYD1 | SET and MYND domain containing | BOP, ZMYND22, | XM_097915 |
| | 1 | KMT3D | |
| SMYD3 | SET and MYND domain containing | KMT3E | NM_022743 |
| | 3 | | |
| DOT1L | DOT1-like histone H3K79 | KIAA1814, DOT1, | NM_032482 |
| | methyltransferase | KMT4 | |
| SETD8 | SET domain containing (lysine | SET8, SET07, PR- | NM_020382 |
| | methyltransferase) 8 | Set7, KMT5A | |
| SUV420H1 | suppressor of variegation 4-20 | CGI-85, KMT5B | NM_017635 |
| | homolog 1 (Drosophila) | | |
| SUV420H2 | suppressor of variegation 4-20 | MGC2705, KMT5C | NM_032701 |
| | homolog 2 (Drosophila) | | |
| EZH2 | enhancer of zeste homolog 2 | EZH1, ENX-1, KMT6, | |
| | (Drosophila) | KMT6A | |
| EZH1 | enhancer of zeste homolog 1 | KIAA0388, KMT6B | NM_001991 |
| | (Drosophila) | | |
| SETD7 | SET domain containing (lysine | KIAA1717, SET7, | NM_030648 |
| | methyltransferase) 7 | SET7/9, Set9, KMT7 | |
| PRDM2 | PR domain containing 2, with ZNF | RIZ, RIZ1, RIZ2, | NM_012231 |
| | domain | KMT8, MTB-ZF, | |
| | | HUMHOXY1 | |
| HAT1 | histone acetyltransferase 1 | KAT1 | NM_003642 |
| KAT2A | K(lysine) acetyltransferase 2A | GCN5, PCAF-b | NM_021078 |
| KAT2B | K(lysine) acetyltransferase 2B | P/CAF, GCN5, | NM_003884 |
| | | GCN5L | |
| CREBBP | CREB binding protein | RTS, CBP, KAT3A | NM_004380 |
| EP300 | E1A binding protein p300 | p300, KAT3B | NM_001429 |
| TAF1 | TAF1 RNA polymerase II, TATA | NSCL2, TAFII250, | NM_004606 |
| | box binding protein (TBP)-associated | KAT4, DYT3/TAF1 | |
| | factor, 250 kDa | | |
| KAT5 | K(lysine) acetyltransferase 5 | TIP60, PLIP, cPLA2, | NM_006388 |
| | | HTATIP1, ESA1, | |
| | | ZC2HC5 | |
| KAT6A | K(lysine) acetyltransferase 6A | MOZ, ZC2HC6A | NM_006766 |
| KAT6B | K(lysine) acetyltransferase 6B | querkopf, qkf, Morf, | NM_012330 |
| | | MOZ2, ZC2HC6B | |
| KAT7 | K(lysine) acetyltransferase 7 | HBOA, HBO1, | NM_007067 |
| | | ZC2HC7 | |
| KAT8 | K(lysine) acetyltransferase 8 | MOF, FLJ14040, | NM_032188 |
| | | hMOF, ZC2HC8 | |
| ELP3 | elongator acetyltransferase complex | FLJ10422, KAT9 | NM_018091 |
| | subunit 3 | | |
| GTF3C4 | general transcription factor IIIC, | TFIIIC90, KAT12 | |
| | polypeptide 4, 90 kDa | | |
| NCOA1 | nuclear receptor coactivator 1 | SRC1, F-SRC-1, | NM_147223 |
| | | NCoA-1, KAT13A, | |
| | | RIP160, bHLHe74 | |
| NCOA3 | nuclear receptor coactivator 3 | RAC3, AIB1, ACTR, | NM_006534 |
| | | p/CIP, TRAM-1, | |
| | | CAGH16, TNRC16, | |
| | | KAT13B, bHLHe42, | |
| | | SRC-3, SRC3 | |
| NCOA2 | nuclear receptor coactivator 2 | TIF2, GRIP1, NCoA-2, | |
| | | KAT13C, bHLHe75 | |
| CLOCK | clock circadian regulator | KIAA0334, KAT13D, | NM_004898 |
| | | bHLHe8 | |
| KDM1A | lysine (K)-specific demethylase 1A | KIAA0601, BHC110, | NM_015013 |
| | | LSD1 | |
| KDM1B | lysine (K)-specific demethylase 1B | FLJ34109, FLJ33898, | NM_153042 |
| | | dJ298J15.2, | |
| | | bA204B7.3, FLJ43328, | |
| | | LSD2 | |
| KDM2A | lysine (K)-specific demethylase 2A | KIAA1004, FBL11, | NM_012308 |
| | | LILINA, | |
| | | DKFZP434M1735, | |
| | | FBL7, FLJ00115, | |
| | | CXXC8, JHDM1A | |
| KDM2B | lysine (K)-specific demethylase 2B | PCCX2, CXXC2, | NM_032590 |
| | | Fbl10, JHDM1B | |
| KDM3A | lysine (K)-specific demethylase 3A | TSGA, KIAA0742, | NM_018433 |
| | | JHMD2A | |
| KDM3B | lysine (K)-specific demethylase 3B | KIAA1082, NET22 | NM_016604 |
| KDM4A | lysine (K)-specific demethylase 4A | KIAA0677, JHDM3A, | NM_014663 |
| | | TDRD14A | |
| KDM4B | lysine (K)-specific demethylase 4B | KIAA0876, TDRD14B | NM_015015 |
| KDM4C | lysine (K)-specific demethylase 4C | GASC1, KIAA0780, | NM_015061 |
| | | TDRD14C | |
| KDM4D | lysine (K)-specific demethylase 4D | FLJ10251 | NM_018039 |
| KDM4E | lysine (K)-specific demethylase 4E | JMJD2E | NM_001161630 |
| KDM5A | lysine (K)-specific demethylase 5A | | NM_005056 |
| KDM5B | lysine (K)-specific demethylase 5B | RBBP2H1A, PLU-1, | NM_006618 |
| | | CT31 | |
| KDM5C | lysine (K)-specific demethylase 5C | DXS1272E, XE169 | NM_004187 |
| KDM5D | lysine (K)-specific demethylase 5D | KIAA0234 | NM_004653 |
| KDM6A | lysine (K)-specific demethylase 6A | | NM_021140 |
| KDM6B | lysine (K)-specific demethylase 6B | KIAA0346 | XM_043272 |
| JHDM1D | jumonji C domain containing histone | KIAA1718 | NM_030647 |
| | demethylase 1 homolog D (S. | | |
| | cerevisiae) | | |
| PHF8 | PHD finger protein 8 | ZNF422, KIAA1111, | NM_015107 |
| | | JHDM1F | |
| PHF2 | PHD finger protein 2 | KIAA0662, JHDM1E, | NM_005392 |
| | | CENP-35 | |
| KDM8 | lysine (K)-specific demethylase 8 | FLJ13798 | NM_024773 |

Modulators of Gene Expression

In an embodiment a payload comprises a modulator of gene expression. A modulator of gene expression can be delivered in vitro, ex vivo, or in vivo.

In an embodiment, the payload comprises a transcription factor. Transcription factors can bind to specific DNA sequences (e.g., an enhancer or promoter region) adjacent to the genes that they regulate. For example, transcription factors can stabilize or inhibit the binding of RNA polymerase to DNA, catalyze the acetylation or deacetylation of histone proteins (e.g., directly or by recruiting other proteins with such catalytic activity), or recruit coactivator or corepressor proteins to the transcription factor/DNA complex. Modulators of gene expression also include, e.g., any proteins that interact with transcription factors directly or indirectly.

In an embodiment, the transcription factor is a general transcription factor, e.g., is ubiquitous and interacts with the core promoter region surrounding the transcription start site(s) of many, most or all class II genes. Exemplary general transcription factors include, e.g., TFIIA, TFIIB, TFIID, TFIIE, TFIIF, and TFIIH. In an embodiment, the transcription factor is an upstream transcription factor, e.g., binds upstream of the initiation site to stimulate or repress transcription. In an embodiment, the transcription factor is a specific transcription factor, e.g., a transcription factor dependent on a recognition sequence present in the proximity of the gene. Exemplary specific transcription factors include, e.g., SP1, AP-1, C/EBP, heat shock factor, ATF/CREB, -Myc, OCT-1, and NF-1.

In an embodiment, the transcription factor is constitutively active, e.g., a general transcription factor, SP1, NF-1, or CCAAT. In other embodiments, the transcription factor is conditionally active, e.g. it requires activation, e.g., developmental (e.g., GATA, HNF, PIT-1, MyoD, Myf5, Hox, Winged Helix), signal-dependent (e.g., extracellular ligand (endocrine or paracrine)-dependent, intracellular ligand (autocrine)-dependent (e.g., SREBP, p53, orphan nuclear receptors), cell membrane receptor-dependent (e.g., resident nuclear factors (e.g., CREB, AP-1, Mef2) or latent cytoplasmic factors (e.g., STAT, R-SMAD, NF-κB, Notch, TUBBY, NFAT).

Other exemplary transcription factors are described herein, e.g., in Tables VI-5 and VI-6. (Table VI-5 Transcription Factors, is provided in Annex VI-5)

TABLE VI-6
Selected Transcription Factors with Anotations

| Transcription | |
|---|---|
| factor family | |
| (# genes/family) | Comments |
| AF-4(4) | Exemplary diseases include acute lymphoblastic leukemia (AF4 and |
| | AFF3) and mental retardation (FMR2). |
| CBF(1) | Exemplary functions include regulator of hematopoiesis. For |
| | example, CBF is also involved in the chondrocyte differentiation and |
| | ossification. |
| CSL(2) | Exemplary functions include universal transcriptional effector of |
| | Notch signaling. For example, Notch signaling is dysregulated in |
| | many cancers and faulty notch signaling is implicated in many |
| | diseases. Exemplary disease include T-ALL (T-cell acute |
| | lymphoblastic leukemia), CADASIL (Cerebral Autosomal-Dominant |
| | Arteriopathy with Sub-cortical Infarcts and Leukoencephalopathy), |
| | MS (Multiple Sclerosis), Tetralogy of Fallot, Alagille syndrome. |
| ETS(29) | Exemplary functions include regulation of cellular differentiation, cell |
| | cycle control, cell migration, cell proliferation, apoptosis |
| | (programmed cell death) and angiogenesis. Exemplary diseases |
| | include dieases associated with cancer, such as through gene fusion, |
| | e.g., prostate cancer. |
| HMGI/HMGY(2) | Overexpression in certain cancers |
| MH1(8) | Exemplary diseases include cancer, fibrosis and autoimmune |
| | diseases. |
| Nuclear orphan | Exemplary functions include superfamily of transcription regulators |
| receptor(3) | that are involved in widely diverse physiological functions, including |
| | control of embryonic development, cell differentiation and |
| | homeostasis. Exemplary diseases include inflammation, cancer, and |
| | metabolic disorders. |
| PC4(1) | Exemplary functions include replication, DNA repair and |
| | transcription. |
| RFX(8) | Exemplary functions include regulation of development and function |
| | of cilia. Exemplary diseases include Bardet-Biedl syndrome. |
| STAT(7) | Exemplary functions include regulation of many aspects of growth, |
| | survival and differentiation in cells. Exemplary diseases include |
| | angiogenesis, enhanced survival of tumors and immunosuppression. |
| Thyroid hormone | Involved in widely diverse physiological functions, including control |
| receptor(25) | of embryonic development, cell differentiation and homeostasis |
| zf-C2HC(6) | Highly transcribed in the developing nervous system. Exemplary |
| | diseases include Duane Radial Ray Syndrome. |
| Androgen | Exemplary functions include diverse physiological functions, |
| receptor(1) | including control of embryonic development, cell differentiation and |
| | homeostasis. Exemplary diseases include X-linked spinal, bulbar |
| | muscular atrophy and prostate cancer. |
| CG-1(2) | Exemplary functions include calcium signaling by direct binding of |
| | calmodulin. |
| CTF/NFI(4) | Exemplary functions include both viral DNA replication and |
| | regulation of gene expression. Exemplary diseases include leukemia, |
| | juvenile myelomonocytic. |
| Fork head(49) | Involvement in early developmental decisions of cell fates during |
| | embryogenesis. Exemplary diseases include lymphedema-distichiasis, |
| | developmental verbal dyspraxia, autoimmune diseases. |
| Homeobox(205) | Exemplary functions include involvement in a wide range of critical |
| | activities during development. Exemplary diseases include limb |
| | malformations, eye disorders, and abnormal head, face, and tooth |
| | development. Additionally, increased or decreased activity of certain |
| | homeobox genes has been associated with several forms of cancer. |
| MYB(25) | Exemplary functions include regulator of proliferation, differentiation |
| | and cell fate. Exemplary diseases include cancer (e.g., oncogenic |
| | disease). |
| Oestrogen | Control of embryonic development, cell differentiation and |
| receptor(l) | homeostasis. Exemplary diseases include estrogen resistance, familial |
| | breast cancer, migrane, myocardial infaction. |
| POU(21) | Wide variety of functions, related to the function of the |
| | neuroendocrine system and the development of an organism. |
| | Exemplary diseases include non-syndromic deafness. |
| RHD(10) | Exemplary diseases include autoimmune arthritis, asthma, septic |
| | shock, lung fibrosis, glomerulonephritis, atherosclerosis, and AIDS. |
| T-box(17) | |
| TSC22(4) | |
| zf-GATA(14) | |
| AP-2(5) | |
| COE(4) | |
| CUT(7) | |
| GCM(2) | |
| HSF(8) | |
| NDT80/PhoG(1) | |
| Other nuclear | |
| receptor(2) | |
| PPAR receptor(3) | |
| ROR receptor(4) | |
| TEA(4) | |
| Tub(5) | |
| zf-LITAF-like(2) | |
| ARID(15) | |
| COUP(3) | |
| DM(7) | |
| GCR(1) | |
| HTH(2) | |
| NF-YA(1) | |
| Others(3) | |
| Progesterone | |
| receptor(1) | |
| Runt(3) | |
| TF_bZIP(46) | |
| ZBTB(48) | |
| zf-MIZ(7) | |
| bHLH(106) | |
| CP2(7) | |
| E2F(11) | |
| GTF2I(5) | |
| IRF(9) | |
| NF-YB/C(2) | |
| P53(3) | |
| Prox1(2) | |
| SAND(8) | |
| TF_Otx(3) | |
| zf-BED(5) | |
| zf-NF-X1(2) | |
| C/EBP(10) | |
| CSD(8) | |
| Ecdystd | |
| receptor(2) | |
| HMG(50) | |
| MBD(9) | |
| Nrf1(1) | |
| PAX(9) | |
| Retinoic acid | |
| receptor(7) | |
| SRF(6) | |
| THAP(12) | |
| zf-C2H2(634) | |
| CRX | Exemplary diseases include dominant cone-rod dystrophy. Repair |
| | mutation. |
| FOCX2 | Exemplary diseases include lymphedema-distichiasis. Repair |
| | mutation. |
| FOXP2 | Exemplary diseases include developmental verbal dyspraxia. Repair |
| | mutation. |
| FOXP3 | Exemplary diseases include autoimmune diseases. Repair mutation. |
| GAT4 | Exemplary diseases include congenital heart defects. Repair mutation. |
| HNF1 through | Exemplary diseases include mature onset diabetes of the young |
| HNF6 | (MODY), hepatic adenomas and renal cysts. Repair mutation. |
| LHX3 | Exemplary diseases include Pituitary disease. Repair mutation. |
| MECP2 | Exemplary diseases include Rett syndrome. Repair mutation. |
| MEF2A | Exemplary diseases include Coronary artery disease. Repair mutation. |
| NARA2 | Exemplary diseases include Parkinson disease. Repair mutation. |
| NF-κB | Exemplary diseases include autoimmune arthritis, asthma, septic |
| Activation | shock, lung fibrosis, glomerulonephritis, atherosclerosis, and AIDS. |
| | Repair mutation. |
| NF-κB | Exemplary diseases include apoptosis, inappropriate immune cell |
| Inhibition | development, and delayed cell growth. Repair mutation. |
| NIKX2-5 | Exemplary diseases include cardiac malformations and |
| | atrioventricular conduction abnormalities. |
| NOTCH1 | Exemplary diseases include aortic valve abnormalities. |

Modulators of Alternative Splicing

In an embodiment, the modulator of gene expression modulates splicing. For example, a modulator can modulate exon skipping or cassette exon, mutually exclusive exons, alternative donor site, alternative acceptor site, intron retention, or a combination thereof. In some embodiments, the modulator is selected from or modulates one or more general or alternative splicing factors, e.g., ASF1. In some embodiments, the modulator modulates alternative splicing (e.g., influences splice site selection) in a concentration-dependent manner.

Modulators of Post-Transcriptional Modification

In an embodiment, the modulator of gene expression modulates post-transcriptional modification. For example, the modulators described herein can promote or inhibit 5′ capping, 3′ polyadenylation, and RNA splicing. In an embodiment, the modulator is selected from, or modulates, one or more factors involved in 5′ capping, e.g., phosphatase and guanosyl transferase. In an embodiment, the modulator is selected from, or modulates, one or more factors involved in 3′ polyadenylation, e.g., polyadenylate polymerase, cleavage and polyadenylation specificity factor (CPSF), and poly(A) binding proteins. In an embodiment, the modulator is selected from, or modulates, one or more factors involved in RNA splicing, e.g., general or alternative splicing factors.

Exemplary endogenous or exogenous modulators of post-transcriptional modification are described herein, e.g., in Table VI-7.

TABLE VI-7
POST-TRANSCRIPTIONAL CONTROL MODULATORS

mRNA processing

Polyadenylation

PARN: polyadenylation specific ribonuclease
PAN: PolyA nuclease
CPSF: cleavage/polyadenylation specificity factor
CstF: cleavage stimulation factor
PAP: polyadenylate polymerase
PABP: polyadenylate binding protein
PAB2: polyadenylate binding protein 2
CFI: cleavage factor I
CFII: cleavage factor II

Capping/Methylation of 5′end

RNA triposphatase
RNA gluanyltransferase
RNA mehyltransferase
SAM synthase
ubiquitin-conjugating enzyme E2R1

Splicing

SR proteins SFRS1-SFR11 which, when bound to
exons, tend to promote
hnRNP proteins: coded by the following
genes: HNRNPA0, HNRNPA1, HNRNPA1L1,
HNRNPA1L2, HNRNPA3, HNRNPA2B1,
HNRNPAB, HNRNPB1, HNRNPC, HNRNPCL1,
HNRNPD, HNRPDL, HNRNPF, HNRNPH1,
HNRNPH2, HNRNPH3, HNRNPK, HNRNPL,
HNRPLL, HNRNPM, HNRNPR, HNRNPU,
HNRNPUL1, HNRNPUL2, HNRNPUL3

Editing protein

ADAR

Nuclear export proteins

Mex67
Mtr2
Nab2
DEAD-box helicase (“DEAD” disclosed as SEQ ID
NO: 40)

TRANSLATION

Initiation

eIF4A, eIF4B, eIF4E, and eIF4G: Eukaryotic initiation
factors
GEF: Guanine exchange factor
GCN2, PKR, HRI and PERK: Kinases involved in
phosphorylating some of the initiation factors

Elongation

eEF1 and eEF2: elongation factors
GCN: kinase

Termination

eRF3: translation termination factor

POST-TRANSLATIONAL CONTROL

mRNA Degradation

ARE-specific binding proteins
EXRN1: exonuclease
DCP1, DCP2: Decapping enzymes
RCK/p54, CPEB, eIF4E: Translation repression
microRNAs and siRNAs: Probably regulate 30% of all
genes
DICER
Ago proteins
Nonsense-mediated mRNA decay proteins

UPF3A
UPF3B
eIF4A3
MLN51
Y14/MAGOH
MG-1
SMG-5
SMG-6
SMG-7

mRNA Modification

Enzymes carry the following functions

Phosphorylation
N-linked glycosylation
Acetylation
Amidation
Hydroxylation
Methylation
O-linked glycosylation
Ubiquitylation

Inhibitors

In an embodiment a payload comprises an inhibitor of a payload described above, e.g., an inhibitor of an enzyme transcription factor. In an embodiment a payload comprises an inhibitor of any of the aforementioned payload molecules, processes, activities or mechanisms. In an embodiment, the inhibitor is an antibody molecule (e.g., a full antibody or antigen binding fragment thereof) specific for one of the payload molecules described herein. In an embodiment the inhibitor is a small molecule compound. In some embodiments, the inhibitor is a nucleic acid (e.g., siRNA, shRNA, ribozyme, antisense-oligonucleotide, and aptamer). For example, the payload is an inhibitor of a target, e.g., a trasnscription factor, a post-translational modification enzyme, a post-transcriptional modification enzyme, etc., or a nucleic acid sequence encoding any of the foregoing.

Orthologs

If a non-human gene or protein is recited herein it is understood that the invention also comprises the human counterpart or ortholog and uses thereof.

### VIIA. Targets: Cells

Cas9 molecules and gRNA molecules, e.g., a Cas9 molecule/gRNA molecule complex, can be used to manipulate a cell (e.g., an animal cell or a plant cell), e.g., to deliver a payload, or edit a target nucleic acid, in a wide variety of cells. Typically an eiCas9 molecule/gRNA molecule complex is used to deliver a payload and an eaCas9 molecule/gRNA complex is used to edit or alter the structure of a target nucleic acid. Delivery or editing can be performed in vitro, ex vivo, or in vivo.

In some embodiments, a cell is manipulated by editing (e.g., introducing a mutation or correcting) one or more target genes, e.g., as described herein. In other embodiments, a cell is manipulated by delivering a payload comprising one or more modulators (e.g., as described herein) to the cell, e.g., to a target sequence in the genome of the cell. In some embodiments, the expression of one or more target genes (e.g., one or more target genes described herein) is modulated, e.g., in vivo. In some embodiments, the expression of one or more target genes (e.g., one or more target genes described herein) is modulated, e.g., ex vivo.

In some embodiments, the cells are manipulated (e.g., converted or differentiated) from one cell type to another. In some embodiments, a pancreatic cell is manipulated into a beta islet cell. In some embodiments, a fibroblast is manipulated into an iPS cell. In some embodiments, a preadipocyte is manipulated into a brown fat cell. Other exemplary cells include, e.g., muscle cells, neural cells, leukocytes, and lymphocytes.

In some embodiments, the cell is a diseased or mutant-bearing cell. Such cells can be manipulated to treat the disease, e.g., to correct a mutation, or to alter the phenotyope of the cell, e.g., to inhibit the growth of a cancer cell. For examples, a cell is associated with one or more diseases or conditions describe herein. In some embodiments, the cell is a cancer stem cell. For example, cancer stem cells can be manipulated by modulating the expression of one or more genes selected from: TWIST (TF), HIF-1α, HER2/neu, Snail (TF), or Wnt.

In some embodiments, the manipulated cell is a normal cell.

In some embodiments, the manipulated cell is a stem cell or progenitor cell (e.g., iPS, embryonic, hematopoietic, adipose, germline, lung, or neural stem or progenitor cells).

In some embodiments, the manipulated cells are suitable for producing a recombinant biological product. For example, the cells can be CHO cells or fibroblasts. In an embodiment, a manipulated cell is a cell that has been engineered to express a protein.

In some embodiments, the cell being manipulated is selected from fibroblasts, monocytic precursors, B cells, exocrine cells, pancreatic progenitors, endocrine progenitors, hepatoblasts, myoblasts, or preadipocytes. In some embodiments, the cell is manipulated (e.g., converted or differentiated) into muscle cells, erythroid-megakaryocytic cells, eosinophils, iPS cells, macrophages, T cells, islet beta-cells, neurons, cardiomyocytes, blood cells, endocrine progenitors, exocrine progenitors, ductal cells, acinar cells, alpha cells, beta cells, delta cells, PP cells, hepatocytes, cholangiocytes, or brown adipocytes.

In some embodiments, the cell is a muscle cell, erythroid-megakaryocytic cell, eosinophil, iPS cell, macrophage, T cell, islet beta-cell, neuron, cardiomyocyte, blood cell, endocrine progenitor, exocrine progenitor, ductal cell, acinar cell, alpha cell, beta cell, delta cell, PP cell, hepatocyte, cholangiocyte, or white or brown adipocyte.

The Cas9 and gRNA molecules described herein can be delivered to a target cell. In an embodiment, the target cell is a normal cell.

In an embodiment, the target cell is a stem cell or progenitor cell (e.g., iPS, embryonic, hematopoietic, adipose, germline, lung, or neural stem or progenitor cells).

In an embodiment, the target cell is a CHO cell.

In an embodiment, the target cell is a fibroblast, monocytic precursor, B cells exocrine cell, pancreatic progenitor, endocrine progenitor, hepatoblast, myoblast, or preadipocyte.

In an embodiment, the target cell is a muscle cell, erythroid-megakaryocytic cell, eosinophil, iPS cell, macrophage, T cell, islet beta-cell, neurons (e.g., a neuron in the brain, e.g., a neuron in the striatum (e.g., a medium spiny neuron), cerebral cortex, precentral gyrus, hippocampus (e.g., a neuron in the dentate gyrus or the CA3 region of the hippocampus), temporal cortex, amygdala, frontal cortex, thalamus, cerebellum, medulla, putamen, hypothalamus, tectum, tegmentum or substantia nigra), cardiomyocyte, blood cell, endocrine progenitor, exocrine progenitor, ductal cell, acinar cell, alpha cell, beta cell, delta cell, PP cell, hepatocyte, cholangiocyte, or brown adipocyte.

In an embodiment, the target cell is manipulated ex vivo by editing (e.g., introducing a mutation or correcting) one or more target genes and/or modulating the expression of one or more target genes, and administered to the subject.

Exemplary cells that can be manipulated and exemplary genes that can be modulated are described in Table VII-8.

TABLE VII-8

| Cell starting | Differentiated | | Exemplary gene(s) to |
|---|---|---|---|
| point | state | Exemplary payload manipulation | modify expression of |
| fibroblasts | Muscle cells | Deliver Cas9-activators to target | MyoD |
| | | activation of transcription factors | |
| | | required for differentiation in vivo. | |
| Monocytic | Erythroid- | Deliver Cas9-activators to target | GATA1 |
| precursors | megakaryocytic | activation of transcription factors | |
| | cells, | required for differentiation in vivo. | |
| | eosinophils | | |
| fibroblasts | iPS cells | Deliver Cas9-activators to target | Oct4 |
| | | activation of transcription factors | Sox2 |
| | | required for differentiation in vivo. | Klf4 |
| | | Multiplex. | Myc |
| B cells | Macrophages | Deliver Cas9-activators to target | C/EBPα |
| | | activation of transcription factors | |
| | | required for differentiation in vivo. | |
| B cells | T cells, | Delivery Cas9-repressors OR | Pax5 |
| | macrophages | deliver Cas9 endonuclease to | |
| | | ablate Pax5 | |
| Exocrine | Islet β-cells | Deliver Cas9-activators to target | Pdx1 |
| cells | | activation of transcription factors | Ngn3 |
| | | required for differentiation in vivo. | MafA |
| | | Multiplex. | |
| Fibroblasts | Neurons | Deliver Cas9-activators to target | Ascl1 |
| | | activation of transcription factors | Brn2 |
| | | required for differentiation in vivo. | Myt1l |
| | | Multiplex. | |
| fibroblasts | cardiomyocytes | Deliver Cas9-activators to target | Gata4 |
| | | activation of transcription factors | Mef2c |
| | | required for differentiation in vivo. | Tbx5 |
| | | Multiplex. | |
| Fibroblasts | Blood cells | Deliver Cas9-activators to target | Oct4 |
| | | activation of transcription factors | |
| | | required for differentiation in vivo. | |
| Fibroblasts | cardiomyocytes | Deliver Cas9-activators to target | Oct4 |
| | | activation of transcription factors | Sox2 |
| | | required for differentiation in vivo. | Klf4 |
| | | Multiplex. | |
| Pancreatic | Endocrine | Deliver Cas9-activators to target | Ngn3 |
| progenitor | progenitor | activation of transcription factors | |
| | | required for differentiation in vivo. | |
| Pancreatic | Exocrine | Deliver Cas9-activators to target | P48 |
| progenitor | progenitor | activation of transcription factors | |
| | | required for differentiation in vivo. | |
| Pancreatic | Duct | Deliver Cas9-activators to target | Hnf6/OC-1 |
| progenitor | | activation of transcription factors | |
| | | required for differentiation in vivo. | |
| Pancreatic | acinar | Deliver Cas9-activators to target | Ptf1a |
| progenitor | | activation of transcription factors | Rpbjl |
| | | required for differentiation in vivo. | |
| | | Multiplex. | |
| Endocrine | α cell | Deliver Cas9-activators to target | Foxa2 |
| progenitor | | activation of transcription factors | Nkx2.2 |
| (to make | | required for differentiation in vivo. | Pax6 |
| glucagon) | | Multiplex. | Arx |
| Endocrine | β cell | Deliver Cas9-activators to target | Mafa |
| progenitor | | activation of transcription factors | Pdx1 |
| (to make | | required for differentiation in vivo. | Hlxb9 |
| insulin) | | Multiplex. | Pax4 |
| | | | Pax6 |
| | | | Isl1 |
| | | | Nkx2.2 |
| | | | Nkx6.1 |
| Endocrine | δ cell | Deliver Cas9-activators to target | Pax4 |
| progenitor | | activation of transcription factors | Pax6 |
| (to make | | required for differentiation in vivo. | |
| somatostatin) | | Multiplex. | |
| Endocrine | PP cell | Deliver Cas9-activators to target | Nkx2.2 |
| progenitor | | activation of transcription factors | |
| (to make | | required for differentiation in vivo. | |
| pancreatic | | | |
| polypeptide) | | | |
| Hepatoblast | hepatocyte | Deliver Cas9-activators to target | Hnf4 |
| | | activation of transcription factors | |
| | | required for differentiation in vivo. | |
| Hepatoblast | Cholangiocyte | Deliver Cas9-activators to target | Hnf6/OC-1 |
| | | activation of transcription factors | |
| | | required for differentiation in vivo. | |
| Myoblasts | Brown | Deliver Cas9-activators to target | PRDM16 |
| | adipocyte | activation of transcription factors | C/EBP |
| | | required for differentiation in vivo. | PGC1α |
| | | Multiplex. | PPARγ |
| preadipocytes | Brown | Deliver Cas9-activators to target | PRDM16 |
| | adipocyte | activation of transcription factors | C/EBP |
| | | required for differentiation in vivo. | |
| | | Multiplex. | |

TABLE VII-9
Exemplary cells for manipulation

Pancreatic cells, e.g., beta cells
Muscle cells
Adipocytes
Pre-adipocytes
Neural cells
Blood cells
Leukocytes
Lymphocyes
B cells
T cells

TABLE VII-10
Exemplary stem cells for manipulation

embryonic stem cells
non-embryonic stem cells
hematopoietic stem cells
adipose stem cells
germline stem cells
lung stem cells
neural stem cells

TABLE VII-11
Exemplary cancer cells for manipulation

lung cancer cells
breast cancer cells
skin cancer cells
brain cancer cells,
pancreatic cancer cells
hematopoietic cancer cells
liver cancer cells
kidney cancer cells
ovarian cancer cells

TABLE VII-12
Exemplary non-human cells for manipulation
Table VII-12 Non-human cells for manipulation

Plant cells, e.g., crop cells, e.g., corn, wheat,
soybean, citrus or vegetable cells
Animal cells, e.g., a cow, pig, horse, goat, dog or cat
cell

Exemplary endogenous or exogenous modulators of cancer stem cells (CSCs) are described herein, e.g., in Table VII-13:

TABLE VII-13
TWIST 1 (TF)
HIF-1α (TF)
HER2/neu
Snail (TF)
Wnt
TGFβ
FGF
EGF
HGF
STAT3 (TF)
Notch
P63 (TF)
PI3K)/AKT
Hedgehog
NF-κB (TF)
ATF2 (TF)
miR-200 and miR-34
P53 (TF)
E-cadherin
Transcription factors that inhibit E-cadherin directly
ZEB1
ZEB2
E47
KLF8
Transcription factors that inhibit E-cadherin directly
TCF4
SIX1
FOXC2
G-CSF and CD34 in AML
PML and FOXO in CML
CD133 in glioblastoma multiforme, osteosarcoma, Ewing's sarcoma,
endometrial, hepatocellular, colon and lung carcinomas and ovarian
and pancreatic adenocarcinoma
CD44 in head and neck cancer, prostate, gastric and colorectal
carcinoma stem cells
CD34 in leukemia
CD38 in leukemia
IL3Rα in leukemia
EpCAM in colon carcinoma and pancreatic adenocarcinoma stem
cells
ALDH in melanoma, colorectal, breast, prostate and squamous cell
carcinomas, pancreatic adenocarcinoma, and osteosarcoma
MAP2 in melanoma
α6-integrin in glioblastoma
SSEA-1 in gliobalstoma
CD24 in breast cancer and other tumors

Cas9 molecules and gRNA molecules, e.g., a Cas9 molecule/gRNA molecule complex, can be used to manipulate a cell (e.g., a cell described herein), e.g., to deliver a payload, or edit a target nucleic acid, e.g., to increase cell engraftment, e.g., to achieve stable engraftment of cells into a native microenvironment. The engrafting cells, the cells in the native microenvironment, or both, can be manipulated. Typically an eiCas9 molecule/gRNA molecule complex is used to deliver a payload and an eaCas9 molecule/gRNA complex is used to edit or alter the structure of a target nucleic acid.

For example, increased efficiency of engraftment of cells can be achieved by: increasing the expression of one or more of the genes described herein, e.g., homing genes, adhesion genes, survival genes, proliferative genes, immune evasion genes, and/or cell protection genes, and/or decreasing the expression of one or more of the genes described herein, e.g., quiescence genes, death/apoptosis genes, and/or immune recognition genes.

In an embodiment, the gene encodes a homing receptor or an adhesion molecule, e.g., that is involved in directing cell migration towards a tissue in association with a tissue-expressed ligand or region rich in soluble cytokine. In an embodiment, the homing receptor or adhesion molecule is expressed on leukocytes, e.g., lymphocytes or hematopoietic stem cells. In an embodiment, the tissue is bone marrow, e.g., extracellular matrix or stromal cells. In an embodiment, the homing receptor or adhesion molecule is C-X-C chemokine receptor type 4 (CXCR4, also known as fusin or CD184). For example, the expression of CXCR4 on hematopoietic stem cells is upregulated. In an embodiment, the ligand is stromal-derived-factor-1 (SDF-1, also known as CXCL12). In an embodiment, the homing receptor or adhesion molecule is CD34. In an embodiment, the ligand is addressin (also known as mucosal vascular addressin cell adhesion molecule 1 (MAdCAM-1)).

In an embodiment, the gene encodes a receptor, e.g., expressed on a stem cell or progenitor cell, that binds to a ligand, e.g., a chemokine or cytokine. For example, the receptor can be associated with stemness of the cell and/or attracting the cell to a desired microenvironment. In an embodiment, the receptor is expressed on a hematopoietic stem cell. In an embodiment, the receptor is expressed on a neural stem cell. In an embodiment, the receptor is mast/stem cell growth factor receptor (SCFR, also known as proto-oncogene c-Kit or tyrosine-protein kinase Kit or CD117). In an embodiment, the ligand is stem cell factor (SCF, also known as steel factor or c-kit ligand). In an embodiment, the receptor is myeloproliferative leukemia virus oncogene (MPL, also known as CD110). In an embodiment, the ligand is thrombopoietin (TPO).

In an embodiment, the gene encodes a marker, e.g., that promotes survival or proliferation of the cells expressing that marker, or allows the cells expressing that marker to evade an immune response or to be protected from an adverse environment, e.g., that leads to cell death. For example, cells expressing CD47 (also known as integrin associated protein (IAP) can avoid phagocytosis, e.g., during cell migration. As another example, cells that express BCL2 can be protected from apoptosis. In an embodiment, the cell is a blood cell, e.g., an erythrocyte or leukocyte. In an embodiment, the cell is a hematopoietic stem cell or progenitor cell.

In an embodiment, the expression of one or more of CXCR4, SDF1, CD117, MPL, CD47, or BCL2, in a stem cell or progenitor cell, e.g., a hematopoietic stem cell or progenitor cell, is upregulated.

Cas9 molecules and gRNA molecules, e.g., a Cas9 molecule/gRNA molecule complex, can be used to manipulate a cell (e.g., a cell described herein), e.g., to deliver a payload, or edit a target nucleic acid, e.g., to manipulate (e.g., dictate) the fate of a targeted cell, e.g., to better target specific cell type of interest and/or as a suicide mechanism. Typically an eiCas9 molecule/gRNA molecule complex is used to deliver a payload and/or an eaCas9 molecule/gRNA complex is used to edit or alter the structure of a target nucleic acid. Exemplary genes that can be modulated include, e.g., one or more of chemotherapy resistance genes, chemotherapy sensitivity genes, antibiotic resistance genes, antibiotic sensitivity genes, and cell surface receptor genes, e.g., as described herein.

In an embodiment, a chemotherapy resistance gene, a chemotherapy sensitivity gene, an antibiotic resistance gene, and/or an antibiotic sensitivity gene is modulated, e.g., such that modified or undesirable cells (e.g., modified or undesirable hematopoietic stem cells (HSCs), e.g., in bone marrow) can be reduced or removed, e.g., by chemotherapeutic or antibiotic treatment.

For example, genes or gene products that modulate (e.g., increase) chemotherapy resistance or antibiotic resistance can be delivered into the cells. Cells modified by the chemotherapy or antibiotic resistance gene or gene product can have a higher (e.g., at least about 2, 3, 4, 5, 6, 7, 8, 9, 10, 25, 50, 75, or 100 fold higher) survival rate than cells without such modification after chemotherapeutic or antibiotic treatment. In an embodiment, the chemotherapeutic or antibiotic treatment is performed in vivo. In an embodiment, the chemotherapeutic or antibiotic treatment is performed in vitro or ex vivo. In an embodiment, the chemotherapy resistance gene is a gene encoding O⁶-alkylguanine DNA alkyltransferase (MGMT). In an embodiment, the chemotherapy comprises temozolomide.

As another example, genes or gene products that modulate (e.g., increase) chemotherapy sensitivity or antibiotic sensitivity can be delivered into the cells. The genes or gene products that confer chemotherapy sensitivity or antibiotic sensitivity can be used as suicide signals, e.g., causing apoptosis of the cells. Cells modified by the chemotherapy or antibiotic sensitivity gene or gene product can have a lower (e.g., at least about 2, 3, 4, 5, 6, 7, 8, 9, 10, 25, 50, 75, or 100 fold lower) survival rate than cells without such modification after chemotherapeutic or antibiotic treatment. In an embodiment, the chemotherapeutic or antibiotic treatment is performed in vivo. In an embodiment, the chemotherapeutic or antibiotic treatment is performed in vitro or ex vivo.

The method described herein can be used to select or enrich cells that have a modified or desired phenotype, e.g., chemotherapy resistance and/or antibiotic resistance. The method described herein can also be used to remove or reduce the number of cells that have a modified or undesired phenotype, e.g., chemotherapy sensitivity and/or antibiotic sensitivity. For example, cells that exhibit an undesired effect, e.g., an off-target effect or a cancer phenotype, e.g., caused by editing of a nucleic acid in an undesired genomic location or cell type, can be removed.

In an embodiment, a cell surface receptor gene is modulated (e.g., the expression of the cell surface receptor is increased or decreased), such that a therapeutic agent (e.g., a therapeutic antibody) can be used to target a cell (e.g., to kill the cell) that has increased or decreased expression of the cell surface receptor. In an embodiment, the cell surface receptor is CD20. In some embodiments, the therapeutic antibody is Rituximab.

In an embodiment, the cell surface receptor is selected from, e.g., CD52, VEGFR, CD30, EGFR, CD33, or ErbB2. In an embodiment, the therapeutic antibody is selected from, e.g., Alemtuzumab, Rituximab, Cetuximab, Panitumumab, Gemtuzamab, and Trastuzumab. In an embodiment, the cell surface receptor is CD52 and the therapeutic antibody is Alemtuzumab. In an embodiment, the gene encodes VEGF and the therapeutic antibody is Rituximab. In an embodiment, the cell surface receptor is EGFR and the therapeutic antibody is Cetuximab or Panitumumab. In an embodiment, the cell surface receptor is CD33 and the therapeutic antibody is Gemtuzamab. In an embodiment, the cell surface receptor is ErbB2 and the therapeutic antibody is Trastuzumab.

In an embodiment, the expression or activity of the Cas9 molecule and/or the gRNA molecule is induced or repressed, e.g., when the cell is treated with a drug, e.g., an antibiotic, e.g., in vivo. For example, the induction or repression of the expression or activity of the Cas9 molecule and/or the gRNA molecule can be used to reduce toxicity and/or off-target effects, e.g., in certain tissues. In an embodiment, the expression of the Cas9 molecule, the gRNA molecule, or both, is driven by an inducible promoter. In an embodiment, binding of a drug (e.g., an antibiotic) to the Cas9 molecule and/or the gRNA molecule activates or inhibits the activity of the Cas9 molecule and/or the gRNA molecule. In an embodiment, the drug (e.g., antibiotic) is administered locally. In an embodiment, the cell treated with the drug (e.g., antibiotic) is located in the eye, ear, nose, mouth, or skin.

Cas9 molecules and gRNA molecules, e.g., a Cas9 molecule/gRNA molecule complex, can be used to manipulate a cell (e.g., a cell described herein), e.g., to deliver a payload, or edit a target nucleic acid, e.g., in directed enzyme prodrug therapy (DEPT). Typically an eiCas9 molecule/gRNA molecule complex is used to deliver a payload and an eaCas9 molecule/gRNA complex is used to edit or alter the structure of a target nucleic acid.

Directed enzyme prodrug therapy (DEPT) uses enzymes artificially introduced into the body to convert prodrugs, which have no or poor biological activity, to the active form in the desired location within the body. For example, directed enzyme prodrug therapy can be used to reduce the systemic toxicity of a drug, by achieving high levels of the active drug only at the desired site.

In an embodiment, an enzyme required for prodrug conversion or a gene encoding such an enzyme is delivered to a target cell, e.g., a cancer cell. For example, the enzymes or genes can be delivered by a method described herein. In an embodiment, the gene encoding the enzyme required for prodrug conversion is delivered by a viral vector.

Cas9 molecules and gRNA molecules, e.g., a Cas9 molecule/gRNA molecule complex, can be used to manipulate a cell (e.g., a cell described herein), e.g., to deliver a payload, or edit a target nucleic acid, e.g., to improve immunotherapy, e.g. cancer immunotherapy. Typically an eiCas9 molecule/gRNA molecule complex is used to deliver a payload and an eaCas9 molecule/gRNA complex is used to edit or alter the structure of a target nucleic acid. Exemplary genes that can be modulated include, e.g., one or more genes described herein, e.g., PD-L1 and/or PD-L2 genes.

### VIIB. Targets: Pathways and Genes

Cas9 molecules and gRNA molecules, e.g., a Cas9 molecule/gRNA molecule complex, can be used to manipulate one, two, three or more, elements or a pathway, e.g., by targeting sequences that encode an RNA or protein of a pathway, or sequences that control the expression of an RNA or protein of a pathway. In an embodiment, an element of a first pathway and an element of a second pathway are manipulated. In an embodiment, manipulation comprises delivery of a payload to, or editing, a target nucleic acid. Typically an eiCas9 molecule/gRNA molecule complex is used to deliver a payload and an eaCas9 molecule/gRNA complex is used to edit or alter the structure of a target nucleic acid. Delivery or editing can be performed in vitro, ex vivo, or in vivo.

An element of a pathway can be up or down regulated, e.g., the expression of a gene encoding a protein of a pathway can be increased or decreased. The increase or decrease can be effected by delivery of a payload (e.g., a transcription factor or inhibitor of a transcription factor) or by editing a target nucleic acid (e.g., the use of a template nucleic acid to alter a sequence, e.g., correct or introduce a mutation, in e.g., a control or coding region).

Exemplary pathways comprise pathways associated with: cell proliferation; cell cycle; carbon metabolism; energy metabolism; glycolysis, anerobic respiration, anerobic respiration; transmembrane signal transduction, angiogenesis, DNA replication or repair, or pain.

Exemplary pathways and genes are discussed herein. It will be understood that a pathway or gene can be associated with one or more aspect of cell or organismal function, e.g., a pathway or gene can be involved in both cancer and energy metabolism. Manipulation of a pathway or gene is not limited to the exemplary cell or organismal function listed below. In an embodiment a pathway is associated with one or more diseases or conditions.

In an embodiment, the pathway is associated with cancer, e.g., associated with proliferation (e.g., RAF pathway), evading growth repressors, resisting cell death, enabling replicative immortality/aging, inducing angiogenesis, activating invasion and metastasis, energy metabolism and evading, cancer stem cells, cytokine-receptor interactions, or tumor suppressors. In some embodiments, the pathway is associated with cell cycle control. In some embodiments, the pathway is associated with angiogenesis.

Pathways and genes associated with cancer are described herein, e.g., include the following:

TABLE VII-14
Target Genes from Selected Pathways

| | | | CRISPR |
|---|---|---|---|
| Protein/Gene | Pathway | Disease | Regulation |
| Cancer | | | |
| PI3K | Proliferation | | Down |
| B-Raf | Proliferation | 66% of all melanoma cancers have a | Down |
| | | single substitution in codon 599 | |
| AKT | Proliferation | | Down |
| PTEN | Proliferation | Germline mutations leading to a | Down |
| | | predisposition to breast and | |
| | | thyroid cancer | |
| | | Mutations found in sporadic | |
| | | brain, breast and prostate | |
| mTOR | Proliferation | | Down |
| JUN | Proliferation | | Down |
| FOS | Proliferation | | Down |
| ERK | Proliferation | | Down |
| MEK | Proliferation | | Down |
| TGF-b | Proliferation | | Down |
| Myc | Proliferation | | Down |
| K-Ras | Proliferation | Mutated in lung cancer (10% of all | Down |
| | | Asians and 30% of all Caucasians) | |
| Src | Proliferation | | Down |
| PYK2 | Proliferation | | Down |
| PAK | Proliferation | | Down |
| FAK | Proliferation | | Down |
| PKA | Proliferation | | Down |
| RAC | Proliferation | | Down |
| ALK | Proliferation | Mutated in a subset (2-7%) of lung | |
| | | cancers | |
| Rb | Evading growth | | Up |
| | suppressors/pro- | | |
| | apoptotic | | |
| P53 | Evading growth | Mutation in colon, lung, esophagus, | Up |
| | suppressors/pro- | breast, liver, brain reticuloendothelial | |
| | apoptotic | tissues, and hemopoietic tissues | |
| APC | Evading growth | Mutations found in colon and intestine | |
| | suppressors/pro- | | |
| | apoptotic | | |
| CDK4/6 | Evading growth | | Up |
| | suppressors/pro- | | |
| | apoptotic | | |
| INK4B | Evading growth | | Up |
| | suppressors/pro- | | |
| | apoptotic | | |
| CDK2 | Evading growth | | Up |
| | suppressors/pro- | | |
| | apoptotic | | |
| WNT | Evading growth | | Up |
| | suppressors/pro- | | |
| | apoptotic | | |
| WAF1 | Evading growth | | Up |
| | suppressors/pro- | | |
| | apoptotic | | |
| Frizzled | Evading growth | | Up |
| | suppressors/pro- | | |
| | apoptotic | | |
| VHL | Evading growth | Mutated in all clear cell renal | Up |
| | suppressors/pro- | carcinomas | |
| | apoptotic | | |
| Fas ligand | Resisting cell death/ | | Down |
| | anti-apoptotic | | |
| Fas receptor | Resisting cell death/ | | Down |
| | anti-apoptotic | | |
| Caspase 8 | Resisting cell death/ | | Down |
| | anti-apoptotic | | |
| Caspase 9 | Resisting cell death/ | | Down |
| | anti-apoptotic | | |
| Bcl-2 | Resisting cell death/ | Correct mutation large deletion in | Down |
| | anti-apoptotic | follicular lymphoma, breast prostate | |
| | | CLL, melanoma | |
| Bcl-xL | Resisting cell death/ | | Down |
| | anti-apoptotic | | |
| Bcl-w | Resisting cell death/ | | Down |
| | anti-apoptotic | | |
| Mcl-1 | Resisting cell death/ | | Down |
| | anti-apoptotic | | |
| Bax | Resisting cell death/ | | Down |
| | anti-apoptotic | | |
| Bak | Resisting cell death/ | | Down |
| | anti-apoptotic | | |
| IGF-1 | Resisting cell death/ | | Down |
| | anti-apoptotic | | |
| Puma | Resisting cell death/ | | Down |
| | anti-apoptotic | | |
| Bim | Resisting cell death/ | | Down |
| | anti-apoptotic | | |
| Beclin-1 | Resisting cell death/ | | Down |
| | anti-apoptotic | | |
| TGF-b | Enabling replicative | | |
| | immortality/aging | | |
| Telomerase/TERT | Enabling replicative | | Down |
| | immortality/aging | | |
| ATAD2 | Enabling replicative | | |
| | immortality/aging | | |
| DAF-2 | Enabling replicative | | |
| | immortality/aging | | |
| SRT | Enabling replicative | | |
| | immortality/aging | | |
| Eph-A/B | Inducing angiogenesis | | Down |
| Robo | Inducing angiogenesis | | Down |
| Neuropilin | Inducing angiogenesis | | Down |
| Notch | Inducing angiogenesis | | Down |
| Endostatin | Inducing angiogenesis | | Down |
| Angiostatin | Inducing angiogenesis | | Down |
| FGF family | Inducing angiogenesis | | Down |
| Extracellular | Inducing angiogenesis | | Down |
| matrix-degrading | | | |
| proteases (e.g., | | | |
| MMP-2 & MMP- | | | |
| 9) | | | |
| VEGF-A | Inducing angiogenesis | | Down |
| TSP-1 | Inducing angiogenesis | | Down |
| VEGFR-1 | Inducing angiogenesis | | Down |
| VEGFR-2 | Inducing angiogenesis | | Down |
| VEGFR-3 | Inducing angiogenesis | | Down |
| NF2 | Activating invasion and | | Down |
| | metastasis | | |
| LKB1 | Activating invasion and | Up- regulated in multiple cancer, | Down |
| | metastasis | including intestine | |
| Snail | Activating invasion and | | Down |
| | metastasis | | |
| Slug | Activating invasion and | | Down |
| | metastasis | | |
| Twist | Activating invasion and | | Down |
| | metastasis | | |
| Zeb1/2 | Activating invasion and | | Down |
| | metastasis | | |
| CCLR5 | Activating invasion and | | Down |
| | metastasis | | |
| cysteine cathepsin | Activating invasion and | | Down |
| protease family | metastasis | | |
| Extracellular | Activating invasion and | | Down |
| matrix-degrading | metastasis | | |
| proteases (e.g., | | | |
| MMP-2 & MMP- | | | |
| 9) | | | |
| EGF | Activating invasion and | | Down |
| | metastasis | | |
| CSF-1 | Activating invasion and | | |
| | metastasis | | |
| PP2 | Energy metabolism | | Down |
| eIF4E | Energy metabolism | | Down |
| RSK | Energy metabolism | | Down |
| PIK3CA | Energy metabolism | Mutated in many breast, bladder | Down |
| | | cancers and hepatocellular carcinoma | |
| BAP1 | Energy metabolism | Mutated in renal cell carcinoma | Down |
| TWIST (TF) | Cancer Stem Cells | | Down |
| HIF-1α | Cancer Stem Cells | Over expressed in renal cell carcinoma | Down |
| HER2/neu | Cancer Stem Cells | | Down |
| Snail (TF) | Cancer Stem Cells | | Down |
| Wnt | Cancer Stem Cells | | Down |
| EPCAM | Cancer Stem Cells | Overexpressed in breast, colon, uterus | Down |
| | | and other cancers | |
| EGF | Cytokine-receptor | | Down |
| | interactions | | |
| TGFa | Cytokine-receptor | | Down |
| | interactions | | |
| PDGF | Cytokine-receptor | | Down |
| IGF-1 | interactions | | |
| KILTLG | | | |
| FLT3LG | Cytokine-receptor | | Down |
| | interactions | | |
| HGF | Cytokine-receptor | | Down |
| | interactions | | |
| FGF | Cytokine-receptor | | Down |
| | interactions | | |
| EGFR | Cytokine-receptor | Mutated in lung cancer (40% of all | Down |
| | interactions | Asians and 10-15% of all Caucasians) | |
| ERBB2 | Cytokine-receptor | | Down |
| | interactions | | |
| PDGFR | Cytokine-receptor | | Down |
| | interactions | | |
| IGFR | Cytokine-receptor | | Down |
| | interactions | | |
| c-KIT | Cytokine-receptor | | Down |
| | interactions | | |
| FLT3 | Cytokine-receptor | | Down |
| | interactions | | |
| MET | Cytokine-receptor | | Down |
| | interactions | | |
| FGFR | Cytokine-receptor | Mutations in bladder cancer | Down |
| | interactions | | |
| DNA damage and | | | |
| genomic instability | | | |
| DNMT1 | Methyl transferases | | |
| DNMT2 | Methyl transferases | | |
| DNMT3a | Methyl transferases | | |
| DNMT3b | Methyl transferases | | |
| H3K9Me3 | Histone methylation | | |
| H3K27Me | Histone methylation | | |
| Lsh | Helicase activity | | |
| BLM | Helicase activity | Bloom's syndrome > Cancer | Correct |
| WRN | Helicase activity | Werner's syndrome > Cancer | Correct |
| RTS | Helicase activity | Rothmund-Thompson > Cancer | Correct |
| XPA through XPG | Nucleotide excision | Xeroderma pigmentosa | |
| | repair | | |
| XPB | Nucleotide excision | Cockayne's syndrome | |
| | repair | | |
| XAB2 | Nucleotide excision | | |
| | repair | | |
| XPD | Nucleotide excision | Cockayne's syndrome | |
| | repair | | |
| TFIIH | Nucleotide excision | | |
| | repair | | |
| RFC | Nucleotide excision | | |
| | repair | | |
| PCNA | Nucleotide excision | | |
| | repair | | |
| LIG 1 | Nucleotide excision | | |
| | repair | | |
| Flap | Nucleotide excision | | |
| endonueclease 1 | repair | | |
| MNAT | Nucleotide excision | | |
| | repair | | |
| MMS19 | Nucleotide excision | | |
| | repair | | |
| RAD23A | Nucleotide excision | | |
| | repair | | |
| RAD23B | Nucleotide excision | | |
| | repair | | |
| RPA1 | Nucleotide excision | | |
| | repair | | |
| RPA2 | Nucleotide excision | | |
| | repair | | |
| CCNH | Nucleotide excision | | |
| | repair | | |
| CDK7 | Nucleotide excision | | |
| | repair | | |
| CETN2 | Nucleotide excision | | |
| | repair | | |
| DDB1 | Nucleotide excision | | |
| | repair | | |
| DDB2 | Nucleotide excision | | |
| | repair | | |
| ERCC1 | Nucleotide excision | | |
| | repair | | |
| ATM | Recombinational repair | | |
| NBN | Recombinational repair | | |
| BRCA1 | Recombinational repair | Breast, ovarian and pancreatic cancer | Correct |
| | | susceptibility | or Up |
| BRCA2 | Recombinational repair | Breast cancer and ovarian | Correct |
| | | susceptibility | or UP |
| RAD51 | Recombinational repair | | |
| RAD52 | Recombinational repair | | |
| WRN | Recombinational repair | | |
| BLM | Recombinational repair | | |
| FANCB | Recombinational repair | | |
| MLH1 | Mismatch repair | Multiple (including colon and uterus) | |
| MLH2 | Mismatch repair | Multiple (including colon and uterus) | |
| MSH2 | Mismatch repair | | |
| MSH3 | Mismatch repair | | |
| MSH4 | Mismatch repair | | |
| MSH5 | Mismatch repair | | |
| MSH6 | Mismatch repair | Multiple (including colon and uterus) | |
| PMS1 | Mismatch repair | | |
| PMS2 | Mismatch repair | Multiple (including colon and uterus) | |
| PMS2L3 | Mismatch repair | | |
| Aging | | | |
| DAF-2 | | | |
| IGF-1 | | | |
| SRT1 | | | |

TABLE VII-15
Genes Mutated in Common Cancers

| | |
|---|---|
| Bladder | FGFR3, RB1, HRAS, KRAS, TP53, TSC1, FGFR3 |
| Breast and Ovarian | BRCA, BRCA 2, BARD1, BRIP1, CHEK2, MRE11A, NBN, |
| | PALB2, PTEN, RAD50, RAD50, RAD51C, RAD51D, PPMID, |
| | TP53, BRIP1, RAD54L, SLC22A1L, PIK3CA, RB1CC1, |
| Cervical | FGFR3 |
| Colon and Rectal | PT53, STK11, PTEN, BMPR1A, SMAD, MLH1, MSH2, |
| | MSH6, PMS, EPCAM, AKT1, APC, MYH, PTPRJ, AXIN2 |
| Endometrial/Uterine | MLH1, MSH2, MSH6, PMS, EPCAM |
| Esophageal | DLEC1, TGFBR2, RNF6, LZT1S1, WWOX |
| Hepatocellular carcinoma | PDGFRL, CTNNB1, TP53, MET, CASP8, PIK3CA |
| Renal | VHL, PBRMQ, BAP1, SETD2, HIF1-α |
| Lung | KRAS, EGFR, ALK, BRAF, ERBB2, FLCN, DIRC2, RNF139, |
| | OGG1, PRCC, TFE, MET, PPP2R1B, RASSF1, SLC22A1L |
| Melanoma | BRAF, CDKA, CDKN2A, CDKN2B, CDKND, MC1R, TERT, |
| | ATF1, CREB1, EWSR1 |
| Non-Hodgkin Lymphoma | CASP10, EGFR, IRF1, PIK3CA |
| Osteosarcoma | CKEK2, LOJ18CR1, RB1 |
| Ovarian | PRKN, AKT1 |
| Pancreatic | KRAS, BRCA2, CDKN2A, MANF, PALB2, SMAD4, TP53, |
| | IPF1 |
| Prostate | MLH1, MSH2, MSH6, and PMS2, BRCA 1, HOXB13, CHEK2, |
| | ELAC2, EPHB2, SDR5A2, PRKAR1A, PMC1 |
| Papillary and Follicular | BRAF, NARAS, ERC1, FOXE1, GOLGA5, NCOA4, NKX2-1, |
| Thyroid | PMC1, RET, TFG, TPR, TRIM24, TRIM27, TRIM33 |
| Erwing Sarcoma | ERG, ETV1, ETV4, EWSR1, FLI1 |
| Leukemia | BRC, AMCR2, GMPS, JAK2, AF10, ARFGEF12, CEBPA, |
| | FLT3, KIT, LPP, MLF1, NPM1, NSD1, NUP214, PICALM, |
| | RUNX1, SH3GL1, WHSC1L1, ETV6, RARA, BCR, |
| | ARHGAP26, NF1, PTPN11, GATA1 |

Any of the following cancer associated genes provided in Table VII-16 can be targeted.

Table VII-16 Exemplary Target Genes Associated With Cancer:

TABLE VII-16
ABL1, ABL2, ACSL3, AF15Q14, AF1Q, AF3p21, AF5q31, AKAP9, AKT1, AKT2, ALDH2, ALK,
ALO17, APC, ARHGEF12, ARHH, ARID1A, ARID2, ARNT, ASPSCR1, ASXL1, ATF1, ATIC,
ATM, ATRX, AXIN1, BAP1, BCL10, BCL11A, BCL11B, BCL2, BCL3, BCL5, BCL6, BCL7A,
BCL9, BCOR, BCR, BHD, BIRC3, BLM, BMPR1A, BRAF, BRCA1, BRCA2, BRD3, BRD4, BRIP1,
BTG1, BUB1B, C12orf9, C15orf21, C15orf55, C16orf75, C2orf44, CAMTA1, CANT1, CARD11,
CARS, CBFA2T1, CBFA2T3, CBFB, CBL, CBLB, CBLC, CCDC6, CCNB1IP1, CCND1, CCND2,
CCND3, CCNE1, CD273, CD274, CD74, CD79A, CD79B, CDH1, CDH11, CDK12, CDK4, CDK6,
CDKN2A, CDKN2a(p14), CDKN2C, CDX2, CEBPA, CEP1, CHCHD7, CHEK2, CHIC2, CHN1, CIC,
CIITA, CLTC, CLTCL1, CMKOR1, CNOT3, COL1A1, COPEB, COX6C, CREB1, CREB3L1,
CREB3L2, CREBBP, CRLF2, CRTC3, CTNNB1, CYLD, D10S170, DAXX, DDB2, DDIT3, DDX10,
DDX5, DDX6, DEK, DICER1, DNM2, DNMT3A, DUX4, EBF1, ECT2L, EGFR, EIF4A2, ELF4,
ELK4, ELKS, ELL, ELN, EML4, EP300, EPS15, ERBB2, ERCC2, ERCC3, ERCC4, ERCC5, ERG,
ETV1, ETV4, ETV5, ETV6, EVI1, EWSR1, EXT1, EXT2, EZH2, EZR, FACL6, FAM22A, FAM22B,
FAM46C, FANCA, FANCC, FANCD2, FANCE, FANCF, FANCG, FBXO11, FBXW7, FCGR2B,
FEV, FGFR1, FGFR1OP, FGFR2, FGFR3, FH, FHIT, FIP1L1, FLI1, FLJ27352, FLT3, FNBP1,
FOXL2, FOXO1A, FOXO3A, FOXP1, FSTL3, FUBP1, FUS, FVT1, GAS7, GATA1, GATA2,
GATA3, GMPS, GNA11, GNAQ, GNAS, GOLGA5, GOPC, GPC3, GPHN, GRAF, H3F3A,
HCMOGT-1, HEAB, HERPUD1, HEY1, HIP1, HIST1H3B, HIST1H4I, HLF, HLXB9, HMGA1,
HMGA2, HNRNPA2B1, HOOK3, HOXA11, HOXA13, HOXA9, HOXC11, HOXC13, HOXD11,
HOXD13, HRAS, HRPT2, HSPCA, HSPCB, IDH1, IDH2, IGH@, IGK@, IGL@, IKZF1, IL2, IL21R,
IL6ST, IL7R, IRF4, IRTA1, ITK, JAK1, JAK2, JAK3, JAZF1, JUN, KCNJ5, KDM5A, KDM5C,
KDM6A, KDR, KIAA1549, KIF5B, KIT, KLF4, KLK2, KRAS, KTN1, LAF4, LASP1, LCK, LCP1,
LCX, LHFP, LIFR, LMO1, LMO2, LPP, LRIG3, LYL1, MADH4, MAF, MAFB, MALT1, MAML2,
MAP2K1, MAP2K2, MAP2K4, MAX, MDM2, MDM4, MDS1, MDS2, MECT1, MED12, MEN1,
MET, MITF, MKL1, MLF1, MLH1, MLL, MLL2, MLL3, MLLT1, MLLT10, MLLT2, MLLT3,
MLLT4, MLLT6, MLLT7, MN1, MPL, MSF, MSH2, MSH6, MSI2, MSN, MTCP1, MUC1, MUTYH,
MYB, MYC, MYCL1, MYCN, MYD88, MYH11, MYH9, MYST4, NACA, NBS1, NCOA1, NCOA2,
NCOA4, NDRG1, NF1, NF2, NFE2L2, NFIB, NFKB2, NIN, NKX2-1, NONO, NOTCH1, NOTCH2,
NPM1, NR4A3, NRAS, NSD1, NT5C2, NTRK1, NTRK3, NUMA1, NUP214, NUP98, OLIG2, OMD,
P2RY8, PAFAH1B2, PALB2, PAX3, PAX5, PAX7, PAX8, PBRM1, PBX1, PCM1, PCSK7,
PDE4DIP, PDGFB, PDGFRA, PDGFRB, PER1, PHF6, PHOX2B, PICALM, PIK3CA, PIK3R1, PIM1,
PLAG1, PML, PMS1, PMS2, PMX1, PNUTL1, POT1, POU2AF1, POU5F1, PPARG, PPP2R1A,
PRCC, PRDM1, PRDM16, PRF1, PRKAR1A, PRO1073, PSIP2, PTCH, PTEN, PTPN11, RAB5EP,
RAC1, RAD51L1, RAF1, RALGDS, RANBP17, RAP1GDS1, RARA, RB1, RBM15, RECQL4, REL,
RET, RNF43, ROS1, RPL10, RPL22, RPL5, RPN1, RUNDC2A, RUNX1, RUNXBP2, SBDS, SDC4,
SDH5, SDHB, SDHC, SDHD, SEPT6, SET, SETBP1, SETD2, SF3B1, SFPQ, SFRS3, SH2B3,
SH3GL1, SIL, SLC34A2, SLC45A3, SMARCA4, SMARCB1, SMARCE1, SMO, SOCS1, SOX2,
SRGAP3, SRSF2, SS18, SS18L1, SSH3BP1, SSX1, SSX2, SSX4, STAT3, STK11, STL, SUFU,
SUZ12, SYK, TAF15, TAL1, TAL2, TCEA1, TCF1, TCF12, TCF3, TCF7L2, TCL1A, TCL6, TERT,
TET2, TFE3, TFEB, TFG, TFPT, TFRC, THRAP3, TIF1, TLX1, TLX3, TMPRSS2, TNFAIP3,
TNFRSF14, TNFRSF17, TNFRSF6, TOP1, TP53, TPM3, TPM4, TPR, TRA@, TRAF7, TRB@,
TRD@, TRIM27, TRIM33, TRIP11, TSC1, TSC2, TSHR, TTL, U2AF1, USP6, VHL, VTI1A, WAS,
WHSC1, WHSC1L1, WIF1, WRN, WT1, WTX, WWTR1, XPA, XPC, XPO1, YWHAE, ZNF145,
ZNF198, ZNF278, ZNF331, ZNF384, ZNF521, ZNF9, or ZRSR2

Exemplary pathways and genes associated with energy metabolism are provided in Table VII-17. Exemplary metabolic targets disclosed herein may be modulated using CRISPR/Cas9 as described herein. Modulation may be used to knockdown a gene of interest, correct a defect or mutation in the gene, or to activate a gene of interest.

TABLE VII-17
Exemplary Metabolic Target List

| Target | How to Modulate |
|---|---|
| ACAT, acyl-CoA: cholesterol | Knock down |
| acyltransferase | |
| AGPAT2, 1-acyl-glcero-3-phos- | Knock down |
| phate acyltransferase 2 | |
| DGAT, diacylglycerol acyltrans- | Knock down |
| ferase | |
| GL, gastric lipase | Knock down |
| PL, pancreatic lipase | Knock down |
| sPLA2, secretory phospholipase | Knock down |
| A2 | |
| ACC, acetyl-CoA carboxylase | Knock down |
| CPT, carnitine palmitoyl trans- | Knock down |
| ferase | |
| FAS, fatty-acid synthase | Knock down |
| MTP, microsomal triglyceride- | Knock down |
| transfer protein | |
| Insulin receptor | Correct defects or activate |
| SU receptor/K+ ATP channel | Activate with mutation |
| a-glucosidase | Knock down |
| PPARy | Activate with mutation |
| Glycogen phosphorylase | Knock down |
| Fructose-1,6-bisphosphatase | Knock down |
| glucose-6-phosphatase | Knock down |
| PTP-1B | Knock down |
| SHIP-2 | Knock down |
| GSK-3 | Knock down |
| lkB kinase | Knock down |
| PKCq | Knock down |
| GLP1R | Correct mutation |
| GIPR | Correct mutation |
| GPR40 | Correct mutation |
| GPR119 | Correct mutation |
| GPR41 | Correct mutation |
| GPR43 | Correct mutation |
| GPR120 | Correct mutation |
| GCGR | Correct mutation |
| PAC1 | Correct mutation |
| VPAC2 | Correct mutation |
| Y1 | Knock down |
| GHSR | Knock down |
| CCKAR | Correct mutation |
| b2 | Correct mutation |
| a2 | Knock down |
| MT1 | Knock down |
| M3 | Correct mutation |
| CB1 | Knock down |
| P2Y | Correct mutation |
| H3 | Inhibit |
| MCH-R1 | Correct mutation |
| MCH-R2 | Correct mutation |
| Ghrelin R | Inhibit |
| FASN | Inhibit |
| Bombesin-R3 | Inhibit |
| CCK-A Receptor | Correct mutation |
| Seratonin System | Correct mutation |
| CBI Cannabinoid Receptors | Inhibit |
| Dopaminergic System | Correct mutation |
| Enterostatin | Mutate to super agonist |
| CNTF | Mutate to super agonist |
| CNTF-R | Correct mutation |
| SOCS-3 | Knock down |
| 46a | Knock down |
| PrPP Receptors | Correct mutation |
| Amylin | Mutate to super agonist |
| CRH System | Mutate to super agonist |
| Galanin Receptors | Knock down |
| Orexin Receptors | Knock down |
| Noradrenalin System | Mutate to super agonist |
| CART | Mutate to super agonist |
| FATP4 | Knock down |
| Pancreatic Lipase | Knock down |
| ACRP30 | Super agonist mutations |
| Thyroid Hormone | Correct mutation |
| B-3 Adrenergic Receptor | Correct mutation |
| UCPs | Upregulate |
| PTP-1B | Knock down |
| MC3 | Correct mutation |
| ACC2 | Knock down |
| Perilipin | Knock down |
| HMGIC | Knock down |
| 11BHSD-1 | Knock down |
| Glucagon R | Knock down |
| Glucocoricoid R | Knock down |
| 11beta-HSD I | Knock down |
| PGC-1 | Correct mutation |
| DPPP-IV | Knock down |
| GLP | Mutate to super agonist |
| GIP | Mutate to super agonist |
| GLP-IR | Correct mutation |
| AMP Kinase | Correct mutation |
| IKK-b | Knock down |
| PPARa/g | Knock down |
| INS-R | Knock down |
| SGLT | Knock down |
| a-glucosidase | Knock down |
| HMGCR | Knock down |
| PCSK9 | Knock down |
| ApoB-100 | Knock down |
| Leptin | Mutate to super agonist |
| Leptin Receptor | Mutate to constitutively active |
| | receptor |
| MC4R | Mutate to constitutively active |
| | receptor |
| VOMC | Mutate MSH region to super |
| | agonist |
| AGRP | Knock down |
| IVPY Receptors | Introduce constitutively active |
| | mutations |
| 5HT2C | Introduce constitutively active |
| | mutations |
| GLP-1 | Mutate to super agonist |
| GLP-1 Receptor | Mutate to constitutively active |
| | receptor |

In an embodiment, the pathways and genes described herein, e.g., in Table VII-17, are also associated with diabetes, obesity, and/or cholesterol and lipids.

Exemplary pathways and genes associated with the cell cycle are provided in Table VII-18.

TABLE VII-18
CELL CYCLE PATHWAYS and REPRESENTATIVE GENES

| DNA Damage | | | Mismatch repair | Apoptosis |
|---|---|---|---|---|
| ATM | | | PMS2 | Fas-L |
| MRE11 | | | MLH1 | FasR |
| NBS1 | | | MSH6 | Trail-L |
| RAD50 | | | MSH2 | Trail-R |
| 53BP1 | | | RFC | TNF-α |
| P53 | | | PCNA | TNF-R1 |
| CHKE | | | MSH3 | FADD |
| E2F1 | | | MutS homolog | TRADD |
| PML | | | MutL homolog | RIPI |
| FANCD2 | | | Exonuclease | MyD88 |
| SMC1 | | | DNA Polymerase | IRAK |
| BLM1 | | | delta | NIL |
| BRCA1 | | | (POLD1, POLD2, | IKK |
| H2AX | | | POLD3, and | NF-Kβ |
| ATR | | | POLD4 -genes | IκBα |
| RPA | | | encoding subunits) | IAP |
| ATRIP | | | Topoisomerase 1 | Caspase 3 |
| RAD9 | | | Topoisomerase 2 | Caspase 6 |
| RAD1 | | | RNAseH1 | Caspase 7 |
| HUS | | | Ligase 1 | Caspase 8 |
| RAD17 | | | DNA polymerase 1 | Caspase 10 |
| RFC | | | DNA polymerase 3 | HDAC1 |
| CHK1 | | | Primase | HDAC2 |
| TLK1 | | | Helicase | Cytochrome |
| CDC25 | | | Single-strand | C |
| | | | binding | Bxl-xL |
| | | | proteins | STAT3 |
| | | | | STAT5 |
| | | | | DFF45 |
| | | | | Vcl-2 |
| | | | | ENDO-G |
| | | | | PI3K |
| | | | | Akt |
| | | | | Calpain |
| | | | | Bad |
| | | | | Bax |
| | | | | Cell Pro- |
| Ubiquitin-mediated proteolysis | | | Hypoxia | liferation |
| E1 | HERC1 | TRAF6 | HIF-1α | MAPK |
| E2 | UBE2Q | MEKK1 | HIF-1β | MAPKK |
| E3 | UBE2R | COP1 | Ref1 | MAPKKK |
| UBLE1A | UBE2S | PIFH2 | HSP90 | c-Met |
| UBLE1B | UBE2U | cIAP | VEGF | HGF |
| UBLE1C | UBE2W | PIAS | PAS | ERKS1/2 |
| UBE2A | UBE2Z | SYVN | ARNT | ATK |
| UBE2B | AFCLLCN | NHLRC1 | VHL | PKCs |
| UBE2C | UBE1 | AIRE | HLF | Paxilin |
| UBE2A | E6AP | MGRN1 | EPF | FAK |
| UBE2E | UBE3B | BRCA1 | VDU2 | Adducin |
| UBE2F | Smurf | FANCL | SUMORESUME | PYK1 |
| UBE2G1 | Itch | MID1 | SENP1 | RB |
| UBE2G2 | HERC2 | Cdc20 | Calcineurin A | RB1 |
| UBE2I | HERC3 | Cdh1 | RACK1 | Raf-1 |
| UBE2J1 | HERC4 | Apc1 | PTB | A-Raf |
| UBE2J2 | UBE4A | Apc2 | Hur | B-raf |
| UBE2L3 | UBE4B | Apc3 | PHD2 | MEK1/2 |
| UBE2L6 | CHIP | Apc4 | SSAT2 | ERK1/2 |
| UBE2M | CYC4 | Apc5 | SSAT1 | Ets |
| UBE2N | PPR19 | Apc6 | GSK3β | Elk1 |
| UBE2O | UIP5 | Apc7 | CBP | SAP1 |
| WWPI | Mdm2 | Apc8 | FOXO4 | cPLA2 |
| WWP2 | Parkin | Apc9 | FIH-1 | |
| TRIP12 | Trim32 | Apc10 | | |
| NEED4 | Trim37 | Apc11 | | |
| ARF-BP1 | SIAH-1 | Apc12 | | |
| EDD1 | PML | | | |

| Cell | |
|---|---|
| survival | Cell cycle arrest |
| SMAD1 | P21 |
| SMAD5 | BAX |
| SAMD8 | MDR |
| LEF1 | DRAIL IGFBP3 |
| TCF3 | GADD45 |
| TCF4 | |
| P300 | |
| HAT1 | |
| PI3K | |
| Akt | |
| GF1 | |

Exemplary cell cycle genes characterized by their function are provided in Table VII-19.

TABLE VII-19
CELL CYCLE GENES

| Translation | | Cyclin- |
|---|---|---|
| initiation | | dependent Kinases |
| factors | Cyclins | (DKs) |
| E2F1 | CCNA1, CCNA2, CCNB1, | CDK1, CDK2, CDK3, CDK5, |
| E2F2 | CCNB2, CCNB3, CCNC, | CDK6, CDK7, CDK8, CDK9, |
| E2F3 | CCND1, CCND2, CCND3, | CDK11, |
| E2F4 | CCNE1, CCNE2, CCNF, | |
| E2F5 | CCNG1, CCNG2, CCNH, | |
| E2F6 | CCNI, CCNI2, , CCNO, | |
| E2F8 | CCNT1, CCNT2, CCNY, | |
| | CCNYL1, CCNYL2, | |
| | CCNYL3 | |
| Cyclin | CDK inhibitory | CDK regulators (both |
| regulators | proteins (CDKIs) | positive and negative) |
| c-Jun | INK4 family | RINGO/Speedy family |
| c-Fos | P15 | P53 |
| | P16 | MDM2 |
| | P18 | RB |
| | P19 | CHK1 |
| | CIP/KIP family | CHk2 |
| | P21 | ATM |
| | P27 | ATR |
| | P57 | CDC2 |
| | | HDAC1 |
| | | HDAC2 |

Exemplary pathways and genes associated with the angiogenesis are described provided in Table VII-20.

TABLE VII-20
ANGIOGENESIS PATHWAY GENES

| | Cell surface | | Transcription |
|---|---|---|---|
| Extracellular ligands | receptors | Signal transduction | factors |
| PLGF | VEGFR1 | PLCγ | c-FOS |
| VEGF | VEGFR2 | SHC | E2F7 |
| VEGFB | VEGFR3 | PI3K | |
| VEGFC | Nrp1 | PIP3 | |
| VEGFD | | IP3 | |
| | | DAG | |
| | | GRB2 | |
| | | SOS | |
| | | Akt | |
| | | PKB | |
| | | PKC | |
| | | Ras | |
| | | RAF1 | |
| | | DAG | |
| | | eNOS | |
| | | NO | |
| | | ERK1 | |
| | | ERK2 | |
| | | cPLA2 | |
| | | MEK1 | |
| | | MEK2 | |

Exemplary pathways and genes associated with the mitochondrial function are provided in Table VII-25.

TABLE VII-25

Pathways and genes associated with mitochondrial function

| | | Mitochondrial | Valine oxidation |
|---|---|---|---|
| B-oxidation | TCA Cycle | apoptosis | pathway |
| acyl CoA | Citrate synthase | | Transaminase |
| dehydrogenase | Aconitase | | BCKADH complex |
| enoyl CoA hydratase | Isocitrate dehydrogenase | | ACAD-8 |
| 3-hydroxyacyl-CoA | Alpha-ketoglutarate | | Crotonoase |
| dehydrogenase | dehydrogenase | | HIBCH |
| β-ketothiolase | Succinyl-CoA synthetase | | HIBADH |
| | Succinate dehydrogenase | | MMSDH |
| | Fumarase | | Aminotransferase |
| | Malate dehydrogenase | | Hydratase |
| | | | Deacylase |
| | | | Dehydrogenase |
| | | | Carboxylase |
| | | | Mutase |

| Fatty acid oxidation | | |
|---|---|---|
| disorders (enzyme | Leucine Oxidation | Isoleucine |
| deficiencies) | Pathway | oxidation pathway |
| OCTN2 | Aminotransferase | Aminotransferase |
| FATP1-6 | Branched chain | Branched chain |
| CPT-1 | aminotransferase 2, | aminotransferase 2, |
| CACT | mitochondrial | mitochondrial |
| CPT-II | Isobutytyl-CoA | 2-methylbutytyl-CoA |
| SCAD | dehydrogenase | Dehydrogenase |
| MCAD | (Branched Chain | (Branched Chain |
| VLCAD | Keto Acid | Keto Acid |
| ETF-DH | Dehydrogase | Dehydrogenase |
| Alpha-ETF | Complex) | Complex) |
| Beta-ETF | Hydratase | Hydratase |
| SCHAD | HMG-CoA lyase | 2-methyl-3-OH- |
| LCHAD | | butyryl-CoA |
| MTP | | dehydrogenase |
| LKAT | | 3-Oxothiolase |
| DECR1 | | |
| HMGCS2 | | |
| HMGCL | | |

Additional mitochondrial genes and related diseases caused by mutations

| | |
|---|---|
| Mt-ND1 | Leber's hereditary optic neuropathy |
| Mt-ND4 | Leber's hereditary optic neuropathy |
| Mt-ND6 | Leber's hereditary optic neuropathy |
| OPA1 | Autosomal dominant optic atrophy |
| CMT2A | Charcot-Marie-Toothhereditary neuropathy type 2A |
| mt-TK | Myoclonic epilepsy with ragged red fibres |

| Mitochondrial | |
|---|---|
| Respiratory chain | |
| genes | Related diseases |
| NADH CoQ | Alpers, Alzheimer's, Parkinsonism, Cardiomyopathy, Deficiency (Barth |
| Reductase | and/or Lethal Infantile), Encephalopathy, Infantile CNS, Leber's, Leigh, |
| | Longevity, MELAS, MERRF, Myopathy ± CNS, PEO, Spinal cord |
| | disorders |
| Succinate-CoQ | Kearns-Sayre, Leigh's, Myopathy (e.g., Infantile ± CNS), Paraganglioma, |
| Reductase | Pheochromocytoma |
| CoQ-Cytochrome C | Cardiomyopathy, Fatal infantile, GRACILE, Leber's, Myopathy (e.g., |
| Reductase | ±CNS, PEO) |
| Cytochrome C | Alper's, Ataxia, Deafness, Leber's, Leigh's, Myopathy (e.g., Infantile (e.g., |
| Oxidase | Fatal, Benign), Adult), Rhabdomyolysis, PEO, KSS, MNGIE, MERRF, |
| | MELAS |
| ATP Synthase | Cardiomyopathy, Encephalopathy, Leber's, Leigh, Multisystem, NARP |

Complex I (NADH-Ubiquinone Oxidoreductase)

| | | | Subunits involved |
|---|---|---|---|
| Nuclear encoded | Mitochondral DNA | Supernumerary | in regulation of |
| proteins | encoded proteins | subunits | Complext I activity |
| NDUFS1: Childhood | ND1 | NDUFAB1 (SDAP): | NDUFS4 (AQDQ) |
| encephalopathy; Most | ND2 | Carrier of fatty acid | Functions: |
| common Complex I | ND3 | chain | Increased Complex |
| mutations (3%) | ND4 | NDUFA1 (MWFE) | I activity with |
| NDUFS2: | ND4L | Primarily expressed | phosphorylation |
| Cardiomyopathy + | ND5 | in heart & skeletal | Disorders: |
| Encephalomyopathy | ND6 | muscle | Multisystem |
| NDUFS3: Leigh | | Disorders: | childhood |
| NDUFS7: Leigh | | Encephalopathies | encephalopathy |
| NDUFS8: Leigh | | NDUFA2: | with Complex I |
| NDUFV1: Childhood | | Encephalopathy & | deficiency; Leigh |
| encephalopathy | | Cardiomyopathy | syndrome |
| NDUFV2: | | NDUFA9: Leigh | |
| Encephalopathy + | | syndrome | |
| Cardiomyopathy | | NDUFA10: Leigh | |
| ELAC2: | | syndrome | |
| Cardiomyopathy, | | NDUFA11 | |
| Hypertrophic | | Disorder: | |
| | | Encephalopathy & | |
| | | Cardiomyopathy | |
| | | NDUFA12: Leigh | |
| | | syndrome | |
| | | NDUFB9: | |
| | | Hypotonia | |
| | | NDUFS6: Lethal | |
| | | Infantile | |
| | | Mitochondrial | |
| | | Disease | |

| Proteins involved in | |
|---|---|
| Complex I assembly | Other |
| NDUFAF1: | NDUFA13: Thyroid |
| Cardiomyopathy + | carcinoma (Hurthle |
| Encephalomyopathy | cell) |
| NDUFAF2 | NDUFB3: Severe |
| (NDUFA12L): | lethal mitochondrial |
| Childhood | complex I deficiency |
| encephalopathy; | MTHFR deficiency |
| Usually null | MGME1: PEO + |
| mutations | Myopathy |
| NDUFAF3: Lethal | |
| neonatal | |
| encephalopathy | |
| NDUFAF4: | |
| Encephalopathy | |
| C6ORF66: | |
| Encephalopathy | |
| C8orf38: Leigh | |
| syndrome | |
| C20orf7: Lethal | |
| neonatal | |
| NUBPL: | |
| Encephalomyopathy | |
| ACAD9: Fatigue & | |
| Exercise intolerance; | |
| Most missense | |
| mutations | |
| FOXRED1: Leigh | |
| syndrome | |
| Ecsit | |
| AIF (AIFM1; | |
| PDCD8) | |
| Ind1 | |

Complex I (NABH-Ubiquinone Oxidoreductase)

| | |
|---|---|
| Flavoprotein: FAD (SDHA; Fp) | Mutations cause Leigh syndrome with |
| | Complex II deficiency |
| | Late onset neurodegenerative disorder) |
| Iron-Sulfur protein: SDHB (Ip) | Mutations cause Reduced tumor suppression |
| | Neoplasms: Pheochromocytoma & |
| | Paraganglioma |
| SDHC; SDHD (cytochrome C subunits) - | mutations lead to paraganglioma |

Complex III (Cytochrome reductase)

| | |
|---|---|
| Cytochrome c1 (CYC1) | |
| Rieske FeS protein (UQCRFS1) | |
| Ubiquinol-cytochrome c reductase core | May mediate formation of complex between |
| protein I (UQCRC1; QCR; Subunit 1) | cytochromes c and c1 |
| Ubiquinol-cytochrome c reductase core | Required for assembly of complex III |
| protein II (UQCRC2; QCR2; Subunit 2) | |
| UQCRH (Subunit 6) | May mediate formation of complex between |
| | cytochromes c and c1 |
| Ubiquinone-binding protein (UQBC; | Redox-linked proton pumping |
| UQPC; UQCRB; UQBP; Subunit 7) | |
| UQCRQ (Subunit 8) | Binds to ubiquinone |
| Ubiquinol-cytochrome C reductase | Interacts with cytochrome c1 |
| complex, 7.2-KD Subunit (UCRC; | |
| UQCR10; Subunit 9) | |
| UQCR (UQCR11; Subunit 10) | function as iron-sulfur protein binding factor |
| Cleavage product of UQCRFS1 | |
| (Cytochrome b-c1 complex subunit 11) | |

Inner membrane proteins and related disorders
ABCB7: Ataxia + Anemia
ACADVL: Myopathy
ADCK3: SACR9
AGK: Sengers
ATP5A1: Encephalopathy, neonatal
ATP5E: Retardation + Neuropathy
BRP44L: Encephalopathy
c12orf62: Encephalocardiomyopathy
Cardiolipin: Barth
COX4I2: Pancreas + Anemia
COX6B1: Encephalomyopathy
CPT2: Myopathy
CRAT: Encephalomyopathy
CYC1: Hyperglycemia & Encephalopathy
CYCS
CYP11A1
CYP11B1
CYP11B2
CYP24A1
CYP27A1: Cerebrotendinous Xanthomatosis
CYP27B1
DHODH
DNAJC19: Cardiac + Ataxia
FASTKD2: Encephalomyopathy
GPD2
HADHA: Multisystem; Myopathy
HADHB: Encephalomyopathy
HCCS: MIDAS
L2HGDH: Encephalopathy
MMAA
MPV17: Hepatocerebral
NDUFA1: Encephalopathy
NDUFA2: Leigh + Cardiac
NDUFA4: Leigh
NDUFA9: Leigh
NDUFA10: Leigh
NDUFA11: Encephalocardiomyopathy
NDUFA12: Leigh
NDUFA13
NDUFB3: Lethal infantile
NDUFB9: Encephalopathy
NDUFV1: Encephalopathy
NDUFV2: Encephalopathy + Cardiac
NDUFS1: Leukodystrophy
NDUFS2: Encephalopathy + Cardiac
NDUFS3: Dystonia
NDUFS4: Encephalopathy
NDUFS6: Lethal infantile
NDUFS7: Encephalopathy
NDUFS8: CNS + Cardiac
OPA1: Optic atrophy
OPA3: Optic atrophy
PDSS1: Coenzyme Q10 deficiency
SDHA: Leigh; Cardiac; Paraganglioma
SDHB: Paraganglioma
SDHC: Paraganglioma
SDHD: Paraganglioma
SLC25A carriers
SLC25A1: Epileptic encephalopathy
SLC25A3: Cardiac; Exercise intolerance
SLC25A4: PEOA2
SLC25A12: Hypomyelination
SLC25A13: Citrullinemia
SLC25A15: HHH
SLC25A19: Microcephaly
SLC25A20: Encephalocardiomyopathy
SLC25A22: Myoclonic epilepsy
SLC25A38: Anemia
Paraplegin: SPG7
TIMM8A: Deaf-Dystonia-Dementia
UCP1
UCP2
UCP3
UQCRB: Hypoglycemia, Hepatic
UQCRC2: Episodic metabolic encephalopathy
UQCRQ: Encephalopathy

Pathways and genes associated with DNA damage and genomic instability include the following methyl transferases, histone methylation, helicase activity, nucleotide excision repair, recombinational repair, or mismatch repair provided in Table VII-21. See also Table VI-22.

TABLE VII-21
PATHWAYS and GENES ASSOCIATED with
DNA DAMAGE and GENOMIC INSTABILITY

| | | | Non-Homologous |
|---|---|---|---|
| Double-stranded Breaks | Replication Stress | DNA Methylation | End-Joining |
| ATM | ATR | DNMT1 | Ku70 |
| RAD50 | RAD17 | DNMT2 | Ku80 |
| MRE119 | ATRIP | DNMT3A | DNA |
| NBS1 | RAD9 | DNMT3B | PKc |
| CRCA1 | RPA | DNMT3L | XRCC4 |
| H2AX | CHK1 | MeCP2 | DNA ligase 4 |
| 53BP1 | BLM | MBD2 | XLF |
| MDC1 | H2AX | | Rad50 |
| SMC1 | 53BP1 | | Artemis |
| P53 | P53 | | Rad27 |
| | | | TdT |

| | Nucleotide-Excision | Homologous | |
|---|---|---|---|
| Base-Excision repair | Repair | Recombination | Mismatch repair |
| APE1 | UvrA | RecA | PMS2 |
| APE2 | UvrB | SSB | MLH1 |
| NEIL1 | UvrC | Mre11 | MSH6 |
| NEIL2 | XPC | Rad50 | MSH2 |
| NEIL3 | Rad23B | Nbs1 | RFC |
| XRCC1 | CEN2 | CtIP | PCNA |
| PNKP | DDB1 | RPA | MSH3 |
| Tdp1 | XPE | Rad51 | MutS |
| APTX | CSA, | Rad52 | MutL |
| DNA polymerase β | CSB | Rad54 | Exonuclease |
| DNA polymerase δ | TFIIH | BRCA1 | Topoisomerase 1 |
| DNA polymerase ε | XPB | BRCA2 | Topoisomerase 2 |
| PCNA | XPD | Exo1 | RNAseH1 |
| FEN1 | XPA | BLM | Ligase 1 |
| RFC | RPA | TopIIIα | DNA polymerase 1 |
| PARP1 | XPG | GEN1 | DNA polymerase 3 |
| Lig1 | ERCC1 | Yen1 | Primase |
| Lig3 | XPF | Slx1 | Helicase |
| UNG | DNA polymerase δ | Slx4 | SSBs |
| MUTY | DNA polymerase ε | Mus8 | |
| SMUG | | Eme1 | |
| MBD4 | | Dss1 | |

Histone Methylation

| | |
|---|---|
| ASH1L | SETD4 |
| DOT1L | SETD5 |
| EHMT1 | SETD6 |
| EHMT2 | SETD7 |
| EZH1 | SETD8 |
| EZH2 | SETD9 |
| MLL | SETDB1 |
| MLL2 | SETDB2 |
| MLL3 | SETMAR |
| MLL4 | SMYD1 |
| MLL5 | SMYD2 |
| NSD1 | SMYD3 |
| PRDM2 | SMYD4 |
| SET | SMYD5 |
| SETBP1 | SUV39H1 |
| SETD1A | SUV39H2 |
| SETD1B | SUV420H1 |
| SETD2 | SUV420H2 |
| SETD3 | |

TABLE VII-22
Selected Transcription FactorsTranscription factors

| | |
|---|---|
| NIKX2-5 | Cardiac malformations and |
| | atrioventricular conduction |
| | abnormalities |
| MECP2 | Rett syndrome |
| HNF1 through | Mature onset diabetes of the young |
| HNF6 | (MODY), hepatic adenomas and renal |
| | cysts |
| FOXP2 | Developmental verbal dyspraxia |
| FOXP3 | Autoimmune diseases |
| NOTCH1 | Aortic valve abnormalities |
| MEF2A | Coronary artery disease |
| CRX | Dominant cone-rod dystrophy |
| FOCX2 | Lymphedema-distichiasis |
| NF-κB | Autoimmune arthritis, asthma, septic |
| Activation | shock, lung fibrosis, |
| | glomerulonephritis, atherosclerosis, |
| | and AIDS |
| NF-κB Inhibition | Apoptosis, inappropriate immune cell |
| | development, and delayed cell growth |
| NARA2 | Parkinson disease |
| LHX3 | Pituitary disease |
| GAT4 | Congenital heart defects |
| P53, APC | Cancer |
| CTCF | Epigenetics and cell growth regulation |
| EGR2 | Congenital hypomyelinating |
| | neuropathy (CHN) and Charcot-Marie- |
| | Tooth type 1 (CMT1) |
| STAT family | Cancer and immunosuppression |
| NF-AT family | Cancer and inflammation |
| AP-1 family | Cancer and inflammation |

A gene including receptors and ionophores relevant to pain in this table can be targeted, by editing or payload delivery. Pathways and genes associated with pain are described herein, e.g., include the following those in Table VII-24.

TABLE VII-24

| | Part of | | | |
|---|---|---|---|---|
| | nervous | | | |
| Type of pain | system | Target | Area | How to affect |
| nociceptive | central | 5-HT | central inhibition | |
| nociceptive | central | 5HT1A | central inhibition | agonists (activation) serve |
| | | | | as analgesic, |
| | | | | antidepressants, anxiolytics, |
| | | | | psychosis |
| nociceptive | central | 5HT1A | central inhibition | antagonists can work as |
| | | | | antidepressants, nootropics |
| nociceptive | central | 5HT1B | central inhibition | migraines |
| nociceptive | central | 5HT1D | central inhibition | migraines |
| nociceptive | central | 5HT1E | central inhibition | |
| nociceptive | central | 5HT1F | central inhibition | agonists-psychedelics |
| nociceptive | central | 5HT1F | central inhibition | antagonists-atypical |
| | | | | antipsychotics, NaSSAsm |
| | | | | treatig sertonin syndrome, |
| | | | | sleeping aid |
| nociceptive | central | 5HT2A | central inhibition | agonists-psychadelics |
| nociceptive | central | 5HT2A | central inhibition | antagonists-atypical |
| | | | | antipsychotics, NaSSAs, |
| | | | | treating seratonin syndrome, |
| | | | | sleeping aid |
| nociceptive | central | 5HT2B | central inhibition | migraines |
| nociceptive | central | 5HT2C | central inhibition | antidepressant, orexigenic, |
| | | | | anorectic, antipsychotic |
| nociceptive | central | 5HT3 | central inhibition | antiemetic |
| nociceptive | central | 5HT4 | central inhibition | gastroproknetics |
| nociceptive | central | 5HT5A | central inhibition | |
| nociceptive | central | 5HT5B | central inhibition | |
| nociceptive | central | 5HT6 | central inhibition | antidepressant (antagonists |
| | | | | and agonists), anxiolytic |
| | | | | (antagonists and agonists), |
| | | | | nootropic (antagonists), |
| | | | | anorectic (antagonists) |
| nociceptive | central | 5HT7 | central inhibition | antidepressant (antagonists), |
| | | | | anxiolytics (antagonists), |
| | | | | nootropic (antagonists) |
| nociceptive | central | CB1 | central inhibition | |
| nociceptive | central | GABA | central inhibition | |
| nociceptive | central | GABAA-$ | central inhibition | |
| nociceptive | central | GABAB-R | central inhibition | |
| nociceptive | central | Glucine-R | central inhibition | |
| nociceptive | central | NE | central inhibition | |
| nociceptive | central | Opiod | central inhibition | |
| | | receptors | | |
| nociceptive | central | c-fos | gene expression | |
| nociceptive | central | c-jun | gene expression | |
| nociceptive | central | CREB | gene expression | |
| nociceptive | central | DREAM | gene expression | |
| nociceptive | peripheral | K+ channel | membrane | |
| | | | excitability of | |
| | | | primary afferents | |
| nociceptive | peripheral | Nav1.8 | membrane | |
| | | | excitability of | |
| | | | primary afferents | |
| nociceptive | peripheral | Nav1.9 | membrane | |
| | | | excitability of | |
| | | | primary afferents | |
| nociceptive | peripheral | CaMKIV | peripheral | |
| | | | sensitization | |
| nociceptive | peripheral | COX2 | peripheral | |
| | | | sensitization | |
| nociceptive | peripheral | cPLA2 | peripheral | |
| | | | sensitization | |
| nociceptive | peripheral | EP1 | peripheral | |
| | | | sensitization | |
| nociceptive | peripheral | EP3 | peripheral | |
| | | | sensitization | |
| nociceptive | peripheral | EP4 | peripheral | |
| | | | sensitization | |
| nociceptive | peripheral | ERK1/2 | peripheral | |
| | | | sensitization | |
| nociceptive | peripheral | IL-1beta | peripheral | |
| | | | sensitization | |
| nociceptive | peripheral | JNK | peripheral | |
| | | | sensitization | |
| nociceptive | peripheral | Nav1.8 | peripheral | |
| | | | sensitization | |
| nociceptive | peripheral | NGF | peripheral | |
| | | | sensitization | |
| nociceptive | peripheral | p38 | peripheral | |
| | | | sensitization | |
| nociceptive | peripheral | PKA | peripheral | |
| | | | sensitization | |
| nociceptive | peripheral | PKC | peripheral | |
| | | isoforms | sensitization | |
| nociceptive | peripheral | TNFalpha | peripheral | |
| | | | sensitization | |
| nociceptive | peripheral | TrkA | peripheral | |
| | | | sensitization | |
| nociceptive | peripheral | TRPV1 | peripheral | |
| | | | sensitization | |
| nociceptive | central | AMPA/kai- | postsynaptic | |
| | | nate-R | transmission | |
| nociceptive | central | K+ channels | postsynaptic | |
| | | | transmission | |
| nociceptive | central | mGlu-$ | postsynaptic | |
| | | | transmission | |
| nociceptive | central | Nav1.3 | postsynaptic | |
| | | | transmission | |
| nociceptive | central | NK1 | postsynaptic | |
| | | | transmission | |
| nociceptive | central | NMDA-R | postsynaptic | |
| | | | transmission | |
| nociceptive | peripheral | Adenosine- | presynaptic | |
| | | R | transmission | |
| nociceptive | peripheral | mGluR | presynaptic | |
| | | | transmission | |
| nociceptive | peripheral | VGCC | presynaptic | |
| | | | transmission | |
| nociceptive | central | ERK | signal | |
| | | | transduction | |
| nociceptive | central | JNK | signal | |
| | | | transduction | |
| nociceptive | central | p38 | signal | |
| | | | transduction | |
| nociceptive | central | PKA | signal | |
| | | | transduction | |
| nociceptive | central | PKC | signal | |
| | | isoforms | transduction | |
| nociceptive | peripheral | ASIC | transduction | |
| nociceptive | peripheral | BK1 | transduction | |
| nociceptive | peripheral | BK2 | transduction | |
| nociceptive | peripheral | DRASIC | transduction | |
| nociceptive | peripheral | MDEG | transduction | |
| nociceptive | peripheral | P2X3 | transduction | |
| nociceptive | peripheral | TREK-1 | transduction | |
| nociceptive | peripheral | TRPM8 | transduction | |
| nociceptive | peripheral | TRPV1 | transduction | |
| nociceptive | peripheral | TRPV2 | transduction | |
| nociceptive | peripheral | TRPV3 | transduction | |
| neuropathic | | | | |
| pain | | | | |
| Inflammatory | | histamine | | |
| pain | | | | |
| Inflammatory | | ATP | | |
| pain | | | | |
| Inflammatory | | bradykinin | | |
| pain | | | | |
| Inflammatory | | CB2 | | |
| pain | | | | |
| Inflammatory | | Endothelins | | |
| pain | | | | |
| Inflammatory | | H+ | | |
| pain | | | | |
| Inflammatory | | Interleukins | | |
| pain | | | | |
| Inflammatory | | NGF | | |
| pain | | | | |
| Inflammatory | | prostaglandins | | |
| pain | | | | |
| Inflammatory | | serotonin | | |
| pain | | | | |
| Inflammatory | | TNFalpha | | |
| pain | | | | |

### VIII. Targets: Disorders Associated with Disease Causing Organisms

Cas9 molecules, typically eiCas9 molecules or eaCas9 molecules, and gRNA molecules, e.g., an eiCas9 molecule/gRNA molecule complex, e.g., an eaCas9 molecule/gRNA molecule complex, can be used to treat or control diseases associated with disease causing organisms, e.g., to treat infectious diseases. In an embodiment, the infectious disease is treated by editing (e.g., correcting) one or more target genes, e.g., of the organism or of the subject. In other embodiments, the infectious disease is treated by delivering one or more payloads (e.g., as described herein) to the cell of a disease causing organism or to an infected cell of the subject, e.g., to a target gene. In some embodiments, the target gene is in the infectious pathogen. Exemplary infectious pathogens include, e.g., viruses, bacteria, fungi, protozoa, or multicellular parasites.

In other embodiments, the target gene is in the host cell. For example, modulation of a target gene in the host cell can result in resistance to the infectious pathogen. Host genes involved in any stage of the life cycle of the infectious pathogen (e.g., entry, replication, latency) can be modulated. In an embodiment, the target gene encodes a cellular receptor or co-receptor for the infectious pathogen. In an embodiment, the infectious pathogen is a virus, e.g., a virus described herein, e.g., HIV. In an embodiment, the target gene encodes a co-receptor for HIV, e.g., CCR5 or CXCR4.

Exemplary infectious diseases that can be treated by the molecules and methods described herein, include, e.g., AIDS, Hepatitis A, Hepatitis B, Hepatitis C, Herpes simplex, HPV infection, or Influenza.

Exemplary targets are provided in Table VIII-1. The disease and causative organism are provided.

TABLE VIII-1

| DISEASE | SOURCE OF DISEASE |
|---|---|
| Acinetobacter infections | Acinetobacter baumannii |
| Actinomycosis | Actinomyces israelii, Actinomyces |
| | gerencseriae and Propionibacterium |
| | propionicus |
| African sleeping sickness | Trypanosoma brucei |
| (African trypanosomiasis) | |
| AIDS (Acquired | HIV (Human immunodeficiency virus) |
| immunodeficiency syndrome) | |
| Amebiasis | Entamoeba histolytica |
| Anaplasmosis | Anaplasma genus |
| Anthrax | Bacillus anthracis |
| Arcanobacterium haemolyticum | Arcanobacterium haemolyticum |
| infection | |
| Argentine hemorrhagic fever | Junin virus |
| Ascariasis | Ascaris lumbricoides |
| Aspergillosis | Aspergillus genus |
| Astrovirus infection | Astroviridae family |
| Babesiosis | Babesia genus |
| Bacillus cereus infection | Bacillus cereus |
| Bacterial pneumonia | multiple bacteria |
| Bacterial vaginosis (BV) | multiple bacteria |
| Bacteroides infection | Bacteroides genus |
| Balantidiasis | Balantidium coli |
| Baylisascaris infection | Baylisascaris genus |
| BK virus infection | BK virus |
| Black piedra | Piedraia hortae |
| Blastocystis hominis infection | Blastocystis hominis |
| Blastomycosis | Blastomyces dermatitidis |
| Bolivian hemorrhagic fever | Machupo virus |
| Borrelia infection | Borrelia genus |
| Botulism (and Infant botulism) | Clostridium botulinum; Note: Botulism is |
| | not an infection by Clostridium botulinum |
| | but caused by the intake of botulinum |
| | toxin. |
| Brazilian hemorrhagic fever | Sabia |
| Brucellosis | Brucella genus |
| Bubonic plague | the bacterial family Enterobacteriaceae |
| Burkholderia infection | usually Burkholderia cepacia and other |
| | Burkholderia species |
| Buruli ulcer | Mycobacterium ulcerans |
| Calicivirus infection (Norovirus | Caliciviridae family |
| and Sapovirus) | |
| Campylobacteriosis | Campylobacter genus |
| Candidiasis (Moniliasis; Thrush) | usually Candida albicans and other |
| | Candida species |
| Cat-scratch disease | Bartonella henselae |
| Cellulitis | usually Group A Streptococcus and |
| | Staphylococcus |
| Chagas Disease (American | Trypanosoma cruzi |
| trypanosomiasis) | |
| Chancroid | Haemophilus ducreyi |
| Chickenpox | Varicella zoster virus (VZV) |
| Chlamydia | Chlamydia trachomatis |
| Chlamydophila pneumoniae | Chlamydophila pneumoniae |
| infection (Taiwan acute | |
| respiratory agent or TWAR) | |
| Cholera | Vibrio cholerae |
| Chromoblastomycosis | usually Fonsecaea pedrosoi |
| Clonorchiasis | Clonorchis sinensis |
| Clostridium difficile infection | Clostridium difficile |
| Coccidioidomycosis | Coccidioides immitis and Coccidioides |
| | posadasii |
| Colorado tick fever (CTF) | Colorado tick fever virus (CTFV) |
| Common cold (Acute viral | usually rhinoviruses and coronaviruses. |
| rhinopharyngitis; Acute coryza) | |
| Creutzfeldt-Jakob disease (CJD) | PRNP |
| Crimean-Congo hemorrhagic | Crimean-Congo hemorrhagic fever virus |
| fever (CCHF) | |
| Cryptococcosis | Cryptococcus neoformans |
| Cryptosporidiosis | Cryptosporidium genus |
| Cutaneous larva migrans (CLM) | usually Ancylostoma braziliense; multiple |
| | other parasites |
| Cyclosporiasis | Cyclospora cayetanensis |
| Cysticercosis | Taenia solium |
| Cytomegalovirus infection | Cytomegalovirus |
| Dengue fever | Dengue viruses (DEN-1, DEN-2, DEN-3 |
| | and DEN-4)-Flaviviruses |
| Dientamoebiasis | Dientamoeba fragilis |
| Diphtheria | Corynebacterium diphtheriae |
| Diphyllobothriasis | Diphyllobothrium |
| Dracunculiasis | Dracunculus medinensis |
| Ebola hemorrhagic fever | Ebolavirus (EBOV) |
| Echinococcosis | Echinococcus genus |
| Ehrlichiosis | Ehrlichia genus |
| Enterobiasis (Pinworm infection) | Enterobius vermicularis |
| Enterococcus infection | Enterococcus genus |
| Enterovirus infection | Enterovirus genus |
| Epidemic typhus | Rickettsia prowazekii |
| Erythema infectiosum (Fifth | Parvovirus B19 |
| disease) | |
| Exanthem subitum (Sixth | Human herpesvirus 6 (HHV-6) and Human |
| disease) | herpesvirus 7 (HHV-7) |
| Fasciolopsiasis | Fasciolopsis buski |
| Fasciolosis | Fasciola hepatica and Fasciola gigantica |
| Fatal familial insomnia (FFI) | PRNP |
| Filariasis | Filarioidea superfamily |
| Food poisoning by Clostridium | Clostridium perfringens |
| perfringens | |
| Free-living amebic infection | multiple |
| Fusobacterium infection | Fusobacterium genus |
| Gas gangrene (Clostridial | usually Clostridium perfringens; other |
| myonecrosis) | Clostridium species |
| Geotrichosis | Geotrichum candidum |
| Gerstmann-Sträussler-Scheinker | PRNP |
| syndrome (GSS) | |
| Giardiasis | Giardia intestinalis |
| Glanders | Burkholderia mallei |
| Gnathostomiasis | Gnathostoma spinigerum and Gnathostoma |
| | hispidum |
| Gonorrhea | Neisseria gonorrhoeae |
| Granuloma inguinale | Klebsiella granulomatis |
| (Donovanosis) | |
| Group A streptococcal infection | Streptococcus pyogenes |
| Group B streptococcal infection | Streptococcus agalactiae |
| Haemophilus influenzae | Haemophilus influenzae |
| infection | |
| Hand, foot and mouth disease | Enteroviruses, mainly Coxsackie A virus and |
| (HFMD) | Enterovirus 71 (EV71) |
| Hantavirus Pulmonary | Sin Nombre virus |
| Syndrome (HPS) | |
| Helicobacter pylori infection | Helicobacter pylori |
| Hemolytic-uremic syndrome | Escherichia coli O157:H7, O111 and |
| (HUS) | O104:H4 |
| Hemorrhagic fever with renal | Bunyaviridae family |
| syndrome (HFRS) | |
| Hepatitis A | Hepatitis A Virus |
| Hepatitis B | Hepatitis B Virus |
| Hepatitis C | Hepatitis C Virus |
| Hepatitis D | Hepatitis D Virus |
| Hepatitis E | Hepatitis E Virus |
| Herpes simplex | Herpes simplex virus 1 and 2 (HSV-1 and |
| | HSV-2) |
| Histoplasmosis | Histoplasma capsulatum |
| Hookworm infection | Ancylostoma duodenale and Necator |
| | americanus |
| Human bocavirus infection | Human bocavirus (HBoV) |
| Human ewingii ehrlichiosis | Ehrlichia ewingii |
| Human granulocytic | Anaplasma phagocytophilum |
| anaplasmosis (HGA) | |
| Human metapneumovirus | Human metapneumovirus (hMPV) |
| infection | |
| Human monocytic ehrlichiosis | Ehrlichia chaffeensis |
| Human papillomavirus (HPV) | Human papillomavirus (HPV) |
| infection | |
| Human parainfluenza virus | Human parainfluenza viruses (HPIV) |
| infection | |
| Hymenolepiasis | Hymenolepis nana and Hymenolepis |
| | diminuta |
| Epstein-Barr Virus Infectious | Epstein-Barr Virus (EBV) |
| Mononucleosis (Mono) | |
| Influenza (flu) | Orthomyxoviridae family |
| Isosporiasis | Isospora belli |
| Kawasaki disease | unknown; evidence supports that it is |
| | infectious |
| Keratitis | multiple |
| Kingella kingae infection | Kingella kingae |
| Kuru | PRNP |
| Lassa fever | Lassa virus |
| Legionellosis (Legionnaires' | Legionella pneumophila |
| disease) | |
| Legionellosis (Pontiac fever) | Legionella pneumophila |
| Leishmaniasis | Leishmania genus |
| Leprosy | Mycobacterium leprae and Mycobacterium |
| | lepromatosis |
| Leptospirosis | Leptospira genus |
| Listeriosis | Listeria monocytogenes |
| Lyme disease (Lyme borreliosis) | usually Borrelia burgdorferi and other |
| | Borrelia species |
| Lymphatic filariasis | Wuchereria bancrofti and Brugia malayi |
| (Elephantiasis) | |
| Lymphocytic choriomeningitis | Lymphocytic choriomeningitis virus |
| | (LCMV) |
| Malaria | Plasmodium genus |
| Marburg hemorrhagic fever | Marburg virus |
| (MHF) | |
| Measles | Measles virus |
| Melioidosis (Whitmore's | Burkholderia pseudomallei |
| disease) | |
| Meningitis | multiple |
| Meningococcal disease | Neisseria meningitidis |
| Metagonimiasis | usually Metagonimus yokagawai |
| Microsporidiosis | Microsporidia phylum |
| Molluscum contagiosum (MC) | Molluscum contagiosum virus (MCV) |
| Monkeypox | Monkeypox virus |
| Mumps | Mumps virus |
| Murine typhus (Endemic typhus) | Rickettsia typhi |
| Mycoplasma pneumonia | Mycoplasma pneumoniae |
| Mycetoma | numerous species of bacteria |
| | (Actinomycetoma) and fungi (Eumycetoma) |
| Myiasis | parasitic dipterous fly larvae |
| Neonatal conjunctivitis | most commonly Chlamydia trachomatis and |
| (Ophthalmia neonatorum) | Neisseria gonorrhoeae |
| (New) Variant Creutzfeldt-Jakob | PRNP |
| disease (vCJD, nvCJD) | |
| Nocardiosis | usually Nocardia asteroides and other |
| | Nocardia species |
| Onchocerciasis (River blindness) | Onchocerca volvulus |
| Paracoccidioidomycosis (South | Paracoccidioides brasiliensis |
| American blastomycosis) | |
| Paragonimiasis | usually Paragonimus westermani and other |
| | Paragonimus species |
| Pasteurellosis | Pasteurella genus |
| Pediculosis capitis (Head lice) | Pediculus humanus capitis |
| Pediculosis corporis (Body lice) | Pediculus humanus corporis |
| Pediculosis pubis (Pubic lice, | Phthirus pubis |
| Crab lice) | |
| Pelvic inflammatory disease | multiple |
| (PID) | |
| Pertussis (Whooping cough) | Bordetella pertussis |
| Plague | Yersinia pestis |
| Pneumococcal infection | Streptococcus pneumoniae |
| Pneumocystis pneumonia (PCP) | Pneumocystis jirovecii |
| Pneumonia | multiple |
| Poliomyelitis | Poliovirus |
| Prevotella infection | Prevotella genus |
| Primary amoebic | usually Naegleria fowleri |
| meningoencephalitis (PAM) | |
| Progressive multifocal | JC virus |
| leukoencephalopathy | |
| Psittacosis | Chlamydophila psittaci |
| Q fever | Coxiella burnetii |
| Rabies | Rabies virus |
| Rat-bite fever | Streptobacillus moniliformis and Spirillum |
| | minus |
| Respiratory syncytial virus | Respiratory syncytial virus (RSV) |
| infection | |
| Rhinosporidiosis | Rhinosporidium seeberi |
| Rhinovirus infection | Rhinovirus |
| Rickettsial infection | Rickettsia genus |
| Rickettsialpox | Rickettsia akari |
| Rift Valley fever (RVF) | Rift Valley fever virus |
| Rocky Mountain spotted fever | Rickettsia rickettsii |
| (RMSF) | |
| Rotavirus infection | Rotavirus |
| Rubella | Rubella virus |
| Salmonellosis | Salmonella genus |
| SARS (Severe Acute | SARS coronavirus |
| Respiratory Syndrome) | |
| Scabies | Sarcoptes scabiei |
| Schistosomiasis | Schistosoma genus |
| Sepsis | multiple |
| Shigellosis (Bacillary dysentery) | Shigella genus |
| Shingles (Herpes zoster) | Varicella zoster virus (VZV) |
| Smallpox (Variola) | Variola major or Variola minor |
| Sporotrichosis | Sporothrix schenckii |
| Staphylococcal food poisoning | Staphylococcus genus |
| Staphylococcal infection | Staphylococcus genus |
| Strongyloidiasis | Strongyloides stercoralis |
| Subacute sclerosing | Measles virus |
| panencephalitis | |
| Syphilis | Treponema pallidum |
| Taeniasis | Taenia genus |
| Tetanus (Lockjaw) | Clostridium tetani |
| Tinea barbae (Barber's itch) | usually Trichophyton genus |
| Tinea capitis (Ringworm of the | usually Trichophyton tonsurans |
| Scalp) | |
| Tinea corporis (Ringworm of the | usually Trichophyton genus |
| Body) | |
| Tinea cruris (Jock itch) | usually Epidermophyton floccosum, |
| | Trichophyton rubrum, and Trichophyton |
| | mentagrophytes |
| Tinea manuum (Ringworm of | Trichophyton rubrum |
| the Hand) | |
| Tinea nigra | usually Hortaea werneckii |
| Tinea pedis (Athlete's foot) | usually Trichophyton genus |
| Tinea unguium (Onychomycosis) | usually Trichophyton genus |
| Tinea versicolor (Pityriasis | Malassezia genus |
| versicolor) | |
| Toxocariasis (Ocular Larva | Toxocara canis or Toxocara cati |
| Migrans (OLM)) | |
| Toxocariasis (Visceral Larva | Toxocara canis or Toxocara cati |
| Migrans (VLM)) | |
| Toxoplasmosis | Toxoplasma gondii |
| Trichinellosis | Trichinella spiralis |
| Trichomoniasis | Trichomonas vaginalis |
| Trichuriasis (Whipworm | Trichuris trichiura |
| infection) | |
| Tuberculosis | usually Mycobacterium tuberculosis |
| Tularemia | Francisella tularensis |
| Ureaplasma urealyticum | Ureaplasma urealyticum |
| infection | |
| Valley fever | Coccidioides immitis or Coccidioides |
| | posadasii.^{[1]} |
| Venezuelan equine encephalitis | Venezuelan equine encephalitis virus |
| Venezuelan hemorrhagic fever | Guanarito virus |
| Viral pneumonia | multiple viruses |
| West Nile Fever | West Nile virus |
| White piedra (Tinea blanca) | Trichosporon beigelii |
| Yersinia pseudotuberculosis | Yersinia pseudotuberculosis |
| infection | |
| Yersiniosis | Yersinia enterocolitica |
| Yellow fever | Yellow fever virus |
| Zygomycosis | Mucorales order (Mucormycosis) and |
| | Entomophthorales order |
| | (Entomophthoramycosis) |

AIDS/HIV

HIV Genomic Structural Elements

Long terminal repeat (LTR) refers to the DNA sequence flanking the genome of integrated proviruses. It contains important regulatory regions, especially those for transcription initiation and polyadenylation.

Target sequence (TAR) for viral transactivation, the binding site for Tat protein and for cellular proteins; consists of approximately the first 45 nucleotides of the viral mRNAs in HIV-1 (or the first 100 nucleotides in HIV-2 and SIV.) TAR RNA forms a hairpin stem-loop structure with a side bulge; the bulge is necessary for Tat binding and function.

Rev responsive element (RPE) refers to an RNA element encoded within the env region of HIV-1. It consists of approximately 200 nucleotides (positions 7327 to 7530 from the start of transcription in HIV-1, spanning the border of gp120 and gp41). The RRE is necessary for Rev function; it contains a high affinity site for Rev; in all, approximately seven binding sites for Rev exist within the RRE RNA. Other lentiviruses (HIV-2, SIV, visna, CAEV) have similar RRE elements in similar locations within env, while HTLVs have an analogous RNA element (RXRE) serving the same purpose within their LTR; RRE is the binding site for Rev protein, while RXRE is the binding site for Rex protein. RRE (and RXRE) form complex secondary structures, necessary for specific protein binding.

Psi elements (PE) are a set of 4 stem-loop structures preceding and overlapping the Gag start codon which are the sites recognized by the cysteine histidine box, a conserved motif with the canonical sequence CysX2CysX4HisX4Cys (SEQ ID NO: 41), present in the Gag p7 MC protein. The Psi Elements are present in unspliced genomic transcripts but absent from spliced viral mRNAs.

SLIP, an TTTTTT slippery site, followed by a stem-loop structure, is responsible for regulating the −1 ribosomal frameshift out of the Gag reading frame into the Pol reading frame.

Cis-acting repressive sequences (CRS) are postulated to inhibit structural protein expression in the absence of Rev. One such site was mapped within the pol region of HIV-1. The exact function has not been defined; splice sites have been postulated to act as CRS sequences.

Inhibitory/Instability RNA sequences (INS) are found within the structural genes of HIV-1 and of other complex retroviruses. Multiple INS elements exist within the genome and can act independently; one of the best characterized elements spans nucleotides 414 to 631 in the gag region of HIV-1. The INS elements have been defined by functional assays as elements that inhibit expression posttranscriptionally. Mutation of the RNA elements was shown to lead to INS inactivation and up regulation of gene expression.

Genes and Gene Products

Essential for Replication

The genomic region (GAG) encoding the capsid proteins (group specific antigens). The precursor is the p55 myristylated protein, which is processed to p17 (MAtrix), p24 (CApsid), p7 (NucleoCapsid), and p6 proteins, by the viral protease. Gag associates with the plasma membrane where the virus assembly takes place. The 55 kDa Gag precursor is called assemblin to indicate its role in viral assembly.

The genomic region, POL, encoding the viral enzymes protease, reverse transcriptase, RNAse, and integrase. These enzymes are produced as a Gag-Pol precursor polyprotein, which is processed by the viral protease; the Gag-Pol precursor is produced by ribosome frameshifting near the end of gag.

Viral glycoproteins (e.g., ENV) produced as a precursor (gp160) which is processed to give a noncovalent complex of the external glycoprotein gp120 and the transmembrane glycoprotein gp41. The mature gp120-gp41 proteins are bound by non-covalent interactions and are associated as a trimer on the cell surface. A substantial amount of gp120 can be found released in the medium. gp120 contains the binding site for the CD4 receptor, and the seven transmembrane do-main chemokine receptors that serve as co-receptors for HIV-1.

The transactivator (TAT) of HIV gene expression is one of two essential viral regulatory factors (Tat and Rev) for HIV gene expression. Two forms are known, Tat-1 exon (minor form) of 72 amino acids and Tat-2 exon (major form) of 86 amino acids. Low levels of both proteins are found in persistently infected cells. Tat has been localized primarily in the nucleolus/nucleus by immunofluorescence. It acts by binding to the TAR RNA element and activating transcription initiation and elongation from the LTR promoter, preventing the LTR AATAAA polyadenylation signal from causing premature termination of transcription and polyadenylation. It is the first eukaryotic transcription factor known to interact with RNA rather than DNA and may have similarities with prokaryotic anti-termination factors. Extracellular Tat can be found and can be taken up by cells in culture.

The second necessary regulatory factor for HIV expression is REV. A 19 kDa phosphoprotein, localized primarily in the nucleolus/nucleus, Rev acts by binding to RRE and promoting the nuclear export, stabilization and utilization of the un-spliced viral mRNAs containing RRE. Rev is considered the most functionally conserved regulatory protein of lentiviruses. Rev cycles rapidly between the nucleus and the cytoplasm.

Others

Viral infectivity factor (VIF) is a basic protein of typically 23 kDa. Promotes the infectivity but not the production of viral particles. In the absence of Vif the produced viral particles are defective, while the cell-to-cell transmission of virus is not affected significantly. Found in almost all lentiviruses, Vif is a cytoplasmic protein, existing in both a soluble cytosolic form and a membrane-associated form. The latter form of Vif is a peripheral membrane protein that is tightly associated with the cytoplasmic side of cellular membranes. In 2003, it was discovered that Vif prevents the action of the cellular APOBEC-3G protein which deaminates DNA:RNA heteroduplexes in the cytoplasm.

Viral Protein R (VPR) is a 96-amino acid (14 kDa) protein, which is incorporated into the virion. It interacts with the p6 Gag part of the Pr55 Gag precursor. Vpr detected in the cell is localized to the nucleus. Proposed functions for Vpr include the targeting the nuclear import of preintegration complexes, cell growth arrest, transactivation of cellular genes, and induction of cellular differentiation. In HIV-2, SIV-SMM, SIV-RCM, SIV-MND-2 and SIV-DRL the Vpx gene is apparently the result of a Vpr gene duplication event, possibly by recombination.

Viral Protein U (VPU)) is unique to HIV-1, SIVcpz (the closest SIV relative of HIV-1), SIV-GSN, SIV-MUS, SIV-MON and SIV-DEN. There is no similar gene in HIV-2, SIV-SMM or other SIVs. Vpu is a 16 kDa (81-amino acid) type I integral membrane protein with at least two different biological functions: (a) degradation of CD4 in the endoplasmic reticulum, and (b) enhancement of virion release from the plasma membrane of HIV-1-infected cells. Env and Vpu are expressed from a bicistronic mRNA. Vpu probably possesses an N-terminal hydrophobic membrane anchor and a hydrophilic moiety. It is phosphorylated by casein kinase II at positions Ser52 and Ser56. Vpu is involved in Env maturation and is not found in the virion. Vpu has been found to increase susceptibility of HIV-1 infected cells to Fas killing.

NEF is amultifunctional 27-kDa myristylated protein produced by an ORF located at the 3 0 end of the primate lentiviruses. Other forms of Nef are known, including nonmyristylated variants. Nef is predominantly cytoplasmic and associated with the plasma membrane via the myristyl residue linked to the conserved second amino acid (Gly). Nef has also been identified in the nucleus and found associated with the cytoskeleton in some experiments. One of the first HIV proteins to be produced in infected cells, it is the most immunogenic of the accessory proteins. The nef genes of HIV and SIV are dispensable in vitro, but are essential for efficient viral spread and disease progression in vivo. Nef is necessary for the maintenance of high virus loads and for the development of AIDS in macaques, and viruses with defective Nef have been detected in some HIV-1 infected long term survivors. Nef downregulates CD4, the primary viral receptor, and MHC class I molecules, and these functions map to different parts of the protein. Nef interacts with components of host cell signal transduction and clathrin-dependent protein sorting pathways. It increases viral infectivity. Nef contains PxxP motifs that bind to SH3 domains of a subset of Src kinases and are required for the enhanced growth of HIV but not for the downregulation of CD4.

VPX is a virion protein of 12 kDa found in HIV-2, SIV-SMM, SIV-RCM, SIV-MND-2 and SIV-DRL and not in HIV-1 or other SIVs. This accessory gene is a homolog of HIV-1 vpr, and viruses with Vpx carry both vpr and vpx. Vpx function in relation to Vpr is not fully elucidated; both are incorporated into virions at levels comparable to Gag proteins through interactions with Gag p6. Vpx is necessary for efficient replication of SIV-SMM in PBMCs. Progression to AIDS and death in SIV-infected animals can occur in the absence of Vpr or Vpx. Double mutant virus lacking both vpr and vpx was attenuated, whereas the single mutants were not, suggesting a redundancy in the function of Vpr and Vpx related to virus pathogenicity.

Hepatitis A Viral Target Sequences
5′ untranslated region contains IRES—internal ribosome entry site

P1 Region of Genome—Capsid Proteins
VP1
VP2
VP3
VP4

P2 Region of Genome
2A
2B
2C

P3 Region of Genome
3A
3B
3C—viral protease
3D—RNA polymerase

Hepatitis B Viral Target Sequences

Precursor Polypeptide encoding all HCV protein is produced and then spliced into functional proteins. The following are the proteins (coding regions) encoded:
C—core protein—coding region consists of a Pre-C and Core coding region
X—function unclear but suspected to play a role in activation of viral transcription process
P—RNA polymerase
S—surface antigen—coding region consists of a Pre-S1, Pre-S2 and Surface antigen coding regions

Hepatitis C Viral Target Sequences

Precursor Polypeptide encoding all HCV protein is produced and then spliced into functional proteins. The following are the proteins (coding regions) encoded:
RES—non-coding internal ribosome entry site (5′ to polyprotein encoding sequence)
3′ non-coding sequences—
C region—encodes p22 a nucleocapsid protein
E1 region—encodes gp35 envelope glycoprotein—important in cell entry
E2 region—encodes gp70 envelope glycoprotein—important in cell entry
NS1—encodes p7—not necessary for replication but critical in viral morphogenesis
NS2—encodes p23 a transmembrane protein with protease activity
NS3—encodes p70 having both serine protease and RNA helicase activities
NS4A—encodes p8 co-factor
NS4B—encodes p27 cofactor—important in recruitment of other viral proteins
NS5A—encodes p56/58 an interferon resistance protein—important in viral replication
NS5B—encodes RNA polymerase

Herpes Simplex Virus Target Sequence

| Gene | Protein | Function/description |
|---|---|---|
| UL1 | Glycoprotein | Surface and membrane |
| | L [1] | |
| UL2 | UL2 [3] | Uracil-DNA glycosylase |
| UL3 | UL3 [5] | unknown |
| UL4 | UL4 [7] | unknown |
| UL5 | UL5 [9] | DNA replication |
| UL6 | Portal | Twelve of these proteins |
| | protein U_{L}-6 | constitute the capsid |
| | | portal ring through which |
| | | DNA enters and exits the |
| | | capsid.^{[12][13][14]} |
| UL7 | UL7 [12] | Virion maturation |
| UL8 | UL8 [14] | DNA helicase/primase |
| | | complex-associated |
| | | protein |
| UL9 | UL9 [16] | Replication origin- |
| | | binding protein |
| UL10 | Glycoprotein | Surface and membrane |
| | M [18] | |
| UL11 | UL11 [20] | virion exit and secondary |
| | | envelopment |
| UL12 | UL12 [22] | Alkaline exonuclease |
| UL13 | UL13 [24] | Serine-threonine protein |
| | | kinase |
| UL14 | UL14 [26] | Tegument protein |
| UL15 | Terminase [28] | Processing and |
| | | packaging of DNA |
| UL16 | UL16 [30] | Tegument protein |
| UL17 | UL17 [32] | Processing and |
| | | packaging DNA |
| UL18 | VP23 [34] | Capsid protein |
| UL19 | VP5 [36] | Major capsid protein |
| UL20 | UL20 [38] | Membrane protein |
| UL21 | UL21 [40] | Tegument protein^{[27]} |
| UL22 | Glycoprotein | Surface and membrane |
| | H [42] | |
| UL23 | Thymidine | Peripheral to DNA |
| | kinase [44] | replication |
| UL24 | UL24 [46] | unknown |
| UL25 | UL25 [48] | Processing and |
| | | packaging DNA |
| UL26 | P40; VP24; | Capsid protein |
| | VP22A [50] | |
| UL27 | Glycoprotein | Surface and membrane |
| | B [52] | |
| UL28 | ICP18.5 [54] | Processing and |
| | | packaging DNA |
| UL29 | UL29; ICP8 | Major DNA-binding |
| | [56] | protein |
| UL30 | DNA | DNA replication |
| | polymerase | |
| | [58] | |
| UL31 | UL31 [60] | Nuclear matrix protein |
| UL32 | UL32 [62] | Envelope glycoprotein |
| UL33 | UL33 [64] | Processing and |
| | | packaging DNA |
| UL34 | UL34 [66] | Inner nuclear membrane |
| | | protein |
| UL35 | VP26 [68] | Capsid protein |
| UL36 | UL36 [70] | Large tegument protein |
| UL37 | UL37 [72] | Capsid assembly |
| UL38 | UL38; | Capsid assembly and DNA |
| | VP19C | maturation |
| UL39 | UL39 | Ribonucleotide reductase |
| | | (Large subunit) |
| UL40 | UL40 | Ribonucleotide reductase |
| | | (Small subunit) |
| UL41 | UL41; VHS | Tegument protein; Virion |
| | | host shutoff^{[18]} |
| UL42 | UL42 | DNA polymerase |
| | | processivity factor |
| UL43 | UL43 | Membrane protein |
| UL44 | Glycoprotein | Surface and membrane |
| | C | |
| UL45 | UL45 | Membrane protein; C-type |
| | | lectin^{[26]} |
| UL46 | VP11/12 | Tegument proteins |
| UL47 | UL47; | Tegument protein |
| | VP13/14 | |
| UL48 | VP16 | Virion maturation; activate |
| | (Alpha-TIF) | IE genes by interacting with |
| | | the cellular transcription |
| | | factors Oct-1 and HCF. |
| | | Binds to the sequence |
| | | ^{5′}TAATGARAT^{3′}. |
| UL49 | UL49A | Envelope protein |
| UL50 | UL50 | dUTP diphosphatase |
| UL51 | UL51 | Tegument protein |
| UL52 | UL52 | DNA helicase/primase |
| | | complex protein |
| UL53 | Glycoprotein | Surface and membrane |
| | K | |
| UL54 | IE63; ICP27 | Transcriptional regulation |
| UL55 | UL55 | Unknown |
| UL56 | UL56 | Unknown |
| US1 | ICP22; IE68 | Viral replication |
| US2 | US2 | Unknown |
| US3 | US3 | Serine/threonine-protein |
| | | kinase |
| US4 | Glycoprotein | Surface and membrane |
| | G | |
| US5 | Glycoprotein | Surface and membrane |
| | J | |
| US6 | Glycoprotein | Surface and membrane |
| | D | |
| US7 | Glycoprotein | Surface and membrane |
| | I | |
| US8 | Glycoprotein | Surface and membrane |
| | E | |
| US9 | US9 | Tegument protein |
| US10 | US10 | Capsid/Tegument protein |
| US11 | US11; | Binds DNA and RNA |
| | Vmw21 | |
| US12 | ICP47; IE12 | Inhibits MHC class I |
| | | pathway by preventing |
| | | binding of antigen to TAP |
| RS1 | ICP4; IE175 | Major transcriptional |
| | | activator. Essential for |
| | | progression beyond the |
| | | immediate-early phase of |
| | | infection. IEG transcription |
| | | repressor. |
| ICP0 | ICP0; IE110; | E3 ubiquitin ligase that |
| | α0 | activates viral gene |
| | | transcription by opposing |
| | | chromatinization of the viral |
| | | genome and counteracts |
| | | intrinsic- and interferon- |
| | | based antiviral responses.^{[28]} |
| LRP1 | LRP1 | Latency-related protein |
| LRP2 | LRP2 | Latency-related protein |
| RL1 | RL1; | Neurovirulence factor. |
| | ICP34.5 | Antagonizes PKR by de- |
| | | phosphorylating eIF4a. |
| | | Binds to BECN1 and |
| | | inactivates autophagy. |
| LAT | none | Latency-associated |
| | | transcript |

HPV Target Sequences

| | |
|---|---|
| E1 | Genome replication: ATP-dependent DNA helicase |
| E2 | Genome replication, transcription, segregation, encapsidation. |
| | Regulation of cellular gene expression; cell cycle and apoptosis |
| | regulation. Several isoforms of the virus replication/transcription |
| | factor E2 have also been noted for a number of HPVs. E2 has an |
| | N-terminal domain that mediates protein-protein interactions, a |
| | flexible hinge region and a C-terminal DNA binding domain. |
| | Truncated E2 proteins may be translated from alternatively spliced |
| | E2 RNAs to generate E1\hat{ }E2 and E8\hat{ }E2 protein isoforms present |
| | in HPV16 and 31-infected cells.^{[10-13]} These E2 isoforms may act in a |
| | dominant-negative manner to modulate the function of full length |
| | E2.^{[10, 12, 13]} For example, a full length E2/E8\hat{ }E2 dimer may bind DNA but |
| | fail to recruit E1 to initiate virus replication. Similarly, such a |
| | dimer may be unable to interact with cellular transcription factors |
| | to alter virus genome transcription. |
| E4 | Remodels cytokeratin network; cell cycle arrest; virion assembly |
| E5 | Control of cell growth and differentiation; immune modulation |
| E6 | Inhibits apoptosis and differentiation; regulates cell shape, |
| | polarity, mobility and signaling. Four mRNA isoforms (FLE6, |
| | E6*I, E6*II, E6*X) have been observed in HPV16 infected |
| | cervical epithelial cells^{[16]} and two in HPV18 infection.^{[7]} A role for the |
| | E6*I isoform in antagonizing FLE6 function has been suggested,^{[7]} |
| | as has opposing roles for FLE6 and E6*I in regulation of |
| | procaspase 8 in the extrinsic apoptotic pathway.^{[18]} More recently, a |
| | stand-alone function of the E6*I isoform has been determined in |
| | cellular protein degradation.^{[9} |
| E7 | Cell cycle control; controls centrosome duplication |
| L1 | Major capsid protein |
| L2 | Minor capsid protein; recruits L1; virus assembly |
| LCR | Viral long control region (location of early promoters) |
| Keratinocyte/ | |
| auxiliary enhancer | |
| P₉₇ Promoter | Early (E) gene promoter for subtype HPV16 |
| P₁₀₅ Promoter | Early (E) gene promoter for subtype HPV18 |
| P₆₇₀ Promoter | Late (L) gene promoter for HPV16 |
| P₇₄₂ Promoter | Late (L) gene promoter for HPV31 |

Influenza A Target Sequences

Influenza A is the most common flu virus that infects humans. The influenza A virion is made up of 8 different single stranded RNA segments which encodes 11-14 proteins. These segments can vary in sequence, with most variation occurring in the hemagglutinin (H or HA) surface protein and neuraminidase (NA or N). The eight RNA segments (and the proteins they encode) are:
HA—encodes hemagglutinin (about 500 molecules of hemagglutinin are needed to make one virion).
NA—encodes neuraminidase (about 100 molecules of neuraminidase are needed to make one virion).
NP encodes nucleoprotein.
M encodes two matrix proteins (the M1 and the M2) by using different reading frames from the same RNA segment (about 3000 matrix protein molecules are needed to make one virion). M42 is produced by alternative splicing, and can partially replace an M2.
NS encodes two distinct non-structural proteins (NS1 and NEP) by using different reading frames from the same RNA segment.
PA encodes an RNA polymerase; an alternate form is sometimes made through a ribosomal skip, with +1 frameshift, reading through to the next stop codon.
PB1 encodes an RNA polymerase, plus two other transcripts read from alternate start sites, named PB1-N40 and PB1-F2 protein (induces apoptosis) by using different reading frames from the same RNA segment.
PB2 encodes an RNA polymerase.

M. tuberculosis Target Sequences

The methods and composition described herein can be used to target M. tuberculosis and treat a subject suffering from an infection with M. tuberculosis.

Other

In some embodiments, the target gene is associated with multiple drug resistance (MDR), e.g., in bacterial infection. Infectious pathogens can use a number of mechanisms in attaining multi-drug resistance, e.g., no longer relying on a glycoprotein cell wall, enzymatic deactivation of antibiotics, decreased cell wall permeability to antibiotics, altered target sites of antibiotic, efflux pumps to remove antibiotics, increased mutation rate as a stress response, or a combination thereof.

### IX. Targets: Gene Editing/Correction

Candidate Cas9 molecules, candidate gRNA molecules, and/or candidate Cas9 molecule/gRNA molecule complexes, can be used to modulate genes (e.g., mutated genes) responsible for diseases. In some embodiments, the gene is modulated by editing or correcting a target gene, e.g., as described herein. In other embodiments, the human gene is modulated by delivery of one or more regulators/effectors (e.g., as described herein) inside cells to the target gene. For example, the genes described herein can be modulated, in vitro, ex vivo, or in vivo.

TABLE IX-1
Selected Diseases in which a gene can be therapeutically targeted.

Kinases (cancer)
Energy metabolism (cancer)
CFTR (cystic fibrosis)
Color blindness
Hemochromatosis
Hemophilia
Phenylketonuria
Polycystic kidney disease
Sickle-cell disease
Tay-Sachs disease
Siderius X-linked mental retardation syndrome
Lysosomal storage disorders, e.g., Alpha-galactosidase A deficiency
Anderson-Fabry disease
Angiokeratoma Corporis Diffusum
CADASIL syndrome
Carboxylase Deficiency, Multiple, Late-Onset
Cerebelloretinal Angiomatosis, familial
Cerebral arteriopathy with subcortical infarcts and leukoencephalopathy
Cerebral autosomal dominant arteriopathy with subcortical infarcts and
leukoencephalopathy
Cerebroside Lipidosis syndrome
Choreoathetosis self-mutilation hyperuricemia syndrome
Classic Galactosemia
Crohn's disease, fibrostenosing
Phenylalanine Hydroxylase Deficiency disease,
Fabry disease
Hereditary coproporphyria
Incontinentia pigmenti
Microcephaly
Polycystic kidney disease
Rett's
Alpha-1 antitrypsin deficiency
Wilson's Disease
Tyrosinemia
Frameshift related diseases
Cystic fibrosis
Triplet repeat diseases (also referred herein as trinucleotide repeat
diseases)

Trinucleotide repeat diseases (also known as triplet repeat disease, trinucleotide repeat expansion disorders, triplet repeat expansion disorders, or codon reiteration disorders) are a set of genetic disorders caused by trinucleotide repeat expansion, e.g., a type of mutation where trinucleotide repeats in certain genes exceed the normal and/or stable threshold. The mutation can be a subset of unstable microsatellite repeats that occur in multiple or all genomic sequences. The mutation can increase the repeat count (e.g., result in extra or expanded repeats) and result in a defective gene, e.g., producing an abnormal protein. Trinucleotide repeats can be classified as insertion mutations or as a separate class of mutations. Candidate Cas9 molecules, candidate gRNA molecules, and/or candidate Cas9 molecule/gRNA molecule complexes, can be used to modulate one or more genes (e.g., mutated genes) associated with a trinucleotide repeat disease, e.g., by reducing the number of (e.g., removing) the extra or expanded repeats, such that the normal or wild-type gene product (e.g., protein) can be produced.

Exemplary trinucleotide repeat diseases and target genes involved in trinucleotide repeat diseases are shown in Table IX-1A.

TABLE IX-1A
Exemplary trinucleotide repeat diseases and target
genes involved in trinucleotide repeat diseases

| Trinucleotide Repeat Diseases | Gene |
|---|---|
| DRPLA (Dentatorubropallidoluysian atrophy) | ATN1 or DRPLA |
| HD (Huntington's disease) | HTT (Huntingtin) |
| SBMA (Spinobulbar muscular atrophy or | Androgen receptor on the |
| Kennedy disease) | X chromosome. |
| SCA1 (Spinocerebellar ataxia Type 1) | ATXN1 |
| SCA2 (Spinocerebellar ataxia Type 2) | ATXN2 |
| SCA3 (Spinocerebellar ataxia Type 3 or | ATXN3 |
| Machado-Joseph disease) | |
| SCA6 (Spinocerebellar ataxia Type 6) | CACNA1A |
| SCA7 (Spinocerebellar ataxia Type 7) | ATXN7 |
| SCA17 (Spinocerebellar ataxia Type 17) | TBP |
| FRAXA (Fragile X syndrome) | FMR1, on the X- |
| | chromosome |
| FXTAS (Fragile X-associated tremor/ | FMR1, on the X- |
| ataxia syndrome) | chromosome |
| FRAXE (Fragile XE mental retardation) | AFF2 or FMR2, on the |
| | X-chromosome |
| FRDA (Friedreich's ataxia) | FXN or X25, (frataxin- |
| | reduced expression) |
| DM (Myotonic dystrophy) | DMPK |
| SCA8 (Spinocerebellar ataxia Type 8) | OSCA or SCA8 |
| SCA12 (Spinocerebellar ataxia Type 12) | PPP2R2B or SCA12 |

Exemplary target genes include those genes involved in various diseases or conditions, e.g., cancer (e.g., kinases), energy metabolism, cystic fibrosis (e.g., CFTR), color blindness, hemochromatosis, hemophilia, phenylketonuria, polycystic kidney disease, Sickle-cell disease, Tay-Sachs disease, Siderius X-linked mental retardation syndrome, Lysosomal storage disorders (e.g., Alpha-galactosidase A deficiency), Anderson-Fabry disease, Angiokeratoma Corporis Diffusum, CADASIL syndrome, Carboxylase Deficiency, Multiple, Late-Onset, Cerebelloretinal Angiomatosis, familial, Cerebral arteriopathy with subcortical infarcts and leukoencephalopathy, Cerebral autosomal dominant arteriopathy with subcortical infarcts and leukoencephalopathy, Cerebroside Lipidosis syndrome, Choreoathetosis self-mutilation hyperuricemia syndrome, Classic Galactosemia, Crohn's disease, fibrostenosing, Phenylalanine Hydroxylase Deficiency disease, Fabry disease, Hereditary coproporphyria, Incontinentia pigmenti, Microcephaly, Polycystic kidney disease, Rett's, Alpha-1 antitrypsin deficiency, Wilson's Disease, Tyrosinemia, Frameshift related diseases, and Triplet repeat diseases.

Exemplary target genes and diseases are described in Table IX-2. The left hand column indentifies the disease and the right hand column identifies a gene for manipulation. (Table IX-2 is provided in Annex IX-2).

Additional exemplary target genes include genes associated with diseases including, e.g., Crigler-Najjer syndrome, Glycogen storage disease type IV (GSD type IV), Familial hemophagocytic lymphohistiocytosis (FHL-Perforin deficiency), Ornithine transcarbamylase deficiency (OTC deficiency) or other Urea Cycle Disorders, Primary Hyperoxaluria, Leber congenital amaurosis (LCA), Batten disease, Chronic Granulomatous Disease, Wiskott-Aldrich syndrome, Usher Syndrome, and hemoglobinoapthies.

Crigler-Najjer syndrome. Crigler-Najjer syndrome is a severe condition characterized by high levels of bilirubin in the blood (hyperbilirubinemia). Bilirubin is produced when red blood cells are broken down. This substance is removed from the body only after it undergoes a chemical reaction in the liver, which converts the toxic form of bilirubin (unconjugated bilirubin) to a nontoxic form (conjugated bilirubin). People with Crigler-Najjar syndrome have a buildup of unconjugated bilirubin in their blood (unconjugated hyperbilirubinemia). Crigler-Najjar syndrome is divided into two types. Type 1 (CN1) is very severe and Type 2 (CN2) is less severe.

Mutations in the UGT1A1 gene can cause Crigler-Najjar syndrome. This gene provides instructions for making the bilirubin uridine diphosphate glucuronosyl transferase (bilirubin-UGT) enzyme, which is found primarily in liver cells and is necessary for the removal of bilirubin from the body. The bilirubin-UGT enzyme is involved in glucuronidation, in which the enzyme transfers glucuronic acid to unconjugated bilirubin, converting it to conjugated bilirubin. Glucuronidation makes bilirubin dissolvable in water so that it can be removed from the body.

Mutations in the UGT1A1 gene that cause Crigler-Najjar syndrome result in reduced or absent function of the bilirubin-UGT enzyme. People with CN1 have no enzyme function, while people with CN2 can have less than 20 percent of normal function. The loss of bilirubin-UGT function decreases glucuronidation of unconjugated bilirubin. This toxic substance then builds up in the body, causing unconjugated hyperbilirubinemia and jaundice.

Glycogen storage disease type IV. Glycogen storage disease type IV (also known as GSD type IV, Glycogenosis type IV, Glycogen Branching Enzyme Deficiency (GBED), polyglucosan body disease, or Amylopectinosis) is an inherited disorder caused by the buildup of a complex sugar called glycogen in the body's cells. The accumulated glycogen is structurally abnormal and impairs the function of certain organs and tissues, especially the liver and muscles.

Mutations in the GBE1 gene cause GSD IV. The GBE1 gene provides instructions for making the glycogen branching enzyme. This enzyme is involved in the production of glycogen, which is a major source of stored energy in the body. GBE1 gene mutations that cause GSD IV lead to a shortage (deficiency) of the glycogen branching enzyme. As a result, glycogen is not formed properly. Abnormal glycogen molecules called polyglucosan bodies accumulate in cells, leading to damage and cell death. Polyglucosan bodies accumulate in cells throughout the body, but liver cells and muscle cells are most severely affected in GSD IV. Glycogen accumulation in the liver leads to hepatomegaly and interferes with liver functioning. The inability of muscle cells to break down glycogen for energy leads to muscle weakness and wasting.

Generally, the severity of the disorder is linked to the amount of functional glycogen branching enzyme that is produced. Individuals with the fatal perinatal neuromuscular type tend to produce less than 5 percent of usable enzyme, while those with the childhood neuromuscular type may have around 20 percent of enzyme function. The other types of GSD IV are usually associated with between 5 and 20 percent of working enzyme. These estimates, however, vary among the different types.

Familial hemophagocytic lymphohistiocytosis. Familial hemophagocytic lymphohistiocytosis (FHL) is a disorder in which the immune system produces too many activated immune cells (lymphocytes), e.g., T cells, natural killer cells, B cells, and macrophages (histiocytes). Excessive amounts of cytokines are also produced. This overactivation of the immune system causes fever and damages the liver and spleen, resulting in enlargement of these organs.

Familial hemophagocytic lymphohistiocytosis also destroys blood-producing cells in the bone marrow, a process called hemophagocytosis. The brain may also be affected in familial hemophagocytic lymphohistiocytosis. In addition to neurological problems, familial hemophagocytic lymphohistiocytosis can cause abnormalities of the heart, kidneys, and other organs and tissues. Affected individuals also have an increased risk of developing cancers of blood-forming cells (leukemia and lymphoma).

Familial hemophagocytic lymphohistiocytosis may be caused by mutations in any of several genes. These genes provide instructions for making proteins that help destroy or deactivate lymphocytes that are no longer needed. By controlling the number of activated lymphocytes, these genes help regulate immune system function.

Approximately 40 to 60 percent of cases of familial hemophagocytic lymphohistiocytosis are caused by mutations in the PRF1 or UNC13D genes. Smaller numbers of cases are caused by mutations in other known genes such as STX11 or STXBP2. The gene mutations that cause familial hemophagocytic lymphohistiocytosis can impair the body's ability to regulate the immune system. These changes result in the exaggerated immune response characteristic of this condition.

Ornithine transcarbamylase deficiency. Ornithine transcarbamylase deficiency (OTC) is an inherited disorder that causes ammonia to accumulate in the blood.

Mutations in the OTC gene cause ornithine transcarbamylase deficiency.

Ornithine transcarbamylase deficiency belongs to a class of genetic diseases called urea cycle disorders. The urea cycle is a sequence of reactions that occurs in liver cells. It processes excess nitrogen, generated when protein is used by the body, to make a compound called urea that is excreted by the kidneys.

In ornithine transcarbamylase deficiency, the enzyme that starts a specific reaction within the urea cycle is damaged or missing. The urea cycle cannot proceed normally, and nitrogen accumulates in the bloodstream in the form of ammonia.

Ammonia is especially damaging to the nervous system, so ornithine transcarbamylase deficiency causes neurological problems as well as eventual damage to the liver.

Other urea cycle disorders and associate genes include, e.g., N-Acetylglutamate synthase deficiency (NAGS), Carbamoyl phosphate synthetase I deficiency (CPS1), “AS deficiency” or citrullinemia (ASS), “AL deficiency” or argininosuccinic aciduria (ASL), and “Arginase deficiency” or argininemia (ARG).

Primary hyperoxaluria. Primary hyperoxaluria, e.g., primary hyperoxaluria type 1 (PH1), is a rare, autosomal recessive inherited genetic condition in which an error in the glyoxylate metabolism pathway in the liver leads to an overproduction of oxalate, which crystalizes in soft tissues including the kidney, bone marrow, and eyes. The disease manifests as progressive deterioration of the kidneys, and treatment is a complicated double transplant of kidney (the damaged organ) and liver (the diseased organ).

Primary hyperoxaluria is caused by the deficiency of an enzyme that normally prevents the buildup of oxalate. There are two types of primary hyperoxaluria, distinguished by the enzyme that is deficient. People with type 1 primary hyperoxaluria have a shortage of a liver enzyme called alanine-glyoxylate aminotransferase (AGXT). Type 2 primary hyperoxaluria is characterized by a shortage of an enzyme called glyoxylate reductase/hydroxypyruvate reductase (GRHPR).

Mutations in the AGXT and GRHPR genes cause primary hyperoxaluria. The breakdown and processing of certain sugars and amino acids produces a glyoxylate. Normally, glyoxylate is converted to the amino acid glycine or to glycolate through the action of two enzymes, alanine-glyoxylate aminotransferase and glyoxylate reductase/hydroxypyruvate reductase, respectively. Mutations in the AGXT or GRHPR gene cause a shortage of these enzymes, which prevents the conversion of glyoxylate to glycine or glycolate. As levels of glyoxylate build up, it is converted to oxalate. Oxalate combines with calcium to form calcium oxalate deposits, which can damage the kidneys and other organs.

In an embodiment, the genetic defect in AGXT is corrected, e.g., by homologous recombination, using the Cas9 molecule and gRNA molecule described herein. For example, the functional enzyme encoded by the corrected AGXT gene can be redirected to its proper subcellular organelle. Though >50 mutations have been identified in the gene, the most common (40% in Caucasians) is a missense G170R mutation. This mutation causes the AGT enzyme to be localized to the mitochondria rather than to the peroxisome, where it must reside to perform its function. Other common mutations include, e.g., I244T (Canary Islands), F152I, G41R, G630A (Italy), and G588A (Italy).

In an embodiment, one or more genes encoding enzymes upstream in the glyoxylate metabolism pathway are targeted, using the Cas9 molecule and gRNA molecule described herein. Exemplary targets include, e.g., glycolate oxidase (gene HAO1, OMIM ID 605023). Glycolate oxidase converts glycolate into glyoxylate, the substrate for AGT. Glycolate oxidase is only expressed in the liver and, because of its peroxisomal localization, makes it a suitable target in this metabolic pathway. In an embodiment, a double-strand break in the HAO1 gene is introduced and upon repair by NHEJ a frame-shift results in a truncated protein. In an embodiment, a transcriptional repressor (e.g., a transcriptional repressor described herein) is delivered as a payload to the HAO1 gene to reduce the expression of HAO1.

Leber congenital amaurosis. Leber congenital amaurosis (LCA) is an eye disorder that primarily affects the retina. People with this disorder typically have severe visual impairment beginning in infancy. The visual impairment tends to be stable, although it may worsen very slowly over time. At least 13 types of Leber congenital amaurosis have been described. The types are distinguished by their genetic cause, patterns of vision loss, and related eye abnormalities.

Leber congenital amaurosis can result from mutations in at least 14 genes, all of which are necessary for normal vision. These genes play a variety of roles in the development and function of the retina. For example, some of the genes associated with this disorder are necessary for the normal development of photoreceptors. Other genes are involved in phototransduction. Still other genes play a role in the function of cilia, which are necessary for the perception of several types of sensory input, including vision.

Mutations in any of the genes associated with Leber congenital amaurosis (e.g., AIPL1, CEP290, CRB1, CRX, GUCY2D, IMPDH1, LCA5, LRAT, RD3, RDH12, RPE65, RPGRIP1, SPATA7, TULP1) can disrupt the development and function of the retina, resulting in early vision loss. Mutations in the CEP290, CRB1, GUCY2D, and RPE65 genes are the most common causes of the disorder, while mutations in the other genes generally account for a smaller percentage of cases.

Batten disease. Batten disease or juvenile Batten disease is an inherited disorder that primarily affects the nervous system. After a few years of normal development, children with this condition develop progressive vision loss, intellectual and motor disability, and seizures.

Juvenile Batten disease is one of a group of disorders known as neuronal ceroid lipofuscinoses (NCLs). These disorders all affect the nervous system and typically cause progressive problems with vision, movement, and thinking ability. Some people refer to the entire group of NCLs as Batten disease, while others limit that designation to the juvenile form of the disorder. The different types of NCLs are distinguished by the age at which signs and symptoms first appear.

Most cases of juvenile Batten disease are caused by mutations in the CLN3 gene. These mutations can disrupt the function of cellular structures called lysosomes. Lysosome malfunction leads to a buildup of lipopigments within these cell structures. These accumulations occur in cells throughout the body, but neurons in the brain seem to be particularly vulnerable to the damage caused by lipopigments. The progressive death of cells, especially in the brain, leads to vision loss, seizures, and intellectual decline in people with juvenile Batten disease.

A small percentage of cases of juvenile Batten disease are caused by mutations in other genes (e.g., ATP13A2, CLN5, PPT1, TPP1). Many of these genes are involved in lysosomal function, and when mutated, can cause this or other forms of NCL.

Chronic granulomatous disease. Chronic granulomatous disease is a disorder that causes the immune system to malfunction, resulting in a form of immunodeficiency. Individuals with chronic granulomatous disease have recurrent bacterial and fungal infections. People with this condition often have areas of inflammation (granulomas) in various tissues that can be damaging to those tissues. The features of chronic granulomatous disease usually first appear in childhood, although some individuals do not show symptoms until later in life.

Mutations in the CYBA, CYBB, NCF1, NCF2, or NCF4 gene can cause chronic granulomatous disease. There are five types of this condition that are distinguished by the gene that is involved. The proteins produced from the affected genes are subunits of NADPH oxidase, which plays an important role in the immune system. Specifically, NADPH oxidase is primarily active in phagocytes. Within phagocytes, NADPH oxidase is involved in the production of superoxide, which plays a role in killing foreign invaders and preventing them from reproducing in the body and causing illness. NADPH oxidase also regulates the activity of neutrophils, which play a role in adjusting the inflammatory response to optimize healing and reduce injury to the body.

Mutations in the CYBA, CYBB, NCF1, NCF2, and NCF4 genes result in the production of proteins with little or no function or the production of no protein at all. Without any one of its subunit proteins, NADPH oxidase cannot assemble or function properly. As a result, phagocytes are unable to kill foreign invaders and neutrophil activity is not regulated. A lack of NADPH oxidase leaves affected individuals vulnerable to many types of infection and excessive inflammation.

Wiskott-Aldrich syndrome. Wiskott-Aldrich syndrome is characterized by abnormal immune system function (immune deficiency) and a reduced ability to form blood clots. This condition primarily affects males. Individuals with Wiskott-Aldrich syndrome have microthrombocytopenia, which is a decrease in the number and size of blood cells involved in clotting (platelets), which can lead to easy bruising or episodes of prolonged bleeding following minor trauma. Wiskott-Aldrich syndrome causes many types of white blood cells to be abnormal or nonfunctional, leading to an increased risk of several immune and inflammatory disorders. Many people with this condition develop eczema, an inflammatory skin disorder characterized by abnormal patches of red, irritated skin. Affected individuals also have an increased susceptibility to infection. People with Wiskott-Aldrich syndrome are at greater risk of developing autoimmune disorders. The chance of developing some types of cancer, such as cancer of the immune system cells (lymphoma), is also greater in people with Wiskott-Aldrich syndrome.

Mutations in the WAS gene cause Wiskott-Aldrich syndrome. The WAS gene provides instructions for making WASP protein, which is found in all blood cells. WASP is involved in relaying signals from the surface of blood cells to the actin cytoskeleton. WASP signaling activates the cell when it is needed and triggers its movement and attachment to other cells and tissues (adhesion). In white blood cells, this signaling allows the actin cytoskeleton to establish the interaction between cells and the foreign invaders that they target (immune synapse).

WAS gene mutations that cause Wiskott-Aldrich syndrome lead to a lack of any functional WASP. Loss of WASP signaling disrupts the function of the actin cytoskeleton in developing blood cells. White blood cells that lack WASP have a decreased ability to respond to their environment and form immune synapses. As a result, white blood cells are less able to respond to foreign invaders, causing many of the immune problems related to Wiskott-Aldrich syndrome. Similarly, a lack of functional WASP in platelets impairs their development, leading to reduced size and early cell death.

Usher syndrome. Usher syndrome is a condition characterized by hearing loss or deafness and progressive vision loss. The loss of vision is caused by retinitis pigmentosa (RP), which affects the layer of light-sensitive tissue at the back of the eye (the retina). Vision loss occurs as the light-sensing cells of the retina gradually deteriorate.

Three major types of Usher syndrome, designated as types I (subtypes IA through IG), II (subtypes IIA, IIB, and IIC), and III, have been identified. These types are distinguished by their severity and the age when signs and symptoms appear.

Mutations in the CDH23, CLRN1, GPR98, MYO7A, PCDH15, USH1C, USH1G, and USH2A genes can cause Usher syndrome. The genes related to Usher syndrome provide instructions for making proteins that play important roles in normal hearing, balance, and vision. They function in the development and maintenance of hair cells, which are sensory cells in the inner ear that help transmit sound and motion signals to the brain. In the retina, these genes are also involved in determining the structure and function of light-sensing cells called rods and cones. In some cases, the exact role of these genes in hearing and vision is unknown. Most of the mutations responsible for Usher syndrome lead to a loss of hair cells in the inner ear and a gradual loss of rods and cones in the retina. Degeneration of these sensory cells causes hearing loss, balance problems, and vision loss characteristic of this condition.

Usher syndrome type I can result from mutations in the CDH23, MYO7A, PCDH15, USH1C, or USH1G gene. Usher syndrome type II can be caused by mutations in, e.g., USH2A or GPR98 (also called VLGR1) gene. Usher syndrome type III can be caused by mutations in e.g., CLRN1.

Hemoglobinopathies. Hemoglobinopathies are a group of genetic defects that result in abnormal structure of one of the globin chains of the hemoglobin molecule. Exemplary hemoglobinopathies include, e.g., sickle cell disease, alpha thalassemia, and beta thalassemia.

In an embodiment, a genetic defect in alpha globulin or beta globulin is corrected, e.g., by homologous recombination, using the Cas9 molecule and gRNA molecule described herein.

In an embodiment, a hemoglobinopathies-associated gene is targeted, using the Cas9 molecule and gRNA molecule described herein. Exemplary targets include, e.g., genes associated with control of the gamma-globin genes. In an embodiment, the target is BCL11A.

Fetal hemoglobin (also hemoglobin F or HbF or a272) is a tetramer of two adult alpha-globin polypeptides and two fetal beta-like gamma-globin polypeptides. HbF is the main oxygen transport protein in the human fetus during the last seven months of development in the uterus and in the newborn until roughly 6 months old. Functionally, fetal hemoglobin differs most from adult hemoglobin in that it is able to bind oxygen with greater affinity than the adult form, giving the developing fetus better access to oxygen from the mother's bloodstream.

In newborns, fetal hemoglobin is nearly completely replaced by adult hemoglobin by approximately 6 months postnatally. In adults, fetal hemoglobin production can be reactivated pharmacologically, which is useful in the treatment of diseases such as hemoglobinopathies. For example, in certain patients with hemoglobinopathies, higher levels of gamma-globin expression can partially compensate for defective or impaired beta-globin gene production, which can ameliorate the clinical severity in these diseases. Increased HbF levels or F-cell (HbF containing erythrocyte) numbers can ameliorate the disease severity of hemoglobinopathies, e.g., beta-thalassemia major and sickle cell anemia.

Increased HbF levels or F-cell can be associated reduced BCL11A expression in cells. The BCL11A gene encodes a multi-zinc finger transcription factor. In an embodiment, the expression of BCL11A is modulated, e.g., down-regulated. In an embodiment, the BCL11A gene is edited. In an embodiment, the cell is a hemopoietic stem cell or progenitor cell.

Sickle Cell Diseases

Sickle cell disease is a group of disorders that affects hemoglobin. People with this disorder have atypical hemoglobin molecules (hemoglobin S), which can distort red blood cells into a sickle, or crescent, shape. Characteristic features of this disorder include a low number of red blood cells (anemia), repeated infections, and periodic episodes of pain.

Mutations in the HBB gene cause sickle cell disease. The HBB gene provides instructions for making beta-globin. Various versions of beta-globin result from different mutations in the HBB gene. One particular HBB gene mutation produces an abnormal version of beta-globin known as hemoglobin S (HbS). Other mutations in the HBB gene lead to additional abnormal versions of beta-globin such as hemoglobin C (HbC) and hemoglobin E (HbE). HBB gene mutations can also result in an unusually low level of beta-globin, i.e., beta thalassemia.

In people with sickle cell disease, at least one of the beta-globin subunits in hemoglobin is replaced with hemoglobin S. In sickle cell anemia, which is a common form of sickle cell disease, hemoglobin S replaces both beta-globin subunits in hemoglobin. In other types of sickle cell disease, just one beta-globin subunit in hemoglobin is replaced with hemoglobin S. The other beta-globin subunit is replaced with a different abnormal variant, such as hemoglobin C. For example, people with sickle-hemoglobin C (HbSC) disease have hemoglobin molecules with hemoglobin S and hemoglobin C instead of beta-globin. If mutations that produce hemoglobin S and beta thalassemia occur together, individuals have hemoglobin S-beta thalassemia (HbSBetaThal) disease.

Alpha Thalassemia

Alpha thalassemia is a blood disorder that reduces the production of hemoglobin. In people with the characteristic features of alpha thalassemia, a reduction in the amount of hemoglobin prevents enough oxygen from reaching the body's tissues. Affected individuals also have a shortage of red blood cells (anemia), which can cause pale skin, weakness, fatigue, and more serious complications.

Two types of alpha thalassemia can cause health problems. The more severe type is hemoglobin Bart hydrops fetalis syndrome or Hb Bart syndrome. The milder form is HbH disease. Hb Bart syndrome is characterized, e.g., by hydrops fetalis, a condition in which excess fluid builds up in the body before birth. HbH disease can cause, e.g., mild to moderate anemia, hepatosplenomegaly, and yellowing of the eyes and skin (jaundice).

Alpha thalassemia typically results from deletions involving the HBA1 and HBA2 genes. Both of these genes provide instructions for making alpha-globin, which is a subunit of hemoglobin. The different types of alpha thalassemia result from the loss of some or all of these alleles.

Hb Bart syndrome can result from the loss of all four alpha-globin alleles. HbH disease can be caused by a loss of three of the four alpha-globin alleles. In these two conditions, a shortage of alpha-globin prevents cells from making normal hemoglobin. Instead, cells produce abnormal forms of hemoglobin, i.e., hemoglobin Bart (Hb Bart) or hemoglobin H (HbH), which cannot effectively carry oxygen to the body's tissues. The substitution of Hb Bart or HbH for normal hemoglobin can cause anemia and the other serious health problems associated with alpha thalassemia.

Two additional variants of alpha thalassemia are related to a reduced amount of alpha-globin. A loss of two of the four alpha-globin alleles can result in alpha thalassemia trait. People with alpha thalassemia trait may have unusually small, pale red blood cells and mild anemia. A loss of one alpha-globin allele can be found in alpha thalassemia silent carriers.

Beta Thalassemia

Beta thalassemia is a blood disorder that reduces the production of hemoglobin. In people with beta thalassemia, low levels of hemoglobin lead to a lack of oxygen in many parts of the body. Affected individuals also have a shortage of red blood cells (anemia), which can cause pale skin, weakness, fatigue, and more serious complications. People with beta thalassemia are at an increased risk of developing abnormal blood clots.

Beta thalassemia is classified into two types depending on the severity of symptoms: thalassemia major (also known as Cooley's anemia) and thalassemia intermedia. Of the two types, thalassemia major is more severe.

Mutations in the HBB gene cause beta thalassemia. The HBB gene provides instructions for making beta-globin. Some mutations in the HBB gene prevent the production of any beta-globin. The absence of beta-globin is referred to as beta-zero (B⁰) thalassemia. Other HBB gene mutations allow some beta-globin to be produced but in reduced amounts, i.e., beta-plus (B⁺) thalassemia. People with both types have been diagnosed with thalassemia major and thalassemia intermedia.

In an embodiment, a Cas9 molecule/gRNA molecule complex targeting a first gene is used to treat a disorder characterized by second gene, e.g., a mutation in a second gene. By way of example, targeting of the first gene, e.g., by editing or payload delivery, can compensate for, or inhibit further damage from, the affect of a second gene, e.g., a mutant second gene. In an embodiment the allele(s) of the first gene carried by the subject is not causative of the disorder.

TABLE IX-3
Selected Disorders and Targets for Compensatory Targeting

| | Indication | | | | | | |
|---|---|---|---|---|---|---|---|
| | | | | | Non- | | Prevention of |
| | | | | | Hodgkin's | | organ |
| | | | | | lymphoma, | | transplant |
| | | | | | Chronic | | rejection, |
| | Age-Related Macular | | Atypical Hemolytic Uremic | | lymphocytic | Rheumatoid | renal cell |
| | Degeneration | | Syndrome | | leukemia | Arthritis | carcinoma |
| | Target | | | | | | |
| | Factor H | C5 | Factor H | C5 | CD20 | CD21 | mTORC1 |
| Up- | up-regulate | down-regulate | up-regulate | down-regulate | down-regulate | down-regulate | down-regulate |
| regulate/ | | | | | | | |
| Down- | | | | | | | |
| regulate | | | | | | | |
| Level of | animal | | Factor H | Eculizumab/ | Rituxan | Rituxan | everolimus |
| evidence: | models | | concentrate | Soliris c5Ab | (Genentech) | (Genentech) | |
| Market | | | | (Alexion) | CD20 | CD20 | |
| proxy or | | | | successful in | antibody | antibody | |
| animal | | | | decreasing | | | |
| model | | | | mortality | | | |

| | | |
|---|---|---|
| Comment | Muti-genetic origin. Factor H | aHUS due to fH deficiency. |
| | deficiency is a risk factor. | C5 antibody has been shown |
| | Controlling the complement | to vastly improve prognosis. |
| | cascade, through fH | Can approach disease directly |
| | upregulation or C5 | through increasing fH levels |
| | downregulation, may have a | or controlling complement |
| | beneficial effect. | through C5 downregulation. |

| | Indication | | | | | | |
|---|---|---|---|---|---|---|---|
| | Devices: | | | | | | |
| | stent, | | | | | | |
| | pacemaker, | | | | | | |
| | hernia mesh- | Graft | orthopedics- | | | | |
| | local delivery | healing/wound | articular | | | | Barrett's |
| | to prevent | healing/ | cartilage | | | | esophagus, |
| | restenosis/ | prevention of | repair, | Parkinson's | Allergic | | Stomach |
| | fibrosis | fibrosis | arthritis | Disease | rhinitis | Epilepsy | ulcer, gastritis |
| | Target | | | | | | |
| | | | | | H1 | | |
| | | | | SNCA, | Receptors | | H2 receptor |
| | mTORC2, | | | LRRK2, | nasal | H1 receptors | pylorus, |
| | others | VEGF | IL-11 | EIF4GI | mucosa | CNS | esophagus |
| Upregulate/ | down-regulate | up-regulate | up-regulate | up-regulate | down-regulate | up-regulate | down-regulate |
| Downregulate | | | | or fix | | | |
| | | | | mutations | | | |
| Level of | everolimus | VEGF local | animal model | | H1-anti- | animal | H2-specific |
| evidence: | | administration | of cartilage | | histamines, | models | antihistamines, |
| Market | | aids in | repair | | e.g. Zyrtec | | e.g. |
| proxy or | | tracheal | | | | | omeprazole, |
| animal | | transplant | | | | | etc. |
| model | | animal | | | | | |
| | | models | | | | | |
| Comment | Embodiments | Useful, e.g., | In | | | . | In |
| | include, e.g., | in the | embodiments, | | | | embodiments, |
| | local delivery | promoting | the subject | | | | the subject is |
| | to tissue via | wound | sufferes from | | | | treated for |
| | device or | healing | arthritis or is | | | | late-stage |
| | injection to | (burns, etc); | in need of | | | | barrett's. |
| | prevent | Embodiments | healing after | | | | |
| | fibrosis, | include, e.g., | injury. In | | | | |
| | restenosis | local delivery | embodiments, | | | | |
| | | of growth | chondrocytes | | | | |
| | | factors | are targeted | | | | |
| | | | post-injury to | | | | |
| | | | promote | | | | |
| | | | healing. | | | | |

In an embodiment, Cas9 molecules, gRNA molecules, and/or Cas9 molecule/gRNA molecule complexes can be used to target, e.g., result in repression of, knockout of, or alteration of promoter for key transcription factors, such as BCL11A and KLF1 for up-regulating of fetal hemoglobin, e.g., for cure for sickle cell anemia and thalassemia.

Candidate Cas9 molecules, candidate gRNA molecules, and/or candidate Cas9 molecule/gRNA molecule complexes, as described herein, can be used to edit/correct a target gene or to deliver a regulator/effector inside cells, e.g., as described herein, at various subcellular locations. In some embodiments, the location is in the nucleus. In some embodiments, the location is in a sub-nuclear domain, e.g., the chromosome territories, nucleolus, nuclear speckles, Cajal bodies, Gems (gemini of Cajal bodies), or promyelocytic leukemia (PML) nuclear bodies. In other embodiments, the location is in the mitochondrion.

Candidate Cas9 molecules, candidate gRNA molecules, and/or candidate Cas9 molecule/gRNA molecule complexes, as described herein, can be used to edit/correct a target gene or to deliver a regulator/effector inside cells, as described herein, at various time points

For example, the editing/correction or delivery can occur at different phases of cell cycle, e.g., G0 phase, Interphase (e.g., G1 phase, S phase, G2 phase), or M phase. As another example, the editing/correction or delivery can occur at different stages of disease progression, e.g., at latent stage or active stage of a disorder (e.g., viral infection), or at any stage or subclassification of a disorder (e.g., cancer).

Methods of the invention allow for the treatment of a disorder characterized by unwanted cell proliferation, e.g., cancer. In an embodiment, cancer cells are manipulated to make them more susceptible to treatment or to endogenous immune surveillance. In an embodiment a cancer cell is modulated to make it more susceptible to a therapeutic. In an embodiment, a cancer cell is manipulated so as to increase the expression of a gene that increases the ability of the immune system to recognize or kill the cancer cell. E.g., a Cas9 molecule/gRNA molecule complex can be used to deliver a payload, or edit a target nucleic acid so as to increase the expression of an antigen, e.g., in the case where the cancer cell has downregulated expression of the antigen. In an embodiment, a payload, e.g., a payload comprising a transcription factor or other activator of expression is delivered to the cancer cell. In an embodiment, an increase in expression is effected by cleavage of the target nucleic acid, e.g., cleavage and correction or alteration of the target nucleic acid by a template nucleic acid. In an embodiment, a payload that overrides epigenetic silencing, e.g., a modulator of methylation, is delivered.

In an embodiment, the treatment further comprises administering a second anti-cancer therapy, e.g., immunotherapy, e.g., an antibody that binds the upregulated antigen.

In an embodiment, methods described herein, e.g., targeting of a genomic signature, e.g., a somatic translocation, can be used to target the Cas9 molecule/gRNA molecule to a cancer cell.

In another aspect, the invention features a method of immunizing a subject against an antigen. The method comprises using a method described herein to promote the expression of the antigen from a cell, e.g., a blood cell, such that the antigen promotes an immune response. In an embodiment, the cell is manipulated ex vivo and then returned or introduced into the subject.

### X. Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:
(i) alteration, e.g., replacement, of one or both of the non-linking phosphate oxygens and/or of one or more of the linking phosphate oxygens in the phosphodiester backbone linkage;
(ii) alteration, e.g., replacement, of a constituent of the ribose sugar, e.g., of the 2′ hydroxyl on the ribose sugar;
(iii) wholesale replacement of the phosphate moiety with “dephospho” linkers;
(iv) modification or replacement of a naturally occurring nucleobase;
(v) replacement or modification of the ribose-phosphate backbone;
(vi) modification of the 3′ end or 5′ end of the oligonucleotide, e.g., removal, modification or replacement of a terminal phosphate group or conjugation of a moiety; and
(vii) modification of the sugar.

The modifications listed above can be combined to provide modified nucleosides and nucleotides that can have two, three, four, or more modifications. For example, a modified nucleoside or nucleotide can have a modified sugar and a modified nucleobase. In an embodiment, every base of a gRNA is modified, e.g., all bases have a modified phosphate group, e.g., all are phosphorothioate groups. In an embodiment, all, or substantially all, of the phosphate groups of a unimolecular or modular gRNA molecule are replaced with phosphorothioate groups.

In an embodiment, modified nucleotides, e.g., nucleotides having modifications as described herein, can be incorporated into a nucleic acid, e.g., a “modified nucleic acid.” In some embodiments, the modified nucleic acids comprise one, two, three or more modified nucleotides. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in one aspect the modified nucleic acids described herein can contain one or more modified nucleosides or nucleotides, e.g., to introduce stability toward nucleases.

In some embodiments, the modified nucleosides, modified nucleotides, and modified nucleic acids described herein can exhibit a reduced innate immune response when introduced into a population of cells, both in vivo and ex vivo. The term “innate immune response” includes a cellular response to exogenous nucleic acids, including single stranded nucleic acids, generally of viral or bacterial origin, which involves the induction of cytokine expression and release, particularly the interferons, and cell death. In some embodiments, the modified nucleosides, modified nucleotides, and modified nucleic acids described herein can disrupt binding of a major groove interacting partner with the nucleic acid. In some embodiments, the modified nucleosides, modified nucleotides, and modified nucleic acids described herein can exhibit a reduced innate immune response when introduced into a population of cells, both in vivo and ex vivo, and also disrupt binding of a major groove interacting partner with the nucleic acid.

### Definitions of Chemical Groups

As used herein, “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 12, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “alkenyl” refers to an aliphatic group containing at least one double bond.

As used herein, “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl.

As used herein, “arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

As used herein, “cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl.

As used herein, “heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl.

As used herein, “heteroaryl” refers to a monovalent radical of a heteroaromatic ring system. Examples of heteroaryl moieties include, but are not limited to, imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

Phosphate Backbone Modifications

The Phosphate Group

In some embodiments, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In some embodiments, the modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

Replacement of the Phosphate Group

The phosphate group can be replaced by non-phosphorus containing connectors. In some embodiments, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Replacement of the Ribophosphate Backbone

Scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include, without limitation, the morpholino, cyclobutyl, pyrrolidine and peptide nucleic acid (PNA) nucleoside surrogates.

Sugar Modifications

The modified nucleosides and modified nucleotides can include one or more modifications to the sugar group. For example, the 2′ hydroxyl group (OH) can be modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative).

“Deoxy” modifications can include hydrogen (i.e. deoxyribose sugars, e.g., at the overhang portions of partially ds RNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the 1′ position on the sugar, e.g., alpha-nucleosides. The modified nucleic acids can also include “abasic” sugars, which lack a nucleobase at C-1′. These abasic sugars can also be further modified at one or more of the constituent sugar atoms. The modified nucleic acids can also include one or more sugars that are in the L form, e.g. L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replaced with α-L-threofuranosyl-(3′→2′)).

Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In some embodiments, the nucleobase can include, for example, naturally-occurring and synthetic derivatives of a base.

Uracil

In some embodiments, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (memo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τcm⁵U), 1-taurinomethyl-pseudouridine, 5-taurinomethyl-2-thio-uridine(τm⁵s2U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s2U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s2U), a-thio-uridine, 2′-O-methyl-uridine (Um), 5,2′-O-dimethyl-uridine (m⁵Um), 2′-O-methyl-pseudouridine (Wm), 2-thio-2′-O-methyl-uridine (s2Um), 5-methoxycarbonylmethyl-2′-O-methyl-uridine (mem⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

Cytosine

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m³C), N4-acetyl-cytidine (act), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k²C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

Adenine

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms2 m⁶A), N6-isopentenyl-adenosine (i⁶A), 2-methylthio-N6-isopentenyl-adenosine (ms²₆A), N6-(cis-hydroxyisopentenyl)adenosine (io⁶A), 2-methylthio-N6-(cis-hydroxyisopentenyl)adenosine (ms2io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g6A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms2hn⁶A), N6-acetyl-adenosine (ac⁶A), 7-methyl-adenine, 2-methylthio-adenine, 2-methoxy-adenine, a-thio-adenosine, 2′-O-methyl-adenosine (Am), N⁶,2′-O-dimethyl-adenosine (m⁶Am), N⁶-Methyl-2′-deoxyadenosine, N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

Guanine

In some embodiments, the modified nucleobase is a modified guanine. Exemplary nucleobases and nucleosides having a modified guanine include without limitation inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m′G), N2-methyl-guanosine (m²G), N2,N2-dimethyl-guanosine (m²₂G), N2,7-dimethyl-guanosine (m²,7G), N2,N2,7-dimethyl-guanosine (m²,2,7G), 8-oxo-guanosine, 7-methyl-8-oxo-guanosine, 1-meth thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m²Gm), N2,N2-dimethyl-2′-O-methyl-guanosine (m² 2Gm), 1-methyl-2′-O-methyl-guanosine (m′Gm), N2,7-dimethyl-2′-O-methyl-guanosine (m²,7Gm), 2′-O-methyl-inosine (Im), 1,2′-O-dimethyl-inosine (m′Im), O⁶-phenyl-2′-deoxyinosine, 2′-O-ribosylguanosine (phosphate) (Gr(p)), 1-thio-guanosine, O⁶-methyl-guanosine, O⁶-Methyl-2′-deoxyguanosine, 2′-F-ara-guanosine, and 2′-F-guanosine.

Modified gRNAs

In some embodiments, the modified nucleic acids can be modified gRNAs. In some embodiments, gRNAs can be modified at the 3′ end. In this embodiment, the gRNAs can be modified at the 3′ terminal U ribose. For example, the two terminal hydroxyl groups of the U ribose can be oxidized to aldehyde groups and a concomitant opening of the ribose ring to afford a modified nucleoside as sown below:

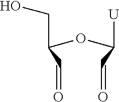

wherein “U” can be an unmodified or modified uridine.

In another embodiment, the 3′ terminal U can be modified with a 2′3′ cyclic phosphate as shown below:

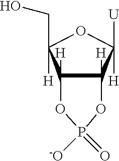

wherein “U” can be an unmodified or modified uridine.

In some embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyl adenosine, can be incorporated into the gRNA. In some embodiments, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In an embodiment, a one or more or all of the nucleotides in single stranded overhang of an RNA molecule, e.g., a gRNA molecule, are deoxynucleotides.

### XI. Linkers

In some embodiments, the payload can be linked to the Cas9 molecules or the gRNA, e.g., by a covalent linker. This linker may be cleavable or non-cleavable. In some embodiments, a cleavable linker may be used to release the payload after transport to the desired target.

Linkers can comprise a direct bond or an atom such as, e.g., an oxygen (O) or sulfur (S), a unit such as —NR— wherein R is hydrogen or alkyl, —C(O)—, —C(O)O—, —C(O)NH—, SO, SO₂, —SO₂NH— or a chain of atoms, such as substituted or unsubstituted alkyl, substituted or unsubstituted alkenyl, substituted or unsubstituted alkynyl, arylalkyl, heteroarylalkyl. In some embodiments, one or more methylenes in the chain of atoms can be replaced with one or more of O, S, S(O), SO₂, —SO₂NH—, —NR—, —C(O)—, —C(O)O—, —C(O)NH—, a cleavable linking group, substituted or unsubstituted aryl, substituted or unsubstituted heteroaryl, and substituted or unsubstituted heterocyclic.

Non-Cleavable Linkages

In some embodiments, the payload is attached to the Cas9 molecule or gRNA through a linker that is itself is stable under physiological conditions, such as an alkylene chain, and does not result in release of the payload from the Cas9 molecule and/or gRNA for at least 2, 3, 4, 5, 10, 15, 24 or 48 hours or for at least 1, 2, 3, 4, 5, or 10 days when administered to a subject. In some embodiments, the payload and the Cas9 molecule and/or gRNA comprise residues of a functional groups through which reaction and linkage of the payload to the Cas9 molecule or gRNA was achieved. In some embodiments, the functional groups, which may be the same or different, terminal or internal, of the payload or Cas9 molecule and/or gRNA comprise an amino, acid, imidazole, hydroxyl, thio, acyl halide, —HC═CH—, —C≡C— group, or derivative thereof. In some embodiments, the linker comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, —C(═X)— (wherein X is NR₁, O or S), —NR₁—, —NR₁C(O)—, —C(O)NR₁—, —S(O)ₙ—, —NR₁S(O)ₙ—, —S(O)ₙNR₁—, —NR₁C(O)—NR₁—; and R₁, independently for each occurrence, represents H or a lower alkyl and wherein n is 0, 1, or 2.

In some embodiments, the linker comprises an alkylene moiety or a heteroalkylene moiety (e.g., an alkylene glycol moiety such as ethylene glycol). In some embodiments, a linker comprises a poly-L-glutamic acid, polylactic acid, poly(ethyleneimine), an oligosaccharide, an amino acid (e.g., glycine), an amino acid chain, or any other suitable linkage. The linker groups can be biologically inactive, such as a PEG, polyglycolic acid, or polylactic acid chain. In certain embodiments, the linker group represents a derivatized or non-derivatized amino acid (e.g., glycine).

Cleavable Linkages

A cleavable linking group is one which is sufficiently stable outside the cell, but which upon entry into a target cell is cleaved to release the two parts the linker is holding together. In one embodiment, the cleavable linking group is cleaved at least 10 times or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond (—S—S—) can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH. A linker can include a cleavable linking group that is cleavable by a particular enzyme.

In general, the suitability of a candidate cleavable linking group can be evaluated by testing the ability of a degradative agent (or condition) to cleave the candidate linking group. The candidate cleavable linking group can also be tested for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals.

In some embodiments, the cleavable linkers include redox cleavable linkers, such as a disulfide group (—S—S—) and phosphate cleavable linkers, such as, e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(OR)—S—, —S—P(S)(OR)—O—, —O—P(O)(R)—O—, —O—P(S)(R)—O—, —S—P(O)(R)—O—, —S—P(S)(R)—O—, —S—P(O)(R)—S—, —OP(S)(R)—S—, wherein R is hydrogen or alkyl.

Acid Cleavable Linking Groups

Acid cleavable linking groups are linking groups that are cleaved under acidic conditions. In some embodiments, acid cleavable linking groups are cleaved in an acidic environment with a pH of about 6.5 or lower (e.g., about 6.0, 5.5, 5.0, or lower), or by agents such as enzymes that can act as a general acid. Examples of acid cleavable linking groups include but are not limited to hydrazones, esters, and esters of amino acids. Acid cleavable groups can have the general formula —C(═N)N—, —C(O)O—, or —OC(O)—.

Ester-Based Linking Groups

Ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—.

### XII. Formulations and Delivery

Exemplary formulations and methods for delivery of the components of a Cas system, e.g., the Cas9 molecule component and the gRNA molecule component are described herein, e.g., in Table XII-1.

TABLE XII-1
DELIVERY SUMMARY

| | | Delivery | Dura- | | |
|---|---|---|---|---|---|
| | | into Non- | tion of | Genome | Type of |
| | Delivery | Dividing | Expres- | Integra- | Molecule |
| | Vector | Cells | sion | tion | Delivered |
| Physical | | YES | Transient | NO | Nucleic |
| | | | | | Acids and |
| | | | | | Proteins |
| Viral | Retrovirus | NO | Stable | YES | RNA |
| | Lentivirus | YES | Stable | YES/NO | RNA |
| | | | | with | |
| | | | | modifi- | |
| | | | | cations | |
| | Adenovirus | YES | Transient | NO | DNA |
| | Adeno- | YES | Stable | NO | DNA |
| | Associated | | | | |
| | Virus (AAV) | | | | |
| | Vaccinia | YES | Very | NO | DNA |
| | Virus | | Transient | | |
| | Herpes | YES | Stable | NO | DNA |
| | Simplex | | | | |
| | Virus | | | | |
| Non-Viral | Cationic | YES | Transient | Depends | Nucleic |
| | Liposomes | | | on what | Acids |
| | | | | is deliv- | Proteins |
| | | | | ered | |
| | Polymeric | YES | Transient | Depends | Nucleic |
| | Nano- | | | on what | Acids |
| | particles | | | is deliv- | Proteins |
| | | | | ered | |
| BIOLOG- | Attenuated | YES | Transient | NO | Nucleic |
| ICAL | Bacteria | | | | Acids |
| NON- | Engineered | YES | Transient | NO | Nucleic |
| VIRAL | Bacterio- | | | | Acids |
| DELIV- | phages | | | | |
| ERY VE- | Mammalian | YES | Transient | NO | Nucleic |
| HICLES | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

### DELIVERY VEHICLES

In an embodiment, the delivery vehicle is a physical vehicle. In an embodiment, the vehicle is low density ultrasound. For example, microbubbles containing payload (e.g., made of biocompatible material such protein, surfactant, or biocompatible polymer or lipid shell) can be used and the microbubbles can be destructed by a focused ultrasound bean during microvascular transit. In embodiments, the vehicle is electroporation. For example, naked nucleic acids or proteins can be delivered by electroporation, e.g., into cell suspensions or tissue environment, such as retina and embryonic tissue. In an embodiment, the vehicle is needle or jet injection. For example, naked nucleic acids or protein can be injected into, e.g., muscular, liver, skin, brain or heart tissue.

In an embodiment, the delivery vehicle is a viral vector. Types of viruses include, e.g., retroviruses, lentiviruses, adenoviruses, adeno-associated viruses (AAV), vaccinia viruses, and herpes simplex viruses.

In an embodiment, the viral vector has the ability of cell type and/or tissue type recognition. For example, the viral vectors can be pseudotyped with different/alternative viral envelope glycoproteins; engineered with cell type-specific receptors (e.g., genetically modification of viral envelope glycoproteins to incorporate targeting ligands such as peptide ligands, single chain antibodies, growth factors); and/or engineered to have a molecular bridge with dual specificities with one end recognizing viral glycoproteins and the other end recognizing a moiety of the target cell surface (e.g., ligand-receptor, monoclonal antibodies, avidin-biotin and chemical conjugation).

In an embodiment, the viral vector achieves cell type specific expression. For example, tissue-specific promoter can be constructed to restrict expression of the transgene (Cas 9 and gRNA) in only the target cells. The specificity of the vectors can also be mediated by microRNA-dependent control of transgene expression. In an embodiment, the viral vector has increased efficiency of fusion of viral vector and target cell membrane. For example, fusion proteins such as fusion-competent hemagglutinin (HA) can be incorporated to increase viral uptake into cells. In an embodiment, the viral vector has the ability of nuclear localization. For example, certain viruses that require the breakdown of the cell wall (during cell division) will not infect non-diving cell. Incorporated nuclear localization peptides into the matrix proteins of the virus allow transduction into non-proliferating cells.

In an embodiment, the delivery vehicle is a non-viral vector. In an embodiment, the non-viral vector is an inorganic nanoparticle (e.g., attached to the payload to the surface of the nanoparticle). Exemplary inorganic nanoparticles include, e.g., magnetic nanoparticles (e.g., Fe₃MnO₂), silica (e.g., can integrate multi-functionality, e.g., conjugate the outer surface of the nanoparticle with a positively charged polymer (e.g., polyethylenimine, polylysine, polyserine) which allows for attachment (e.g., conjugation or entrapment) of payload and internal magnetic component, mesaporous silica nanoparticles with a positive charged polymer loaded with chloroquine to enhance transfection of the non-viral vector in vitro, high density lipoproteins and gold nanoparticles, gold nanoparticles coated with payload which gets released when nanoparticles are exposed to increased temperature by exposure to near infrared light, gold, iron or silver nanoparticles with surface modified with polylysine or another charge polymer to capture the nucleic acid cargo. In an embodiment, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids and polymers for gene transfer are shown below in Tables XII-2 and XII-3.

Exemplary lipids for gene transfer are shown below in Table XII-2.

TABLE XII-2
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Exemplary polymers for gene transfer are shown below in Table XII-3.

TABLE XII-3
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In an embodiment, the vehicle has targeting modifications to increase target cell update of nanoparticles and liposomes, e.g., cell specific antigens, monoclonal antibodies, single chain antibodies, aptamers, polymers, sugars, and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimuli-cleavable polymer is used, e.g., for release in a cellular compartment. For example, disulfide-based cationic polymers that are cleaved in the reducing cellular environment can be used.

In an embodiment, liposomes are used for delivery, e.g., to blood or bone marrow, e.g., as a way of targeting hematopoietic stem cells (HSCs) and progenitors. For example, long-term treatment can be enabled by direct delivery using liposomes for conditions where obtaining HSCs is difficult (e.g., HSCs are not stable or HSCs are rare). These conditions can include, e.g., sickle cell anemia, Fanconi anemia, and aplastic anemia. In an embodiment, liposomes are used for delivery to localized specific tissues, e.g., to liver or lung, via intravenous delivery or via localized injection to target organ or its blood flow. For example, long-term treatment can be enable to concentrate effect in that specific organ or tissue type. These conditions can include urea cycle disorders, alpha-1-anti-trypsin or cystic fibrosis.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In an embodiment, the vehicle is a genetically modified bateriophase (e.g., engineered phages having large packaging capacity, less immunogenic, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the target patient (e.g., tissue targeting can be achieved by attachment of various tissue or cell-specific ligands), or secretory exosomes—patient derived membrane-bound nanovesicle (30-100 nm) of endocytic origin (e.g., can be produced from various cell types and can therefore be taken up by cells without the need of for targeting ligands).

In an embodiment, delivery of Cas by nanoparticles in the bone marrow is an in vivo approach to curing blood and immune diseases.

In an embodiment, the components of a Cas system, e.g., the Cas9 molecule component and the gRNA molecule component described herein is delivered by nucleofection. For example, Nucleofector™ (Lonza Cologne AG) is a transfection technology that can be used for delivery to primary cells and difficult-to-transfect cell lines. It is a non-viral method based on a combination of electrical parameters and cell-type specific solutions. It allows transfected nucleic acids to directly enter the nucleus (e.g., without relying on cell division for the transfer of nucleic acids into the nucleus), providing the ability to transfect non-dividing cells, such as neurons and resting blood cells. In an embodiment, nucleofection is used as an ex vivo delivery method.

In an embodiment, the components of a Cas system, e.g., the Cas9 molecule component and the gRNA molecule component described herein is delivered by methods utilizing endogenous receptor-mediate transporters, e.g., antibody-based molecular Trojan Horses (ArmaGen). Such methods can allow for non-invasive delivery of therapeutics to locations that are otherwise difficult to reach, e.g., brain (e.g., to cross blood brain barrier (BBB), e.g., via endogenous receptor-mediated transport processes).

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the compoments of the Cas system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In an embodiment, the nucleic acid molecule is delivered by a different means than one or more of the components of the Cas system, e.g., the Cas9 molecule component and/or the gRNA molecule compoment, are delivered. The nucleic acid molecule can be delivered by any of the delivery methods described herein. For example, the nucleic acid molecule can be delivered by a viral vector, e.g., an integration-deficient lentivirus, and the Cas9 molecule component and/or the gRNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g, an RNA molecule described herein.

### XIII. Bi-Modal or Differential Delivery of Components

Separate delivery of the components of a Cas system, e.g., the Cas9 molecule component and the gRNA molecule component, and more particularly, delivery of the components by differing modes, can enhance performance, e.g., by improving tissue specificity and safety.

In an embodiment, the Cas9 molecule and the gRNA molecule are delivered by different modes, or as sometimes referred to herein as differential modes. Different or differential modes, as used herein, refer modes of delivery, that confer different pharmacodynamic or pharmacokinetic properties on the subject component molecule, e.g., a Cas9 molecule, gRNA molecule, template nucleic acid, or payload. E.g., the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., adeno associated virus or lentivirus, delivery.

By way of example, the components, e.g., a Cas9 molecule and a gRNA molecule, can be delivered by modes that differ in terms of resulting half life or persistent of the delivered component the body, or in a particular compartment, tissue or organ. In an embodiment, a gRNA molecule can be delivered by such modes. The Cas9 molecule component can be delivered by a mode which results in less persistence or less exposure of its to the body or a particular compartment or tissue or organ.

More generally, in an embodiment, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In an embodiment, the first pharmacodynamic or pharmacokinetic property, e.g., distribution, persistence or exposure, is more limited than the second pharmacodynamic or pharmacokinetic property.

In an embodiment, the first mode of delivery is selected to optimize, e.g., minimize, a pharmacodynamic or pharmacokinetic property, e.g., distribution, persistence or exposure.

In an embodiment, the second mode of delivery is selected to optimize, e.g., maximize, a pharmacodynamic or pharmcokinetic property, e.g., distribution, persistence or exposure.

In an embodiment, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In an embodiment, the second mode of delivery comprises a relatively transient element, e.g., an RNA or protein.

In an embodiment, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a Cas9 molecule, is delivered in a transient manner, for example as mRNA or as protein, ensuring that the full Cas9 molecule/gRNA molecule complex is only present and active for a short period of time.

Furthermore, the components can be delivered in different molecular form or with different delivery vectors that complement one another to enhance safety and tissue specificity.

Use of differential delivery modes can enhance performance, safety and efficacy. For example, the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in an embodiment, a first component, e.g., a gRNA molecule is delivered by a first delivery mode that results in a first spatial, e.g., tissue, distribution. A second component, e.g., a Cas9 molecule is delivered by a second delivery mode that results in a second spatial, e.g., tissue, distribution. In an embodiment, the first mode comprises a first element selected from a liposome, nanoparticle, e.g., polymeric nanoparticle, and a nucleic acid, e.g., viral vector. The second mode comprises a second element selected from the group. In an embodiment, the first mode of delivery comprises a first targeting element, e.g., a cell specific receptor or an antibody, and the second mode of delivery does not include that element. In an embodiment, the second mode of delivery comprises a second targeting element, e.g., a second cell specific receptor or second antibody.

When the Cas9 molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule and the Cas9 molecule are packaged in separated delivery vehicles with distinct but overlapping tissue tropism, the fully functional complex is only be formed in the tissue that is targeted by both vectors.

### XIV. Targeting of Genomic Signatures

Cas9 molecules, gRNA molecules, and in particular, Cas9 molecule/gRNA molecule complexes, can be used to target a cell by virtue of sequence specific interaction with a target nucleic acid comprising a selected genomic signature. This provides for targeted destruction of cells having a selected genomic signature. Method and compositions disclosed herein can be used to treat disorders characterized by a selected genomic signature, e.g., a genomic signature present in the germline or a genomic signature that arise as a result of a sporadic or somatic change in the genome, e.g., a germline or acquired mutation in a cancer cell, a viral infection, or other germline or acquired changes to the genome.

While not wishing to be bound by theory, it is believed that complementarity between the targeting domain of a gRNA molecule and the target sequence of a target nucleic acid mediates target sequence-specific interaction of the Cas9 molecule/gRNA molecule complex with the target sequence. This allows targeting of specific sequences or genomic signatures, e.g., rearrangements, e.g., translocations, insertions, deletions, and inversions, and other mutations. A Cas9 molecule/gRNA molecule complex can be used to target specific sequence, e.g., mutations, that are germline, mitochondrial, or somatic. Depending on the Cas9 molecule/gRNA molecule complex used, specific editing, the delivery of a payload, or both, can be effected. In an embodiment, both cleavage and delivery of a payload is effected.

In an embodiment, the Cas9 molecule/gRNA molecule complex that promotes cell death upon recognition of its target genomic sequence. In an embodiment, an eaCas9 molecule/gRNA molecule complex cleaves the target nucleic acid. In an embodiment, it does not deliver a payload. While not wishing to be bound by theory is it believed that endogenous cellular elements, e.g., elements of the DNA damage apoptosis signaling cascade promote apoptosis in these embodiments.

In an embodiment, an eaCas9 molecule/gRNA molecule complex cleaves the target nucleic acid and delivers a payload. The payload can comprises a compound that inhibits growth or cell division, or promotes apoptosis, e.g., an element of the DNA damage apoptosis signaling cascade. In an embodiment, a second Cas9 molecule/gRNA molecule complex is used to deliver a payload comprising a second compound that inhibits growth or cell division, or promotes apoptosis, e.g., an element of the DNA damage apoptosis signaling cascade. The Cas9 molecule/gRNA molecule complex that delivers the second payload can comprise an eiCas9 molecule or an eaCas9 molecule. An additional, e.g., third or fourth, Cas9 molecule/gRNA molecule complex, can be used to deliver additional payload, e.g., an additional compound that inhibits growth or cell division, or promotes apoptosis, e.g., an additional element of the DNA damage apoptosis signaling cascade promote.

In an embodiment, the Cas9 molecule/gRNA molecule complex delivers a payload comprising a compound that inhibits growth or cell division, or promotes apoptosis, e.g., an element of the DNA damage apoptosis signaling cascade, but does not cleave the target nucleic acid. While not wishing to be bound by theory is it believed that endogenous cellular elements, e.g., elements of the DNA damage apoptosis signaling cascade promote apoptosis in these embodiments.

Exemplary compounds that inhibit growth or cell division, or promote apoptosis, e.g., an element of the DNA damage apoptosis signaling cascade, are described herein, e.g., in Table XIV-1.

TABLE XIV-1
ATM kinases (double-strand breaks)
ATR kinases (single-strand breaks)
RF-C related protein (RAD17)
The 9-1-1 Complex: RAD1, RAD9, and HUS1
Checkpoint proteins CHK1, CHK2
P53
ZIP Kinase (ZIPK)
Fast Death-Domain Associated Protein XX (DAXX)
Promyelocytic leukemia protein (PML)
Apoptosis-inducing factor (AIF)
Caspase-activated DNAse (CAD) (in the absence of its inhibitor ICAD)

In an embodiment, a Cas9 molecule/gRNA molecule complex targets a sequence that includes or is near the breakpoint of a rearrangement, e.g., a translocation, inversion, insertion, or deletion. In an embodiment, the rearrangement confers unwanted properties, e.g., unwanted proliferation, on the cell. In an embodiment, the cell harboring the rearrangement is a cancer cell. In an embodiment, the rearrangement comprises a kinase gene and results in unwanted, increased, or constitutive expression of the kinase activity. In an embodiment, the rearrangement disrupts the expression of a tumor suppressor.

In an embodiment, the Cas9 molecule/gRNA molecule complex:
specifically targets, and e.g., cleaves, the genome of a cell comprising a rearrangement, e.g., by targeting a mutation, e.g., a breakpoint or junction of a rearrangement; or
targets, e.g., for cleavage or payload delivery, a nucleotide sequence within 200, 100, 150, 100, 50, 25, 10, or 5 nucleotides of a mutation, e.g., a rearrangement breakpoint.

The invention includes a method of manipulating a cell comprising a genomic signature, comprising:
administering a Cas9 molecule/gRNA molecule complex that targets said genomic signature, thereby manipulating said cell.

In an embodiment, manipulating comprises inhibiting the growth or division of, or killing, said cell.

In an embodiment, said cell is a cancer cell or cell having a viral infection.

In an embodiment, the method comprises treating a subject, e.g., a human subject, for a disorder characterized by a cell having said genomic signature, e.g., a cancer or a viral infection.

In an embodiment, a Cas9 molecule/gRNA molecule complex disrupts a rearrangement, e.g., by introduction of a stop codon from a template nucleic acid, e.g., a stop codon is inserted into a fusion protein, e.g., a fusion protein comprising kinase activity.

The invention includes a method of treating a cancer having a translocation of a kinase gene to a non-kinase gene, which places the kinase domain under the control of the non-kinase gene control region comprising:
administering a Cas9 molecule/gRNA molecule complex that targets the translocation. In an embodiment, the control region, e.g., the promoter, or the coding sequence, of the kinase translocation, is edited to reduce expression.

### XV. Combination Therapy

The Cas9 molecules, gRNA molecules, and in particular, Cas9 molecule/gRNA molecule complexes, can be used in combination with a second therapeutic agent, e.g., a cancer drug. In some embodiments, the second therapeutic agent (e.g., a cancer drug) and the Cas9 molecule, gRNA molecule, and in particular, Cas9 molecule/gRNA molecule complex target different (e.g., non-overlapping) pathways. In other embodiments, the second therapeutic agent (e.g., a cancer drug) and the Cas9 molecule, gRNA molecule, and in particular, Cas9 molecule/gRNA molecule complex target a same or overlapping pathway.

Exemplary combination therapies include, e.g.:
mTOR inhibitors (e.g., Temsirolimus (Torisel®) or Everolimus (Afinitor®)) together with a AKT-specific Cas9/gRNA molecule;
Tyrosine kinase inhibitors such as Imatinib mesylate (Gleevec®); Dasatinib (Sprycel®); Bosutinib (Bosulif®); Trastuzumab (Herceptin®); Pertuzumab (Perjeta™); Lapatinib (Tykerb®); Gefitinib (Iressa®); Erlotinib (Tarceva®) together with a HDAC-specific Cas9/gRNA molecule; and
Any chemotherapeutic agent together with one or more Cas9/gRNAs against multidrug resistance genes such as MDR1 gene.

### XVI. Treatment of Genetic Disorder, e.g., Duchenne Muscular Dystrophy (DMD)

In another aspect, the invention features, a method of altering a cell, e.g., reducing or abolishing the effect of a genetic signature, e.g., a stop codon, e.g., a premature stop codon. The method comprises contacting said cell with:
a Cas9 molecule/gRNA molecule complex that cleaves at or upstream from the genetic signature, e.g., a premature stop codon,
thereby altering the cell.

While not wishing to be bound by theory it is believed that, in an embodiment, cleavage and subsequent exonuclease activity, and non-homologous end joining results in an altered sequence in which the genetic signature, e.g., a premature stop codon is eliminated, e.g., by being placed in a different frame. In an embodiment, the same series of events restores the proper reading frame to the sequence that follows the signature, e.g., premature stop codon.

When the method is carried out to correct a frameshift mutation in order to remove a premature stop codon, repair can be carried out at various sites in the DNA. One may direct cleavage at the mutation, thereby correcting the frameshift entirely and returning the protein to its wild-type (or nearly wild-type) sequence. One may also direct cleavage at or near the premature stop codon, so that all (or nearly all) amino acids of the protein C-terminal of the codon where repair was effected are wild-type. In the latter case, the resulting protein may have one or more frameshifted amino acids between the mutation and the repair site; however the protein may still be functional because it is full-length and has wild-type sequence across most of its length.

A genetic signature is a particular DNA sequence at a particular portion of the genome, that causes a phenotype (such as a genetic disease or a symptom thereof). For instance, the genetic signature may be a premature stop codon that prevents expression of a protein. In this scenario, the premature stop codon can arise from a mutation that directly creates a stop codon, or from a mutation that causes a frameshift leading to a premature stop codon being formed downstream. A genetic signature may also be a point mutation that alters the identity of an important amino acid in a protein, disrupting the protein's function.

In an embodiment, the Cas9 molecule/gRNA molecule complex mediates a double stranded break in said target nucleic acid.

In certain embodiments, the genetic signature, e.g., a premature stop codon, results from a point mutation, an insertion, a deletion, or a rearrangement. In some embodiments, a mutation causes a frameshift, resulting in a genetic signature, e.g., a premature stop codon downstream of the mutation.

In certain embodiments, the premature stop codon is within the target nucleic acid. In other embodiments, the target nucleic acid is upstream of the premature stop codon. The mutation may be upstream of the target nucleic acid, within the target nucleic acid, or downstream of the target nucleic acid.

In some embodiments the double stranded break is within 500, 200, 100, 50, 30, 20, 10, 5, or 2 nucleotides of the mutation. In some embodiments the double stranded break is within 500, 200, 100, 50, 30, 20, 10, 5, or 2 nucleotides of the genetic signature, e.g., a premature stop codon.

In certain embodiments, the Cas9 molecule/gRNA molecule complex mediates exonuclease digestion of the target nucleic acid. In certain embodiments, the Cas9 molecule/gRNA molecule complex removes 1, 2, 3, 4, or 5 nucleotides at the double stranded break.

In some embodiments, the double stranded break is resolved by non-homologous end joining.

In some embodiments the mutation and/or genetic signature, e.g., premature stop codon is in the dystrophin gene, e.g., in exon 51, or in the intron preceding or following exon 51. The premature stop codon may also be caused by a mutation in the dystrophin gene at one or more of codons 54, 645, 773, 3335, and 3340. In some embodiments, the premature stop codon in the dystrophin gene results from a deletion of codons 2305 through 2366.

In some embodiments, contacting the cell with a Cas9 molecule/gRNA molecule complex comprises contacting the cell with a nucleic acid encoding a Cas9 molecule. In certain embodiments, contacting the cell with a Cas9 molecule/gRNA molecule complex comprises transfecting the cell with a nucleic acid, e.g., a plasmid, or using a viral vector such as adeno-associated virus (AAV).

In certain embodiments, the method results in increased levels of the protein in which the genetic signature, e.g., a premature stop codon, was previously located. For instance, protein levels (e.g., dystrophin levels) may be increased by at least 3%, 4%, 5%, 10%, 15%, 20%, 25%, or 30% in a cell or in a tissue. In some embodiments, the method results in increased levels of the mRNA in which the premature stop codon was previously located, for instance by preventing the mRNA from undergoing nonsense-mediated mRNA decay.

In some embodiments, one or more of the target nucleic acid, the genetic signature, e.g., premature stop codon, and the mutation are located in the dystrophin gene (which is mutated in DMD). One or more of the target nucleic acid, the genetic signature, e.g., premature stop codon, and the mutation may also be located in the COL7A1 gene (mutated in type VII-associated dystrophic epidermolysis bullosa), the FKTN gene (mutated in Fukuyama congenital muscular dystrophy), the dysferlin gene (mutated in limb-girdle muscular dystrophy type 2B), the CFTR gene (mutated in cystic fibrosis), HEXA (mutated in Tay-Sachs disease), the IDS gene (mutated in Hunter syndrome), the FVIII gene (mutated in hemophilia), the IDUA gene (mutated in Hurler syndrome), the PPT1 gene (mutated in infantile neuronal ceroid lipofuscinosis), a tumor suppressor such as the ATM gene (mutated in cancers like gliomas and B-Cell Chronic Lymphocytic Leukemia), RP2 (mutated in X-linked retinitis pigmentosa), the CTNS gene (mutated in nephropathic cystinosis), and the AVPR2 gene (mutated in Congenital nephrogenic diabetes insipidus).

In some embodiments, the method is performed in cultured cells. In some embodiments, the method further comprises administering the cell to a patient. The cell may be, for example, an induced pluripotent stem cell, a bone marrow derived progenitor, a skeletal muscle progenitor, a CD133+ cell, a mesoangioblast, or a MyoD-transduced dermal fibroblast.

In some embodiments, the method comprises contacting the cell with a template nucleic acid under conditions that allow for homology-directed repair between the target nucleic acid and the template nucleic acid to correct the mutation or the premature stop codon.

In another aspect, the invention features a method of treating a human subject having a disorder associated with a genetic signature, e.g., premature stop codon, e.g., DMD, comprising providing to the human subject:
1) a Cas9 molecule/gRNA molecule complex that cleaves at or upstream from the premature stop codon or
2) a cell that has been contacted with such complex,
thereby treating the subject.

In an embodiment, the Cas9 molecule/gRNA molecule complex mediates a double stranded break in said target nucleic acid.

In certain embodiments, genetic signature, e.g., premature stop codon results from a point mutation, an insertion, a deletion, or a rearrangement. In some embodiments, a mutation causes a frameshift, resulting in a premature stop codon downstream of the mutation.

In some embodiments the double stranded break is within 500, 200, 100, 50, 30, 20, 10, 5, or 2 nucleotides of the mutation. In some embodiments the double stranded break is within 500, 200, 100, 50, 30, 20, 10, 5, or 2 nucleotides of the premature stop codon.

In certain embodiments, the genetic signature, e.g., premature stop codon is within the target nucleic acid of the Cas9 molecule/gRNA molecule complex. In other embodiments, the target nucleic acid is upstream of the genetic signature, e.g., premature stop codon. The mutation may be upstream of the target nucleic acid, within the target nucleic acid, or downstream of the target nucleic acid.

In certain embodiments, the Cas9 molecule/gRNA molecule complex mediates exonuclease digestion of the target nucleic acid. In certain embodiments, the Cas9 molecule/gRNA molecule complex removes 1, 2, 3, 4, or 5 nucleotides at the double stranded break.

In some embodiments the double stranded break is resolved by non-homologous end joining.

In some embodiments the mutation and/or genetic signature, e.g., premature stop codon is in the dystrophin gene, e.g., in exon 51, or in the intron preceding or following exon 51. The premature stop codon may also be caused by a mutation in the dystrophin gene at one or more of codons 54, 645, 773, 3335, and 3340. In some embodiments, the premature stop codon in the dystrophin gene results from a deletion of codons 2305 through 2366.

In some embodiments, contacting the cell with a Cas9 molecule/gRNA molecule complex comprises contacting the cell with a nucleic acid encoding a Cas9 molecule. In certain embodiments, contacting the cell with a Cas9 molecule/gRNA molecule complex comprises transfecting the cell with a nucleic acid, e.g., a plasmid, or using a viral vector such as adeno-associated virus (AAV).

In certain embodiments, the method results in increased levels of the protein in which the genetic signature, e.g., premature stop codon was previously located. For instance, protein levels (e.g., dystrophin levels) may be increased by at least 3%, 4%, 5%, 10%, 15%, 20%, 25%, or 30% in a cell or in a tissue. In some embodiments, the method results in increased levels of the mRNA in which the premature stop codon was previously located, for instance by preventing the mRNA from undergoing nonsense-mediated mRNA decay.

In some embodiments, one or more of the target nucleic acid, the genetic signature, e.g., premature stop codon, and the mutation are located in the dystrophin gene (which is mutated in DMD). One or more of the target nucleic acid, the genetic signature, e.g., premature stop codon, and the mutation may also be located in the COL7A1 gene (mutated in type VII-associated dystrophic epidermolysis bullosa), the FKTN gene (mutated in Fukuyama congenital muscular dystrophy), the dysferlin gene (mutated in limb-girdle muscular dystrophy type 2B), the CFTR gene (mutated in cystic fibrosis), HEXA (mutated in Tay-Sachs disease), the IDS gene (mutated in Hunter syndrome), the FVIII gene (mutated in hemophilia), the IDUA gene (mutated in Hurler syndrome), the PPT1 gene (mutated in infantile neuronal ceroid lipofuscinosis), a tumor suppressor such as the ATM gene (mutated in cancers like gliomas and B-Cell Chronic Lymphocytic Leukemia), RP2 (mutated in X-linked retinitis pigmentosa), the CTNS gene (mutated in nephropathic cystinosis), and the AVPR2 gene (mutated in Congenital nephrogenic diabetes insipidus).

In some embodiments, the method is performed in cultured cells. In some embodiments, the method further comprises administering the cell to a patient. The cell may be, for example, an induced pluripotent stem cell, a bone marrow derived progenitor, a skeletal muscle progenitor, a CD133+ cell, a mesoangioblast, or a MyoD-transduced dermal fibroblast.

In some embodiments the method comprises contacting the cell with a template nucleic acid under conditions that allow for homology-directed repair between the target nucleic acid and the template nucleic acid to correct the mutation or the premature stop codon.

In some embodiments, the subject has a disorder selected from Duchenne Muscular Dystrophy (DMD), collagen type VII-associated dystrophic epidermolysis bullosa, Fukuyama congenital muscular dystrophy, and limb-girdle muscular dystrophy type 2B, cystic fibrosis, lysosomal storage disorders (such as Tay-Sachs disease, Hunter syndrome, and nephropathic cystinosis), hemophilia, Hurler syndrome, infantile neuronal ceroid lipofuscinosis, X-linked retinitis pigmentosa (RP2), cancers (such as gliomas and B-Cell Chronic Lymphocytic Leukemia), and Congenital nephrogenic diabetes insipidus.

### XVII. Treatment of Disorders Characterized by Lack of Mature Specialized Cells, e.g., Impaired Hearing, with Loss of Hair Cells, Supporting Cells, or Spiral Ganglion Neurons; or for Diabetes, with Loss of Beta Islet Cells

In another aspect, the invention features, a method of altering a cell, e.g., to promote the development of other mature specialized cells, e.g, in regeneration therapy. For example, proliferation genes can be upregulated and/or checkpoint inhibitors can be inhibited, e.g., to drive down one or more differenation pathways.

In an embodiment, the method includes induction of proliferation and specified lineage maturation.

In an embodiment, the method comprises, e.g., for restoration or improvement of hearing, contacting said cell with:
a Cas9 molecule/gRNA molecule complex that up-regulates a gene that promotes the development of hair cells, or down-regulates a gene that inhibits the development of hair cells thereby altering the cell.

In an embodiment, the Cas9 molecule/gRNA molecule delivers a payload that up-regulates a gene that promotes hair cell development.

In an embodiment, the Cas9 molecule/gRNA molecule delivers a payload that down-regulates a gene that inhibits hair growth.

In an embodiment, the Cas9 molecule/gRNA molecule complex edits the genome of a cell to up-regulate a gene that promotes hair growth. In an embodiment, a template nucleic acid is used to effect a Cas9 molecule/gRNA molecule complex alteration to the genome that up-regulates a gene that promotes hair growth.

In an embodiment, the Cas9 molecule/gRNA molecule complex edits the genome of a cell to down-regulate a gene that inhibits hair growth. In an embodiment, a template nucleic acid is used to effect a Cas9 molecule/gRNA molecule complex alteration to the genome that down-regulates a gene that promotes hair growth.

In an embodiment, said cell is an iPS cell, a native hair cell progenitor, or a mature hair cell.

In an embodiment, the Cas9 molecule/gRNA molecule and modifies expression of a gene, e.g., by modifying the structure of the gene (e.g., by editing the genome) or by delivery of a payload that modulates a gene. In an embodiment, the gene is a transcription factor or other regulatory gene.

In an embodiment, for hair cell or other mature cell regeneration, the method includes one or more or all of the following:
contacting the cell with a Cas9 molecule/gRNA molecule complex that results in up-regulation one or more of the following for cell proliferation: c-Myc, GATA3, Oct4, Sox2, Wnt, TCF3;
contacting the cell with a Cas9 molecule/gRNA molecule complex that results in downregulation one or more of the following for check point: BCL2, BMP, Hes1, Hes5, Notch, p27, Prox1, TGFβ; and
contacting the cell with a Cas9 molecule/gRNA molecule complex that results in turning on a maturation pathway. For hair cells this would include one or more of the following: Atoh1 (Math 1), Barh11, Gfi1, Myo7a, p63, PAX2, PAX8, Pou4f3 and for neurons would include one or more of the following: NEFH, Neurod1, Neurog1, POU4F1.

In an embodiment, the method comprises generation of inner ear hair cells, outer ear hair cells, spiral ganglion neurons, and ear supporting cells.

In an embodiment, one or more growth factors can be modulated, e.g., upregulated, e.g., TPO can be upregulated for production of platelets and GCSF can be upregulated for production of neutrophils.

In another aspect, the invention provides altered cell described herein, e.g., in this Section XVII.

In another aspect, the invention features a method of treating impaired hearing. The method comprises administering to said subject, an altered cell described herein, e.g., in this section XVII. In an embodiment, the cell is autologous. In an embodiment, the cell is allogeneic. In an embodiment, the cell is xenogeneic.

In another aspect, the invention features a method of treating subject, e.g., for impaired hearing. The method comprises administering to said subject:
a Cas9 molecule/gRNA molecule complex that up-regulates a gene that promotes the growth of hair, or down-regulates a gene that inhibits the growth of hair thereby altering the cell.

In an embodiment, the Cas9 molecule/gRNA molecule delivers a payload that up-regulates a gene that promotes hair growth.

In an embodiment, the Cas9 molecule/gRNA molecule delivers a payload that down-regulates a gene that inhibits hair growth.

In an embodiment, the Cas9 molecule/gRNA molecule complex edits the genome of a cell to up-regulate a gene that promotes hair growth. In an embodiment, a template nucleic acid is used to effect a Cas9 molecule/gRNA molecule complex alteration to the genome that up-regulates a gene that promotes hair growth.

In an embodiment, the Cas9 molecule/gRNA molecule complex edits the genome of a cell to down-regulate a gene that inhibits hair growth. In an embodiment, a template nucleic acid is used to effect a Cas9 molecule/gRNA molecule complex alteration to the genome that down-regulates a gene that promotes hair growth.

In an embodiment, the Cas9 molecule/gRNA molecule and modifies expression of a gene, e.g., by modifying the structure of the gene (e.g., by editing the genome) or by delivery of a payload that modulates a gene. In an embodiment, the gene is a transcription factor or other regulatory gene.

In an embodiment, the method includes one or more or all of the following:
administering a Cas9 molecule/gRNA molecule complex that results in up-regulation one or more of the following: c-Mye, GATA3, Oct4, Sox2, Wnt, TCF3;
administering a Cas9 molecule/gRNA molecule complex that results in turning on a maturation pathway. For hair cells this would include one or more of the following: Atohl (Mathl), Barh11, Gfi1, Myo7a, p63, PAX2, PAX8, Pou4f3 and for neurons would include one or more of the following: NEFH, Neurod1, Neurog1, POLJ4F1.
   Annexes are included as part of the application.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

We claim:

1. An ex vivo method of editing a BCL11A gene in a human cell, the method comprising:
introducing a gene editing complex into the human cell by electroporation, wherein the gene editing complex comprises:
(a) a guide RNA (gRNA) comprising a targeting sequence complementary to a target sequence of the BCL11A gene, wherein the targeting sequence comprises two consecutive nucleotides modified with a 2′-O-methyl group, and wherein the two consecutive nucleotides are within five nucleotides of the 5′ end of the targeting sequence; and
(b) a Cas9 protein;
wherein the human cell is a human hematopoietic stem cell (HSC).

2. The method of claim 1, wherein the HSC comprises a genetic sequence driving expression of fetal hemoglobin in an erythrocyte differentiated from said HSC.

3. The method of claim 1, wherein the targeting sequence is 20 nucleotides in length.

4. The method of claim 1, wherein the targeting sequence comprises a third nucleotide modified with a 2′-O-methyl group within five nucleotides of the 5′ end of the targeting sequence.

5. The method of claim 1, wherein the gRNA comprises two further consecutive nucleotides modified with a 2′-O-methyl group, wherein the two further consecutive nucleotides are each within five nucleotides of the 3′ end of the gRNA.

6. The method of claim 1, wherein at least one of the two consecutive nucleotides is selected from the group consisting of 2′-O-methyl uridine, 2′-O-methyl cytidine and 2′-O-methyl adenosine.

7. The method of claim 1, wherein the targeting sequence further comprises at least one nucleotide modified with a phosphorothioate group.

8. The method of claim 7, wherein the phosphorothioate group links the two consecutive nucleotides.

9. The method of claim 5, wherein a phosphorothioate group links the two further consecutive nucleotides.

10. The method of claim 1, wherein the Cas9 protein comprises a nuclear localization sequence (NLS).

11. The method of claim 10, wherein the NLS is an SV40 NLS.

12. The method of claim 1, wherein the target sequence is within an enhancer of the BCL11A gene.

13. The method of claim 1, wherein the Cas9 protein is from S. pyogenes.

14. The method of claim 1, wherein the gRNA directs the Cas9 protein to the target sequence and the Cas9 protein cleaves the target sequence.

15. A human hematopoietic stem cell (HSC) comprising a modified BCL11A gene produced by a method comprising:
introducing a gene editing complex into the human HSC by electroporation, wherein the gene editing complex comprises:
a guide RNA (gRNA), wherein the gRNA comprises a targeting sequence complementary to a target sequence in an enhancer of a BCL11A gene of the HSC, wherein the targeting sequence is 20 nucleotides in length, wherein the targeting sequence comprises three consecutive nucleotides modified with a 2′-O-methyl group, wherein the three consecutive nucleotides are within five nucleotides of the 5′ end of the targeting sequence, wherein at least one of the three consecutive nucleotides is selected from the group consisting of 2′-O-methyl uridine, 2′-O-methyl cytidine and 2′-O-methyl adenosine, wherein at least two of the three consecutive nucleotides are linked by a phosphorothioate group, wherein the gRNA comprises two further consecutive nucleotides modified with a 2′ O-methyl group, and wherein the two further consecutive nucleotides are linked by a phosphorothioate group and within five nucleotides of the 3′ end of the gRNA; and
a Cas9 protein complexed to the guide RNA,
wherein the Cas9 protein is S. pyogenes Cas9 and comprises a nuclear localization signal.

16. The HSC of claim 15, wherein a nucleotide sequence of the enhancer of the BCL11A gene of the HSC comprises an insertion or deletion relative to the nucleotide sequence prior to introducing the gene editing complex.

17. The HSC of claim 15, wherein expression of the BCL11A gene of the HSC is downregulated relative to its expression prior to introducing the gene editing complex.

18. An erythrocyte differentiated from the HSC of claim 15.

19. The erythrocyte of claim 18, wherein the HSC comprises a genetic sequence driving expression of fetal hemoglobin in the erythrocyte differentiated from the HSC.

20. A method of treating a subject having sickle cell disease comprising administering the HSC of claim 15 to the subject.

21. An ex vivo human hematopoietic stem cell (HSC) comprising:
a guide RNA (gRNA), wherein the gRNA comprises a targeting sequence complementary to a target sequence in an enhancer of a BCL11A gene of the HSC, wherein the targeting sequence is 20 nucleotides in length, wherein the targeting sequence comprises three consecutive nucleotides modified with a 2′-O-methyl group, wherein the three consecutive nucleotides are within five nucleotides of the 5′ end of the targeting sequence, wherein at least one of the three consecutive nucleotides is selected from the group consisting of 2′-O-methyl uridine, 2′-O-methyl cytidine and 2′-O-methyl adenosine, wherein at least two of the three consecutive nucleotides are linked by a phosphorothioate group, wherein the gRNA comprises two further consecutive nucleotides modified with a 2′ O-methyl group, and wherein the two further consecutive nucleotides are linked by a phosphorothioate group and within five nucleotides of the 3′ end of the gRNA; and
a Cas9 protein complexed to the gRNA, wherein the Cas9 protein is S. pyogenes Cas9 and comprises a nuclear localization sequence.

22. The HSC of claim 21, wherein the gRNA-Cas9 complex targets the target sequence for cleavage by the Cas9 protein.

23. The HSC of claim 21, wherein a nucleotide sequence of the enhancer of the BCL11A gene comprises an insertion or deletion relative to a corresponding nucleotide sequence of an HSC that does not comprise the gRNA.

24. The HSC of claim 21, wherein the HSC has downregulated expression of the BCL11A gene relative to expression of a BCL11A gene in an HSC that does not comprise the gRNA.

25. An erythrocyte differentiated from the HSC of claim 21.

26. The erythrocyte of claim 25, wherein the HSC comprises a genetic sequence driving expression of fetal hemoglobin in the erythrocyte differentiated from the HSC.

27. A method of treating a subject having sickle cell disease comprising administering the HSC of claim 21 to the subject.
